SEQUENCE LISTING (8 sequences)

SEQ ID NO: 1 - protein, 7 aa, synthetic construct
  VARIANT 2: note = X is any amino acid residue
  VARIANT 6: note = X is any amino acid residue
PXPLRXD

SEQ ID NO: 2 - protein, 60 aa, Homo sapiens
HGAQKAALVLLSACLVTLWGLGEPPEHTLRYLVLHLASLQLGLLLNGVCSLAEELRHIHS

SEQ ID NO: 3 - protein, 60 aa, Mus musculus
HRSKYVALIFLVASLMILWVAKDPPNHTLKYLALHLASHELGLLLKNLCCLAEELCHVQS

SEQ ID NO: 4 - protein, 60 aa, Gallus gallus
GRARHAACVLLAVCFVVLFLSGEPLAPITRRVCTQLAALQLGVLLKGCCCLAEEIFHLHS

SEQ ID NO: 5 - protein, 59 aa, Homo sapiens
RYRGSYWRTVRACLGCPLRRGALLLLSIYFYYSLPNAVGPPETWMLALLGLSQALNILL

SEQ ID NO: 6 - protein, 58 aa, Mus musculus
RYQGSYWKAVRACLGCPIHCMAMILLSSYFYFLQNTADIYLSWMFGLLVLYKSLSMLL

SEQ ID NO: 7 - protein, 58 aa, Gallus gallus
RHHGSLWQVLCSCFPPRWHLALLLVGGSAYLDPPEDNGHSPRLALTLSCLCQLLVLAL

SEQ ID NO: 8 - protein, 324 aa, Homo sapiens
HSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLLLPELQARIRTYNQHYGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPSFSLSQEVLRHLRQ

(19) United States
(12) Patent Application Publication
(10) Pub. No.: US 2025/0170120 A1
(43) Pub. Date: May 29, 2025

(54) ALLOSTERIC STING MODULATORS AND METHODS OF USE

(71) Applicant: THE BOARD OF REGENTS OF THE UNIVERSITY OF TEXAS SYSTEM, Austin, TX (US)

(72) Inventors: Yong LU, Dallas, TX (US); Liping LI, Dallas, TX (US); Jie LI, Flower Mound, TX (US); Xuewu ZHANG, Addison, TX (US); Xiaochen BAI, Dallas, TX (US); Chuo CHEN, Dallas, TX (US)

(21) Appl. No.: 18/834,412

(22) PCT Filed: Jan. 31, 2023
(86) PCT No.: PCT/US2023/061685

§ 371 Date: Jul. 30, 2024

Related U.S. Application Data
(60) Provisional application No. 63/305,060, filed on Jan. 31, 2022.

(51) Int. Cl. A61K 31/4709; A61K 31/404; A61K 31/4155; A61K 31/4192; A61K 31/422; A61K 31/4704; A61K 31/517; C07D 209/34; C07D 403/12; C07D 403/14; C07D 405/12; C07D 409/14; C07D 413/12; C07D 491/056

(52) U.S. Cl. CPC A61K 31/4709; A61K 31/404; A61K 31/4155; A61K 31/4192; A61K 31/422; A61K 31/4704; A61K 31/517; C07D 209/34; C07D 403/12; C07D 403/14; C07D 405/12; C07D 409/14; C07D 413/12; C07D 491/056

(57) ABSTRACT

Provided are various Stimulator of Interferon Genes (STING) allosteric modulators as well as methods of making and use thereof. Specifically provided herein are highly active STING inhibitors (antagonists) and activators (agonists). The compounds provide herein may be prepared as pharmaceutical compositions and used in methods of treating conditions related to disrupted or defective STING signaling.

### CROSS-REFERENCE TO RELATED APPLICATIONS

This application claims priority to U.S. Provisional Application No. 63/305,060, filed Jan. 31, 2022, which is incorporated by reference herein in its entirety.

### ACKNOWLEDGEMENT OF GOVERNMENT SUPPORT

This invention was made with government support under Grant Nos. CA226419 and GM130289 awarded by the National Institutes of Health. The government has certain rights in this invention.

### SEQUENCE LISTING

This application contains a Sequence Listing that has been submitted in XML format via Patent Center and is hereby incorporated by reference in its entirety. The XML file, created Jan. 30, 2023, is named UTSD4040_106546-748634_Sequence_Listing.xml and is 9 kilobytes in size.

### BACKGROUND

### 1. Field

The present disclosure is generally directed to compositions and methods for modulating STING. Also provided are methods of use thereof.

### 2. Discussion of Related Art

Stimulator of interferon genes (STING) is an essential adaptor protein in innate immunity against DNA viruses or bacteria. STING is a transmembrane (TM) dimeric protein located in the endoplasmic reticulon (ER) or the Golgi apparatus. STING is activated by binding of its cytoplasmic ligand-binding domain (LBD) to cyclic dinucleotides, produced by either the cytosolic DNA-sensor cyclic-GMP-AMP (cGAMP) synthase (cGAS) or invading bacteria. Cyclic dinucleotides induce the formation of high-order oligomers of STING, which is essential for triggering the downstream signaling pathways. cGAMP induces a conformational change in the STING LBD, which promotes its oligomerization. However, the cGAMP-induced STING oligomers appeared weak and have not been resolved to high resolution, hampering the understanding of the activation mechanism.

The present disclosure is based, at least in part, on the surprising discovery that a small molecular agonist, compound 53 (C53), promotes human STING oligomerization and activation through a mechanism orthogonal to that of cGAMP. Accordingly, C53 and related compounds may be useful to modulate STING-mediated immunity for vaccines or cancer immune-therapies. In various exemplary embodiments described herein, high-resolution cryo-EM structures and mutational analysis reveal that cGAMP and C53 can synergistically induce the oligomerization and activation of STING, thereby allowing for modulation of STING without use of cGAMP.

### BRIEF SUMMARY

In some aspects, the present disclosure provides a method of allosterically inhibiting activity of a stimulator of interferon genes protein (STING), the method comprising contacting a compound of Formula (I), Formula (II) or Formula III to STING:

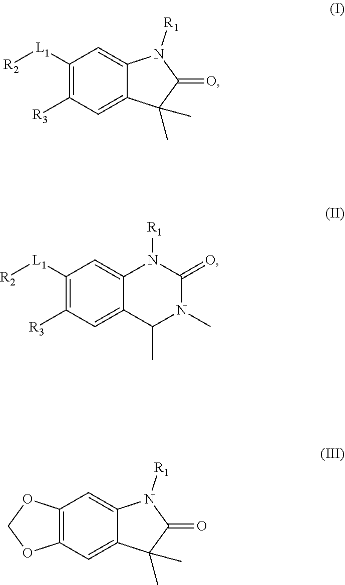

or a salt thereof,
wherein L₁ is absent or is selected from the group consisting of

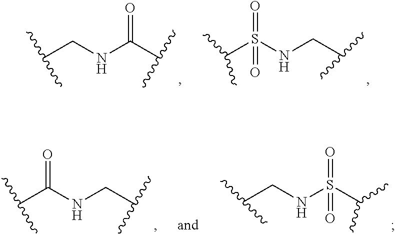 [SMILES: *CNC(*)=O ; *CNS(*)(=O)=O ; *CNC(*)=O ; *CNS(*)(=O)=O]

R₁ is selected from the group consisting of

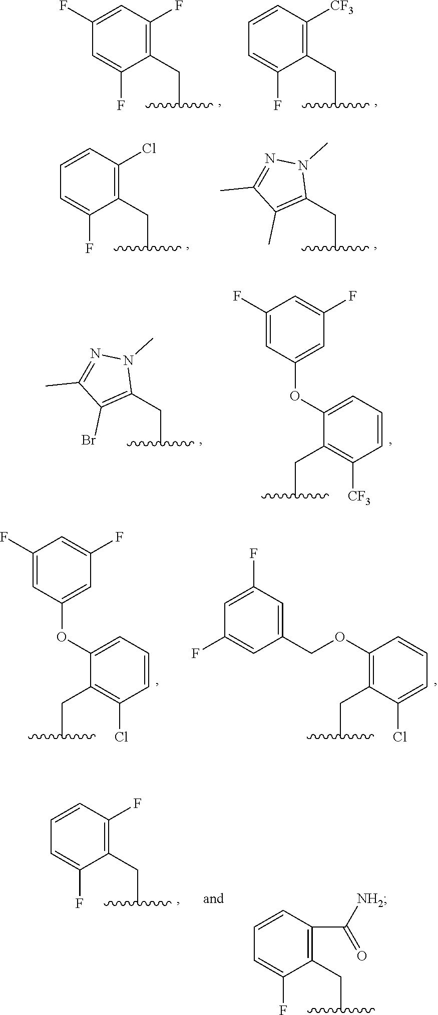

R₂ is selected from the group consisting of H, CF₃, F, CN,

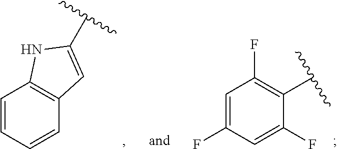 [SMILES: *c1cc2ccccc2[nH]1 ; *c1c(F)cc(F)cc1F]

and R₃ is selected from the group consisting of H, CN or CF₃;
with the provisos that
(i) at least one of R₂ and R₃ is not hydrogen and
(ii) R₁ is not

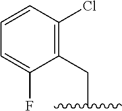 [SMILES: *Cc1c(F)cccc1Cl]

when L1 is

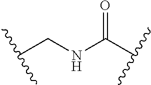 [SMILES: *CNC(*)=O]

### and R₂ is

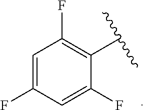 [SMILES: *c1c(F)cc(F)cc1F]

In some aspects, the method of allosterically inhibiting activity of STING is an in vitro method. In other aspects, the method of allosterically inhibiting activity of STING is an in vivo method.

In further aspects, the present disclosure also provides for a method of treating a condition caused or related to disrupted STING signaling in a subject in need thereof, the method comprising administering a STING antagonist to the subject, wherein the STING antagonist is a compound of Formula (I), Formula (II) or Formula III:

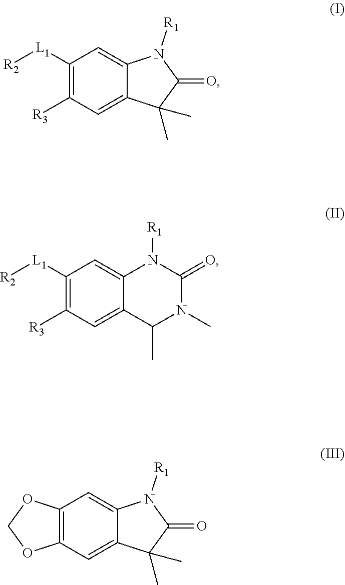

or a salt thereof,
wherein L₁ is absent or is selected from the group consisting of

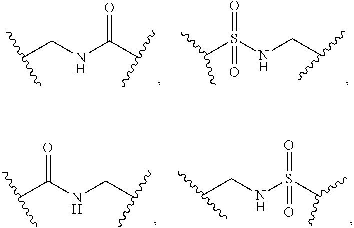 [SMILES: *CNC(*)=O ; *CNS(*)(=O)=O ; *CNC(*)=O ; *CNS(*)(=O)=O]

and;
R₁ is selected from the group consisting of

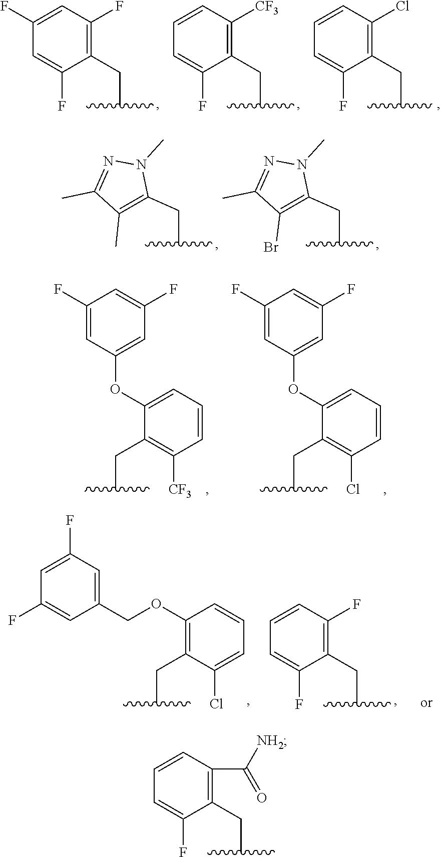

R₂ is selected from the group consisting of H, CF₃, F, CN,

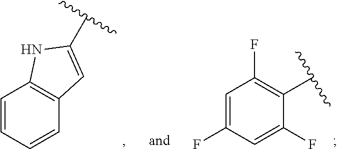 [SMILES: *c1cc2ccccc2[nH]1 ; *c1c(F)cc(F)cc1F]

and R₃ is selected from the group consisting of H, CN or CF₃; with the provisos that (i) at least one of R₂ and R₃ is not hydrogen and (ii) R₁ is not

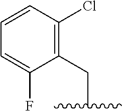 [SMILES: *Cc1c(F)cccc1Cl]

when L1 is

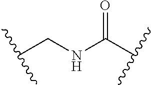 [SMILES: *CNC(*)=O]

and R₂ is

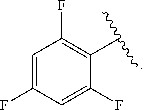 [SMILES: *c1c(F)cc(F)cc1F]

In some aspects, the condition caused or related to disrupted STING signaling in a subject can comprise inflammation, allergies, an autoimmune condition, an infectious disease, a neurodegenerative disease, a liver disease, a cancer, and/or a renal disease. The disclosed method further comprises administering the STING antagonist in a pharmaceutical composition comprising at least one carrier or excipient. In some aspects, the subject in need thereof is a mammal. In some aspects, the subject in need thereof is human.

In any of the methods provided herein, L1 may be absent or

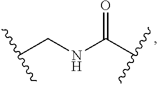 [SMILES: *CNC(*)=O]

Further, in any of the methods provided herein, R₁ may be selected from the group consisting of

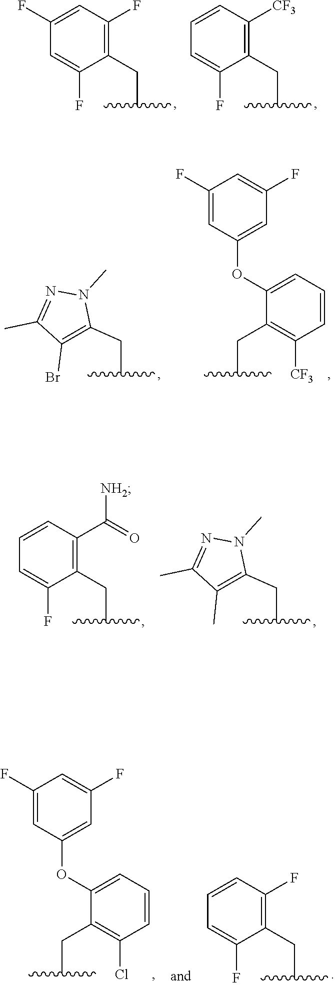

For example, in some aspects, R₁ may be selected from the group consisting of

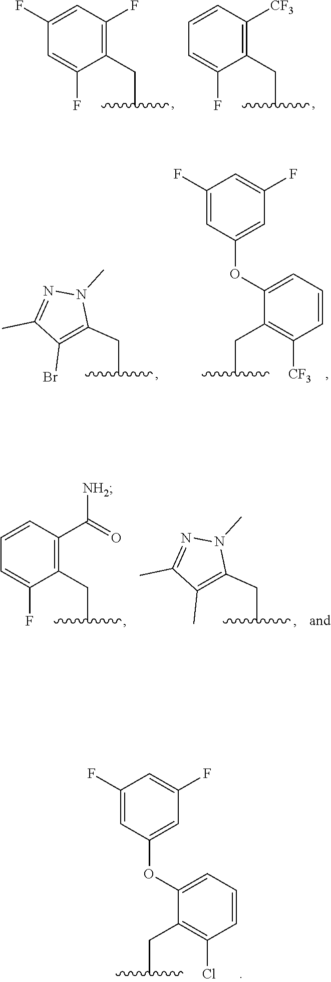

In another non-limiting example, R₁ may be selected from the group consisting of

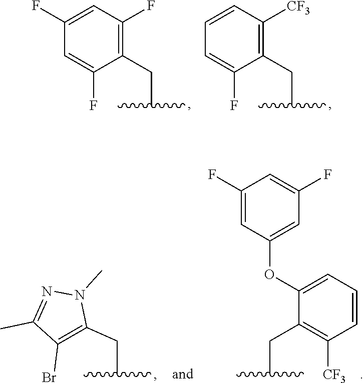

In various methods provided herein, R₂ may be selected from the group consisting of

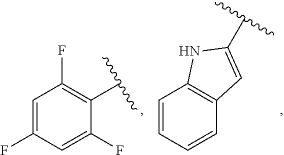

—CN, H, and —CF₃. For example, in certain aspects, R₂ can be selected from the group consisting of

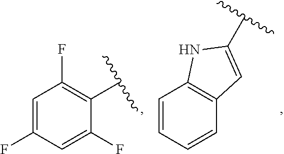

### and —CN.

In further aspects, R₃ can be hydrogen.

In any of the methods provided herein, the compound and/or STING antagonist may be selected from the group consisting of:

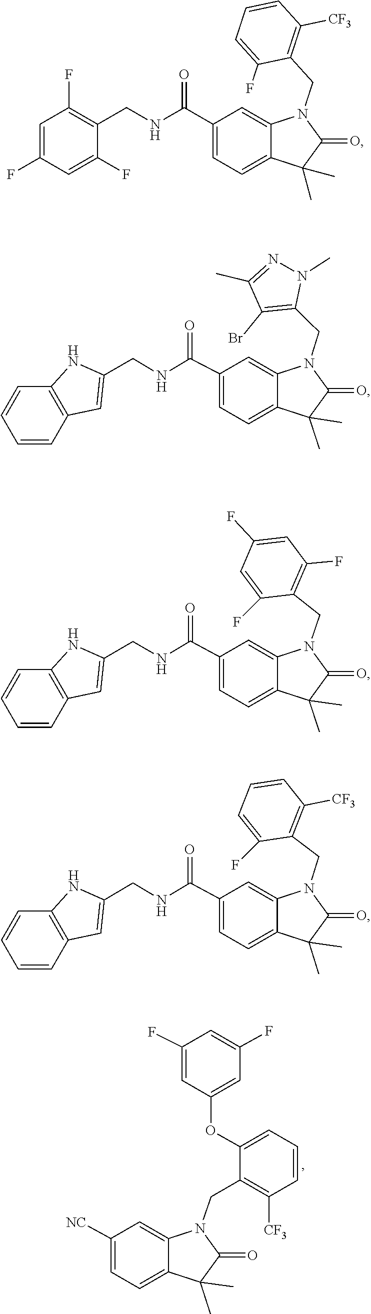 [SMILES: CC1(C)C(=O)N(Cc2c(F)cccc2C(F)(F)F)c2cc(C(=O)NCc3c(F)cc(F)cc3F)ccc21 ; Cc1nn(C)c(CN2C(=O)C(C)(C)c3ccc(C(=O)NCc4cc5ccccc5[nH]4)cc32)c1Br ; CC1(C)C(=O)N(Cc2c(F)cc(F)cc2F)c2cc(C(=O)NCc3cc4ccccc4[nH]3)ccc21 ; CC1(C)C(=O)N(Cc2c(F)cccc2C(F)(F)F)c2cc(C(=O)NCc3cc4ccccc4[nH]3)ccc21 ; CC1(C)C(=O)N(Cc2c(Oc3cc(F)cc(F)c3)cccc2C(F)(F)F)c2cc(C#N)ccc21]

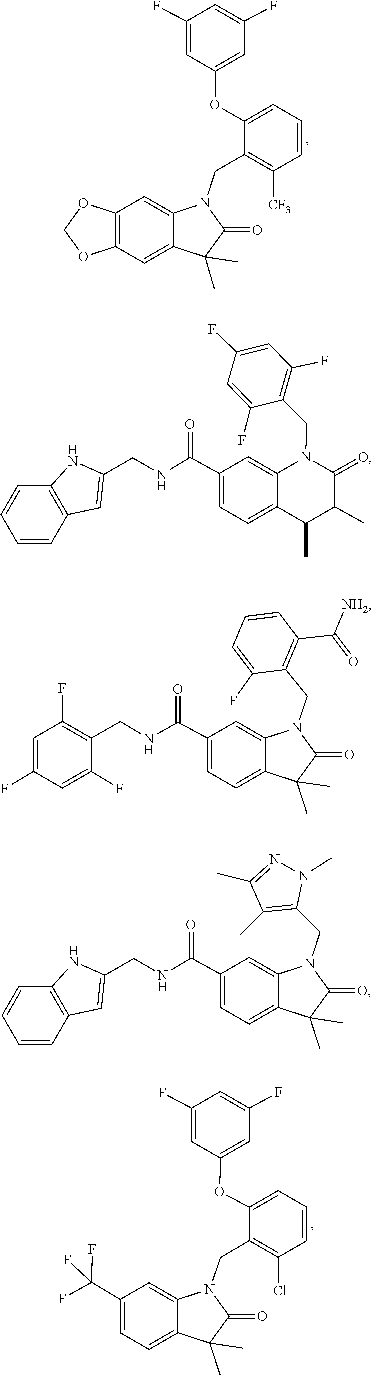

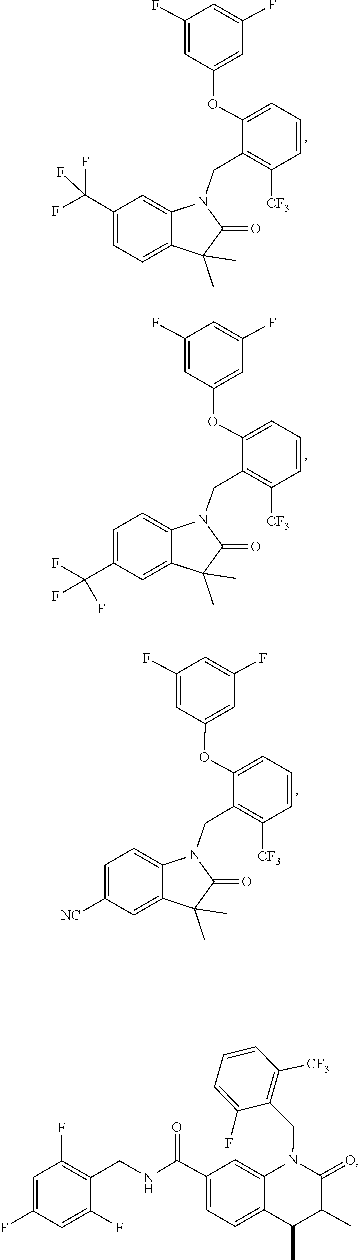

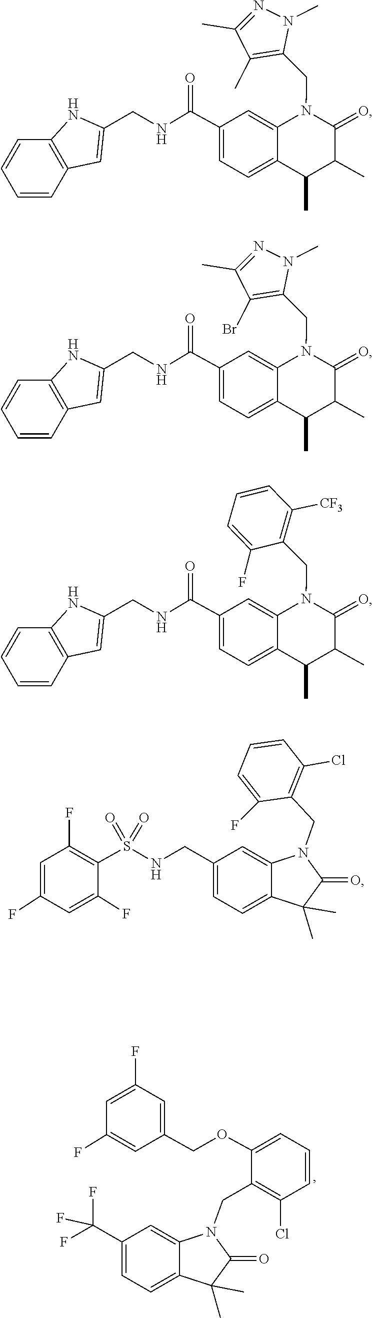

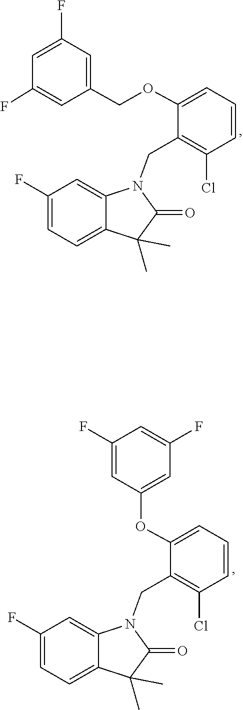 [SMILES: CC1(C)C(=O)N(Cc2c(Cl)cccc2OCc2cc(F)cc(F)c2)c2cc(F)ccc21 ; CC1(C)C(=O)N(Cc2c(Cl)cccc2Oc2cc(F)cc(F)c2)c2cc(F)ccc21]

and any salt thereof.

For example, the compound or STING antagonist may be selected from the group consisting of:

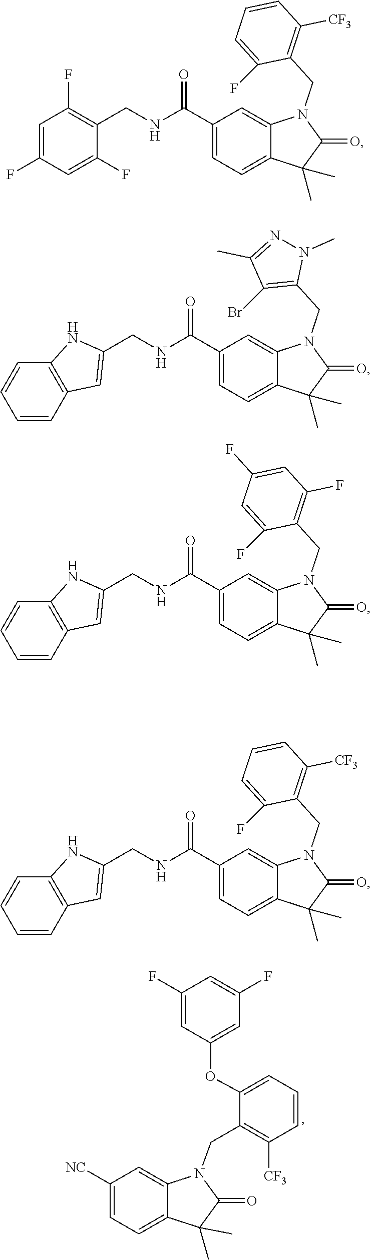

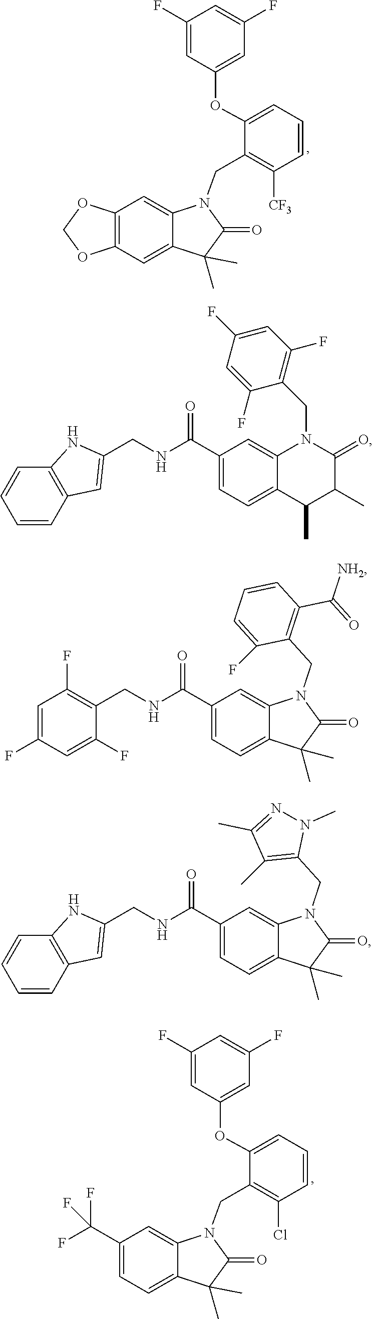

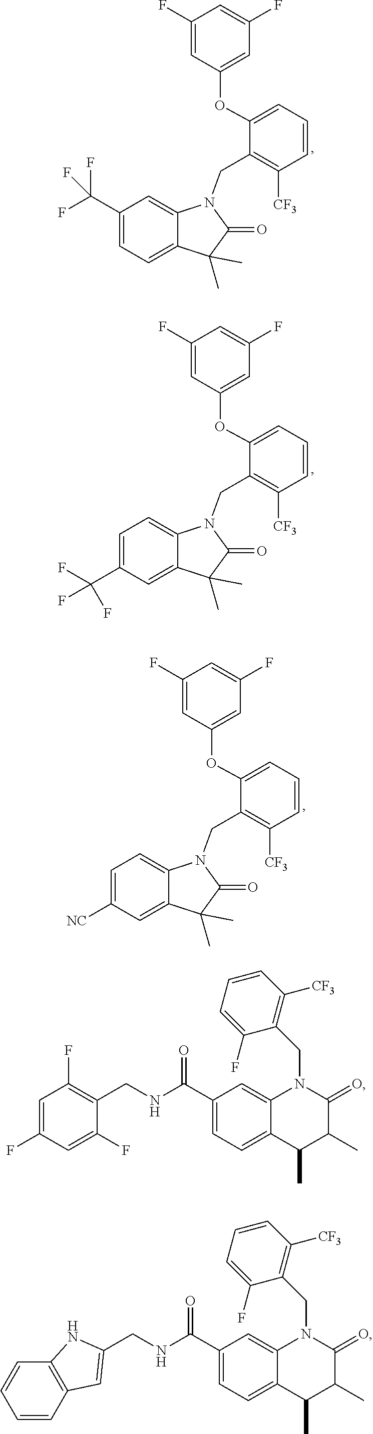

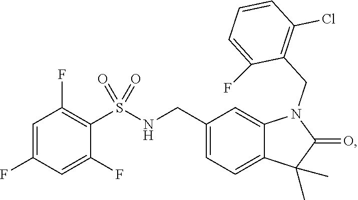 [SMILES: CC1(C)C(=O)N(Cc2c(F)cccc2Cl)c2cc(CNS(=O)(=O)c3c(F)cc(F)cc3F)ccc21]

and any salt thereof.

In still further aspects, the compound and/or STING antagonist can be selected from the group consisting of:

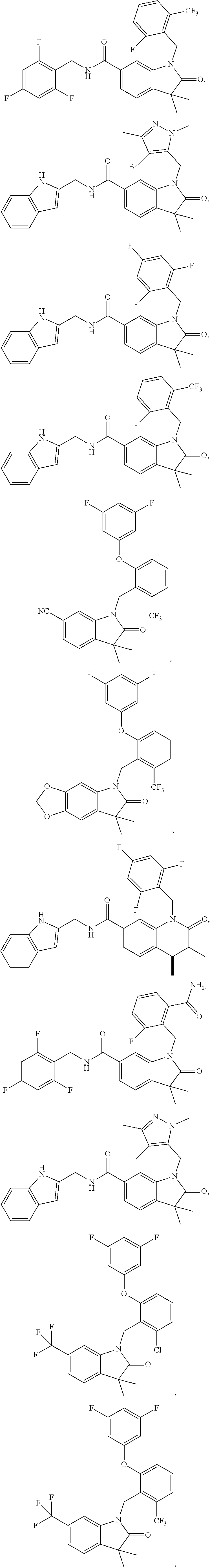

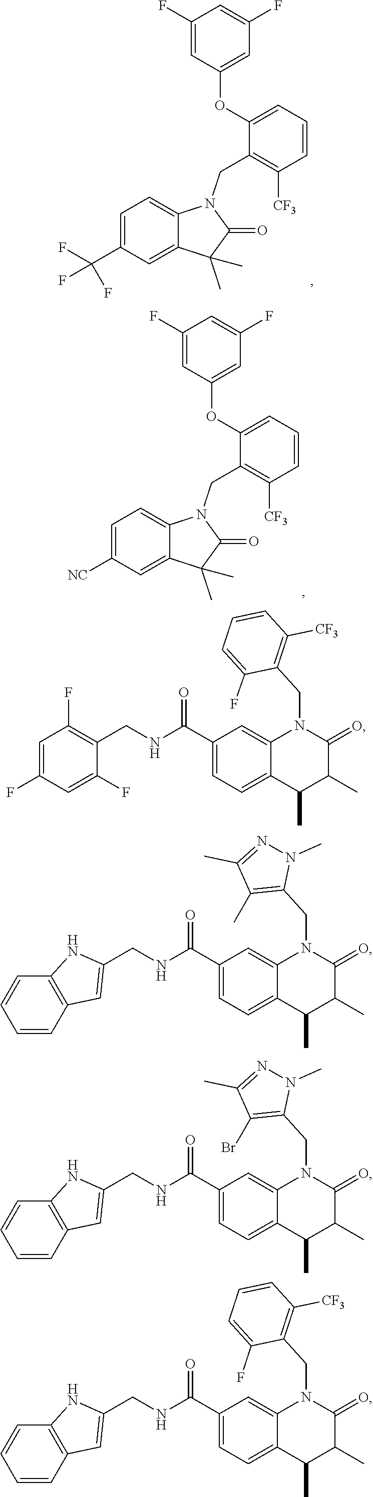

In various aspects, the compound or STING antagonist may be selected from the group consisting of:

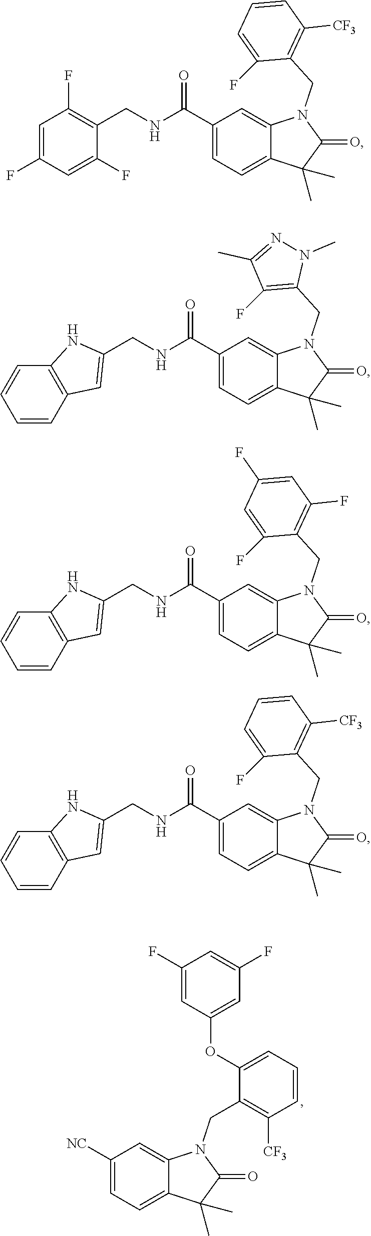

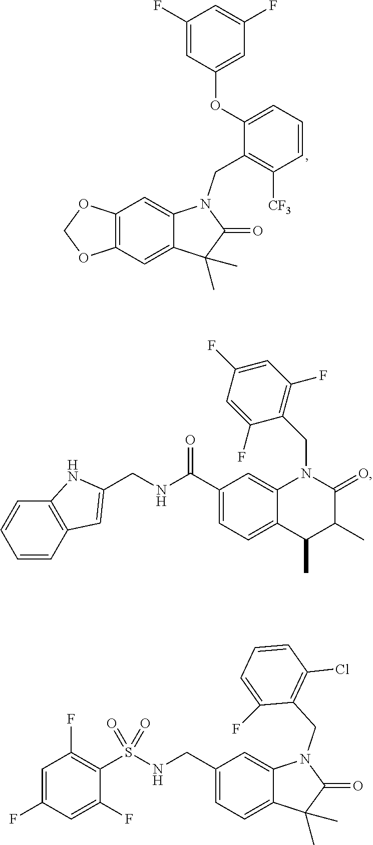 [SMILES: CC1(C)C(=O)N(Cc2c(Oc3cc(F)cc(F)c3)cccc2C(F)(F)F)c2cc3c(cc21)OCO3 ; CC1C(=O)N(Cc2c(F)cc(F)cc2F)c2cc(C(=O)NCc3cc4ccccc4[nH]3)ccc2[C@@H]1C ; CC1(C)C(=O)N(Cc2c(F)cccc2Cl)c2cc(CNS(=O)(=O)c3c(F)cc(F)cc3F)ccc21]

and any salt thereof.

In some aspects, the compound or STING antagonist may be selected from the group consisting of:

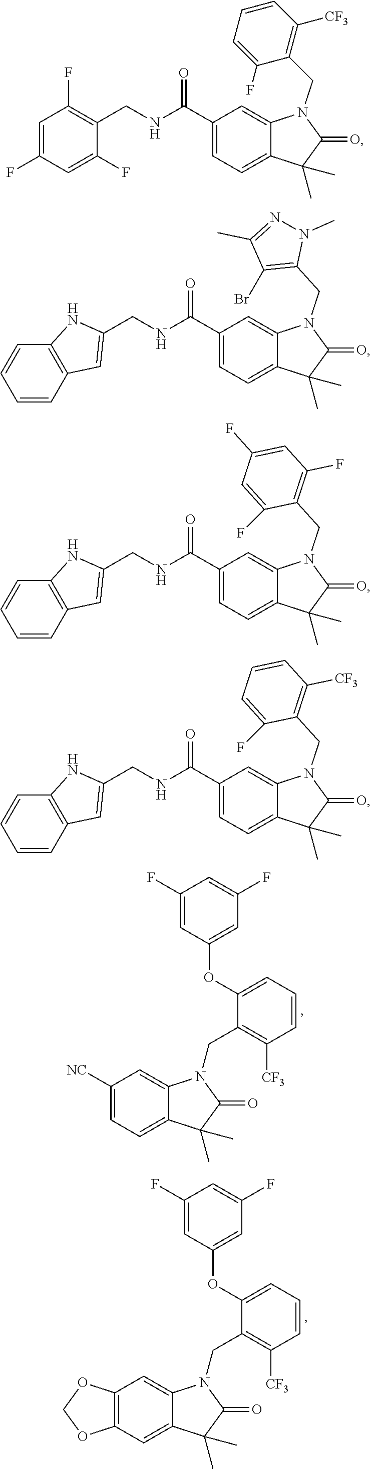

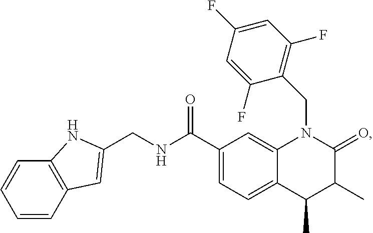 [SMILES: CC1C(=O)N(Cc2c(F)cc(F)cc2F)c2cc(C(=O)NCc3cc4ccccc4[nH]3)ccc2[C@@H]1C]

and any salt thereof.

Further aspects of the disclosure provides for a compound of Formula I, Formula II or Formula III:

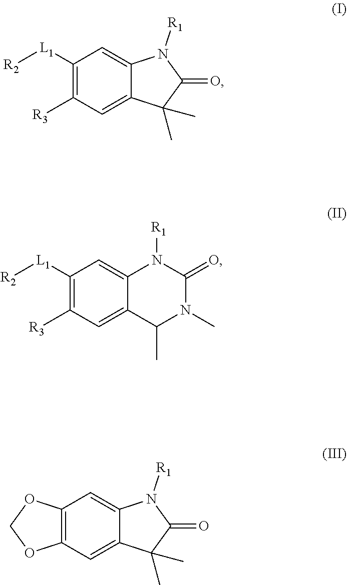

or a salt thereof,
wherein L₁ is absent or is selected from the group consisting of

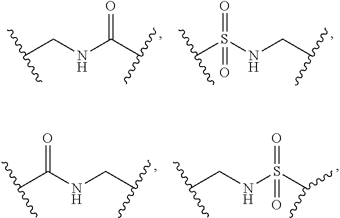 [SMILES: *CNC(*)=O ; *CNS(*)(=O)=O ; *CNC(*)=O ; *CNS(*)(=O)=O]

and;
   R₁ is selected from the group consisting of

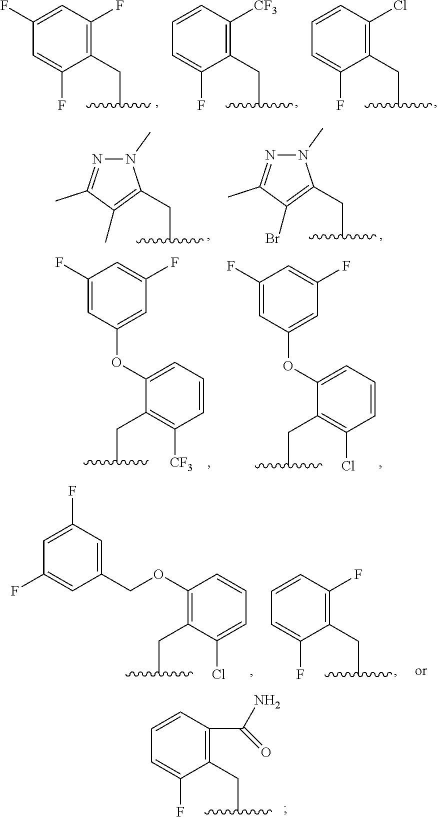

R₂ is selected from the group consisting of H, CF₃, F, CN,

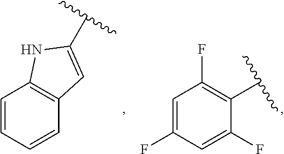 [SMILES: *c1cc2ccccc2[nH]1 ; *c1c(F)cc(F)cc1F]

and R₃ is selected from the group consisting of H, CN or CF₃; with the provisos that (i) at least one of R₂ and R₃ is not hydrogen and (ii) R₁ is not

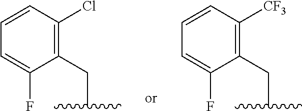 [SMILES: *Cc1c(F)cccc1Cl ; *Cc1c(F)cccc1C(F)(F)F]

when L1 is

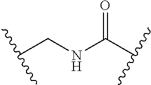 [SMILES: *CNC(*)=O]

### and R₂ is

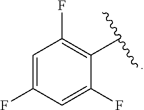 [SMILES: *c1c(F)cc(F)cc1F]

In various aspects, L1 may be absent,

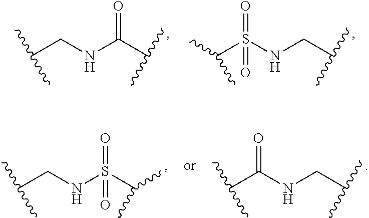 [SMILES: *CNC(*)=O ; *CNS(*)(=O)=O ; *CNS(*)(=O)=O ; *CNC(*)=O]

For example, L1 can be absent or

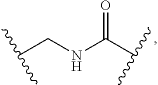 [SMILES: *CNC(*)=O]

In some aspects, R₁ may be selected from the group consisting of

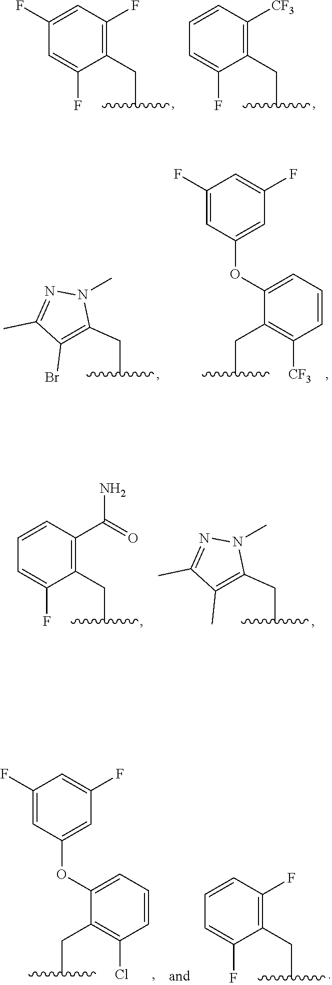

For example, in various embodiments, R₁ may be selected from the group consisting of

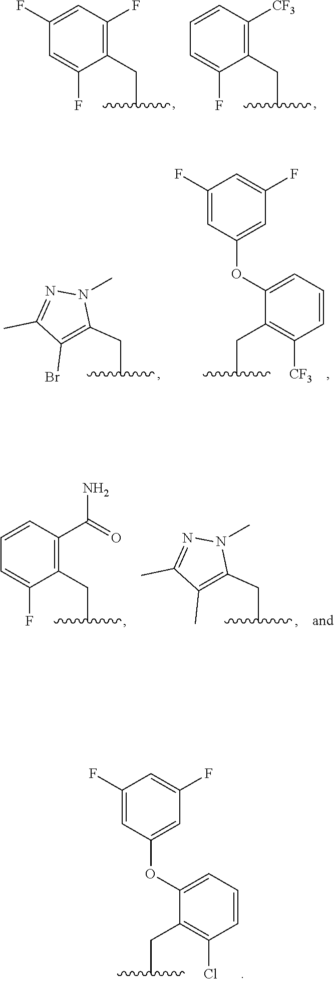

For example, R₁ may be selected from the group consisting of

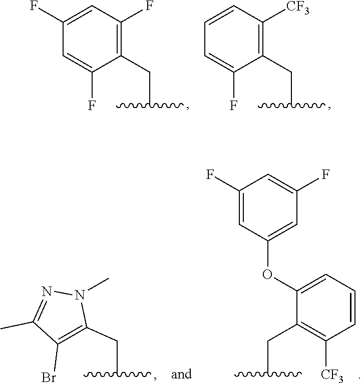

In various aspects, R₂ may be selected from the group consisting of

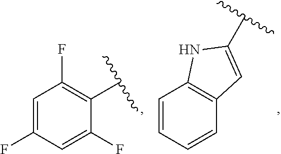

—CN, H, and —CF₃. For example, in various aspects, R₂ may be selected from the group consisting of

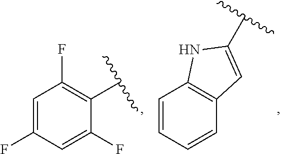

### and —CN.

In any of the foregoing or related aspects R₃ may be hydrogen.

In some aspects the compound provided herein is selected from the group consisting of:

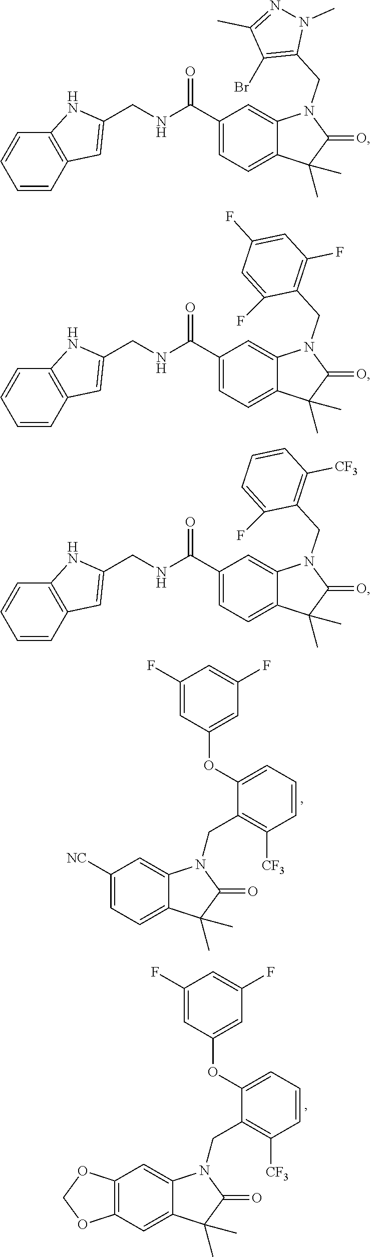

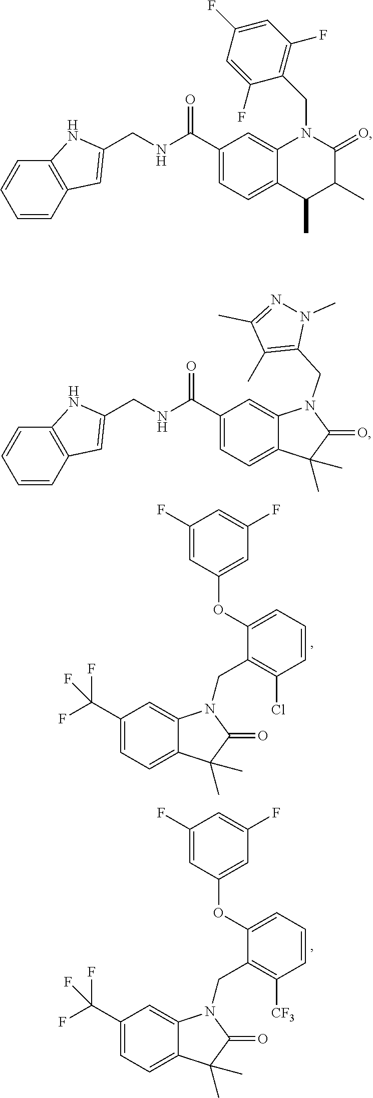

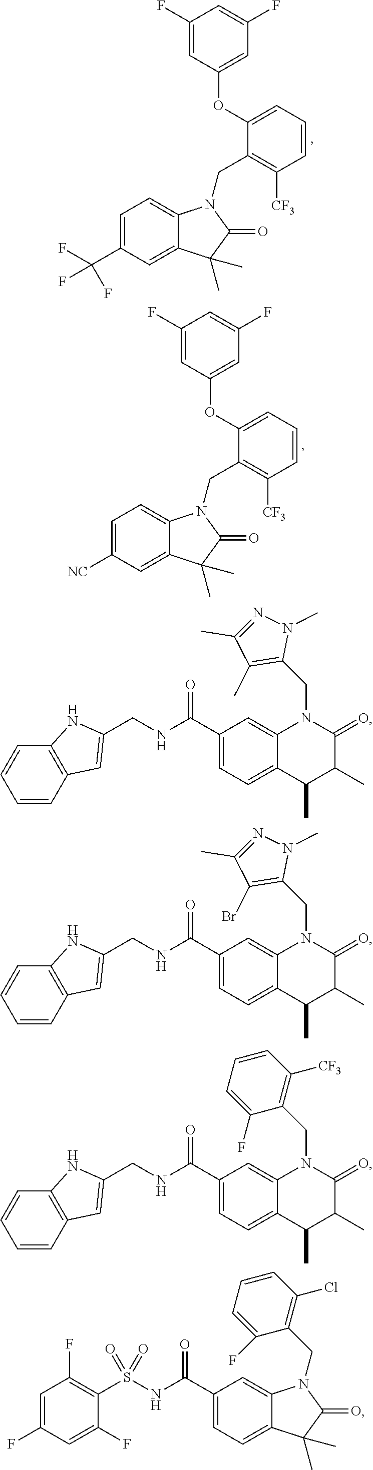

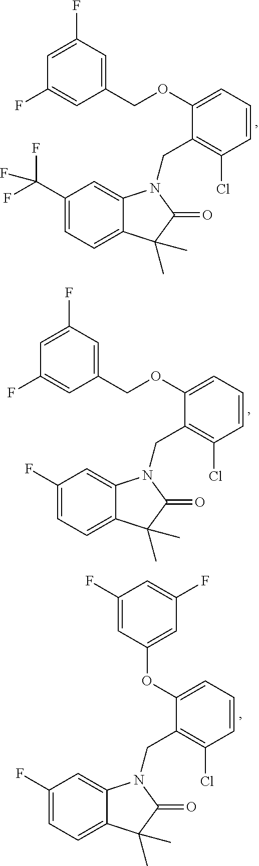

and any salt thereof.

In further aspects, the present disclosure further provides for a pharmaceutical composition comprising any one of the compounds (i.e., STING antagonists) provided herein and at least one carrier or excipient.

### BRIEF DESCRIPTION OF THE DRAWINGS

Embodiments of the present inventive concept are illustrated by way of example in which like reference numerals indicate similar elements and in which:

FIG. 1. Structure of the high-order oligomer of human STING bound to both cGAMP and C53. (FIG. 1A), Overall cryo-EM map of the oligomer containing four STING dimers. (FIG. 1B), High-resolution cryo-EM map and atomic model of the STING tetramer. (FIG. 1C), Expanded view of the inter-dimer interfaced mediated by the LBDα2-LBDα3 loop. The color scheme is the same as in (FIG. 1B). (FIG. 1D), C53-binding pocket in the TMD of STING viewed from the luminal side of ER/Golgi. The color scheme is the same as in (FIG. 1B). (FIG. 1E), Comparison of the human STING dimer bound to both cGAMP and C53 with the STING apo-state. The two subunits in the dimer are colored green and yellow respectively.

FIG. 2. Interaction between STING and C53. (FIG. 2A, FIG. 2B), Overview of the C53-binding site in the TMD of one STING dimer, viewed from the cytosolic and luminal sides, respectively. Cryo-EM density for C53 is shown in semi-transparent white. (FIG. 2C, FIG. 2D), Details of the binding site in two different views. Helices in front of C53 are omitted for clarity. (FIG. 2E), Two-dimensional diagram of the interactions between C53 and STING. Hydrogen atoms are omitted.

FIG. 3. Mutational analyses of the C53-binding site in human STING. (FIG. 3A), Effects of mutations in the C53-binding site on STING oligomerization induced by cGAMP and C53. Native gel results shown are representatives of three biological repeats. (FIG. 3B), Effects of mutations in the C53-binding site on phosphorylation of STING, TBK1 and IRF3 induced by cGAMP and C53 in cells. HEK293T cells were transfected with human STING wild type or the mutants. Cells were stimulated with cGAMP, C53 or both and lysates were subjected to western blot analyses. The results shown are representatives of three biological repeats.

FIG. 4. TMD-mediated interactions contribute to STING oligomerization. (FIG. 4A), C53 induces dilation of the TMD binding pocket in the STING dimer. A comparison with apo-STING shows that the TM helices in the C53-bound structure shift away from each other to enlarge the binding pocket. The structure is viewed from the ER/Golgi luminal side. (FIG. 4B), Two orthogonal views of the TMD-TMD interface between the two STING dimers. TM3 from one dimer joins one set of TM1, TM2 and TM4 from the other dimer to form a four-helix bundle. (FIG. 4C), Detailed views of the TMD-TMD interface. (FIG. 4D), Effects of mutations in the TMD-TMD interface on STING oligomerization induced by cGAMP and C53. Native gel results shown are representatives of three biological repeats. (FIG. 4E), Effects of mutations in the TMD-TMD interface on phosphorylation of STING, TBK1 and IRF3 induced by cGAMP and C53 in cells. HEK293T cells were transfected with human STING wild type or the mutants. Cells were stimulated with cGAMP, C53 or both and lysates were subjected to western blot analyses. The results shown are representatives of three biological repeats.

FIG. 5. Purification of human STING and its interaction with C53 and cGAMP. (FIG. 5A), Gel filtration profile of human STING on a Superdex S200 10/300 column. (FIG. 5B), SDS-PAGE analyses of fractions indicated by the bracket in a. (FIG. 5C), Analyses of STING oligomerization by native gel. C53 and cGAMP together induced robust high-order oligomerization of purified STING solubilized in detergent solution, while either one alone failed to do so under the same condition.

FIG. 6. Image processing procedure of human STING tetramer bound to both cGAMP and C53. (FIG. 6A), Motion corrected micrograph. Red arrows highlight high-order oligomers of STING. The curved overall shape of the oligomers is evident from these examples. (FIG. 6B), 2D class averages of high-order oligomers of human STING. Large oligomers were segmented into particles containing four dimers at the maximum. (FIG. 6C), Final 3D reconstruction of the tetramer colored based on local resolution. (FIG. 6D), Gold-standard FSC curve of the final 3D reconstruction. (FIG. 6E), FSC between the final map and the atomic model. (FIG. 6F), Image processing procedure.

FIG. 7. Sample density maps of various parts of the structure.

FIG. 8. C53-induced dilation of the binding pocket in the STING-TMD. (FIG. 8A), Comparison of the TMD of human STING in the C53-bound and the apo states. (FIG. 8B), Sequence alignment of the TMD of STING from human (h), mouse (m) and chicken (ch). Black circles highlight residues in the C53-binding pocket. Stars highlight residues in the TMD-TMD interface that contribute to the oligomerization of STING.

FIG. 9. Effects of mutations in the C53-binding site on STING oligomerization in cells. (FIG. 9A) and (FIG. 9C), Representative images of cells expressing STING wild type or the mutants in the binding pocket and the TM3-TM4 loop, respectively. Hela cells were transfected with GFP-tagged human STING wild type or mutants. Cells were stimulated with cGAMP, C53 or both. Localization of STING-GFP in cells was monitored by the fluorescence signal of GFP. Nuclei were stained with DAPI. The experiments were repeated three times. The images are representatives from these experiments. (FIG. 9B) and (FIG. 9D), Quantification of STING puncta formation in cells expressing the wild type or mutants. The bar graph shows the individual data points, mean and s.e.m. of the percentage of cells with STING forming large puncta from the three biological repeats. Statistical significance p-values between the wild type and mutants were calculated by two-tailed Student's t-test. *, p<0.05; **, p<0.01; ***, p<0.001. Scale bar, 10 μm.

FIG. 10. Effects of mutations in the TMD-TMD interface on STING oligomerization in cells. (FIG. 10A), Representative images of cells expressing STING wild type or the mutants in the TMD-TMD interface. Hela cells were transfected with GFP-tagged human STING wild type or mutants. Cells were stimulated with cGAMP, C53 or both. Localization of STING-GFP in cells was monitored by the fluorescence signal of GFP. Nuclei were stained with DAPI. The experiments were repeated three times. The images are representatives from these experiments. (FIG. 10B), Quantification of STING puncta formation in cells expressing the wild type or mutants. The bar graph shows the individual data points, mean and s.e.m. of the percentage of cells with STING forming large puncta from the three biological repeats. Statistical significance p-values between the wild type and mutants were calculated by two-tailed Student's t-test.*, p<0.05; **, p<0.01; ***, p<0.001; ****, p<0.0001. Scale bar, 10 μm.

FIG. 11. Data collection and model refinement statistics.

FIG. 12 shows a schematic of the synthesis of Compound UT009.

FIG. 13 depicts a representative synthesis pathway to prepare Compound UT019.

FIG. 14 depicts a representative synthesis pathway to prepare Compound UT073.

FIG. 15 depicts a representative synthesis pathway to prepare UT122.

FIG. 16A-16D depict plots of interferon levels induced by increasing cGAMP levels in the presence of different concentrations of (FIG. 16A) UT009, (FIG. 16B) UT019, (FIG. 16C) UT073 or (FIG. 16D) UT122. Structures of each compound are also provided.

FIG. 17 depicts a plot of interferon levels induced by increasing concentrations of C1 and C2, structures shown.

FIG. 18 depicts a representative synthesis pathway to prepare Compound UT017.

FIG. 19 depicts a representative synthesis pathway to prepare Compound UT018.

FIG. 20 depicts a representative synthesis pathway to prepare Compound UT065.

FIG. 21 depicts a representative synthesis pathway to prepare Compound UT066.

FIG. 22 depicts a representative synthesis pathway to prepare Compound UT071.

FIG. 23 depicts a representative synthesis pathway to prepare Compound UT072.

FIG. 24 depicts a representative synthesis pathway to prepare Compound UT074.

FIG. 25 depicts a representative synthesis pathway to prepare Compound UT114.

FIG. 26 depicts a representative synthesis pathway to prepare Compound UT115.

FIG. 27 depicts a representative synthesis pathway to prepare Compound UT117.

FIG. 28 depicts a representative synthesis pathway to prepare Compound UT118.

FIG. 29 depicts a representative synthesis pathway to prepare Compound UT120.

FIG. 30 depicts a representative synthesis pathway to prepare Compound UT121.

FIG. 31 depicts a representative synthesis pathway to prepare Compound UT126.

FIG. 32 depicts a representative synthesis pathway to prepare Compound C1.

FIG. 33 depicts a representative synthesis pathway to prepare Compound C2.

FIG. 34 is a three-dimensional structure of the STING protein and is the illustrative depiction of the sequence and the 3D image.

FIG. 35A-35C depict plots of interferon levels induced by different concentrations of compounds C53 (FIG. 35A) and UT014 in the presence of MS2 (FIG. 35B) or cGAMP (FIG. 35C). Structures of each compound are also provided.

FIG. 36A-36C depict plots of suppression of interferon levels by different concentrations of compound UT014 (FIG. 36A) and UT122 in the presence of MS2 (FIG. 36B) or cGAMP (FIG. 36C). Structures of each compound are also provided.

FIG. 37A-37B depicts cryo-EM structure of human STING in complex with agonist compound C53 or antagonist compound UT009 and overlay of compound bound to STING. (FIG. 37A) shows the overall view of the structure and (FIG. 37B) shows enlarged view of the binding site.

FIG. 38 depicts plots of suppression of interferon levels by different concentrations of compounds UT017, UT018, UT019 and UT122 and inhibition of cGAMP induced interferon production by UT122.

FIG. 39A-G depicts plots of suppression of interferon levels in the presence of MSA2 by different concentrations of compounds UT141 (FIG. 39A), UT142 (FIG. 39B), UT157 (FIG. 39C), UT153 (FIG. 39D), UT156 (FIG. 39E) or UT158 (FIG. 39F) and comparison of suppression of interferon production by compounds H151 and UT122 (FIG. 39G).

FIG. 40 depicts a representative synthesis pathway to prepare Compound UT156.

FIG. 41 depicts a representative synthesis pathway to prepare Compound UT141.

FIG. 42 depicts a representative synthesis pathway to prepare Compound UT142.

FIG. 43 depicts a representative synthesis pathway to prepare Compound UT153.

FIG. 44 depicts a representative synthesis pathway to prepare Compound UT151.

FIG. 45 depicts a representative synthesis pathway to prepare Compound UT157.

FIG. 46 depicts a representative synthesis pathway to prepare Compound UT160.

FIG. 47 depicts a representative synthesis pathway to prepare Compound UT149.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, at least in part, on an allosteric strategy to modulate STING activities, including small molecule STING antagonist and agonist compounds that bind to the corresponding allosteric site, methods of preparation of the compounds, pharmaceutical compositions comprising the compounds, and their use in medical therapy. In particular, the present disclosure provides STING modulator compounds, which find utility as inhibitors or activators of STING. An advantage of the compounds provided herein is that a broad range of pharmacological activities is possible, ranging from antagonizing STING activity induced by cyclic dinucleotide cGAMP to acting alone or synergizing with cGAMP to activate STING. In addition, the disclosure provides methods of using the antagonist compounds described herein for the treatment of inflammatory, allergic, autoimmune, and infectious diseases, and agonist compounds to treat cancer. The antagonist compounds can also be used for the treatment of senescence- or age-related diseases, such as neurodegenerative diseases, cardiovascular diseases, liver and renal diseases, and premature aging.

### 1. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all value from 16.6 degrees to 150 degrees. For example, they can refer to less than or equal to ±5%, such as less than or equal to ±2%, such as less than or equal to 1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

### 1. STING Allosteric Modulators

Various aspects of the present disclosure are directed to STING allosteric modulators. As demonstrated herein, high-resolution cryo-EM structure of human STING in complex with both cGAMP and compounds of the present disclosure define a novel binding site in the transmembrane domain (TMD) of STING, paving the way for developing more modulators that target this site. Compounds that target the TMD site might be better STING modulators than cGAMP mimetics for therapeutic purposes because they are more hydrophobic and therefore more permeable to the cell membrane. In addition, the structural analyses clarify the direct role of the TMD in the oligomerization and activation of STING. The coupling between the induced opening of the TMD pocket by an illustrative agonist (C53) and the high-order oligomerization of human STING suggests that this conformational change might be an integral part of its activation mechanism in vivo. Surprisingly, it has been found that minor changes in a structure of a compound can effectively interfere with this conformational change, while still binding at the same site—turning a potent agonist into an antagonist in an unpredictable and surprising way.

In view of these discoveries, the present disclosure is directed to particular methods of allosterically inhibiting activity of a STING protein by contacting the STING protein with a STING antagonist described herein. In certain aspects, the STING antagonist may be a compound of Formula (I), Formula (II), or Formula (III):

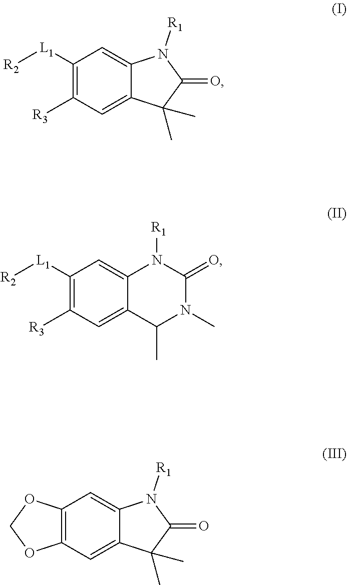

or a salt thereof,
wherein L₁ is absent or is selected from the group consisting of

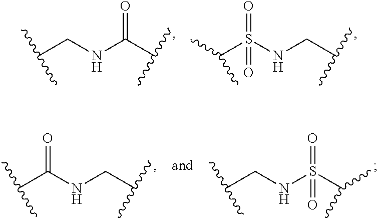 [SMILES: *CNC(*)=O ; *CNS(*)(=O)=O ; *CNC(*)=O ; *CNS(*)(=O)=O]

R₁ is selected from the group consisting of

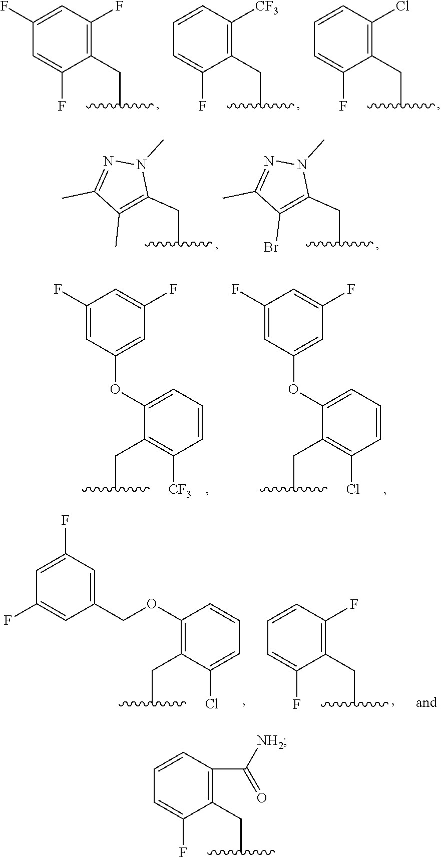

R₂ is selected from the group consisting of H, CF₃, F, CN,

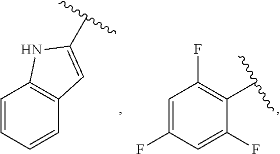 [SMILES: *c1cc2ccccc2[nH]1 ; *c1c(F)cc(F)cc1F]

and
R₃ is selected from the group consisting of H, CN or CF₃. In accordance with various aspects herein, the compounds are further provided with the provisos that (i) at least one of R₂ and R₃ is not hydrogen and (ii) R₁ is not

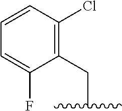 [SMILES: *Cc1c(F)cccc1Cl]

when L1 is

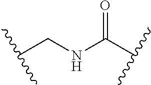 [SMILES: *CNC(*)=O]

and R₂ is

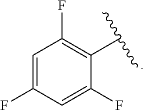 [SMILES: *c1c(F)cc(F)cc1F]

In certain further aspects, the compounds are provided with the additional proviso that (iii) L1 is absent when R₂ is hydrogen, —CN, —F, or —CF₃.

In various aspects, L₁ may be absent

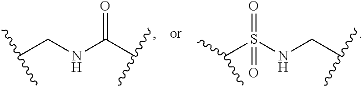 [SMILES: *CNC(*)=O ; *CNS(*)(=O)=O]

For example, in some aspects L₁ may be absent. In other aspects, L₁ may be

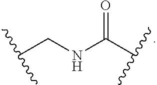 [SMILES: *CNC(*)=O]

In other aspects, L₁ may be

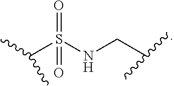 [SMILES: *CNS(*)(=O)=O]

In further aspects of the present disclosure, R₁ can be selected from the group consisting of

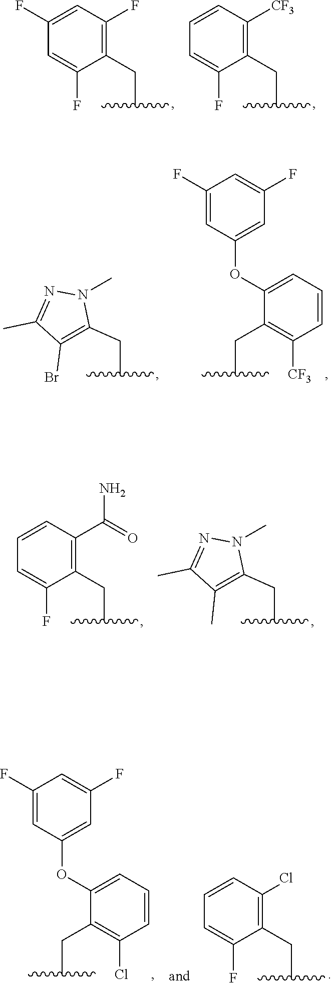

For example, R₁ may be selected from the group consisting of

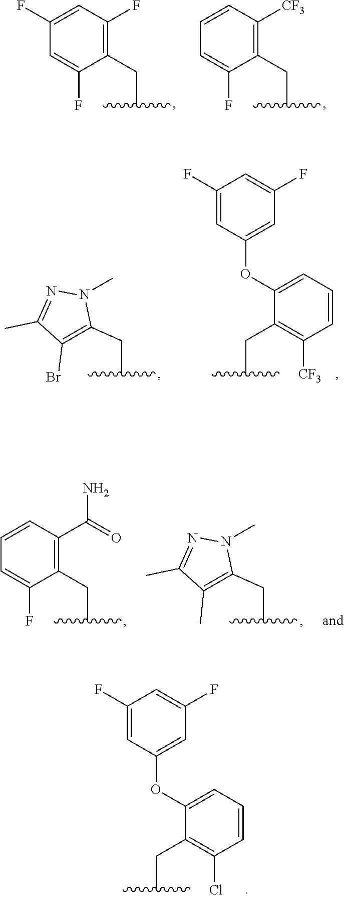

As another example, R₁ may be selected from the group consisting of

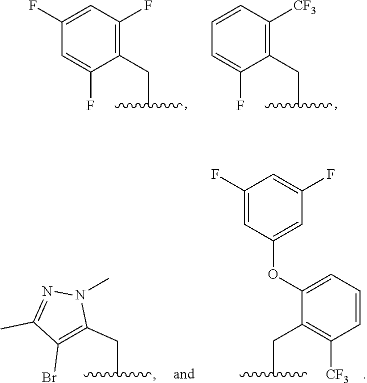

In still further aspects of the present disclosure, R₂ may be selected from the group consisting of

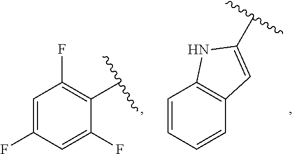 [SMILES: *c1c(F)cc(F)cc1F ; *c1cc2ccccc2[nH]1]

—CN, H, and —CF₃. For example, in some aspects, R₂ is selected from the group consisting of

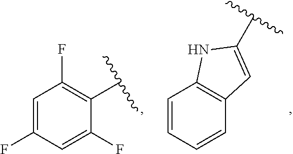 [SMILES: *c1c(F)cc(F)cc1F ; *c1cc2ccccc2[nH]1]

### and —CN.

In certain aspects, R₃ can be hydrogen, CN or CF₃. In certain aspects, R₃ is hydrogen.

In accord with the above, the allosteric inhibitors of the present disclosure may be selected from the group consisting of

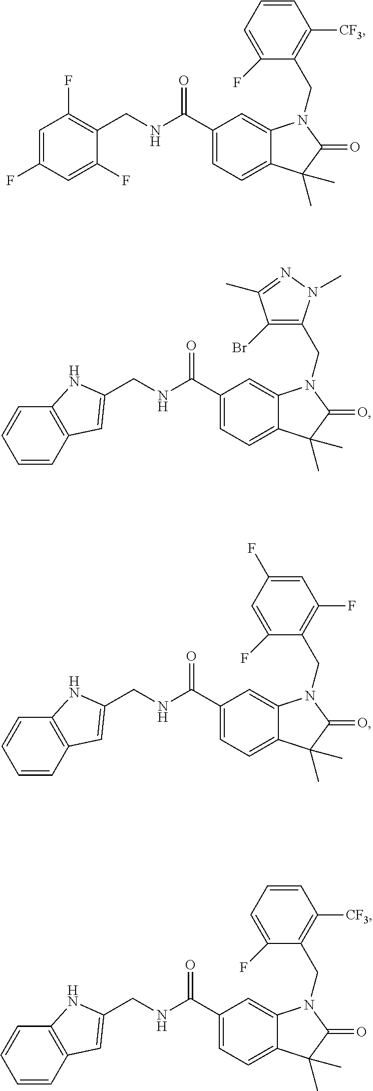 [SMILES: CC1(C)C(=O)N(Cc2c(F)cccc2C(F)(F)F)c2cc(C(=O)NCc3c(F)cc(F)cc3F)ccc21 ; Cc1nn(C)c(CN2C(=O)C(C)(C)c3ccc(C(=O)NCc4cc5ccccc5[nH]4)cc32)c1Br ; CC1(C)C(=O)N(Cc2c(F)cc(F)cc2F)c2cc(C(=O)NCc3cc4ccccc4[nH]3)ccc21 ; CC1(C)C(=O)N(Cc2c(F)cccc2C(F)(F)F)c2cc(C(=O)NCc3cc4ccccc4[nH]3)ccc21]

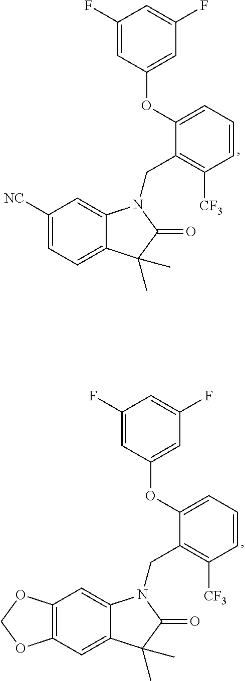 [SMILES: CC1(C)C(=O)N(Cc2c(Oc3cc(F)cc(F)c3)cccc2C(F)(F)F)c2cc(C#N)ccc21 ; CC1(C)C(=O)N(Cc2c(Oc3cc(F)cc(F)c3)cccc2C(F)(F)F)c2cc3c(cc21)OCO3]

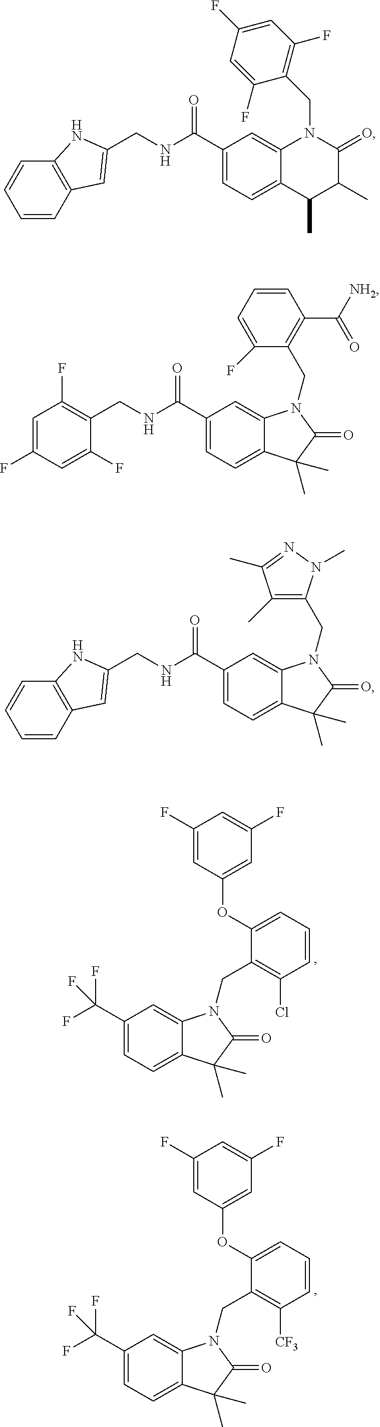

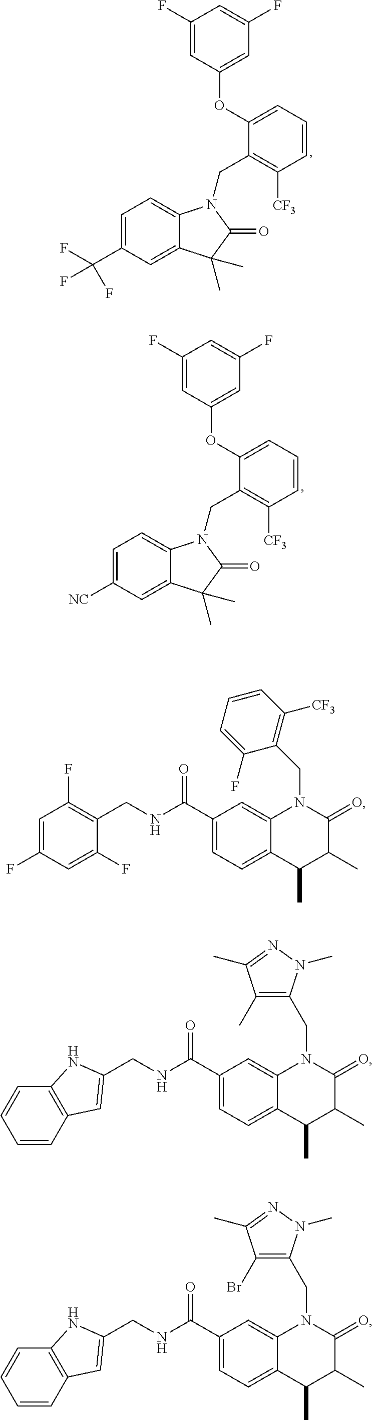 [SMILES: CC1(C)C(=O)N(Cc2c(Oc3cc(F)cc(F)c3)cccc2C(F)(F)F)c2ccc(C(F)(F)F)cc21 ; CC1(C)C(=O)N(Cc2c(Oc3cc(F)cc(F)c3)cccc2C(F)(F)F)c2ccc(C#N)cc21 ; CC1C(=O)N(Cc2c(F)cccc2C(F)(F)F)c2cc(C(=O)NCc3c(F)cc(F)cc3F)ccc2[C@@H]1C ; Cc1nn(C)c(CN2C(=O)C(C)[C@@H](C)c3ccc(C(=O)NCc4cc5ccccc5[nH]4)cc32)c1C ; Cc1nn(C)c(CN2C(=O)C(C)[C@@H](C)c3ccc(C(=O)NCc4cc5ccccc5[nH]4)cc32)c1Br]

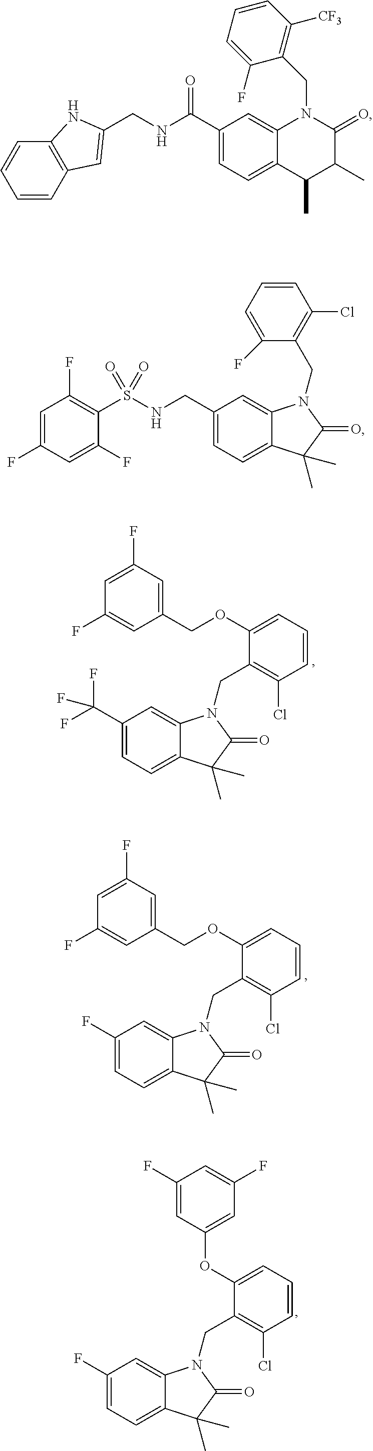 [SMILES: CC1C(=O)N(Cc2c(F)cccc2C(F)(F)F)c2cc(C(=O)NCc3cc4ccccc4[nH]3)ccc2[C@@H]1C ; CC1(C)C(=O)N(Cc2c(F)cccc2Cl)c2cc(CNS(=O)(=O)c3c(F)cc(F)cc3F)ccc21 ; CC1(C)C(=O)N(Cc2c(Cl)cccc2OCc2cc(F)cc(F)c2)c2cc(C(F)(F)F)ccc21 ; CC1(C)C(=O)N(Cc2c(Cl)cccc2OCc2cc(F)cc(F)c2)c2cc(F)ccc21 ; CC1(C)C(=O)N(Cc2c(Cl)cccc2Oc2cc(F)cc(F)c2)c2cc(F)ccc21]

and any salt thereof.

For example, in certain aspects, the allosteric inhibitor of the STING protein (i.e., a STING antagonist) may be selected from the group consisting of

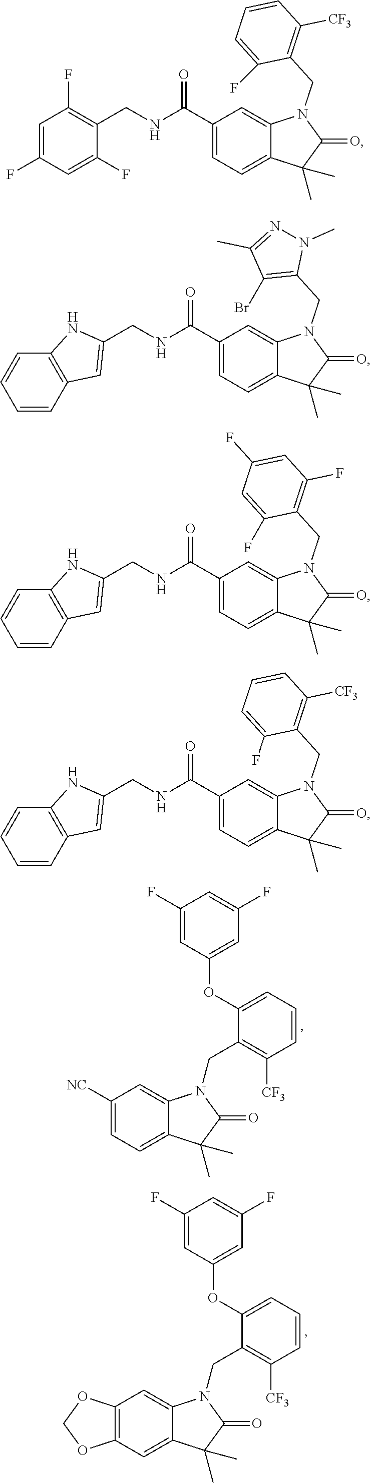

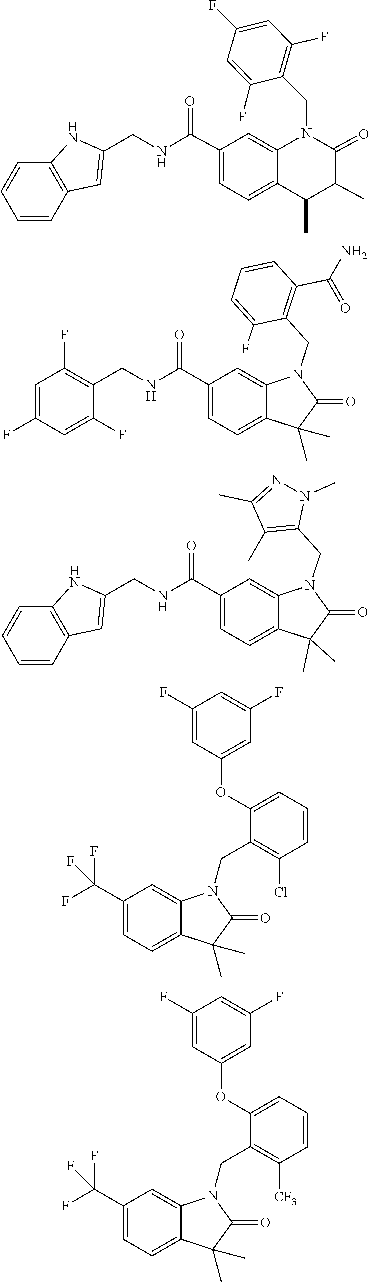

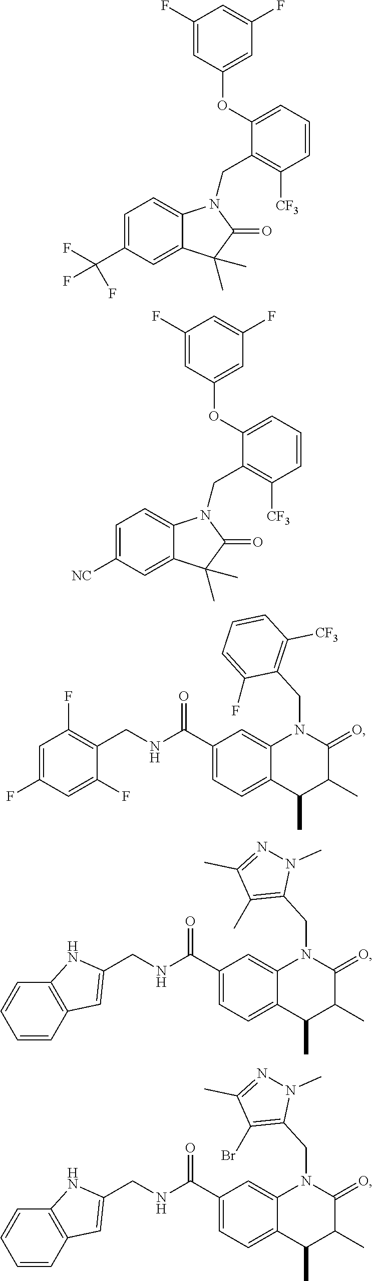

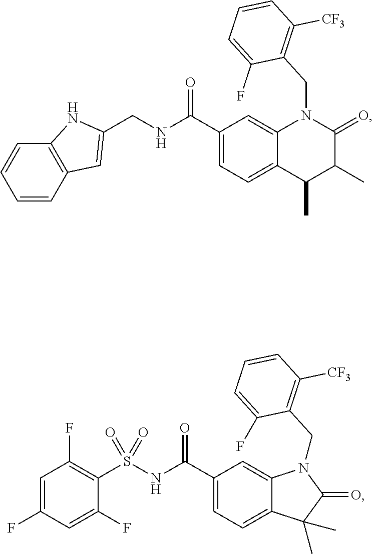 [SMILES: CC1C(=O)N(Cc2c(F)cccc2C(F)(F)F)c2cc(C(=O)NCc3cc4ccccc4[nH]3)ccc2[C@@H]1C ; CC1(C)C(=O)N(Cc2c(F)cccc2C(F)(F)F)c2cc(C(=O)NS(=O)(=O)c3c(F)cc(F)cc3F)ccc21]

For example, in certain aspects, the compounds presented herein may be selected from the group consisting of

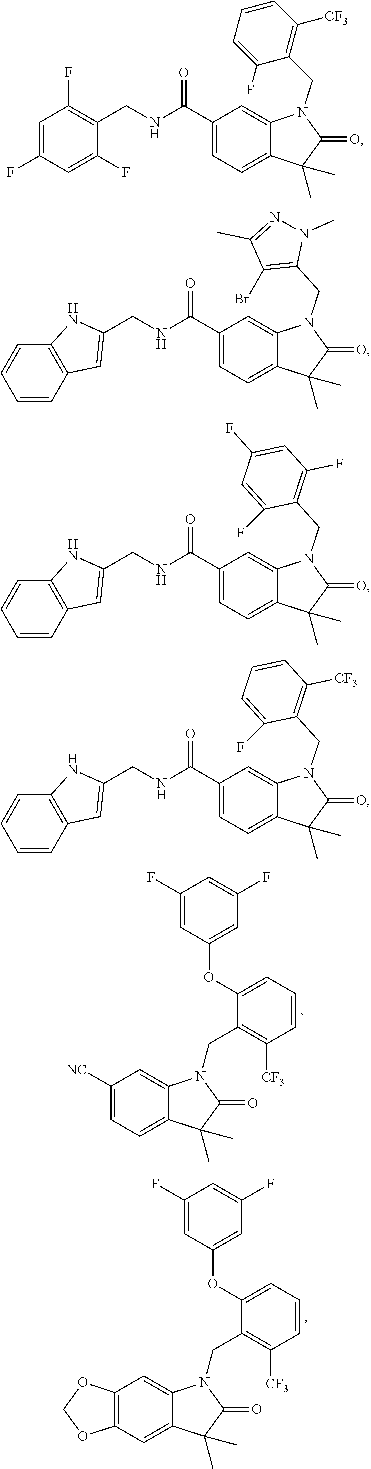

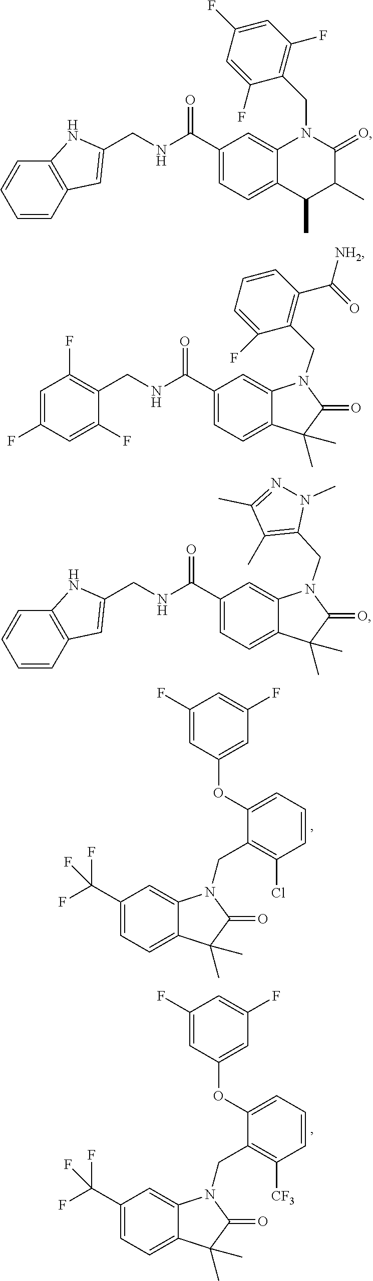

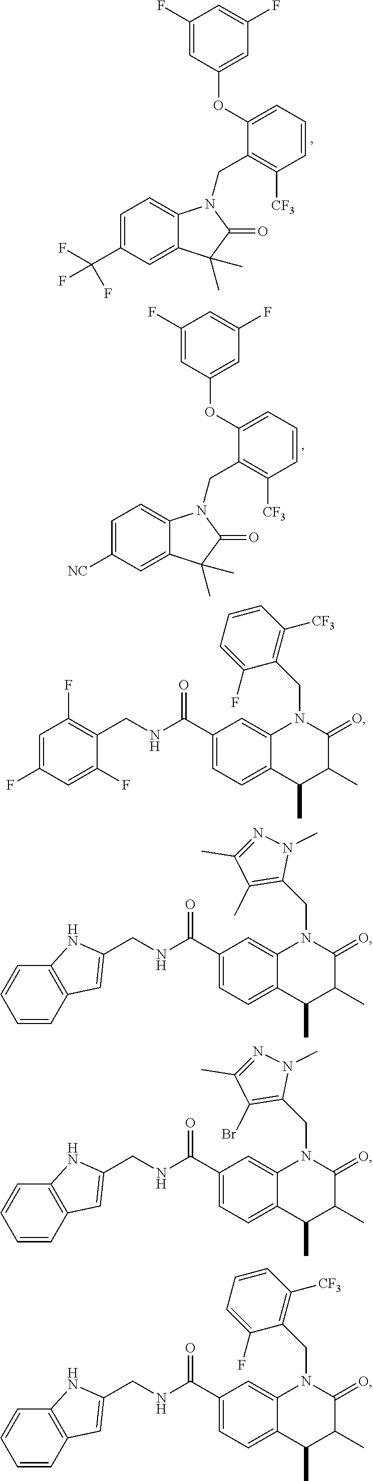

and any salt thereof.

In various aspects, a STING antagonist provided herein may be selected from the group consisting of:

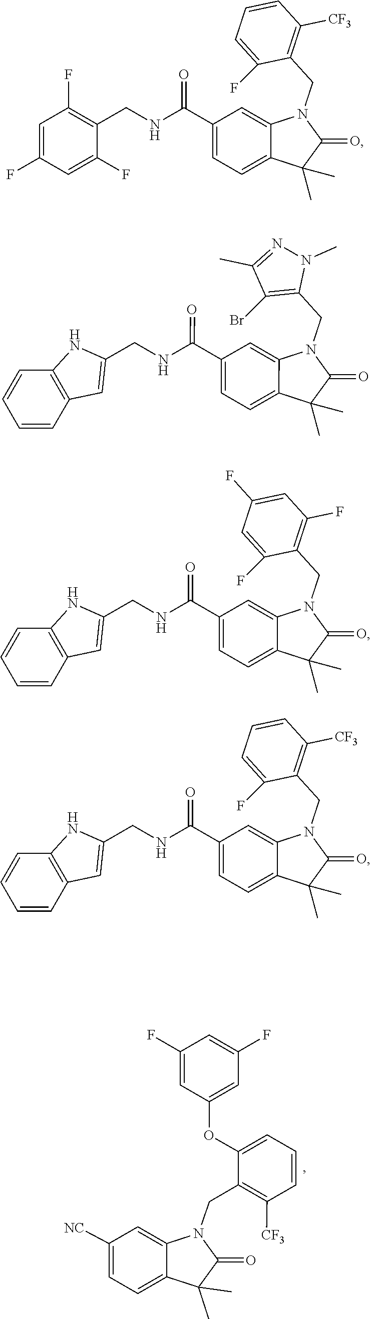

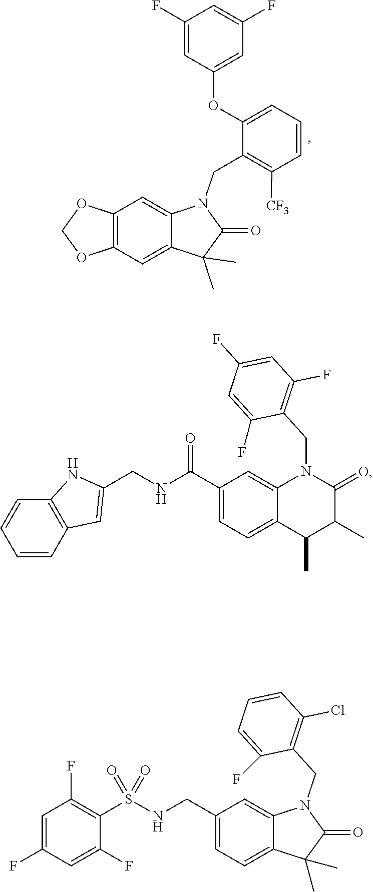 [SMILES: CC1(C)C(=O)N(Cc2c(Oc3cc(F)cc(F)c3)cccc2C(F)(F)F)c2cc3c(cc21)OCO3 ; CC1C(=O)N(Cc2c(F)cc(F)cc2F)c2cc(C(=O)NCc3cc4ccccc4[nH]3)ccc2[C@@H]1C ; CC1(C)C(=O)N(Cc2c(F)cccc2Cl)c2cc(CNS(=O)(=O)c3c(F)cc(F)cc3F)ccc21]

and any salt thereof.

In still further aspects, a STING antagonist may be selected from the group consisting of:

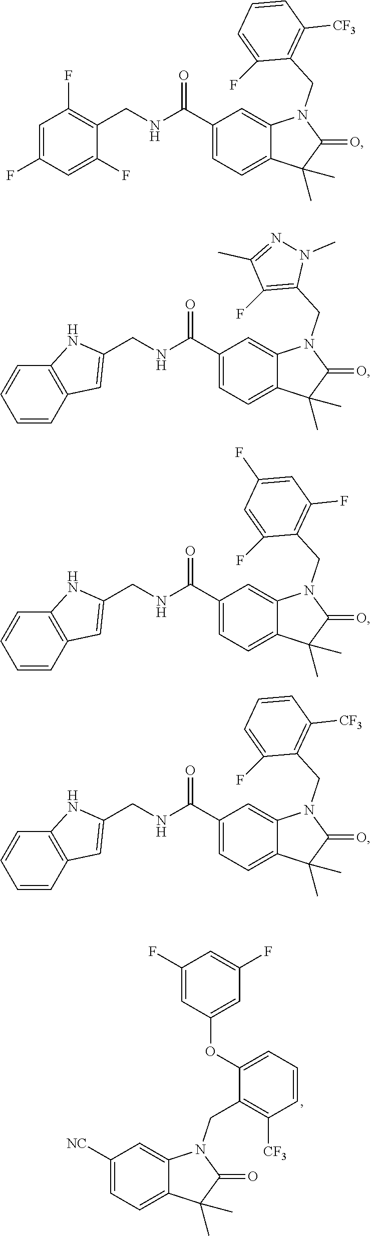

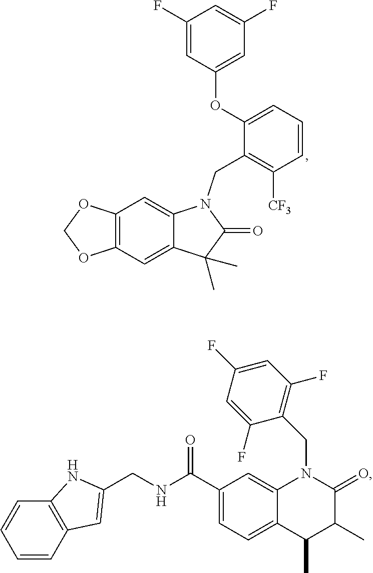 [SMILES: CC1(C)C(=O)N(Cc2c(Oc3cc(F)cc(F)c3)cccc2C(F)(F)F)c2cc3c(cc21)OCO3 ; CC1C(=O)N(Cc2c(F)cc(F)cc2F)c2cc(C(=O)NCc3cc4ccccc4[nH]3)ccc2[C@@H]1C]

and any salt thereof.

In various aspects, the compounds provided herein may be active inhibitors of STING signaling. For example, in some aspects, the compounds may show >50% (i.e., >50%, greater than 60%, greater than 70%, greater than 80% or greater than 90%) inhibition at 10 μM upon cGAMP (100 μM) or MSA-2 (10 μM) stimulation in THP-1 cells. In other aspects, the compounds may show less than 50% inhibition at 10 μM upon cGAMP (100 μM) or MSA-2 (10 μM) stimulation in THP-1 cells. In still other aspects, the compounds may show greater than 50% inhibition at 32 μM under basal conditions (no external stimulation) in THP-1 cells. In other aspects, the compounds may show less than 50% inhibition at 32 μM under basal conditions (no external stimulation) in THP-1 cells.

Methods of measuring STING activity and by extension, the activity of the compounds discussed herein, are known in the art. As an example, the STING pathway activity may be evaluated using reporter cell lines where a reporter gene (i.e., luciferase, GFP) is expressed under direct or indirect control of interferon response elements. For example, in an exemplary embodiment, the STING pathway can be measured using THP1-Dual™ cells (InvivoGen) which express the Lucia gene (a secreted luciferase reporter gene) under the control of an ISG54 minimal promoter in conjunction with five IFN-stimulated response elements. The STING pathway, when activated, stimulates interferon release which, in this model, will increase luciferase expression in a detectable manner. When inhibited, interferon release will be decreased, reducing luciferase expression in a detectable manner. The compounds can optionally be tested in the presence of external stimulation (i.e., using cGAMP, a natural agonist of STING, or MSA-2).

As described in more detail below, the inhibitor compounds provided herein may be used to decrease interferon production and thereby treat or ameliorate various conditions. Without being bound by theory, it is considered that the hydrophobic nature of these and related compounds may make these compounds more suitable as therapeutic agents since they can more easily partition across the plasma membrane and reach the target protein on the endoplasmic reticulum.

### II. Pharmaceutical Compositions

In further aspects of the present disclosure, pharmaceutical compositions are provided. The pharmaceutical compositions may comprise any of the agonist or antagonist compounds described herein and a pharmaceutically suitable carrier or excipient.

The composition may comprise at least one excipient. Suitable excipients include pharmaceutically acceptable excipients, such as diluents, binders, fillers, buffering agents, pH modifying agents, disintegrants, dispersants, preservatives, lubricants, taste-masking agents, flavoring agents, coloring agents, or combinations thereof. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, maltitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, or saccharides.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

The compositions disclosed herein may be formulated into various dosage forms and administered by a number of different means that will deliver a therapeutically effective amount of the active ingredient. The excipients included in the compositions comprising the STING agonist or antagonists described herein, acids thereof, and/or salts thereof may be based on the form of administering such compositions. Such compositions may be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term, “parenteral,” as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980).

### II. Methods of Use

In various aspects, methods of using the STING agonists and/or antagonists described herein are provided. In various aspects, the STING modulators may be used to treat various diseases related to hyper or hypo active native immunity (e.g., interferon production).

As described above, the present disclosure describes a novel allosteric binding site in the transmembrane domain (TMD) of the STING protein. Accordingly, in various aspects, a method is provided for modulating (i.e., antagonizing or activating) STING by targeting the allosteric binding site in the transmembrane domain (TMD).

In various aspects, the methods comprise activating the STING protein. This may occur, for example, by applying a compound which targets the allosteric binding site in the TMD and facilitates the oligomerization and/or phosphorylation of the STING complex—thereby triggering downstream signaling pathways that trigger interferon release and activation of innate immunity. In various aspects, the methods may comprise applying a STING agonist, described herein, to a cell. In various aspects the STING agonist may comprise

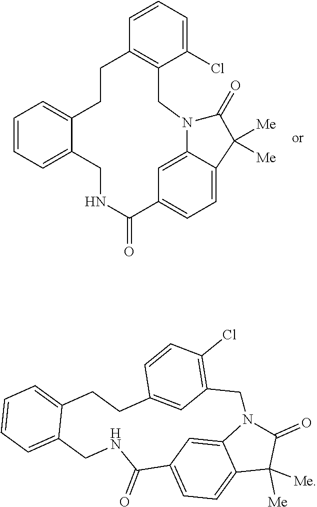 [SMILES: CC1(C)C(=O)N2Cc3c(Cl)cccc3CCc3ccccc3CNC(=O)c3ccc1c2c3 ; CC1(C)C(=O)N2Cc3cc(ccc3Cl)CCc3ccccc3CNC(=O)c3ccc1c2c3]

In additional aspects, the method may comprise antagonizing the STING protein. This may be done by applying a compound that also targets the allosteric binding site, but which blocks oligomerization and/or blocks binding of a native agonist (e.g., cGAMP) to its binding site. In various aspects, the STING antagonist may be a compound of Formula I, Formula II or Formula III as defined above.

In various aspects, the methods of allosterically modulating the STING protein may comprise contacting a cell with any of the compounds described herein. In various aspects, the cell may be in vitro or in vivo. Accordingly, in various aspects a method of treating a subject is provided, the method comprising administering a therapeutically effective amount of any of the STING antagonists or agonists provided herein to the subject. In some aspects, the subject may be suffering from an inflammatory, allergic, autoimmune, and/or infectious diseases, atherosclerosis, arthritis (e.g., osteoarthritis or rheumatoid arthritis), an inflammatory bowel disease (e.g., ulcerative colitis or Crohn's disease). In some aspects, the subject may have senescence- or age-related diseases, such as neurodegenerative diseases, cardiovascular diseases, liver and renal diseases, or be afflicted with premature aging. In some aspects, the subject may have cancer. In some aspects, the method may comprise administering a therapeutically effective amount of a STING antagonist to a subject with an inflammatory, allergic, autoimmune, and/or infectious disease. In some aspects, the method may comprise administering a therapeutically effective amount of a STING antagonist to a subject with senescence- or age-related diseases, such as neurodegenerative diseases, cardiovascular diseases, liver and renal diseases, or premature aging. In some aspects, the method may comprise administering a therapeutically effective amount of a STING agonist to a subject with cancer.

Dosage amounts of the disclosed compounds can be in the range of from about 0.0001 mg/kg/day, 0.001 mg/kg/day or 0.01 mg/kg/day to about 100 mg/kg/day. Dosage may be adjusted based on the stage and severity of cancer and subject characteristics. In some aspects, the subject is administered at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, or 100 mg/kg (or any range derivable therein) of the STING modulator, sufficient to effect treatment of the disease. The effective amount to be administered depends upon a number of factors including, for example, the age and weight of the subject (e.g., a mammal such as a human), the precise condition requiring treatment and its severity, the route of administration, and will ultimately be at the discretion of the attendant physician or veterinarian. It will be understood, however, that the specific dose level for any particular subject will depend on a variety of factors including the activity of the specific compound employed, the age, body weight, general health, sex and diet of the subject being treated; the time and route of administration; the rate of excretion; other drugs which have previously been administered; and the severity of the particular condition undergoing therapy, as is well understood by those skilled in the art.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of disease are exhibited or felt by a subject or after a clinician evaluates the subject for an infection. In some aspects, the subject is administered a first dose of a regimen 1, 2, 3, 4, 5, 6, 7, 8, 9, 10, 11, 12 hours (or any range derivable therein) or 1, 2, 3, 4, or 5 days after the subject experiences or exhibits signs or symptoms of a disease (or any range derivable therein). The subject may be treated for 1, 2, 3, 4, 5, 6, 7, 8, 9, 10 or more days (or any range derivable therein) or until symptoms of the disease have disappeared or been reduced or after 6, 12, 18, or 24 hours or 1, 2, 3, 4, or 5 days after symptoms of a disease have disappeared or been reduced.

In some aspects, the disclosure provides methods for modulating an immune response in a subject having a disease or disorder associated with altered STING function. These methods can include the step of administering to the subject an amount of a pharmaceutical composition including a disclosed STING modulator compound and a pharmaceutically acceptable carrier, wherein amount the pharmaceutical composition is effective to ameliorate the altered STING function in the subject.

Also described herein are methods of treating cancer in a subject having a cancer or tumor with altered STING function and/or infiltrated with inflammatory immune cells. These methods comprise administering to the subject in need thereof, an amount of a pharmaceutical composition including a disclosed STING modulator and a pharmaceutically acceptable carrier, wherein amount the pharmaceutical composition is effective for treating cancer. Treating cancer can comprise inhibition of the proliferation, growth, and/or spread of cancer or tumor cells, inhibition of cancer progression and/or metastases, inhibition of an increase in tumor volume, a reduction in tumor volume, a reduction in tumor growth, an eradication of a tumor and/or cancer cell, or any combinations thereof. The method can also result in a prolonging survival of a subject or improving the quality of the life for the subject. Further, the method can include reducing the number of inflammatory immune cells infiltrating the cancer or tumor (e.g., by at least 20, 30, 40, 50, 60, 70, 80, or 90%, or until reduction of inflammatory cell infiltration is detectably reduced by histology or scanning).

Methods disclosed herein may be used in combination with additional cancer therapy. In some aspects, the distinct cancer therapy comprises surgery, radiotherapy, chemotherapy, toxin therapy, immunotherapy, cryotherapy or gene therapy. In some aspects, the cancer is a chemotherapy-resistant or radio-resistant cancer. Combination therapy may be achieved by use of a single pharmaceutical composition that includes both agents, or by administering two distinct compositions at the same time, wherein one composition includes the STING modulator and the other includes the second agent(s).

As such, one aspect of the disclosure encompasses treatment of any STING related cancer or neoplasm. As it will be recognized by individuals skilled in the art, cancer as used throughout the instant disclosure may be one or more neoplasm or cancer. The neoplasm may be malignant or benign, the cancer may be primary or metastatic; the neoplasm or cancer may be early stage or late stage. Non-limiting examples of neoplasms or cancers that may be treated include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenstrom), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenstrom macroglobulinemia, and Wilms tumor (childhood).

In some aspects, administration of STING modulator disclosed herein, prevents (neuroprotective) or treats neurological disorders. These methods comprise administering to the subject in need thereof, an amount of a pharmaceutical composition including a disclosed STING modulator and a pharmaceutically acceptable carrier, wherein amount the pharmaceutical composition is effective for treating the neurological disorder. In such cases, the administration of disclosed STING modulators can improve cognitive function, suppress neuronal apoptosis, suppress amyloidosis of cranial nerves, lower a total count of immune microglia in brain, reduce inflammation in brain, and/or prevent the progression of neurological disorder. Neurological disorder refers to any disorder of the nervous system and/or visual system. “Neurological disorders” include disorders that involve the central nervous system (brain, brainstem and cerebellum), the peripheral nervous system (including cranial nerves), and the autonomic nervous system (parts of which are located in both central and peripheral nervous system). Neurodegenerative diseases, include, for example, Alzheimer's Disease, stroke, multiple sclerosis etc.

Several neurological disorders, symptoms, signs and syndromes that can be treated using compositions and methods according to the present invention include: acquired epileptiform aphasia; acute disseminated encephalomyelitis; adrenoleukodystrophy; age-related macular degeneration; agenesis of the corpus callosum; agnosia; Aicardi syndrome; Alexander disease; Alpers' disease; alternating hemiplegia; Alzheimer's disease; Vascular dementia; amyotrophic lateral sclerosis; anencephaly; Angelman syndrome; angiomatosis; anoxia; aphasia; apraxia; arachnoid cysts; arachnoiditis; Anronl-Chiari malformation; arteriovenous malformation; Asperger syndrome; ataxia telegiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome; causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; Dandy-Walker syndrome; Dawson disease; De Morsier's syndrome; Dejerine-Klumke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia and other “tauopathies”; Gaucher's disease; Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile phytanic acid storage disease; infantile refsum disease; infantile spasms; inflammatory myopathy; intracranial cyst; intracranial hypertension; Joubert syndrome; Kearns-Sayre syndrome; Kennedy disease Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gustaut syndrome; Lesch-Nyhan syndrome; leukodystrophy; Lewy body dementia; Lissencephaly; locked-in syndrome; Lou Gehrig's disease (i.e., motor neuron disease or amyotrophic lateral sclerosis); lumbar disc disease; Lyme disease—neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; milti-infarct dementia; multifocal motor neuropathy; multiple sclerosis and other demyelinating disorders; multiple system atrophy with postural hypotension; p muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocal leukoencephalopathy; progressive sclerosing poliodystrophy; progressive supranuclear palsy; pseudotumor cerebri; Ramsay-Hunt syndrome (types I and II); Rasmussen's encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia; shaken baby syndrome; shingles; Shy-Drager syndrome; Sjögren's syndrome; sleep apnea; Soto's syndrome; spasticity; spina bifida; spinal cord injury; spinal cord tumors; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal arteritis; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau disease; Wallenberg's syndrome; Werdnig-Hoffman disease; West syndrome; whiplash; Williams syndrome; Wildon's disease; and Zellweger syndrome.

In another aspect, disclosed herein are methods of administration of STING modulators to prevent or treat subjects suffering from an autoimmune disease. Examples of autoimmune diseases comprise: rheumatoid arthritis, systemic lupus erythematosus, inflammatory bowel diseases (IBDs) comprising Crohn disease (CD), ulcerative colitis (UC) psoriasis, diabetes and as agents to suppress transplant rejection. The method comprises administering to a subject, an amount of a disclosed STING modulator, or a pharmaceutically acceptable salt thereof, for curing, reversing, alleviating, palliative and/or prophylactic treatment of the autoimmune disease or one or more symptoms associated with the disease. Examples of other autoimmune diseases which may be treated using the compositions of the present invention include, but are not limited to, alopecia areata, autoimmune hemolytic anemia, autoimmune hepatitis, dermatomyositis, autoimmune juvenile idiopathic arthritis, glomerulonephritis, Graves' disease, Guillain-Barre syndrome, idiopathic thrombocytopenic purpura, lupus, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, some forms of thyroiditis, some forms of uveitis, vitiligo, and granulomatosis with polyangiitis (Wegener's).

In a further aspect provided herein is a method of treating inflammation, and/or allergy comprising administering to a subject in need thereof a therapeutically effective amount of a disclosed STING modulator or a pharmaceutically acceptable salt thereof. Inflammatory and/or allergic conditions which may be treated with the disclosed STING modulators include, for example, asthma, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome. Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, astopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autroimmine) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. The STING modulators may additionally be used to treat T-cell mediated hypersensitivity diseases having an inflammatory component. Such conditions include contact hypersensitivity, contact dermatitis (including that due to poison ivy), uticaria, skin allergies, respiratory allergies (hayfever, allergic rhinitis) and gluten-sensitive enteropathy (Celliac disease).

In some aspects this disclosure provides a method for delaying onset or progression of senescence or an age-related disease or condition in a subject comprising administering to the subject a STING modulator provided herein, or a pharmaceutically acceptable salt thereof. In some aspects the method delays the onset of senescence or an age-related disease or condition. In some aspects the method delays the progression of senescence or an age-related disease or condition. In some aspects the age-related disease or condition is selected from atherosclerosis, cardiovascular disease, cancer, arthritis, dementia, cataract, osteoporosis, diabetes, hypertension, age-related fat loss, vertebral disc degeneration, age-related muscular atrophy and kidney disease.

In some aspects, provided herein, is a method of treating infections using disclosed STING modulators. The method comprises administering an amount of a STING modulator to a subject in need thereof, to reduce or inhibit one or more symptoms associated with infection. Infections can be viral infections or bacterial infections. In some aspects, the method inhibits viral or bacterial replication in a subject. In other aspects, the disclosed STING modulators reduce the viral or bacterial load in the subject. In some aspects, STING modulators disclosed herein, can be used as a vaccine adjuvant, to enhance the potency of the vaccine. In some aspects, the infection is a hepatitis B viral (HBV) infection. In some aspects, the infection is a Alphaviral infection, such as the ones caused by West Nile Virus (WNV), Vaccinia Virus (VACV), and Chikungunya virus (CHIKV), Venezuelan Equine Encephalitis Virus (VEEV), Eastern equine encephalitis (EEE). Administration of the STING modulators can elicit the desired activity or biological response in the subject, such as, can decrease the sign or symptom by, for example by at least 20%, at least 40%, at least 50%, at least 80%, at least 90%, at least 95%, at least 98%, or even at least 100%, as compared to how the sign or symptom would have progressed in the absence of the composition.

Typical subjects include animals (e.g., mammals, birds, amphibians, reptiles, etc.). In various embodiments, the subject is a mammal. Any suitable mammal can be treated by a method or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In certain embodiments a mammal is a human. In certain embodiments a mammal is a non-rodent mammal (e.g., human, pig, goat, sheep, horse, dog, or the like). In certain embodiments a non-rodent mammal is a human. A mammal can be any age or at any stage of development (e.g., an adult, teen, child, infant, or a mammal in utero). A mammal can be male or female. In certain embodiments a mammal can be an animal disease model. In an aspect, a subject is a human. In some aspects, a subject may be suffering from, and/or susceptible to a disease, disorder, and/or condition associated with altered STING activity (e.g., cancer, neurodegenerative diseases, cardiovascular diseases etc.).

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Introduction to Examples

Innate immunity provides the first-line defense against infection. A major innate immunity pathway for detecting invading viruses or bacteria is mediated by cyclic GMP-AMP (cGAMP) synthase (cGAS). cGAS serves as a DNA sensor by directly binding to pathogen DNA in the cytosol and uses GTP and ATP as substrates to produce the second messenger cGAMP. cGAMP binds and activates the downstream adaptor protein STING (Stimulator of interferon genes), which initiates several downstream signaling pathways, including the type-I interferon pathway, the NFκB pathway and autophagy, to eliminate the pathogen. cGAS/STING signaling can be activated by DNA from tumor cells and launch antitumor immunity by promoting the production of type I interferons, cell senescence and the adaptive immunity against tumor cells. Targeting cGAS-STING dependent signaling has shown promise in clinical application on anti-tumor treatment. In the past few years, a surge effort has been made in the development of STING agonists as novel anti-cancer therapeutics.

STING is a transmembrane protein containing four TM helices that forms the TM domain (TMD), which is followed by a cytoplasmic ligand-binding domain (LBD) that binds cGAMP. In addition, STING has a C-terminal tail that contains the PXPLRXD (SEQ ID NO: 1 wherein X is any residue) motif that recruits the TANK-binding kinase 1 (TBK1). Binding of TBK1 to this motif promotes the phosphorylation of Ser366 in the STING tail, which subsequently recruits and promotes the phosphorylation of the transcription factor interferon regulatory factor 3 (IRF3), ultimately leading to the expression of type I interferons. Structural approaches have been used to elucidate the mechanisms that control these activation steps of STING. STING exists as a constitutive domain-swapped dimer that is stabilized by interactions contributed by both the TMD and LBD. cGAMP binds the cleft at the center of the butterfly-shaped LBD dimer, inducing concerted conformational changes in the LBD that include inward rotation of the two wings and closure of the binding pocket. These conformational changes were coupled to a 180°-rotation of the LBD relative to the TMD, as well as a downward tilt of the LBDα2-LBDα3 loop, which mediated the formation of the side-by-side high-order oligomer of STING that is essential for the recruitment of TBK1 and the subsequent phosphorylation events. STING oligomerization and activation were also coupled to cGAMP-induced translocation from the ER to the Golgi apparatus, for which the mechanism is not well understood.

Despite the high affinity of STING for cGAMP, STING oligomers induced by cGAMP alone appeared weak in solution, which contradicts the cell-based imaging results that cGAMP induced robust puncta formation of STING on ER or Golgi membrane. In addition, previous cryo-EM structures of the chicken STING tetramer at low resolution (6.5 Å) show inter-dimer interactions in the LBD, but the role of the TMD in the oligomerization remains unclear. These observations together suggested that the stability of the high-order oligomer of STING may involve additional factors interacting with the TMD. In search for such factors, the interaction of a STING activator compound 53 (C53) with human STING was examined. A cryo-EM structure of human STING bound with both cGAMP and C53, was solved, revealing a novel agonist binding pocket in the TMD of STING. These structural and functional analyses further showed that the concurrent bindings of cGAMP and C53 to LBD and TMD of STING, respectively, promoted the formation of higher-order STING oligomers, and thereby efficiently boost the activation of STING.

### Example 1—Materials and Methods

Protein expression and purification. The expression constructs and expression and purification procedures used for human STING are similar to those described previously (e.g., Shang, G. et al., Cryo-EM structures of STING reveal its mechanism of activation by cyclic GMP-AMP. Nature 567, 389-393, (2019), incorporated herein by reference in its entirety). The coding sequence for human STING residues 1-343, excluding the C-terminal tail that is not a part of the folded structure of the protein, fused to a cleavage site for the human rhinovirus 3C protease and T6SS immunity protein 3 (Tsi3) from Pseudomonas aeruginosa at the C terminus in tandem were inserted into the pEZT-BM vector. Mutations were introduced by PCR-based mutagenesis. The plasmids were transfected using polyethylenimine (PEI) into HEK293F cells cultured in suspension in FreeStyle293 Expression medium (Gibco, Cat #12338-018), with 1000 μg DNA and 4 ml PEI at 1 mg/ml for 1 L cells. These and other cells used were assumed to be authenticated by the commercial sources, and therefore were not authenticated in the study. DAPI (4′,6-diamidino-2-phenylindole) staining and the e-Myco Mycoplasma PCR Detection Kit (Bulldog Bio) were used to ensure cells not contaminated by mycoplasma. Cells were harvested 72 hours after transfection, re-suspended in buffer A (containing 20 mM HEPES pH 7.5, 150 mM NaCl, 5 mM CaCl2, 1% DNase I, 0.2 mM AEBSF and 0.5 mM TECP) and disrupted by French press. The lysates were centrifuged for 10 min at 5000 g to remove debris. Membrane fraction was pelleted by centrifugation at 100,000 g for 1 hour. Proteins in lipid membrane were extracted by 1% n-Dodecyl-B-D-Maltoside (DDM) and 0.2% cholesteryl hemisuccinate tris salt (CHS) in buffer A. The sample was subjected to another round of centrifugation to remove insoluble fraction. The affinity purification step of human STING was based on the high-affinity interaction between the C-terminal Tsi3 tag and the T6SS effector protein Tse3⁸. Detergent solubilized STING was captured by Tse3-conjugated Sepharose 4B resin (GE Healthcare) equilibrated in buffer B (20 mM HEPES pH 7.5, 150 mM NaCl, 5 mM CaCl2, 20 mM imidazole, 0.5 mM TECP, 0.03% DDM and 0.006% CHS). Unbound proteins were removed by extensive wash with buffer B. STING was eluted by cleavage from the Tsi3 tag with the 3C protease on resin at 4° C. for 12 hours. The eluted protein was further purified on a Superdex S200 increase 10/300 column (GE healthcare) in buffer C (25 mM HEPES pH 7.5, 150 mM NaCl, 0.5 mM TECP, 0.03% DDM and 0.006% CHS). Peak fractions were pooled, concentrated and kept at −80° C. before use.

Synthesis of C53. All solvents for synthesis (N,N′-dimethylformamide (DMF), tetrahydrofuran (THF) and methylene chloride (DCM)) were obtained by passing commercially available pre-dried, oxygen-free formulations through activated alumina columns. All reagents were purchased at high commercial quality (Sigma-Aldrich, Oakwood and AK Scientific) and used without further purification, unless otherwise stated. Reactions were monitored by thin-layer chromatography (TLC) carried out on 0.25 mm E. Merck silica gel plates (60F-254) and visualized under UV light and/or by appropriate staining method (an ethanolic solution of phosphomolybdic acid or cerium sulphate). Flash column chromatography was performed using E. Merck silica gel (60, particle size 0.04-0.063 mm). NMR spectra were recorded on a Bruker Ascend 400 and calibrated using residual not perdeuterated solvent (DMSO-d6: δH=2.50 ppm, δC=39.52 ppm) or using an external reference for ¹⁹F NMR [δF=0 (CCl3F) ppm] at 298 K. All ¹³C NMR spectra were broadband 1H decoupled. The chemical shifts of the peaks of the major rotamer were reported and coupling constants were given in Hz only for the major rotamer. LC-MS was performed on Agilent 1260 Infinity II Single Quadrupole with an Agilent Eclipse XDB-C18 5 μm 4.6×150 mm column. Buffer A was 0.1% CF3CO2H in H2O, and buffer B was 0.1% CF3CO2H in MeCN. Analytical gradient (0.0-7.0 min, buffer B from 10% to 60%; 7.0-10.0 min, buffer B from 60% to 100%; 10.0-15.0 min, buffer B maintains 100%) was performed with flow of 0.8 mL/min. For details of the chemical synthesis and characterization, as described below.

Native gel analyses of STING oligomerization. Purified human STING wild type or mutants in buffer C at 50 μM were incubated with DMSO as control, cGAMP at 100 μM, C53 at 100 μM or both at 4° C. for 2 hrs. Samples (15 μg protein) were mixed with the Native gel loading buffer (Invitrogen, Cat #BN20032) and resolved using 3-12% gradient native gel (Invitrogen, Cat #BN1003BOX).

Cryo-EM data collection and image processing. Purified wild type human STING at 50 μM were incubated with cGAMP at 100 μM and C53 at 100 μM for formation of the protein/ligands complex. The complex was purified using a Superose 6 10/300 gel filtration column (GE healthcare) in buffer C. Peak fractions were collected and concentrated to 2.9 mg/ml. Additional cGAMP (100 μM) and C53 (100 μM) were added to ensure saturation of the protein by the ligands. The sample was applied to a glow-discharged Quantifoil R1.2/1.3 300-mesh gold holey carbon grid (Quantifoil, Micro Tools GmbH, Germany), blotted under 100% humidity at 4° C. and plunged into liquid ethane using a Mark IV Vitrobot (FEI).

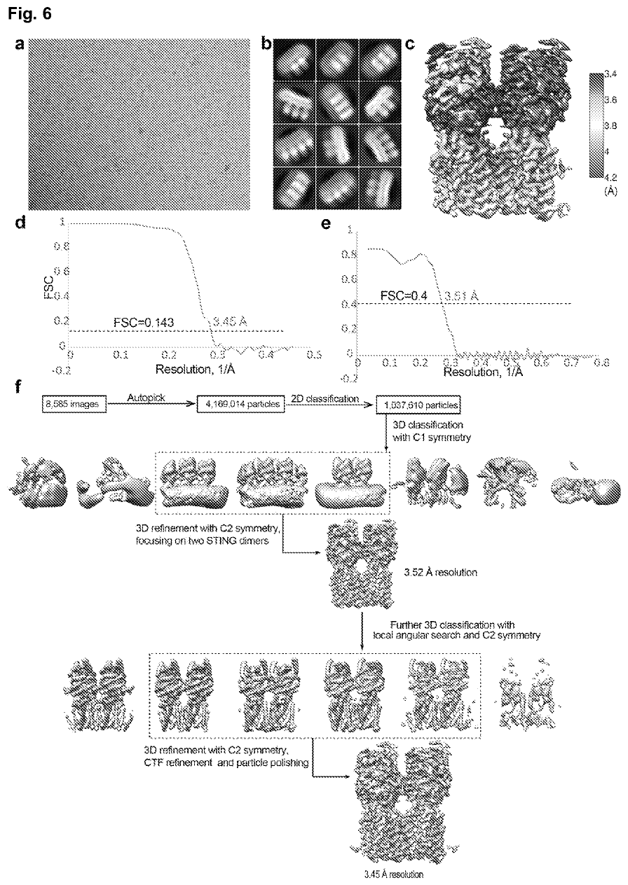

Micrographs were collected on a Titan Krios microscope (FEI) with a K3 Summit direct electron detector (Gatan) in the super-resolution counting mode operated at 300 kV. The slit width of the GIF-Quantum energy filter was set to 20 eV. The nominal magnification was 81,000× and the pixel size of 1.08 Å. Micrographs were dose-fractioned into 36 frames with a total exposure time of 7.2 s at the dose rate of 1.6 e⁻/Å²/frame in the correlated double sampling (CDS) mode. Movie frames were motion-corrected and dose-weighted using the Motioncorr2 program (version 1.2). GCTF 1.06 was used for CTF correction. Two sets of Particles were picked by using Topaz 0.2 and template-based picking in RELION 3.1, respectively. The two sets were combined with duplicates removed. The rest of the image processing was done in RELION (FIG. 6D). Particles were initially extracted with a box size of 160 pixel, which is large enough to accommodate four STING dimers, and binned by a factor of 4 for 2D classification. Particles from good 2D classes were re-extracted with a binning factor 2 and subjected to 3D classification. The initial model was generated with the chicken STING tetramer. A total of 288,021 particles in good 3D classes were selected and re-extracted to the original pixel size for 3D refinement. The 3D reconstructions of the STING tetramer, with C2 symmetry relating the two dimers, reached resolution of −3.52 Å. An additional round 3D classification with local angular search and the C2 symmetry were performed and two classes showing poor density was removed. The resulting 231,556 particles were subjected to further arounds of 3D refinement, CTF refinement and Bayesian polishing, leading to the final 3D reconstruction with resolution of 3.45 Å. Resolution was estimated by applying a soft mask around the protein density, using the Fourier Shell Correlation (FSC) 0.143 criterion (FIG. 6C). Local resolution was calculated in RELION (FIG. 6B).

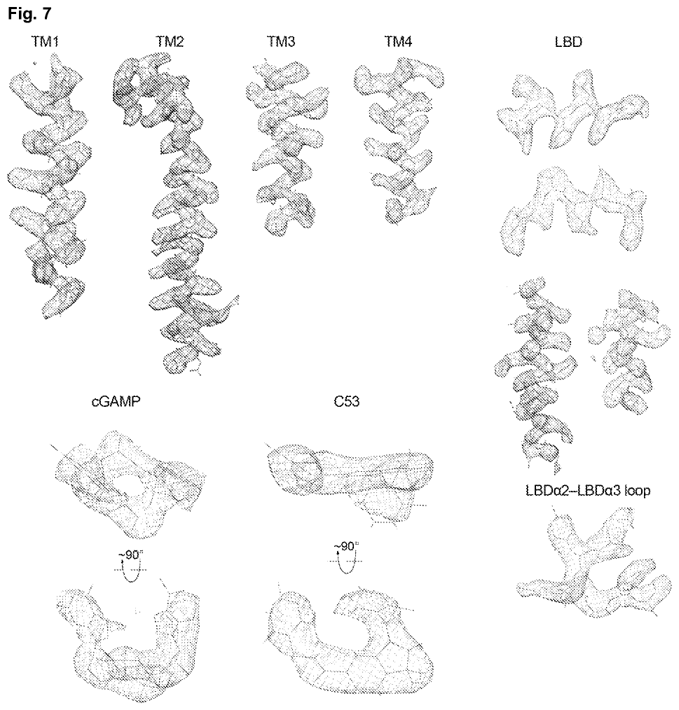

Model building, refinement and analyses. Model building was initiated by docking the structure of the human STING dimer in the apo state (PDB ID: 6NT5) into the cryo-EM density, followed by manual adjustments in Coot 0.94 (Emsley, P. et al., Features and development of Coot. Acta crystallographica 66, 486-501, (2010), incorporated herein by reference in its entirety). The high quality of the density allows most of the residue sidechains to be clearly identified. C53 and cGAMP were manually fit into the density in Coot. cGAMP is an asymmetric molecule with a 3′-5′ and 2′-5′ phosphodiester bond linking the AMP and GMP moieties, which could bind to the symmetric STING dimer in two alternative orientations. The subtle asymmetric of cGAMP however do not usually cause obvious asymmetric in the STING LBD. As a result, either way of fitting cGAMP into its binding pocket was equally valid. One orientation was arbitrarily chosen to fit cGAMP into the density (FIG. 7). In contrast, the asymmetry of C53 is much more pronounced, leading to clear asymmetry in the STING-TMD. The well-defined density for C53 allowed us to dock it to the binding pocket without ambiguity (FIG. 7). Real-space refinement of the model was carried out with Phenix 1.18 (Adams, P. D. et al. PHENIX: a comprehensive Python-based system for macromolecular structure solution. Acta crystallographica 66, 213-221, (2010), incorporated herein by reference in its entirety). Molprobity as a part of the Phenix validation tool was used for assessing the quality of the model (Chen, V. B. et al. MolProbity: all-atom structure validation for macromolecular crystallography. Acta crystallographica 66, 12-21, (2010), incorporated herein by reference in its entirety)

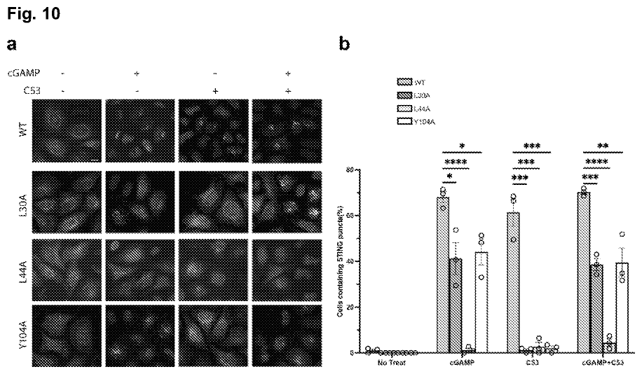

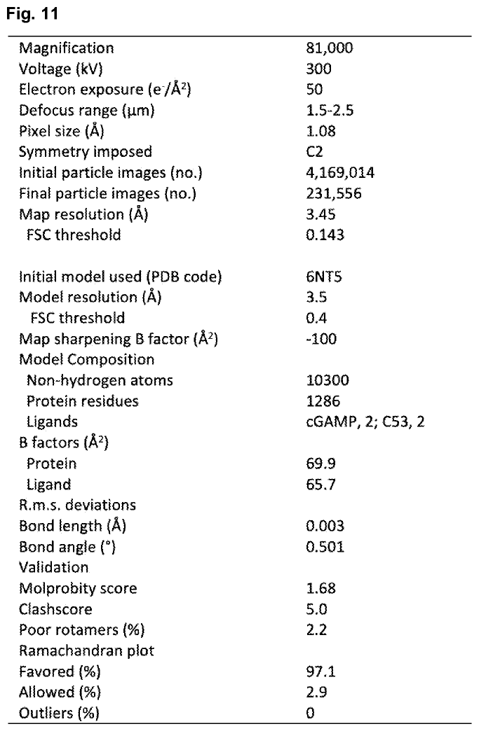

Statistics of the refined model are summarized in FIG. 11. Structural figures were rendered in Coot, PyMOL2.4 (The PyMOL Molecular Graphics System, Schrödinger) or Chimera 1.16 (Pettersen, E. F. et al. UCSF Chimera—a visualization system for exploratory research and analysis. J Comput Chem 25, 1605-1612, doi:10.1002/jcc.20084 (2004), incorporated herein by reference in its entirety).

Sequence alignment was rendered with ESPript 3 (Robert, X. & Gouet, P. Deciphering key features in protein structures with the new ENDscript server. Nucleic Acids Res 42, W320-324, doi:10.1093/nar/gku316 (2014), incorporated herein by reference in its entirety). Two-dimensional interaction diagram between C53 and STING was generated with LigPLot+2.2 (Laskowski, R. A. & Swindells, M. B. LigPlot+: multiple ligand-protein interaction diagrams for drug discovery. J Chem Inf Model 51, 2778-2786, doi:10.1021/ci200227u (2011), incorporated herein by reference in its entirety).

Western blot analyses of phosphorylation of STING, TBK1 and IRF3. The coding sequence for full-length human STING fused with a C-terminal FLAG-tag was cloned into the pcDNA3.1A(+) vector (Invitrogen). Mutations were introduced by PCR-based mutagenesis. Plasmids were transfected with 500 ng into HEK293T cells in 6-well plates and cultured for additional 24 hours. Stimulation of cells with cGAMP and C53 was carried out by using digitonin-mediated permeabilization that allows cGAMP to penetrate the cell membrane and enter the cell. cGAMP (1 μM), C53 (10 μM) or both in a buffer containing 50 mM HEPES pH7.5, 100 mM KCl, 3 mM MgCl₂, 0.1 mM DTT, 85 mM sucrose, 0.2% BSA, 1 mM ATP, 0.1 mM GTP and 10 μg/ml digitonin were used to treat cells for 1 hour. Cells were lysed in RIPA buffer and lysates were subjected to western blot analyses. Human STING protein was detected by anti-FLAG primary antibody (Bimake, Cat #A5712; 3000× dilution) and anti-Mouse IgG HRP-linked secondary antibody (Cell Signaling Technology, Cat #7076S; 3000× dilution). Phosphorylated STING was detected by Rabbit anti-phospho-STING (S366) antibody (Cell Signaling Technology, Cat #19781S; 1000× dilution) and anti-Rabbit IgG HRP-linked secondary antibody (Cell Signaling Technology, Cat #7074S; 3000× dilution). TBK1 was detected by Mouse anti-TBK1/NAK antibody (Cell Signaling Technology, Cat #51872S; 1000× dilution). Phosphorylated TBK1 was detected by Rabbit anti-phospho-TBK1/NAK antibody (Cell Signaling Technology, Cat #5483S; 1000× dilution). IRF3 was detected by Mouse anti-IRF3 antibody (Abcam, Cat #ab50772; 100× dilution) and phosphorylated IRF was detected by Rabbit anti-phospho-IRF3 (S386) (Abcam, Cat #ab76493; 1000× dilution).

Fluorescence microscopy of STING oligomerization in cells. The coding sequence for full-length human STING fused with a C-terminal GFP-tag was cloned into the pmEGFP-N1 vector (Addgene). Mutations were introduced by PRC-based mutagenesis. Plasmids were transfected into Hela cells cultured on glass coverslips in 6-well plates and cultured for additional 24 hours. Stimulation of cells with cGAMP and C53 was performed in the same manner as described above. One hour after stimulation, cells were washed in PBS and fixed in 4% paraformaldehyde. STING-GFP was imaged by using a DeltaVision fluorescence microscope with a 40× objective. Nuclei were stained with DAPI. The experiment for each STING constructs was repeated three times. The percentage of cells containing STING puncta were obtained by counting number of GFP-positive cells with or without STING puncta respectively in images taken from random fields of coverslips. The number of cells counted for each STING construct in each repeat ranged from 60-452. The data was plotted with GraphPad Prism 9.

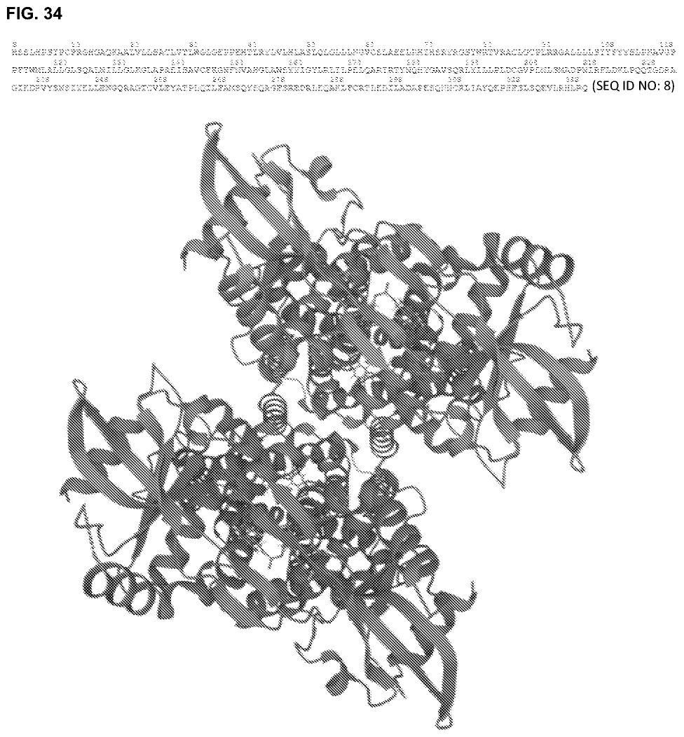

The atomic coordinates and the cryo-EM map will be deposited into the RCSB and EMD databases and released to the public upon. Additionally, see FIG. 34.

### Example 2—C53 Induces the Oligomerization of Human STING in Synergy with cGAMP

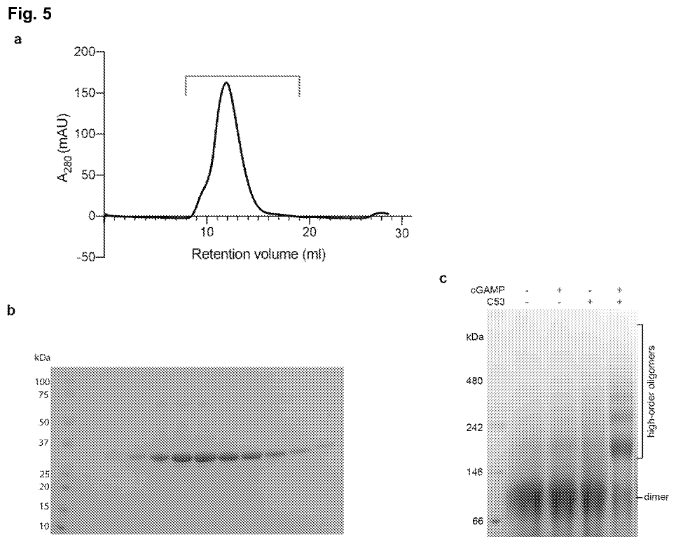

To stabilize the cGAMP-induced active oligomer of human STING for mechanistic analyses, STING agonists were identified that are chemically distinct from cGAMP and less likely act as cGAMP mimetics. To this end, a series of studies were performed on small mostly hydrophobic benzothiazinone-like compounds that activate human STING selectively and potently but lack the negative charge of cGAMP. One of these agonists—C53, induces STING-mediated secretion of interferons with an EC₅₀ of 185 nM. A native gel assay was used to assess the effect of C53 on oligomerization of purified human STING in detergent solution (FIG. 5). cGAMP and C53 together induced robust oligomerization of STING, while either one alone failed to do so under the same condition (FIG. 5c). Consistently, cryo-EM images of the STING sample in the presence of both C53 and cGAMP showed a large number of tetramers and higher-order oligomers formed by side-by-side packing of the individual dimers (FIG. 6). These results suggested that C53 binds a different site on STING and acts synergistically with cGAMP to promote STING oligomerization. Intriguingly, these higher-order STING oligomers display a curved overall shape, suggesting a preference for membrane with positive curvature (FIG. 6A). This preference might concentrate the STING oligomers at the rim of ER and Golgi and facilitate their anterograde transport, which is known to be important for STING signaling in cells.

### Example 3: Cryo-EM Structure of STING Oligomer Induced by cGAMP and C53

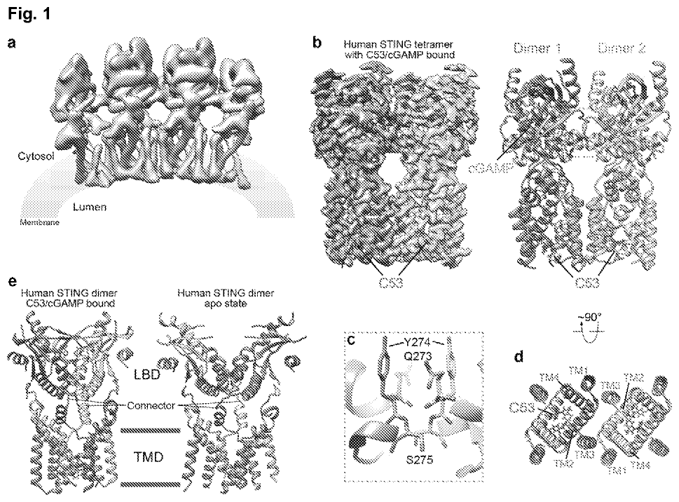

To understand how C53 activates STING, single-particle cryo-EM was used to determine the structure of human STING oligomer with both C53 and cGAMP bound (FIG. 6A-6F). Long oligomeric particles were separated into units containing four dimers for structure determination. This approach allowed the determination of the structure of four STING dimers packed side-by-side in an approximately linear arrangement, which contained all the structural formation for reconstructing higher-order oligomers. The cryo-EM map of the two STING dimers at the center of this structure was further improved to 3.4 Å resolution, which allowed for building a precise model for both the STING tetramer and the bound compounds (FIG. 1).

Previous structural analyses of chicken STING in both the apo- and cGAMP-bound states have shown that cGAMP induces a 180°-rotation of the LBD with respect to the TMD, converting the two connectors linking the LBD and TMD in the STING dimer from the crossover to parallel configuration. The human STING dimer bound to both cGAMP and C53 here exhibited the parallel configuration of the connector, in contrast to the crossover configuration in the apo-state solved previously (FIG. 1E). These observations together supported the notion that the 180°-rotation of LBD was an evolutionarily conserved step in the STING activation mechanism. cGAMP adopted the typical U-shape and bound to the central pocket between the two LBD subunits, consistent with all previous STING/cGAMP complex structures (FIG. 7).

The cryo-EM structure of human STING tetramer showed that two STING dimers bound with both C53 and cGAMP assemble in a side-by-side fashion similar to the chicken STING tetramer (FIG. 1). Both the LBD-LBD and TMD-TMD interactions between the two human STING dimers contributed to the assembly of the tetramer. The LBD-LBD contact between the two STING dimers was mediated largely by the loops connecting the α2 and α3 of LBD (LBDα2-LBDα3 loop) (FIG. 1C). This interface was resolved to low resolution in the cryo-EM map of chicken STING tetramer (PDB ID: 6NT8). The high resolution of the cryo-EM map of human STING tetramer allowed this interface to be resolved in detail. Interestingly, the interaction of the LBDα2-LBDα3 loops was mostly mediated by the backbone hydrogen bonds, with minimal involvement of sidechains. As a result, this interface appeared small and weak, and therefore may not be sufficient for supporting the formation of large STING oligomers. The human STING tetramer was further stabilized by extensive interactions made by the TMD (FIG. 1D), in sharp contrast to the chicken STING tetramer where the TMD barely made any inter-dimer contact. C53 induced conformational changes to the TMD of human STING, and thereby promoting the TMD-TMD interactions. These results explained the synergy of C53 and cGAMP in promoting the oligomerization of human STING.

### Example 4: C53 Binds at a Cryptic Agonist-Binding Site in the TMD of STING

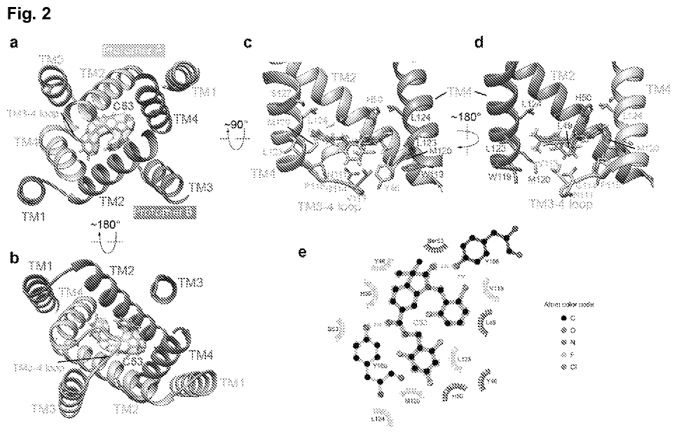

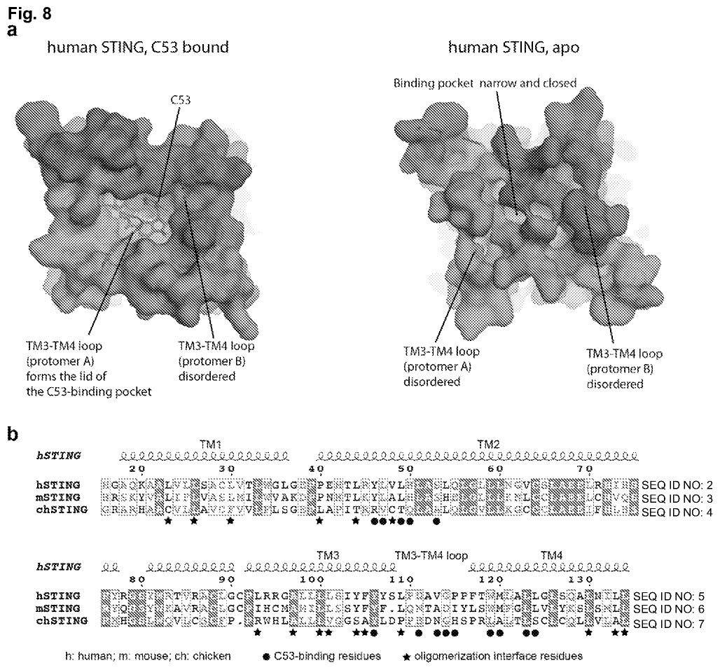

Strikingly, the high-resolution cryo-EM map showed a strong density peak at the luminal side of the TMD of each STING dimer, which was assigned to C53 unequivocally based on clear asymmetric “C”-shape and local chemical environment (FIG. 2 and FIG. 7). This agonist binding mode at STING-TMD had not been observed in any of previous STING structures. In each STING dimer, a single “C”-shaped C53 sat approximately at the C2 axis of the STING dimer, in a deep pocket formed among two TM2s and two TM4s. This pocket in the apo-state of human STING was much smaller and cannot accommodate C53 due to tighter packing among these TM helices (FIG. 8A). Therefore, C53 bound to a cryptic site in human STING TMD through an induced-fit mechanism.

The 2-Cl-6-F-phenyl ring at one end of C53 is juxtaposed closely with the trifluoro-phenyl ring at the other end, resulting in the overall C-shape (FIG. 2A-2B). This compact conformation allowed C53 to fit into the narrow rectangular-shaped cavity between TM2 and TM4 from each subunit of the STING dimer. On one side of the binding pocket, the oxindole core made edge-on packing interactions with TM2 from protomer A of the STING dimer. The outer edge of the oxindole core was accommodated in the space surrounded by Y46, L49, H50 and S53 in TM2 from protomer A (FIG. 2C-2E). Meanwhile, the two methyl groups on the oxindole core were placed close to W119, M120, L123 and L124 in TM4 from protomer B. On the other side, the 2-Cl-6-F-phenyl and trifluoro-phenyl rings of C53 interacted with TM2 from protomer B and TM4 from protomer A. The 2-Cl-6-F-phenyl ring packs its flat face onto a patch composed of Y46, L49, H50 and S53 in TM2 from protomer B. The trifluoro-phenyl wedges between Y46, L47 and H50 of TM2 from protomer B and M120, L123 and L124 of TM4 from protomer A. TM3 contributed to the binding pocket by filling the gap between TM2 and TM4 near the ER/Golgi luminal side. Y106 in TM3 from each protomer made a hydrogen bond with C53.

The mouth of the binding pocket facing the ER/Golgi lumen was sealed by the TM3-TM4 loop from protomer A of the STING dimer (FIGS. 2C and 2D). V113, G114 and P115 in this loop pack closely with the bottom face of C53. N111 is partially buried in the binding pocket, contributing to the stability of the loop conformation by making hydrogen bonds with the backbone of the loop. The TM3-TM4 loop from protomer B was mostly disordered, while modelling it to the same conformation as that in protomer A led to severe clashes between the two loops. Therefore, C53 as an asymmetric molecule induced obvious asymmetry in the TMD of the STING dimer, in contrast to cGAMP, for which its binding site in the LBD was overall symmetric or shows subtle asymmetry in some cases. While most C53-contacting residues from the TM helices were conserved between human and mouse STING, those in the TM3-TM4 loop were not (FIG. 8B). This divergence in the TM3-TM4 loop provides an explanation for the observation that C53 activated human STING but not mouse STING. Similar species specificity had been displayed by DMXAA (5,6-dimethylxanthenone-4-acetic acid), which acts as an agonist for mouse STING by targeting the cGAMP-binding pocket but does not activate human STING.

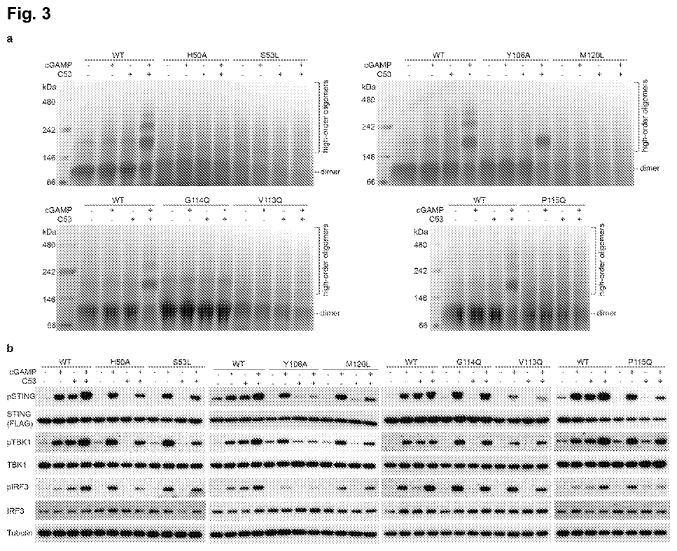

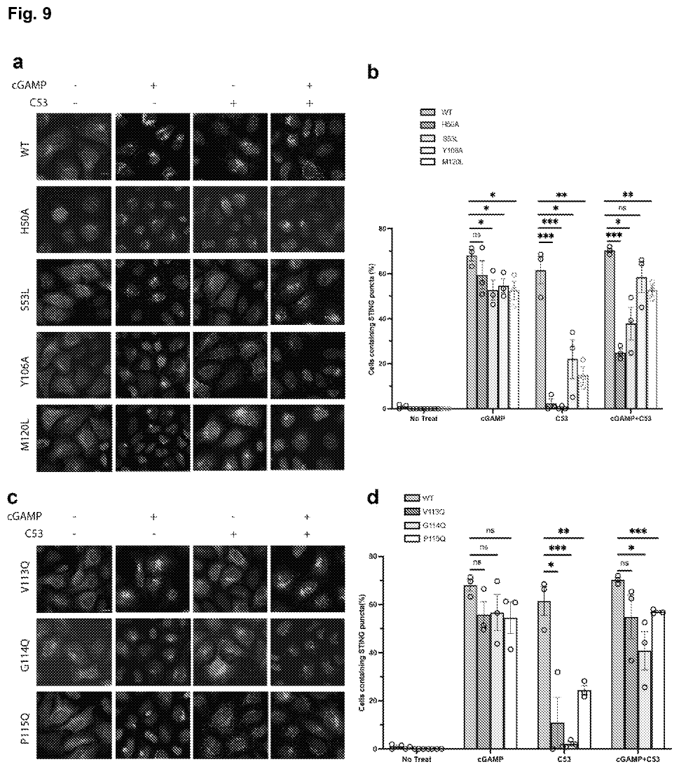

To validate this binding mode of C53, mutations were introduced to residues in the binding pocket (H50A, S53L, Y106A, and M120L) and the TM3-TM4 loop (V113Q, G114Q and P115Q) of STING. Native-gel results showed that, compared to STING wild type (STING-WT), these mutants showed no or greatly reduced high-order oligomerization upon stimulation by C53 and cGAMP (FIG. 3A). Consistently, these mutations also led to significantly decreased C53-induced puncta formation of STING in cells, which reflects the oligomerization of STING on ER/Golgi membrane (FIG. 9). As expected, STING showed a diffuse pattern in cells in the absence of an agonist. In addition, the cell-based functional assay showed that C53 and cGAMP together triggered higher levels of phosphorylation of STING-WT, TBK1 and IRF3 than either agonist alone, suggesting that STING could reach higher activation levels when bound to both C53 and cGAMP simultaneously (FIG. 3B). The mutations in the C53-binding pocket abrogated the phosphorylation of these protein triggered by C53, but cGAMP-induced phosphorylation remained largely unaffected (FIG. 3B). These results together confirmed that C53 activates STING by targeting the binding site in the TMD.

### Example 5: C53 Induces Conformational Change of the TMD to Promote STING Oligomerization

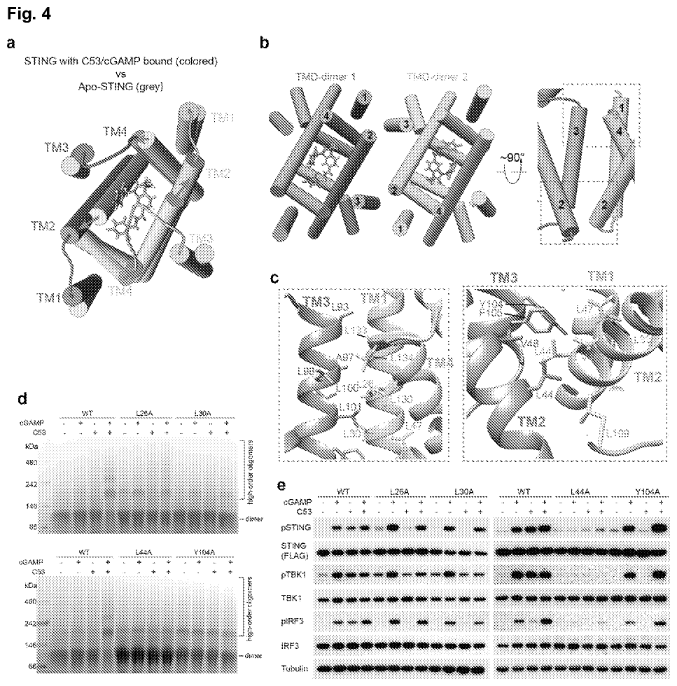

Structural comparison of STING without or with C53 bound revealed that TMs that surround C53 in the STING dimer, especially TM2 and TM4, undergo substantial sideway expansion upon C53 binding (FIG. 4A and FIG. 8A). Such conformational rearrangements of TM2 and TM4 were necessary for providing sufficient space for C53 binding. As mentioned above, in the tetrameric structure of chicken STING with cGAMP bound, the two STING dimers packed side-by-side mainly through the interaction between the LBDs, while no direct interactions were observed between the TMDs. In contrast, C53-induced dilation of the TMD in human STING allowed the TM helices to be engaged in the TMD-TMD interaction between two STING dimers (FIG. 4B, 4c). The TMD interface contained two four-helix bundles that were related by the C2-symmetry of the tetramer. Each four-helix bundle was formed by one TM3 from one STING dimer and one set of TM1, TM2 and TM4 from the other dimer (FIG. 4B). Extensive packing interactions were made by hydrophobic residues from the four helices, including L23, L26 and L30 from TM1, L44 from TM2 as well as L93, A97, L100, L101, Y104, F105 and L109 from TM3 (FIG. 4C). The two four-helix bundles were connected at the ER/Golgi luminal side by the N-terminal residues L44 and V48 from TM2. Most of the residues in the TMD-TMD interaction were conserved between human and mouse STING, but not in chicken STING (FIG. 8B), consistent with the observation that chicken STING appeared less dependent on the TMD interaction for the high-order oligomerization. The TMD interface buries ˜2600 Å² solvent accessible area, much larger than the LBD interface (˜740 Å²). Notably, the TMDs between the two STING dimers associated more closely on the luminal side than on the cytosolic side, leading to the overall curvature of high-order STING oligomers.

The open-ended nature of the STING tetramer allowed assembly of larger oligomers by adding additional dimeric units to both sides, enabling STING to serve as a signaling platform for efficient recruitment and activation of TBK1 and IRF3. The induction of the TMD-TMD interaction therefore likely underlies the activating effect of C53 on STING. To test this model, mutations were introduced to the C53-induced TMD-TMD interface, including L26A, L30A, L44A and Y104A. Compared to STING-WT, these mutants showed substantially reduced oligomerization in solution in the presence of both C53 and cGAMP (FIG. 4D). Likewise, the mutations led to reduced puncta formation of STING in cells upon stimulation by C53 (FIG. 10). The mutations also largely abolished C53-induced phosphorylation of STING, TBK1 and IRF3 in cells (FIG. 4E). cGAMP-induced phosphorylation of these proteins was abrogated by L44A, but unaffected by the other three mutations (FIG. 4E). These results suggested that the TMD-TMD interface is essential for C53-mediated STING activation. Activation of STING by cGAMP also involved this interface, but it may be less dependent on it, especially when the concentration of cGAMP is high (1 μM in these experiments).

### Discussion of the Examples 2-5

In this work, high-resolution cryo-EM structure of human STING in complex with both cGAMP and C53 defined a novel agonist binding site in the TMD of STING, paving the way for developing more agonists that target this site. Compounds that target the TMD site might be better STING agonists than cGAMP mimetics for therapeutic purposes because they are more hydrophobic and therefore more permeable to the cell membrane. In addition, structural analyses clarified the direct role of the TMD in the oligomerization and activation of STING. The coupling between the induced opening of the TMD pocket by C53 and the high-order oligomerization of human STING suggested that this conformational change might be an integral part of its activation mechanism in vivo. This notion raises the question how this conformational change is triggered in the cell. It is possible that cGAMP-induced 180°-rotation of the LBD may allosterically induce the open conformation of the TMD on the ER/Golgi membrane in the cell, which could not be recapitulated with purified protein in vitro. Alternatively, a cellular endogenous ligand may bind to the TMD pocket and act as a secondary activator to facilitate cGAMP-induced STING activation.

### Example 6: Synthesis of STING Modulator—UT009

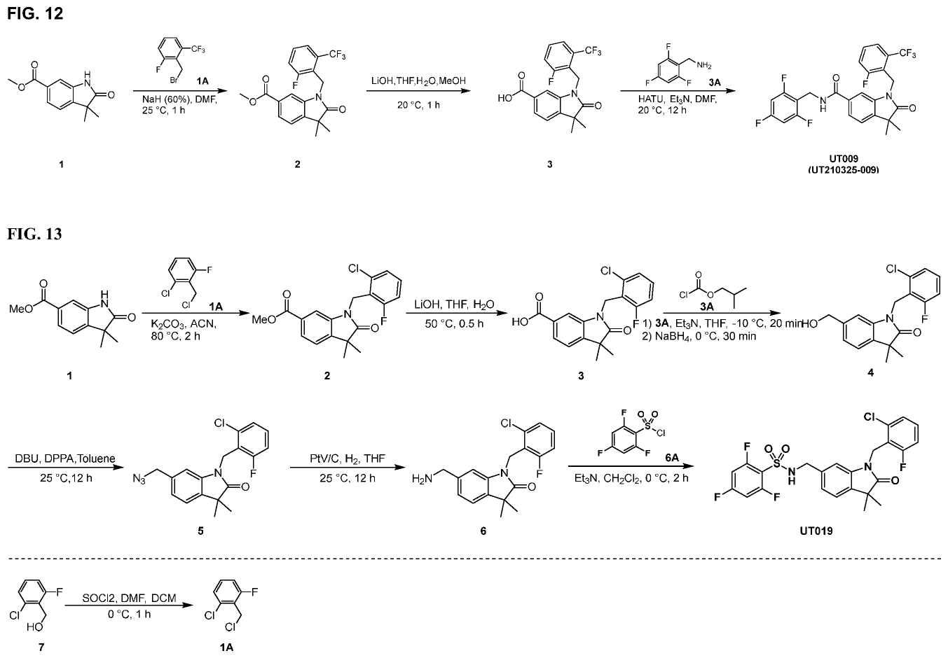

Compound UT009 was synthesized according to the scheme shown in FIG. 12. Individual steps to synthesize intermediate compounds are shown below as Schemes 1-3.

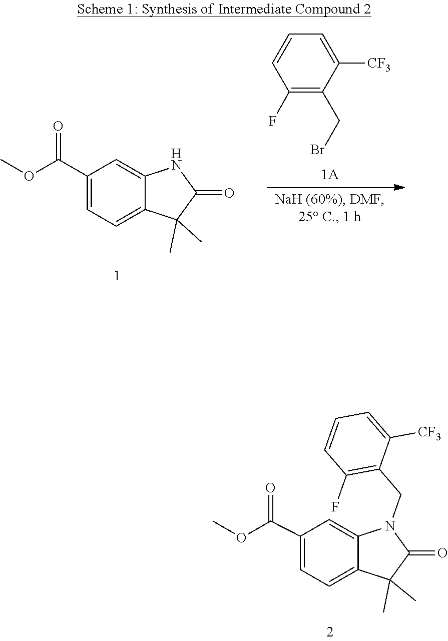 [SMILES: COC(=O)c1ccc2c(c1)NC(=O)C2(C)C ; Fc1cccc(C(F)(F)F)c1CBr ; COC(=O)c1ccc2c(c1)N(Cc1c(F)cccc1C(F)(F)F)C(=O)C2(C)C]

To a solution of Compound 1 (300 mg, 1.37 mmol, 1 eq) in DMF (3 mL) was added NaH (82.10 mg, 2.05 mmol, 60% purity, 1.5 eq) and stirred at 0° C. for 0.5 hour. Then to the mixture was added compound 1A (422.05 mg, 1.64 mmol, 1.2 eq), the mixture was stirred at 25° C. for 0.5 hour. The reaction mixture was diluted with water (5 mL) and extracted with EtOAc (5 mL×3). The combined organic layers were washed with brine (10 mL), dried over anhydrous Na2SO4, filtered and concentrated under reduced pressure to give a residue which was purified by flash silica gel chromatography (ISCO®; 4 g Sepa Flash® Silica Flash Column, Eluent of 0˜20% Ethyl acetate/Petroleum ether gradient @40 mL/min) to give Compound 2 (400 mg, 1.01 mmol, 73.94% yield) as a white solid.

LCMS: MS (ESI) Retention time: 0.577 min, [M+1]⁺=396.3

¹H NMR (400 MHz, DMSO-d₆) δ=7.63 (dd, J=7.6, 0.8 Hz, 1H), 7.45 (d, J=8.0 Hz, 1H), 7.34-7.26 (m, 1H), 7.19 (s, 1H), 7.16-7.13 (m, 1H), 7.12-7.05 (m, 1H), 5.10 (s, 2H), 3.73 (s, 3H), 1.30 (s, 6H).

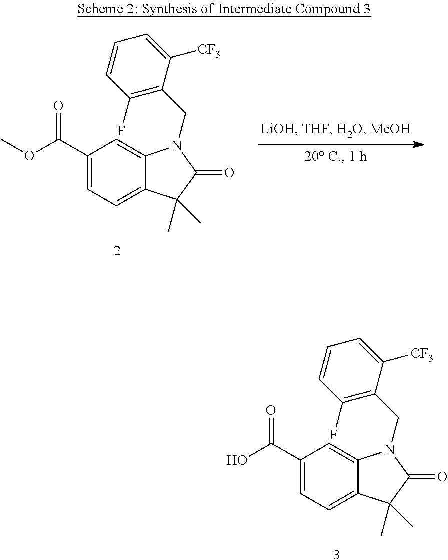

To a solution of Compound 2 (400 mg, 1.01 mmol, 1 eq) in THE (1 mL) and MeOH (1 mL) was added LiOH (121.15 mg, 5.06 mmol, 5 eq) in H2O (1 mL). The mixture was stirred at 20° C. for 1 hour. The reaction mixture was concentrated under the reduced pressure to obtain Compound 3 (530 g, crude) as a white solid and the mixture was used directly for the next step.

LCMS: MS (ESI) Retention time: 0.468 min, [M+1]⁺=382.0

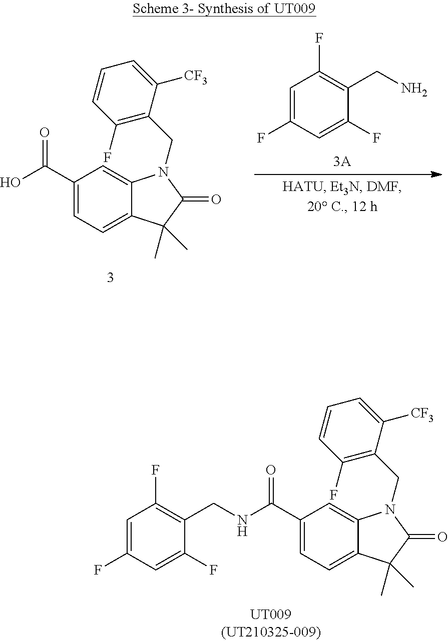

To a solution of Compound 3 (100 mg, 262.25 umol, 1 eq) and Compound 3A (42.25 mg, 262.25 umol, 1 eq) in DMF (2 mL) was added HATU (149.57 mg, 393.37 umol, 1.5 eq) and DIEA (101.68 mg, 786.74 umol, 137.04 μL, 3 eq). The mixture was stirred at 20° C. for 12 hour. The reaction mixture was concentrated under the reduced pressure to give the crude product which was purified by prep-HPLC (column: Phenomenex Luna C18 100*30 mm*5 um; mobile phase: [water (FA)-ACN]; B %: 47%-87%, 8 min) and lyophilized to obtain UT009 (6.86 mg, 12.74 μmol, 4.86% yield, 97.4% purity) as a yellow solid.

LCMS: LCMS: MS (ESI) Retention time: 0.528 min, [M+1]⁺=525.1

HPLC: Retention time: 3.684 min, Purity=98.11%, 220 nm

¹H NMR (400 MHz, DMSO-d₆) δ=8.73 (t, J=5.2 Hz, 1H), 7.72-7.64 (m, 1H), 7.64-7.56 (m, 1H), 7.55-7.47 (m, 2H), 7.45-7.40 (m, 1H), 7.26 (d, J=0.8 Hz, 1H), 7.17 (t, J=8.8 Hz, 2H), 5.10 (s, 2H), 4.42 (d, J=5.0 Hz, 2H), 1.27 (s, 6H)

### Example 7: Synthesis of STING Modulator—UT019

Compound UT019 was synthesized according to the scheme illustrated in FIG. 13. Intermediate compounds were synthesized according to Schemes 4 to 10 below.

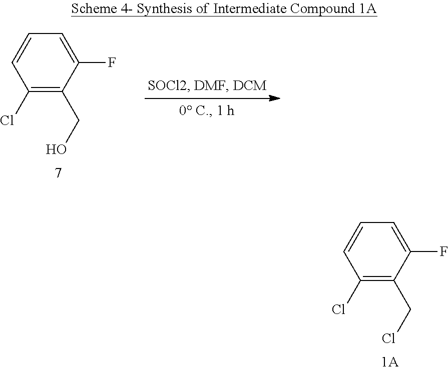 [SMILES: OCc1c(F)cccc1Cl ; Fc1cccc(Cl)c1CCl]

To a solution of Compound 7 (3 g, 18.68 mmol, 1 eq) in DCM (30 mL) was added SOCl₂ (4.45 g, 37.37 mmol, 2.71 mL, 2 eq), Then was added DMF (136.56 mg, 1.87 mmol, 143.75 uL, 0.1 eq). The mixture was stirred at 0° C. for 1 h. The reaction mixture was concentrated under the reduced pressure to obtain Compound 1A (4.49 g, crude) as a colorless liquid.

¹H NMR (400 MHz, DMSO-d₆): δ=7.51-7.44 (m, 1H), 7.43-7.37 (m, 1H), 7.37-7.27 (m, 1H), 5.75 (s, 1H), 4.82 (s, 2H)

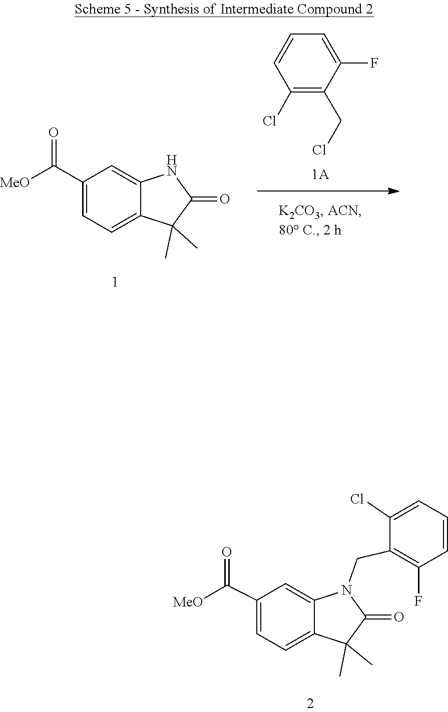 [SMILES: COC(=O)c1ccc2c(c1)NC(=O)C2(C)C ; Fc1cccc(Cl)c1CCl ; COC(=O)c1ccc2c(c1)N(Cc1c(F)cccc1Cl)C(=O)C2(C)C]

To a solution of Compound 1 (2 g, 9.12 mmol, 1 eq) and Compound 1A (1.63 g, 9.12 mmol, 1.17 mL, 1 eq) in ACN (30 mL) was added K2CO3 (7.56 g, 54.74 mmol, 6 eq). The mixture was stirred at 80° C. for 2 h. The reaction mixture was filtered and the filtrate was concentrated under the reduced pressure to give the product. The residue was purified by flash silica gel chromatography (ISCO®; 20 g SepaFlash® Silica Flash Column, Eluent of 0˜13% Ethyl acetate/Petroleum ether gradient @80 mL/min) and concentrated under the reduced pressure to obtain Compound 2 (3.60 g, crude) as an off-white solid.

LCMS: MS (ESI) Retention time: 0.653 min, [M+1]⁺=361.9 1H NMR (400 MHz, DMSO-d6) (EC2637-135-P1A): δ=7.65 (dd, J=1.2, 7.6 Hz, 1H), 7.51 (d, J=8.0 Hz, 1H), 7.45-7.34 (m, 3H), 7.30-7.21 (m, 1H), 5.08 (s, 2H), 3.81 (s, 3H), 1.30 (s, 6H)

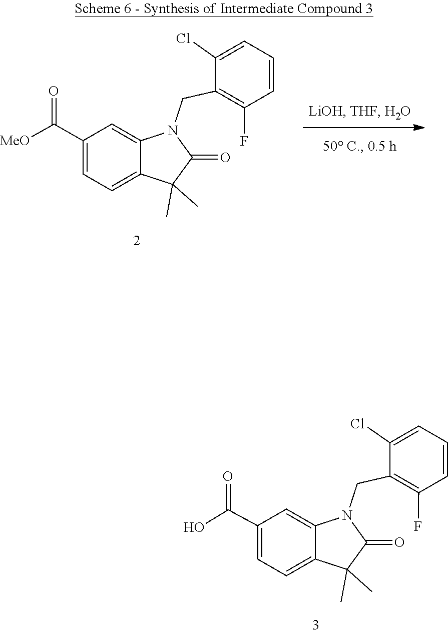 [SMILES: COC(=O)c1ccc2c(c1)N(Cc1c(F)cccc1Cl)C(=O)C2(C)C ; CC1(C)C(=O)N(Cc2c(F)cccc2Cl)c2cc(C(=O)O)ccc21]

To a solution of Compound 2 (3.27 g, 9.04 mmol, 1 eq) in THF (15 mL) was added LiOH·H2O (568.92 mg, 13.56 mmol, 1.5 eq) in H2O (15 mL). The mixture was stirred at 60° C. for 2 h. Acidify the reaction mixture by adding, with shaking, 10 mL of HCl until pH around 3, and then extracted with EtOAc (20 ml×3). The combined organic layers were dried over anhydrous Na2SO4, filtered and concentrated to obtain Compound 3 (3.10 g, 8.92 mmol, 98.69% yield) as a yellow solid.

LCMS: MS (ESI) Retention time: 0.563 min, [M+1]⁺=348.0

¹H NMR (400 MHz, DMSO-d₆): δ=13.26-12.47 (m, 1H), 7.64 (dd, J=1.2, 7.6 Hz, 1H), 7.50-7.33 (m, 4H), 7.25 (ddd, J=1.2, 8.0, 10.0 Hz, 1H), 5.08 (s, 2H), 1.30 (s, 6H)

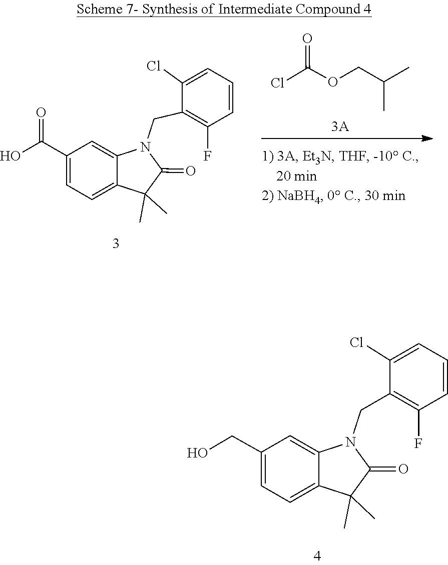

To a solution of Compound 3 (500 mg, 1.44 mmol, 1 eq) and TEA (174.58 mg, 1.73 mmol, 240.14 uL, 1.2 eq) in THF (10 mL) was cooled to −10° C. and Compound 3A (216.00 mg, 1.58 mmol, 207.69 uL, 1.1 eq) was added. After 20 min was added NaBH4 (435.15 mg, 11.50 mmol, 8 eq) at 0° C. The mixture was stirred at 0° C. for 30 min. The reaction was quenched by the addition of 10% citric acid (50 mL). The mixture was extracted with ethyl acetate (20 mL×3). The combined organic extracts were washed with brine (20 mL×3), dried, filtered and concentrated. The residue was purified by flash silica gel chromatography (ISCO®; 12 g SepaFlash® Silica Flash Column, Eluent of 0˜23% Ethyl acetate/Petroleum ether gradient @50 mL/min) and concentrated to obtain Compound 4 (337 mg, 1.01 mmol, 70.22% yield) as a white solid.

LCMS: MS (ESI) Retention time: 0.436 min, [M+1]⁺=333.9

¹H NMR (400 MHz, DMSO-d₆): δ=7.49-7.33 (m, 2H), 7.33-7.16 (m, 2H), 7.07-6.73 (m, 2H), 5.10 (t, J=5.6 Hz, 1H), 5.02 (s, 1H), 4.37 (d, J=5.2 Hz, 2H), 1.26 (s, 6H)

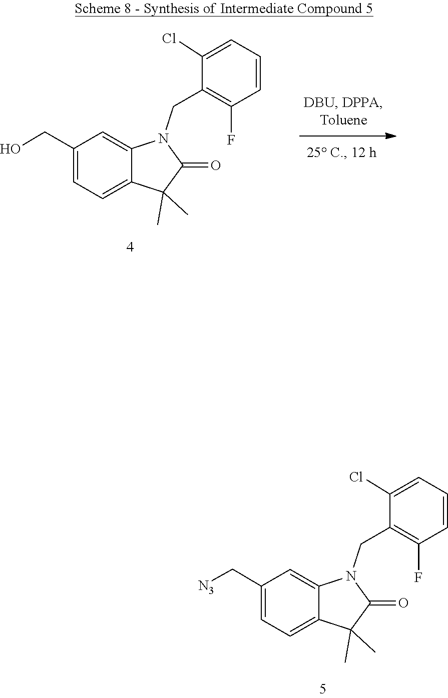 [SMILES: CC1(C)C(=O)N(Cc2c(F)cccc2Cl)c2cc(CO)ccc21 ; CC1(C)C(=O)N(Cc2c(F)cccc2Cl)c2cc(CN=[N+]=[N-])ccc21]

To a solution of Compound 4 (180 mg, 539.27 umol, 1 eq) in toluene (2 mL) was added DBU (106.73 mg, 701.05 umol, 105.67 uL, 1.3 eq) and DPPA (178.09 mg, 647.13 umol, 140.23 uL, 1.2 eq). The mixture was stirred at 25° C. for 12 h. The mixture was extracted with ethyl acetate (10 mL×3). The combined organic extracts were washed with brine (10 mL×3), dried, filtered and concentrated. The residue was purified by prep-TLC (SiO₂, Petroleum ether:Ethyl acetate=5:1, Rf=0.2) and eluted with acetonitrile (5 mL) to obtain Compound 5 (160 mg, 436.94 μmol, 81.02% yield, 97.982% purity) as a white solid.

LCMS: MS (ESI) Retention time: 0.536 min, [M+1]⁺=360.0

¹H NMR (400 MHz, DMSO-d₆): δ=7.48-7.34 (m, 3H), 7.28-7.20 (m, 1H), 6.99 (d, J=7.2 Hz, 1H), 6.80 (s, 1H), 5.04 (s, 2H), 4.36 (s, 2H), 1.28 (s, 6H)

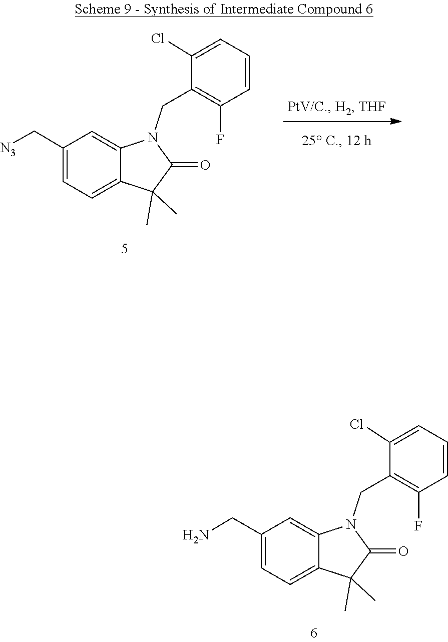 [SMILES: CC1(C)C(=O)N(Cc2c(F)cccc2Cl)c2cc(CN=[N+]=[N-])ccc21 ; CC1(C)C(=O)N(Cc2c(F)cccc2Cl)c2cc(CN)ccc21]

To a solution of Compound 5 (140 mg, 390.19 μmol, 1 eq) in THE (2 mL) was added PtV/C (10.19 mg, 39.02 umol, 0.1 eq). The mixture was stirred at 25° C. for 12 h under H2 (15 psi) atmosphere. The mixture was filtered and concentrated under reduced pressure to give Compound 6 (129 mg, 387.62 μmol, 99.34% yield) as an off-white solid.

LCMS: MS (ESI) Retention time: 0.342 min, [M+1]⁺=315.9.

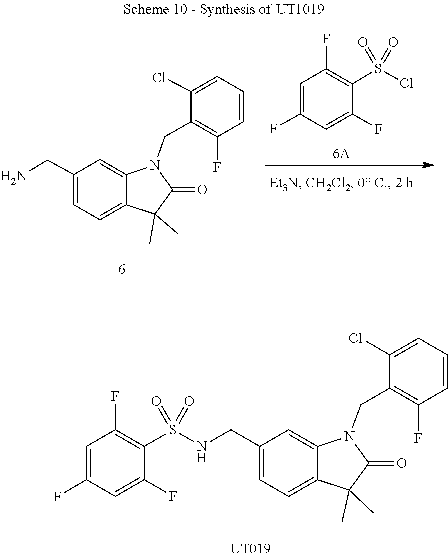

A mixture of Et3N (45.61 mg, 450.72 umol, 62.73 uL, 3 eq) in CH2Cl2 (1 mL) was Compound 6 (50 mg, 150.24 umol, 1 eq), and was degassed and purged with N₂ for 3 times, then was added Compound 6A (34.64 mg, 150.24 umol, 1 eq) in CH2Cl2 (1 mL) and then the mixture was stirred at 0° C. for 2 h under N₂ atmosphere. The mixture was concentrated under reduced pressure to give a residue. The residue was purified by prep-HPLC (column: Phenomenex Luna C18 100*30 mm*5 um; mobile phase: [water (FA)-ACN]; B %: 43%-73%, 8 min) and lyophilized to obtain UT019 (25.97 mg, 48.10 μmol, 32.01% yield, 97.591% purity) as a white solid.

LCMS: MS (ESI) Retention time: 0.511 min, [M+1]⁺=527.2.

HPLC: Retention time: 0.511 min, Purity=97.59%, 220 nm

¹H NMR (400 MHz, DMSO-d₆) δ=8.78 (s, 1H), 7.45-7.34 (m, 2H), 7.29-7.21 (m, 1H), 7.19-7.11 (m, 3H), 6.86 (d, J=6.8 Hz, 1H), 6.63 (s, 1H), 4.96 (s, 2H), 4.06 (s, 2H), 1.21 (s, 6H)

### Example 8—Synthesis of STING Modulator—UT073

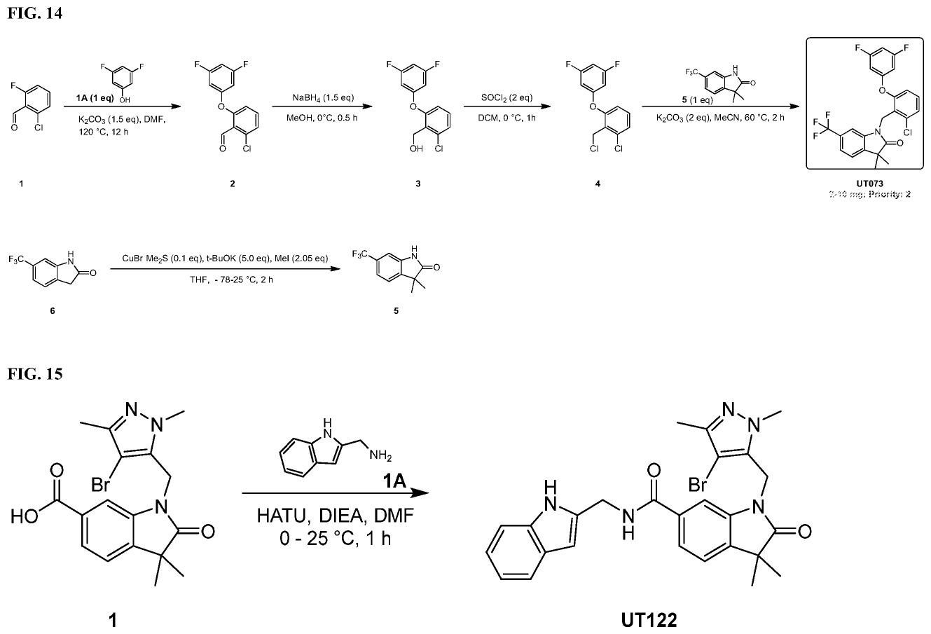

Synthesis of UT073 occurred according to the scheme shown in FIG. 14, which is broken down into Schemes 11-15 below.

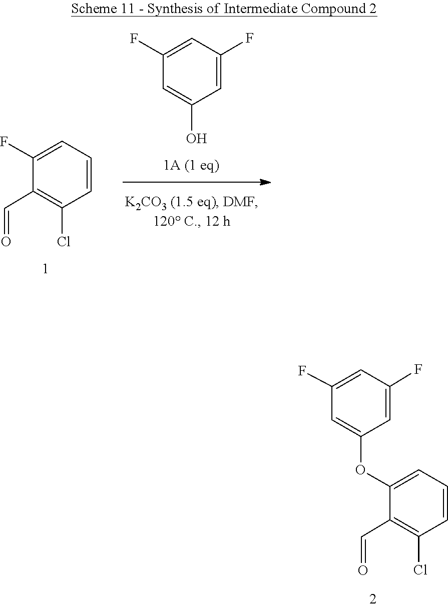 [SMILES: Oc1cc(F)cc(F)c1 ; O=Cc1c(F)cccc1Cl ; O=Cc1c(Cl)cccc1Oc1cc(F)cc(F)c1]

To a solution of compound 1 (5.24 g, 33.05 mmol, 1 eq) and compound 1A (4.3 g, 33.05 mmol, 1 eq) in DMF (50 mL) was added K₂CO₃ (6.85 g, 49.58 mmol, 1.5 eq). The mixture was stirred at 120° C. for 12 h. The reaction mixture was partitioned between EtOAc (100 mL×3) and water (100 mL). The combined organic layers were dried over anhydrous Na₂SO₄, filtered and concentrated under reduced pressure to give Compound 2 (3.5 g, crude) as a blackish brown solid.

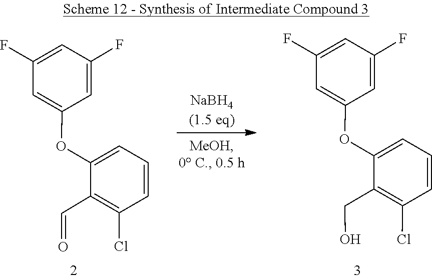 [SMILES: O=Cc1c(Cl)cccc1Oc1cc(F)cc(F)c1 ; OCc1c(Cl)cccc1Oc1cc(F)cc(F)c1]

To a solution of Compound 2 (3.5 g, 13.03 mmol, 1 eq) in MeOH (40 mL) was added NaBH₄ (739.35 mg, 19.54 mmol, 1.5 eq) at 0° C. The mixture was stirred at 0° C. for 0.5 h. The reaction was quenched by MeOH (100 mL). The mixture was filtered and concentrated under the reduced pressure to obtain Compound 3 (3.46 g, crude) as a yellow oil.

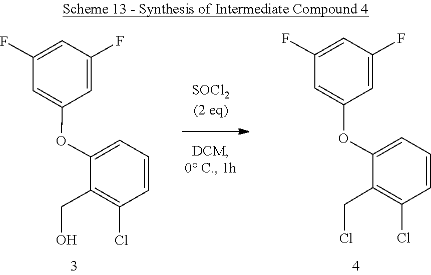 [SMILES: OCc1c(Cl)cccc1Oc1cc(F)cc(F)c1 ; Fc1cc(F)cc(Oc2cccc(Cl)c2CCl)c1]

To a solution of Compound 3 (500 mg, 2.39 mmol, 1 eq) in THE (10 mL) was added Pd (127.15 mg, 119.48 μmol, 10% purity, 0.05 eq) under N₂ atmosphere. The suspension was degassed and purged with H₂ for 3 times. The mixture was stirred under H₂ (15 Psi) at 25° C. for 2 h. The residue was filtered and the filtrate was concentrated in vacuum to obtain Compound 4 (400 mg, crude) as a yellow oil.

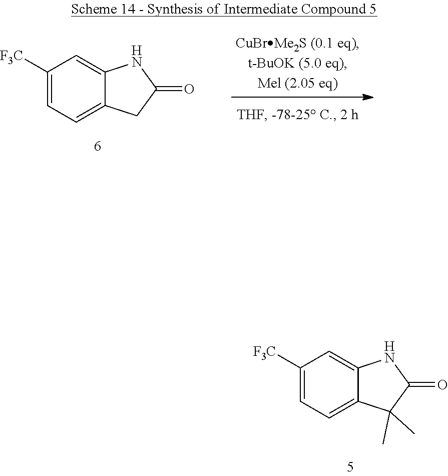 [SMILES: O=C1Cc2ccc(C(F)(F)F)cc2N1 ; CC1(C)C(=O)Nc2cc(C(F)(F)F)ccc21]

To a solution of Compound 6 (1.0 g, 4.97 mmol, 1 eq) in THE (20 mL) was added bromocopper;methylspLfanylmethane (102.21 mg, 497.15 μmol, 0.1 eq) and t-BuOK (2.79 g, 24.86 mmol, 5.0 eq) at −78° C. After addition, mixture was added CH₃I (1.45 g, 10.19 mmol, 634.47 μL, 2.05 eq) in THE (20 mL). And the mixture was stirred at 25° C. for 2 h. The mixture was poured into 5% of citric acid (100 mL) and extracted with EtOAc (100×3). The combined organic phase was washed with brine (100 mL), dried with anhydrous Na₂SO₄, filtered and concentrated in vacuum. The residue was purified by prep-HPLC (column: Phenomenex Luna C18 200*40 mm*10 um; mobile phase: [water (FA)-ACN]; B %: 35%-65%, 10 min) to obtain Compound 5 (427 mg, 1.86 mmol, 37.47% yield) as a white solid.

¹H NMR (400 MHz, CDCl₃) δ=8.66 (s, 1H), 7.37-7.32 (m, 1H), 7.32-7.28 (m, 1H), 7.19 (s, 1H), 1.44 (s, 6H)

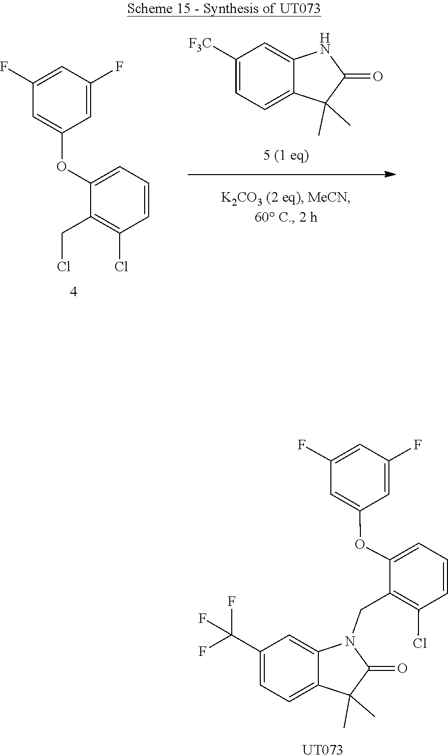

A mixture of Compound 4 (100 mg, 345.90 μmol, 1 eq), Compound 5 (79.28 mg, 345.90 μmol, 1 eq), K₂CO₃ (95.61 mg, 691.79 μmol, 2.0 eq) in MeCN (4 mL) was degassed and purged with N₂ for 3 times, and then the mixture was stirred at 60° C. for 2 h under N₂ atmosphere. The mixture was filtered and concentrated in vacuum to give a residue.

The residue was purified by prep-HPLC (column: Phenomenex luna C18 150*25 mm*10 um; mobile phase: [water (FA)-ACN]; B %: 70%-100%, 9 min) to give Compound UT073 (34.63 mg, 70.43 μmol, 20.36% yield, 98% purity) was obtained as a off-white solid.

LCMS: MS (ESI) Retention time: 0.625 min, [M+1]⁺=482.0

HPLC: Retention time: 4.835 min, Purity=98.07%, 220 nm

¹H NMR (400 MHz, DMSO-d₆) δ=7.51 (d, J=7.6 Hz, 1H), 7.44-7.37 (m, 2H), 7.34 (dd, J=0.8, 7.6 Hz, 1H), 7.23 (s, 1H), 7.04 (dd, J=2.0, 7.2 Hz, 1H), 6.96 (tt, J=2.4, 9.6 Hz, 1H), 6.46 (dd, J=2.2, 8.4 Hz, 2H), 5.12 (s, 2H), 1.20 (s, 6H)

### Example 9—Synthesis of STING Modulator—UT122

Compound UT122 was synthesized according to FIG. 15, reproduced as Scheme 16, below.

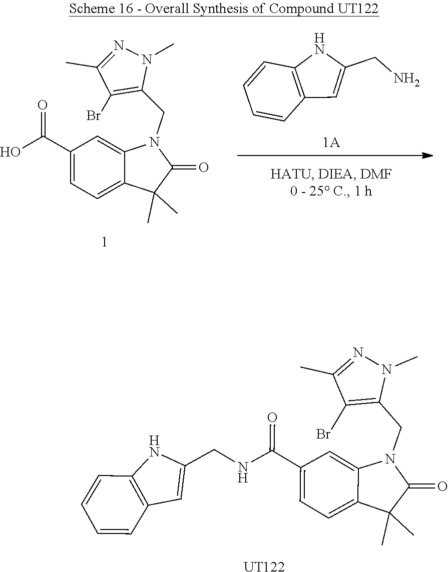 [SMILES: Cc1nn(C)c(CN2C(=O)C(C)(C)c3ccc(C(=O)O)cc32)c1Br ; NCc1cc2ccccc2[nH]1 ; Cc1nn(C)c(CN2C(=O)C(C)(C)c3ccc(C(=O)NCc4cc5ccccc5[nH]4)cc32)c1Br ; "UT122"]

To a solution of Compound 1 (100 mg, 254.94 umol, 1 eq) in DMF (1.5 mL) was added HATU (145.40 mg, 382.41 umol, 1.5 eq) at 0° C., and the mixture was stirred at 0° C. for 30 min. After was added Compound 1A (37.27 mg, 254.94 umol, 1 eq) and DIEA (98.85 mg, 764.83 umol, 133.22 uL, 3 eq), and the mixture was stirred at 25° C. for 30 min. The residue was purified by Prep-HPLC (41%-71% MeCN in water (FA), 9 min), to obtain UT122 (10.04 mg, 19.24 μmol, 7.55% yield, 99.74% purity) as a white solid.

LCMS: MS (ESI) Retention time: 0.613 min, [M+1]⁺=521.8

HPLC: Retention time: 3.651 min, Purity=99.74%, 220 nm

¹H NMR (400 MHz, DMSO-d₆)=10.92 (s, 1H), 8.88 (t, J=5.6 Hz, 1H), 7.64 (dd, J=7.6, 1.2 Hz, 1H), 7.53-7.39 (m, 3H), 7.38-7.28 (m, 1H), 7.05-6.98 (m, 1H), 6.97-6.90 (m, 1H), 6.27 (d, J=0.8 Hz, 1H), 4.97 (s, 2H), 4.60 (d, J=5.6 Hz, 2H), 3.75 (s, 3H), 2.06 (s, 3H), 1.34 (s, 6H).

### Example 10: Activity of STING Antagonists

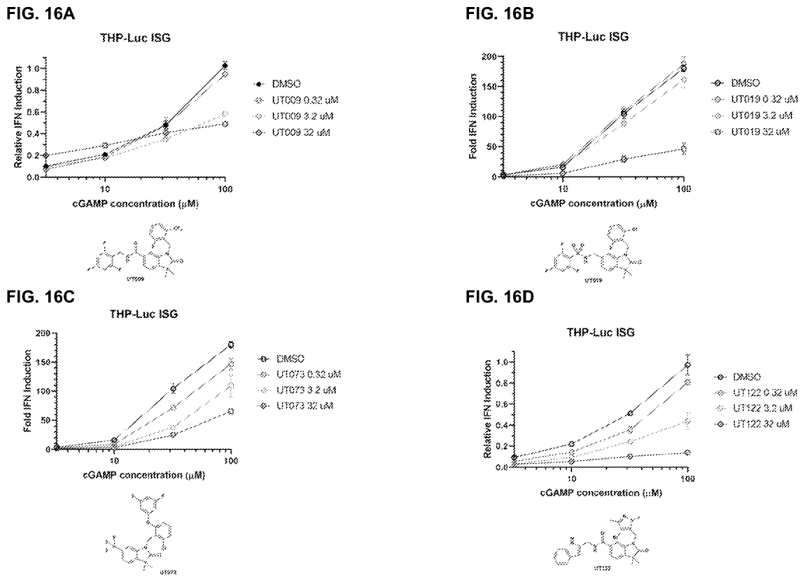

Compounds UT009, UT019, UT073 and UT122 were tested for their ability to interfere with cGAMP induced STING signaling. FIGS. 16A-16D show dose response plots of cGAMP induced IFN induction in the presence of increasing concentrations of each compound. A dose-dependent suppression of interferon production is observed in each of the four compounds tested.

### Example 11: Activity of STING Agonists

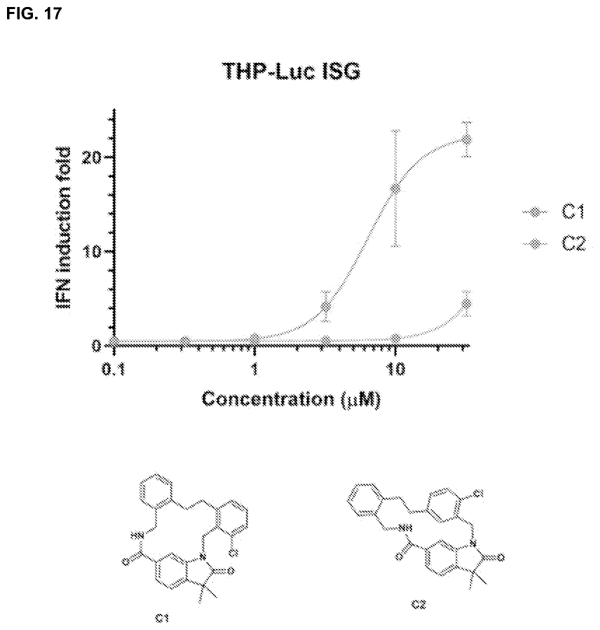

Two compounds were also tested for their ability to induce STING signaling (e.g., act as agonists or activators). Agonist activity was measured by measuring interferon levels in the presence of increasing concentrations of each compound. Dose response curves for each of the two compounds (C1 and C2) are shown in FIG. 17.

### Example 12: Synthesis of STING Modulator—UT017

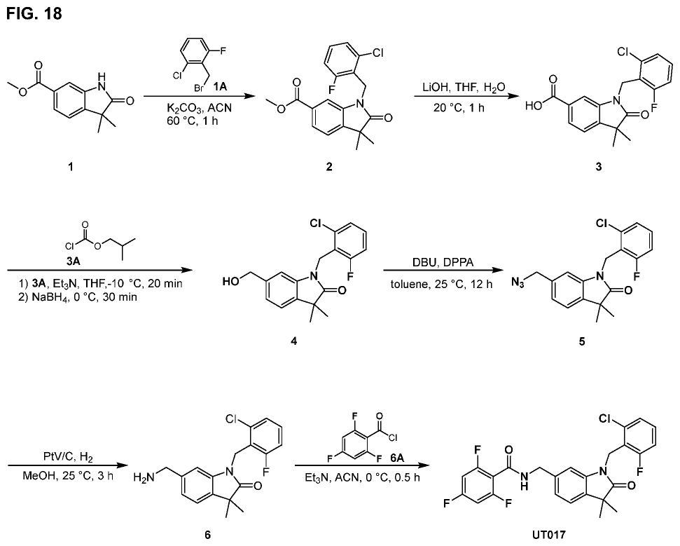

Compound UT017 was synthesized according to FIG. 18, which is broken down into Scheme 17-22, below.

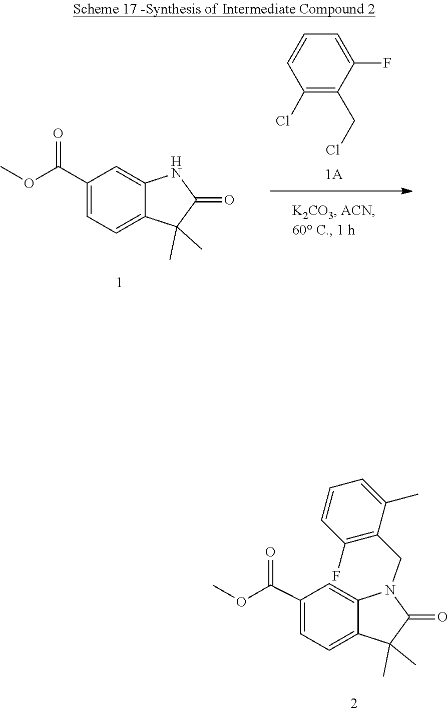 [SMILES: COC(=O)c1ccc2c(c1)NC(=O)C2(C)C ; Fc1cccc(Cl)c1CCl ; COC(=O)c1ccc2c(c1)N(Cc1c(C)cccc1F)C(=O)C2(C)C]

To a solution of Compound 1 (1.6 g, 7.30 mmol, 1 eq) and Compound 1A (3.26 g, 14.60 mmol, 2 eq) in ACN (16 mL) was added K2CO3 (2.02 g, 14.60 mmol, 2 eq). The mixture was stirred at 60° C. for 1 h. The residue was purified by flash silica gel chromatography (ISCO®; 40 g SepaFlash® Silica Flash Column, Eluent of 0˜35% Ethyl acetate/Petroleum ether gradient @80 mL/min) to obtain Compound 2 (800 mg, 2.15 mmol, 29.49% yield, 97.316% purity) as a white solid.

LCMS: [M+H]⁺=362.0

1H NMR (400 MHz, DMSO-d6) δ=7.66 (d, J=7.6 Hz, 1H), 7.51 (d, J=7.6 Hz, 1H), 7.41-7.35 (m, 3H), 7.25 (t, J=8.0 Hz, 1H), 5.08 (s, 2H), 3.81 (s, 3H), 1.30 (s, 6H).

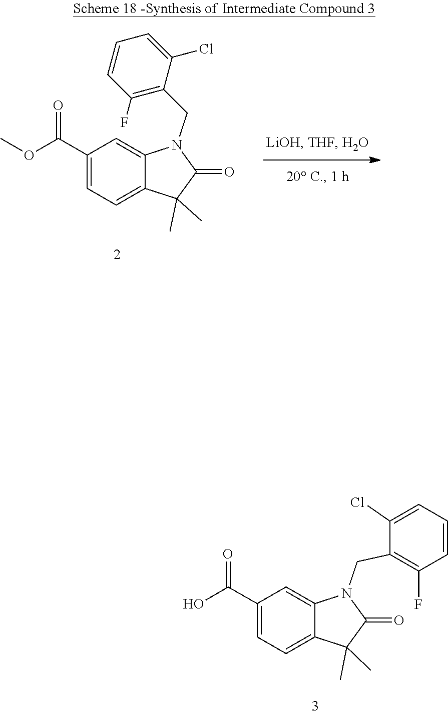 [SMILES: COC(=O)c1ccc2c(c1)N(Cc1c(F)cccc1Cl)C(=O)C2(C)C ; CC1(C)C(=O)N(Cc2c(F)cccc2Cl)c2cc(C(=O)O)ccc21]

To a solution of Compound 2 (800 mg, 2.21 mmol, 1 eq) in THF (1.6 mL) and MeOH (1.6 mL) was added LiOH·H2O (371.16 mg, 8.84 mmol, 4 eq) in H2O (0.8 mL). The mixture was stirred at 20° C. for 1 h. Acidify the reaction mixture by adding, with shaking, 6 mL of HCL until pH around 3, and then extracted with EtOAc (20 ml×3). The combined organic layers were dried over anhydrous Na₂SO4, filtered and concentrated to give a residue to obtain Compound 3 (654 mg, 1.88 mmol, 85.05% yield) as an off-white solid.

LCMS: [M+H]⁺=348.0

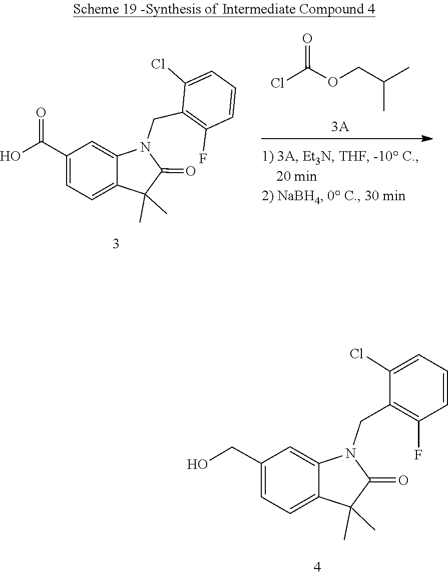

To a solution of Compound 3 ((400 mg, 1.15 mmol, 1 eq) and TEA (139.66 mg, 1.38 mmol, 192.11 uL, 1.2 eq) in THE (20 mL) was cooled to −10° C. and Compound 3A (172.80 mg, 1.27 mmol, 166.15 uL, 1.1 eq) was added. After 20 min was added NaBH₄ (130.54 mg, 3.45 mmol, 3 eq) at 0° C., and the mixture was stirred at 0° C. for 30 min. Acidify the reaction mixture by adding, with shaking, 10 mL of HCl until pH around 7. The solvent was evaporated and the residue triturated under water. The product was filtered and dried under vacuum to obtain Compound 4 (376 mg, crude) as a yellow oil.

LCMS: [M+H]⁺=334.1

¹H NMR (400 MHz, DMSO-d₆) δ=7.48-7.32 (m, 2H), 7.30-7.18 (m, 2H), 6.97-6.90 (m, 1H), 6.79 (s, 1H), 5.17-5.07 (m, 1H), 5.03 (s, 2H), 4.37 (d, J=5.6 Hz, 2H), 1.26 (s, 6H).

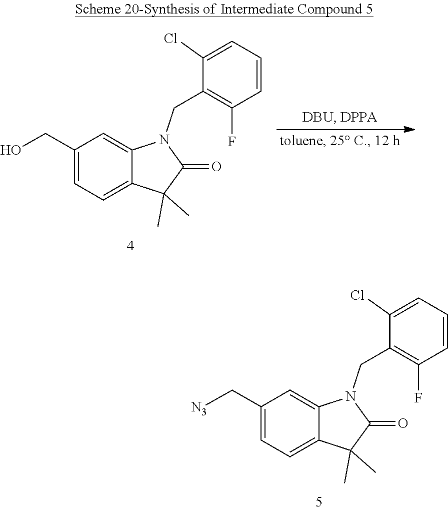 [SMILES: CC1(C)C(=O)N(Cc2c(F)cccc2Cl)c2cc(CO)ccc21 ; CC1(C)C(=O)N(Cc2c(F)cccc2Cl)c2cc(CN=[N+]=[N-])ccc21]

To a solution of Compound 4 (260 mg, 778.95 umol, 1 eq) in toluene (3 mL) was added DBU (154.16 mg, 1.01 mmol, 152.63 uL, 1.3 eq) and DPPA (257.24 mg, 934.74 umol, 202.55 uL, 1.2 eq). The mixture was stirred at 25° C. for 12 h. The reaction mixture was filtered and the filtrate was concentrated under the reduced pressure to give Compound 5 (470 mg, crude) was obtained as a yellow oil.

LCMS: [M+H]⁺=359.1

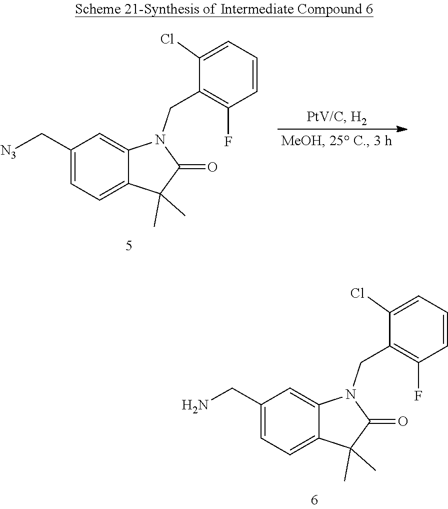 [SMILES: CC1(C)C(=O)N(Cc2c(F)cccc2Cl)c2cc(CN=[N+]=[N-])ccc21 ; CC1(C)C(=O)N(Cc2c(F)cccc2Cl)c2cc(CN)ccc21]

To a solution of Compound 5 (400 mg, 1.11 mmol, 1 eq) in MeOH (5 mL) was added PtV/C (29.10 mg, 111.48 umol, 0.1 eq) and the mixture was stirred at 25° C. for 3 h under H₂ (2.25 mg, 1.11 mmol) atmosphere (15 PSI). The reaction mixture was filtered and the filtrate was concentrated under the reduced pressure to obtain compound 6 (340 mg, crude) as a yellow solid.

LCMS: [M+H]⁺=359.1

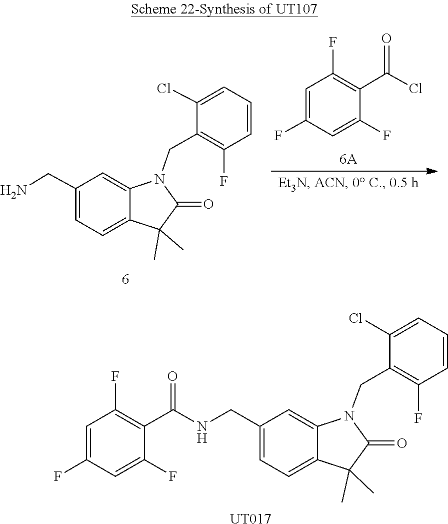

To a solution of Compound 6 (160 mg, 480.77 umol, 1 eq) and Et3N (48.65 mg, 480.77 umol, 66.92 uL, 1 eq) in ACN (1 mL) was added Compound 6A (93.53 mg, 480.77 umol, 1 eq) at 0° C. The mixture was stirred at 0° C. for 0.5 h. The reaction mixture was filtered and the filtrate was concentrated under the reduced pressure to give the product. The residue was purified by Prep-HPLC (44%-74% MeCN in water (FA), 9 min), to obtain UT017 (19.68 mg, 40.09 μmol, 8.34% yield, 100% purity) as a white solid.

LCMS: [M+H]⁺=491.2

¹H NMR (400 MHz, DMSO-d₆) δ=9.15 (t, J=6.0 Hz, 1H), 7.51-7.25 (m, 5H), 7.24-7.13 (m, 1H), 6.97 (d, J=7.6 Hz, 1H), 6.78 (s, 1H), 5.01 (s, 2H), 4.36 (d, J=6.0 Hz, 2H), 1.27 (s, 6H).

### Example 13: Synthesis of STING Modulator—UT018

Compound UT018 was synthesized according to FIG. 19, which is broken down into Scheme 23-27, below.

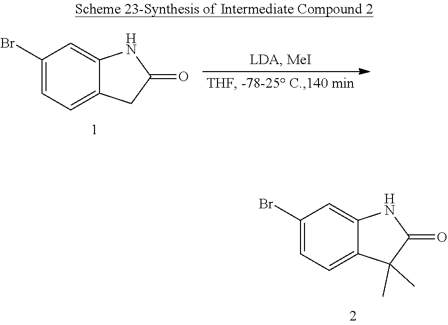

To a solution of Compound 1 (2 g, 9.43 mmol, 1 eq) in THF (20 mL) was colded to −78° C., and was added LDA (2 M, 9.43 mL, 2 eq) dropwise there into followed by stirring for 10 min. After adding LDA (2 M, 4.72 mL, 1 eq), the reaction mixture was brought to 25° C. and stirred for 1 h. Then the reaction solution was cooled to −78° C. again and Mel (1.34 g, 9.43 mmol, 587.18 uL, 1 eq) was added dropwise there into followed by stirring for 10 min. After adding Mel (1.34 g, 9.43 mmol, 587.18 uL, 1 eq), the reaction mixture was brought to 25° C. and stirred for 1 h. The reaction mixture was diluted with NH₄Cl and extracted with EtOAc (30 mL×2), the combined organic layers dried over anhydrous sodium sulfate, filtered and the filtrate was concentrated under reduced pressure to give a residue. The residue was purified by reversed-phase HPLC (44% ACN in water (0.1% FA), 33 min), to obtain Compound 2 (870 mg, 3.62 mmol, 38.42% yield) as a white solid.

LCMS: [M+H]⁺=239.7

¹H NMR (400 MHz, CHLOROFORM-d₆) δ=9.00 (s, 1H), 7.23-7.15 (m, 1H), 7.13 (d, J=1.6 Hz, 1H), 7.06 (d, J=8.0 Hz, 1H), 1.40 (s, 6H).

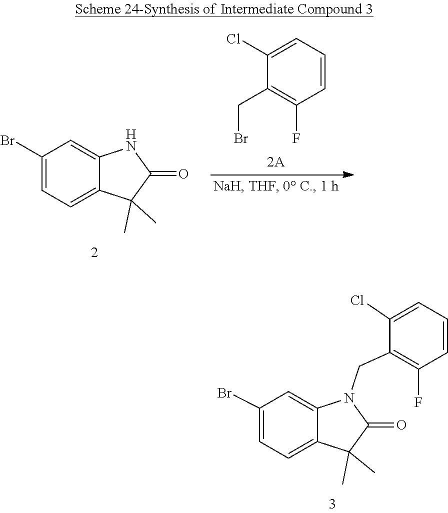 [SMILES: Fc1cccc(Cl)c1CBr ; CC1(C)C(=O)Nc2cc(Br)ccc21 ; CC1(C)C(=O)N(Cc2c(F)cccc2Cl)c2cc(Br)ccc21]

To a solution of Compound 2 (870 mg, 3.62 mmol, 1 eq) in THF (10 mL) was added NaH (173.91 mg, 4.35 mmol, 60% purity, 1.2 eq) at 0° C. for 15 min, and then was added Compound 2A (1.21 g, 5.44 mmol, 1.5 eq). The mixture was stirred at 0° C. for 45 min. The residue was diluted with H₂O (20 mL) and extracted with EtOAc (20 mL×3). The combined organic layers were washed with brine (20 mL×3), the combined organic layers were dried over anhydrous Na₂SO₄, filtered and concentrated under reduced pressure to give a residue. The residue was purified by flash silica gel chromatography (ISCO®; 12 g SepaFlash® Silica Flash Column, Eluent of 0˜15% Ethyl acetate/Petroleum ether gradient @80 L/min), to obtain Compound 3 (1.19 g, 3.11 mmol, 85.82% yield) as an off-white solid.

LCMS: [M+H]⁺=383.9

¹H NMR (400 MHz, CHLOROFORM-d₆) δ=7.24-7.11 (m, 2H), 7.06 (dd, J=8.0, 1.2 Hz, 1H), 7.01-6.88 (m, 2H), 6.83 (d, J=1.2 Hz, 1H), 5.01 (s, 2H), 1.30 (s, 6H).

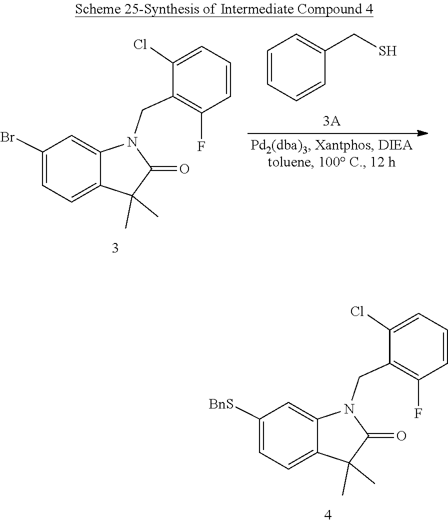

A mixture of Compound 3 (990 mg, 2.59 mmol, 1 eq), Compound 3A (642.67 mg, 5.17 mmol, 606.30 uL, 2 eq), Pd₂(dba)₃ (47.38 mg, 51.74 umol, 0.02 eq), Xantphos (59.88 mg, 103.49 umol, 0.04 eq) and DIEA (668.75 mg, 5.17 mmol, 901.28 uL, 2 eq) in toluene (10 mL) was degassed and purged with N₂ for 3 times, and then the mixture was stirred at 100° C. for 12 h. The reaction mixture was concentrated under the reduced pressure, and then the mixture was quenched by addition water (50 mL), and extracted with EtOAc (50 mL×3). The combined organic phase was washed with brine (50 mL), dried with anhydrous Na2SO4, filtered and concentrated in vacuum. The residue was purified by flash silica gel chromatography (ISCO®; 12 SepaFlash® Silica Flash Column, Eluent of 0˜20% thylacetate/Petroleum ether gradient @80 mL/min), to obtain Compound 4 (800 mg, 1.88 mmol, 72.60% yield) as a yellow oil.

LCMS: [M+H]⁺=426.1

¹H NMR (400 MHz, CHLOROFORM-d) δ=7.22-7.10 (m, 7H), 6.97 (d, J=7.6 Hz, 1H), 6.94-6.80 (m, 2H), 6.67 (s, 1H), 5.01 (s, 2H), 3.94 (s, 2H), 1.30 (d, J=0.8 Hz, 6H).

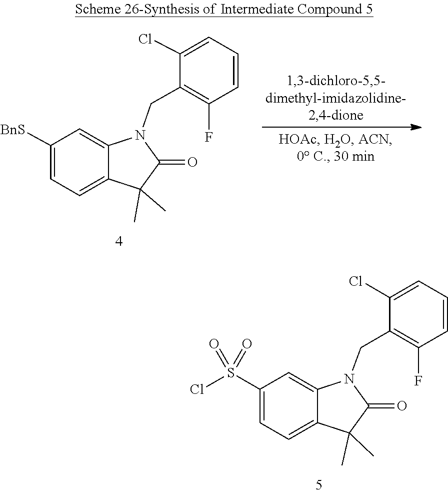

To a solution of Compound 4 (100 mg, 234.77 umol, 1 eq), HOAc (0.15 mL), H2O (0.1 mL) and 1,3-dichloro-5,5-dimethyl-imidazolidine-2,4-dione (92.51 mg, 469.54 umol, 2 eq) in ACN (4 mL) was degassed and purged with N₂ for 3 times, and then the mixture was stirred at 0° C. for 30 min under N₂ atmosphere. The reaction mixture was quenched by addition water (10 mL), and extracted with DCM (10 mL×3). The combined organic layers were dried over anhydrous Na₂SO₄, filtered and concentrated under reduced pressure to obtain Compound 5 (95 mg, crude) as a yellow oil.

LCMS: [M+H]⁺=402.0

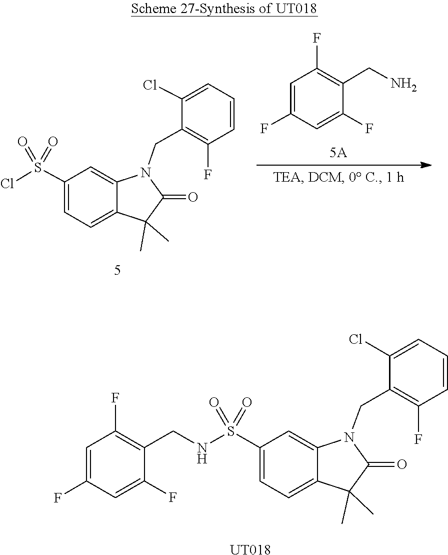

To a solution of Compound 5 (95 mg, 236.16 umol, 1 eq) in DCM (2 mL) was added TEA (71.69 mg, 708.49 umol, 98.61 uL, 3 eq) and Compound 5A (57.08 mg, 354.24 umol, 1.5 eq). The mixture was stirred at 0° C. for 1 h. The residue was purified by Prep-HPLC (47%-77% ACN in water (FA), 9 min), to obtain UT018 (51.14 mg, 94.85 umol, 40.16% yield, 97.73% purity) as a white solid.

LCMS: [M+H]⁺=527.2

¹H NMR (400 MHz, DMSO-d₆) δ=7.52 (dd, J=7.6, 1.2 Hz, 1H), 7.38-7.29 (m, 2H), 7.29-7.20 (m, 2H), 7.17-7.06 (m, 1H), 6.50 (t, J=8.0 Hz, 2H), 5.19 (s, 2H), 5.16 (t, J=6.8 Hz, 1H), 4.25 (d, J=6.8 Hz, 2H), 1.47 (s, 6H).

### Example 14: Synthesis of STING Modulator—UT065

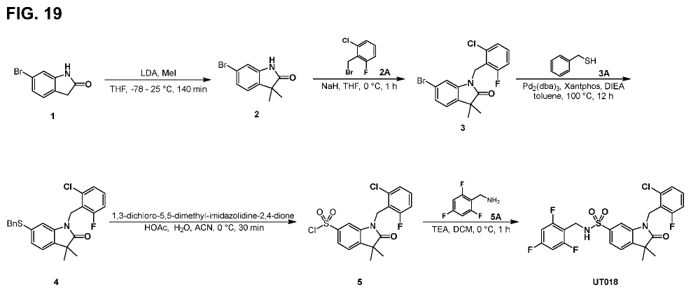

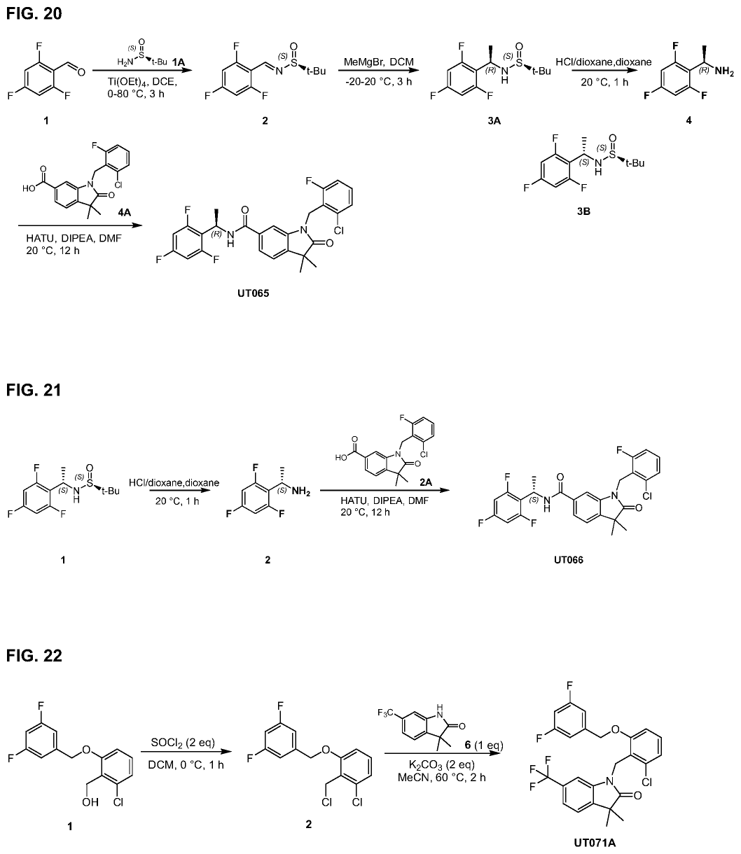

Compound UT65 was synthesized according to FIG. 20, which is broken down into Scheme 28-31 below.

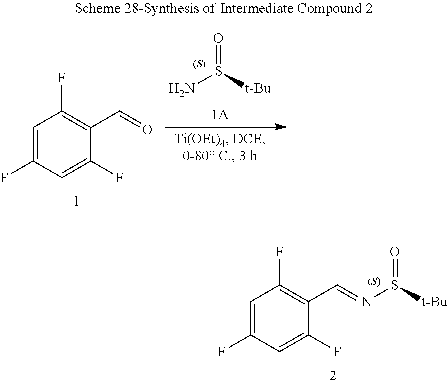

To a solution of Compound 1 (3 g, 18.74 mmol, 1 eq) in DCE (45 mL) was added Compound 1A (2.27 g, 18.74 mmol, 1 eq). To the mixture was added Ti(i-PrO)4 (8.52 g, 29.98 mmol, 8.85 mL, 1.6 eq) gradually at 0° C. The reaction mixture was stirred at 80° C. for 3 h. The stirred mixture was cooled down to 0° C. To the vigorously stirred cold solution was added Celite (30 g) then added water (15 mL). The slurry is vigorously stirred at 20° C. for 30 min. The mixture was filtered and the Celite cake was washed with DCM (100 mL). The filtrate was concentrated. The residue was dissolved with EtOAc (100 mL), washed with brine (100 mL), dried, filtered and concentrated under the reduced pressure to obtain Compound 2 (4.4 g, 16.71 mmol, 89.18% yield) as a yellow oil.

LCMS: [M+H]⁺=207.9

¹H NMR (400 MHz, CHLOROFORM-d) δ=8.74 (s, 1H), 6.78 (t, J=8.4 Hz, 2H), 1.27 (s, 9H).

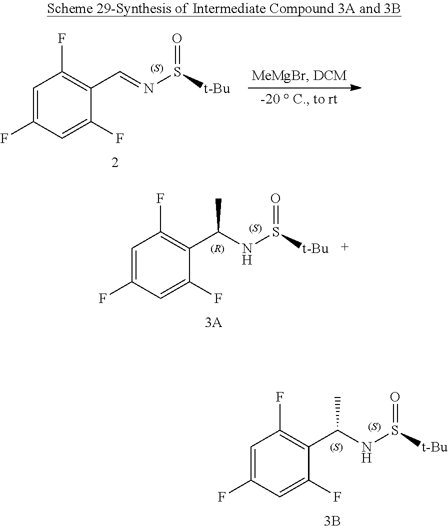

To a solution of Compound 2 (1 g, 3.80 mmol, 1 eq) in DCM (15 mL) was added MeMgBr (3 M, 3.04 mL, 2.4 eq) dropwise at −20° C. The reaction was stirred at −20° C. for 1 h then 20° C. for 2 h. The reaction was quenched by the addition of aq. NH4Cl (30 mL). The aqueous phase was extracted with DCM (30 mL). The combined organic extracts were washed with brine (30 mL), dried, filtered and concentrated. The residue was purified by flash silica gel chromatography (ISCO®; 4 g SepaFlash® Silica Flash Column, Eluent of 0˜25% Ethylacetate/Petroleum ether gradient @80 mL/min) and concentrated under the reduced pressure to obtain Compound 3A (190 mg, 680.22 μmol, 17.91% yield) as a yellow oil and Compound 3B (400 mg, 1.43 mmol, 37.65% yield) as a yellow oil.

LCMS: [M+H]⁺=280.1

¹H NMR (400 MHz, CHLOROFORM-d) δ=6.65 (t, J=8.8 Hz, 2H), 4.88-4.74 (m, 1H), 3.78 (d, J=9.6 Hz, 1H), 1.57 (d, J=7.2 Hz, 3H), 1.21 (s, 9H).

¹H NMR (400 MHz, CHLOROFORM-d) δ=6.65 (t, J=8.8 Hz, 2H), 4.93 (m, 1H), 3.63 (d, J=6.8 Hz, 1H), 1.65 (d, J=6.8 Hz, 3H), 1.15 (s, 9H).

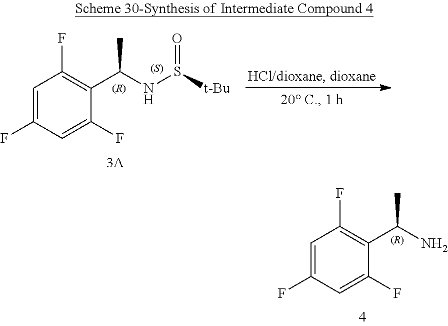

To a solution of Compound 3A (190 mg, 680.22 umol, 1 eq) in dioxane (1.5 mL) was added HCl/dioxane (4 M, 170.06 uL, 1 eq). The mixture was stirred at 20° C. for 1 hr. The reaction mixture was concentrated under the reduced pressure to obtain Compound 4 (400 mg, crude, HCl) as an off-white solid.

LCMS: [M+H]⁺=176.1

¹H NMR (400 MHz, DMSO-d₆) δ=8.57 (s, 3H), 7.32 (t, J=9.2 Hz, 2H), 5.75 (s, 1H), 4.61 (s, 1H), 1.60-1.55 (m, 3H).

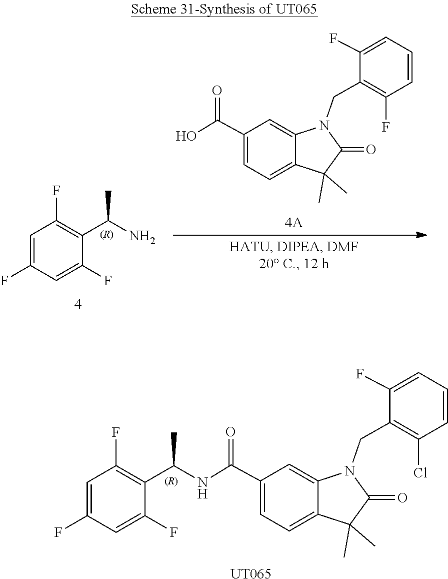

To a solution of Compound 4 (50.00 mg, 236.28 umol, 1 eq, HCl) and Compound 4A (123.26 mg, 354.42 umol, 1.5 eq) in DMF (1 mL) was added HATU (134.76 mg, 354.42 umol, 1.5 eq) and DIEA (152.69 mg, 1.18 mmol, 205.78 uL, 5 eq). The mixture was stirred at 20° C. for 12 hr. The residue was purified by prep-HPLC (column: Phenomenex Luna C18 150*25 mm*10 um; mobile phase: [water (FA)-ACN]; B %: 53%-83%, 10 min) and lyophilized to obtain UT065 (48.09 mg, 90.76 μmol, 38.41% yield, 95.293% purity) as a white solid.

LCMS: [M+H]⁺=505.0

¹H NMR (400 MHz, CHLOROFORM-d) δ=7.45 (d, J=7.6 Hz, 1H), 7.24 (dd, J=1.6, 5.2 Hz, 3H), 7.16 (s, 1H), 7.05-6.98 (m, 1H), 6.68 (t, J=8.4 Hz, 2H), 6.55 (d, J=8.4 Hz, 1H), 5.75-5.66 (m, 1H), 5.16 (d, J=3.2 Hz, 2H), 1.59 (s, 3H), 1.41 (s, 6H).

### Example 15: Synthesis of STING Modulator—UT066

Compound UT066 was synthesized according to FIG. 21, which is broken down into Scheme 31-32 below.

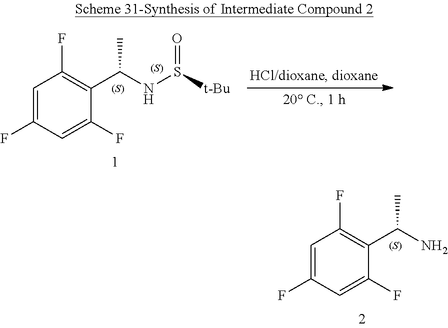

To a solution of Compound 1 (190 mg, 680.22 umol, 1 eq) in dioxane (1.5 mL) was added HCl/dioxane (4 M, 170.06 μL, 1 eq). The mixture was stirred at 20° C. for 1 hr. The reaction mixture was concentrated under the reduced pressure to give Compound 2 (160 mg, crude, HCl) as an off-white oil.

LCMS: [M+H]⁺=176.1

¹H NMR (400 MHz, DMSO-d₆) δ=8.47 (s, 2H), 7.37-7.32 (m, 1H), 4.69-4.56 (m, 1H), 4.68-4.56 (m, 1H), 1.56 (d, J=7.6 Hz, 3H).

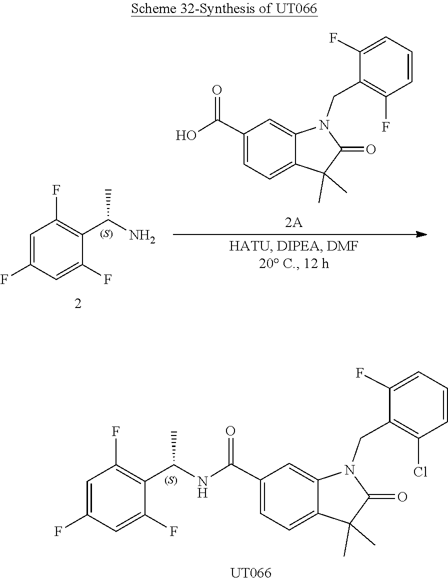

To a solution of Compound 2 (50.00 mg, 236.28 umol, 1 eq, HCl) and Compound 2A (123.26 mg, 354.42 umol, 1.5 eq) in DMF (1 mL) was added HATU (134.76 mg, 354.42 umol, 1.5 eq) and DIEA (152.69 mg, 1.18 mmol, 205.78 uL, 5 eq). The mixture was stirred at 20° C. for 12 hr. The residue was purified by prep-TLC (SiO₂, Petroleum ether:Ethyl acetate=3:1, Rf=0.4) and eluted with acetonitrile (5 mL) to give UT066 (39.25 mg, 76.43 μmol, 32.35% yield, 98.316% purity) was obtained as an off-white solid.

LCMS: [M+H]⁺=505.2

¹H NMR (400 MHz, DMSO-d₆) δ=8.72 (d, J=6.4 Hz, 1H), 7.55 (dd, J=1.2, 7.6 Hz, 1H), 7.45-7.33 (m, 3H), 7.26-7.20 (m, 2H), 7.12 (t, J=8.8 Hz, 2H), 5.27 (t, J=6.8 Hz, 1H), 5.10-5.00 (m, 2H), 1.52 (d, J=7.2 Hz, 3H), 1.28 (d, J=2.4 Hz, 6H).

### Example 16: Synthesis of STING Modulator—UT071

Compound UT071 was synthesized according to FIG. 22, which is broken down into Scheme 33-34 below.

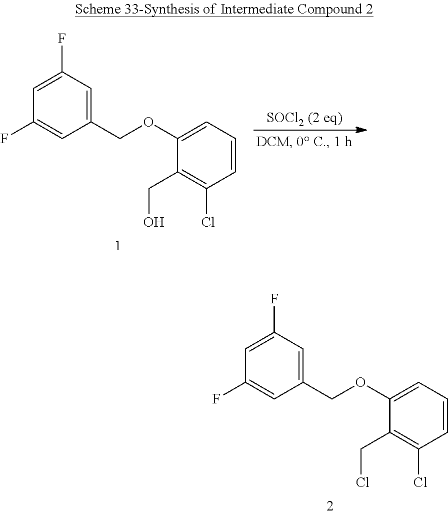 [SMILES: OCc1c(Cl)cccc1OCc1cc(F)cc(F)c1 ; Fc1cc(F)cc(COc2cccc(Cl)c2CCl)c1]

A mixture of Compound 1 (100 mg, 351.27 μmol, 1 eq), SOCl₂ (83.58 mg, 702.53 μmol, 50.96 uL, 2.0 eq) in DCM (3 mL) was degassed and purged with N₂ for 3 times, and then the mixture was stirred at 0° C. for 1 h under N₂ atmosphere. The mixture was concentrated in vacuum to obtain Compound 2 (100 mg, crude) as a white solid.

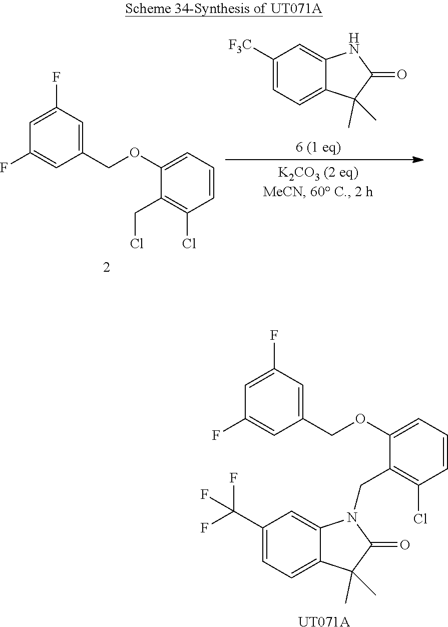

A mixture of Compound 2 (100 mg, 329.89 μmol, 1 eq), Compound 6 (75.61 mg, 329.89 μmol, 1 eq), K₂CO₃ (91.19 mg, 659.78 μmol, 2.0 eq) in MeCN (3 mL) was degassed and purged with N₂ for 3 times, and then the mixture was stirred at 60° C. for 2 h under N₂ atmosphere. The mixture was filtered and concentrated in vacuum to give a residue. The residue was purified by prep-HPLC (column: Phenomenex Luna C18 100*30 mm*5 um; mobile phase: [water (FA)-ACN]; B %: 65%-95%, 8 min) to obtain Compound UT071A (18.97 mg, 38.05 μmol, 11.53% yield, 99.46% purity) as a off-white solid.

LCMS: [M+H]⁺=496.1

¹H NMR (400 MHz, DMSO-d₆) δ=7.53 (d, J=7.6 Hz, 1H), 7.39-7.28 (m, 2H), 7.22-7.01 (m, 6H), 5.18 (s, 2H), 5.10 (s, 2H), 1.25 (s, 6H).

### Example 17: Synthesis of STING Modulator—UT072

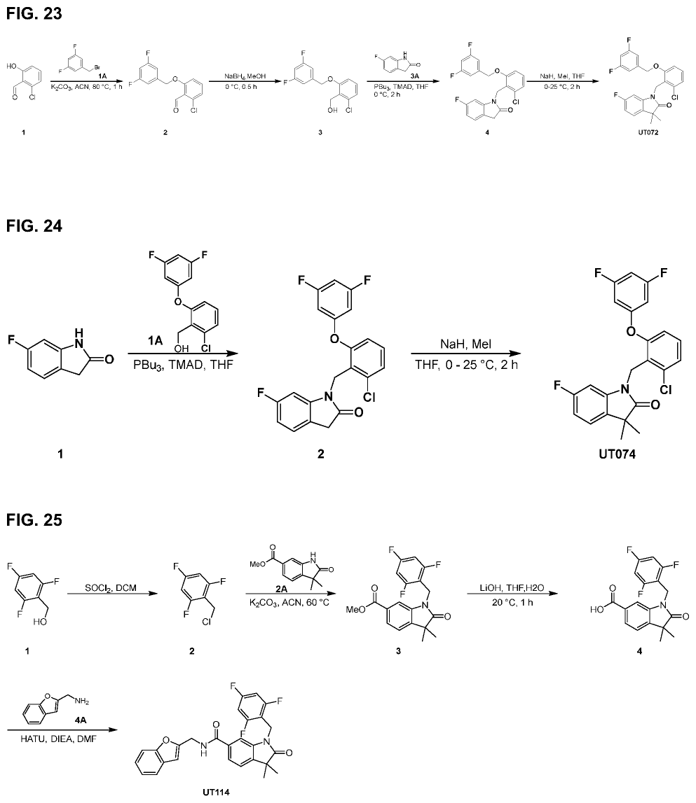

Compound UT072 was synthesized according to FIG. 23, which is broken down into Scheme 35-38 below.

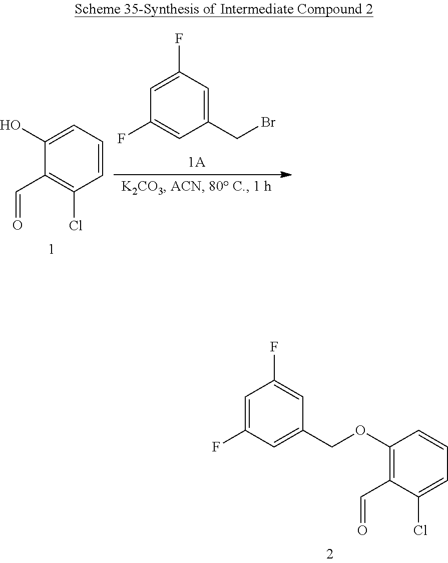

To a solution of Compound 1 (5 g, 31.94 mmol, 1 eq) and Compound 1A (6.61 g, 31.94 mmol, 4.13 mL, 1 eq) in ACN (30 mL) was added K2CO3 (5.30 g, 38.32 mmol, 1.2 eq). The mixture was stirred at 80° C. for 1 h. The reaction mixture was filtered and the filtrate was concentrated under the reduced pressure to give the product. The residue was purified by column chromatography (SiO₂, Petroleum ether/Dichloromethane=1/1 to 1/1) was concentrated under the reduced pressure to yield Compound 2 (6.79 g, 24.02 mmol, 75.22% yield) as a white solid.

LCMS: [M+H]⁺=283.0

¹H NMR (400 MHz, DMSO-d₆) δ=10.45 (s, 1H), 7.59 (t, J=8.4 Hz, 1H), 7.30-7.14 (m, 5H), 5.30 (s, 2H).

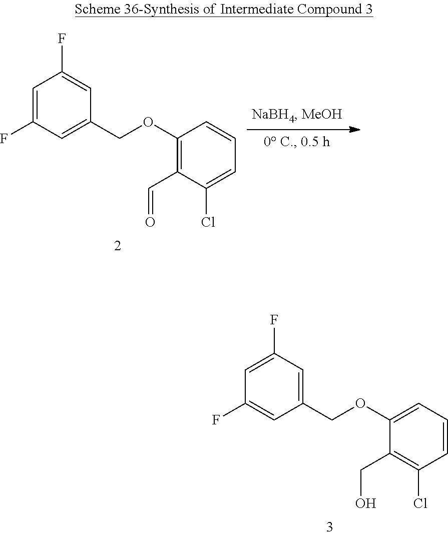 [SMILES: O=Cc1c(Cl)cccc1OCc1cc(F)cc(F)c1 ; OCc1c(Cl)cccc1OCc1cc(F)cc(F)c1]

To a solution of Compound 2 (6.7 g, 23.70 mmol, 1 eq) in MeOH (70 mL) was added NaBH4 (896.73 mg, 23.70 mmol, 1 eq) at 0° C. The mixture was stirred at 0° C. for 0.5 hr. The reaction was quenched by the addition of water (200 mL). The mixture was extracted with ethyl acetate (200 mL×2). The combined organic extracts were washed with brine (200 mL), dried, filtered and concentrated under the reduced pressure to yield Compound 3 (6.58 g, 23.12 mmol, 97.54% yield) as a white solid.

LCMS: [M+H]⁺=267.0

¹H NMR (400 MHz, DMSO-d₆) δ=7.32-7.12 (m, 4H), 7.08-6.97 (m, 2H), 5.20 (s, 2H), 4.92 (t, J=5.2 Hz, 1H), 4.66 (d, J=5.2 Hz, 2H).

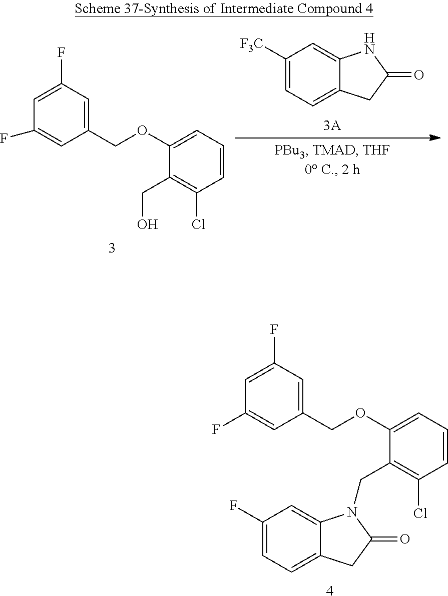

To a solution of TMAD (2.28 g, 13.23 mmol, 2.5 eq) in THE (10 mL) was added PBu3 (3.47 g, 13.23 mmol, 2.5 eq) at 0° C. and the mixture was stirred at 0° C. for 30 min, then Compound 3A (800 mg, 5.29 mmol, 1.08 mL, 1 eq) and Compound 3 (1.51 g, 5.29 mmol, 1 eq) in THE (10 mL) was added and the mixture was stirred at 0° C. for 1.5 h. The reaction was quenched by the addition of water (30 mL). The mixture was extracted with ethyl acetate (30 mL×2). The combined organic extracts were washed with brine (30 mL), dried, filtered and concentrated. The residue was purified by flash silica gel chromatography (ISCO®; 40 g SepaFlash® Silica Flash Column, Eluent of 0˜10% Ethyl acetate/Petroleum ether gradient @80 mL/min) and concentrated under the reduced pressure to yield Compound 4 (689 mg, crude) as an off-white solid.

LCMS: [M+H]⁺=418.1

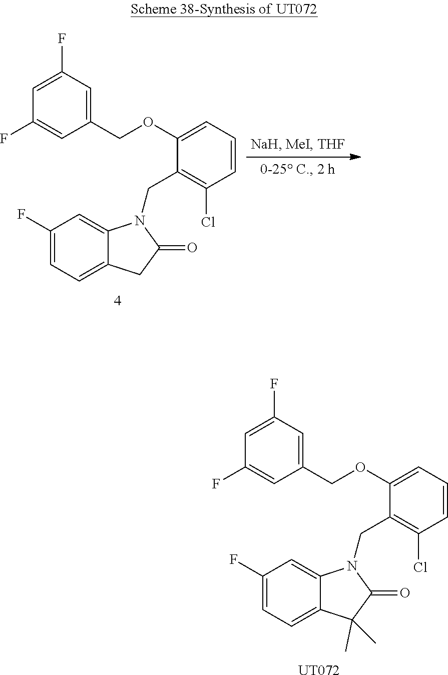

To a solution of Compound 4 (300 mg, 718.03 umol, 1 eq) in THE (5 mL) was added NaH (34.46 mg, 861.58 umol, 60% purity, 1.20 eq) and Mel (203.83 mg, 1.44 mmol, 89.40 uL, 2 eq) at 0° C. The mixture was stirred at 25° C. for 2 h. The reaction mixture was diluted with NH4Cl and extracted with EtOAc (10 mL×2), the combined organic layers dried over anhydrous sodium sulfate, filtered and the filtrate was concentrated under reduced pressure to give a residue. The residue was purified by prep-HPLC (column: Phenomenex luna C18 150*25 mm*10 um; mobile phase: [water (FA)-ACN]; B %: 56%-86%, 10.5 min) and lyophilized to yield UT072 (14.71 mg, 32.78 μmol, 4.57% yield, 99.366% purity) as an off-white solid.

LCMS: [M+H]⁺=446.1

¹H NMR (400 MHz, DMSO-d₆) δ=7.31-7.31 (m, 1H), 7.37-7.26 (m, 1H), 7.20-7.03 (m, 5H), 6.72 (ddd, J=2.4, 8.0, 10.0 Hz, 1H), 6.56 (dd, J=2.4, 10.0 Hz, 1H), 5.18 (s, 2H), 5.03 (s, 2H), 1.19 (s, 6H).

### Example 18: Synthesis of STING Modulator—UT074

Compound UT074 was synthesized according to FIG. 24, which is broken down into Scheme 39-40 below.

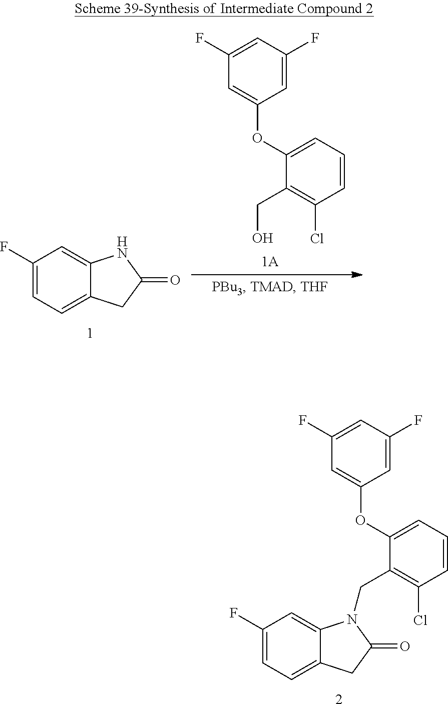

To a solution of TMAD (2.28 g, 13.23 mmol, 2.5 eq) in THE (8 mL) was added tributylphosphane (2.68 g, 13.23 mmol, 3.26 mL, 2.5 eq) at 0° C. and the mixture was stirred at 0° C. for 30 min, then Compound 1 (800 mg, 5.29 mmol, 1.08 mL, 1 eq) and Compound 1A (1.43 g, 5.29 mmol, 1 eq) in THE (8 mL) was added and the mixture was stirred at 0° C. for another 90 min. The mixture was quenched by H₂O (20 mL) and extracted with EtOAc (30 mL), the organic layer was concentrated under reduced pressure to give a residue. The residue was purified by column chromatography (SiO₂, Petroleum ether/Ethyl acetate=20/1 to 10/1) to yield Compound 2 (320 mg, 792.51 μmol, 14.97% yield) as a yellow solid.

LCMS: [M+H]⁺=404.0

1H NMR (400 MHz, DMSO-d₆) δ=7.46-7.44 (m, 2H), 7.43-7.41 (m, 1H), 7.20-7.12 (m, 1H), 7.06 (t, J=1.2 Hz, 1H), 6.72-6.70 (m, 2H), 6.39-6.36 (m, 2H), 5.03 (s, 2H), 3.23 (s, 2H).

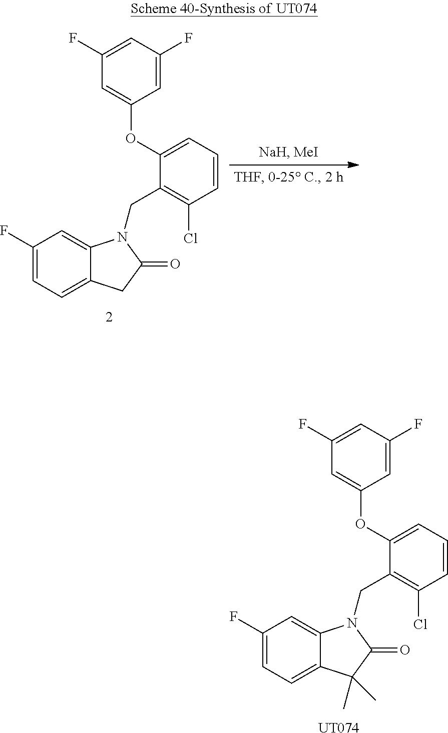

To a solution of Compound 2 (300.00 mg, 742.98 umol, 1 eq) in THE (5 mL) was added NaH (59.43 mg, 1.49 mmol, 60% purity, 2 eq) and Mel (210.91 mg, 1.49 mmol, 92.51 uL, 2 eq) at 0° C. The mixture was stirred at 25° C. for 2 h. The reaction mixture was diluted with NH₄Cl and extracted with EtOAc (10 mL×2), the combined organic layers dried over anhydrous sodium sulfate, filtered and the filtrate was concentrated under reduced pressure to give a residue. The residue was purified by prep-HPLC (62%-92% MeCN in water (FA), 9 min), to yield UT074 (135.81 mg, 312.43 μmol, 42.05% yield, 99.34% purity) as a yellow solid.

LCMS: [M+H]⁺=432.1

¹H NMR (400 MHz, DMSO-d₆) δ=7.45-7.37 (m, 2H), 7.30 (dd, J=8.4, 5.6 Hz, 1H), 7.05 (dd, J=6.4, 2.8 Hz, 1H), 6.97 m, 1H), 6.84-6.66 (m, 2H), 6.53 (dd, J=8.4, 2.0 Hz, 2H), 5.04 (s, 2H), 1.15 (s, 6H).

### Example 19: Synthesis of STING Modulator—UT114

Compound UT114 was synthesized according to FIG. 25, which is broken down into Scheme 41-44 below.

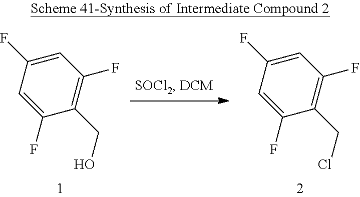 [SMILES: OCc1c(F)cc(F)cc1F ; Fc1cc(F)c(CCl)c(F)c1]

To a solution of Compound 1 (5 g, 30.84 mmol, 1 eq) in DCM (50 mL) was added SOCl₂ (7.34 g, 61.69 mmol, 4.47 mL, 2 eq) at 0° C. The mixture was stirred at 0° C. for 1 h. The reaction mixture was concentrated under the reduced pressure to give the product. The residue was purified by flash silica gel chromatography (ISCO®; 20 g SepaFlash® Silica Flash Column, Eluent of 0˜10% Ethylacetate/Petroleum ether gradient @80 mL/min) and was concentrated under the reduced pressure to give Compound 2 (3.7 g, 20.49 mmol, 66.44% yield) as a colorless liquid.

¹H NMR (400 MHz, DMSO-d₆) δ=7.35-7.17 (m, 2H), 4.74 (s, 2H).

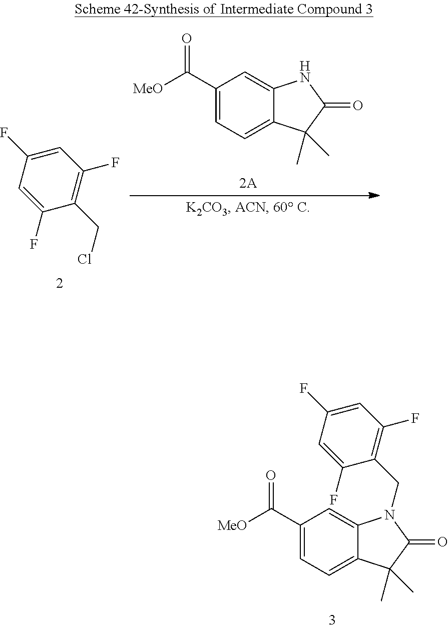

To a solution of methyl Compound 2A (2 g, 9.12 mmol, 1 eq) and Compound 2 (1.65 g, 9.12 mmol, 1 eq) in ACN (20 mL) was added K₂CO₃ (7.56 g, 54.74 mmol, 6 eq). The mixture was stirred at 65° C. for 2 h. The reaction mixture was filtered and the filtrate was concentrated under the reduced pressure to give the product. The residue was purified by flash silica gel chromatography (ISCO®; 20 g SepaFlash® Silica Flash Column, Eluent of 0˜20% Ethylacetate/Petroleum ether gradient @80 mL/min) and was concentrated under the reduced pressure to obtain Compound 3 (1.7 g, 4.68 mmol, 51.29% yield) as a white solid.

¹H NMR (400 MHz, DMSO-d₆) δ=7.68 (dd, J=1.6, 8.0 Hz, 1H), 7.52 (d, J=7.6 Hz, 1H), 7.46 (d, J=1.2 Hz, 1H), 7.22 (t, J=9.2 Hz, 2H), 4.98 (s, 2H), 3.83 (s, 3H), 1.29 (s, 6H).

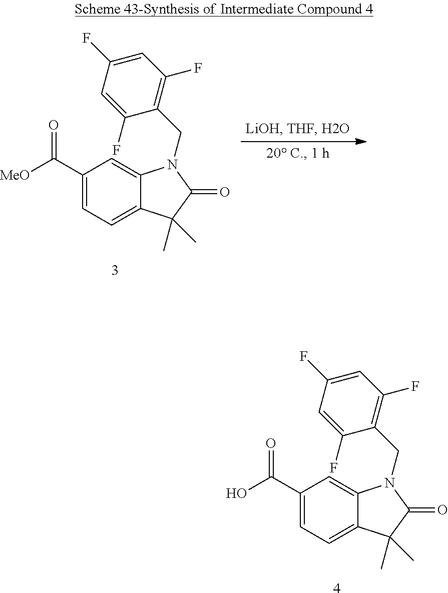

To a solution of Compound 3 (1.7 g, 4.68 mmol, 1 eq) in THE (10 mL) was added LiOH (294.52 mg, 7.02 mmol, 1.5 eq) in H₂O (10 mL). The mixture was stirred at 20° C. for 1 h. Acidify the reaction mixture by adding, with shaking, 10 mL of HCl until pH around 3, and then extracted with EtOAc (20 mL×3). The combined organic layers were dried over anhydrous Na₂SO₄, filtered and concentrated to obtain Compound 4 (1.61 g, 4.60 mmol, 98.33% yield) as a white solid.

LCMS: [M+H]⁺=350.0

¹H NMR (400 MHz, DMSO-d₆) δ=14.08-11.88 (m, 1H), 7.67 (dd, J=1.2, 7.6 Hz, 1H), 7.52-7.42 (m, 2H), 7.22 (t, J=8.8 Hz, 2H), 4.98 (s, 2H), 1.29 (s, 6H).

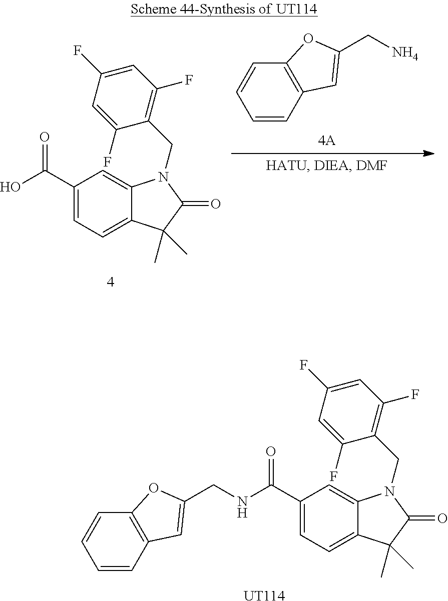

To a solution of Compound 4 (200 mg, 572.57 umol, 1 eq) in DMF (2 mL) was added HATU (326.56 mg, 858.85 umol, 1.5 eq) at 0° C. The mixture was stirred at this temperature for 30 min. Then was added Compound 4A (84.27 mg, 572.57 umol, 1 eq) and DIEA (222.00 mg, 1.72 mmol, 299.19 uL, 3 eq) at 0° C. The mixture was stirred at 25° C. for 1.5 h. The residue was purified by prep-HPLC (column: Phenomenex Luna C18 100*30 mm*5 um; mobile phase: [water (FA)-ACN]; B %: 47%-77%, 8 min) and lyophilized to yield UT114 (123.55 mg, 253.34 μmol, 44.25% yield, 98.107% purity) as an off-white solid.

LCMS: [M+H]⁺=479.0

¹H NMR (400 MHz, DMSO-d₆) δ=9.04 (t, J=5.6 Hz, 1H), 7.64-7.54 (m, 2H), 7.50 (d, J=8.0 Hz, 1H), 7.45 (d, J=7.6 Hz, 1H), 7.41 (s, 1H), 7.36-7.02 (m, 4H), 6.71 (s, 1H), 4.94 (s, 2H), 4.60 (d, J=5.6 Hz, 2H), 1.26 (s, 6H).

### Example 20: Synthesis of STING Modulator—UT115

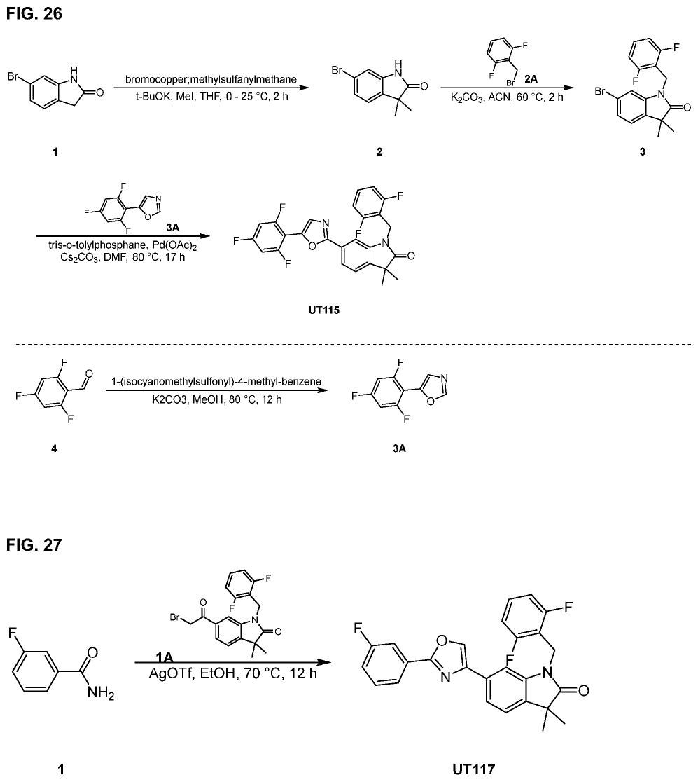

Compound UT115 was synthesized according to FIG. 26, which is broken down into Scheme 45-48 below.

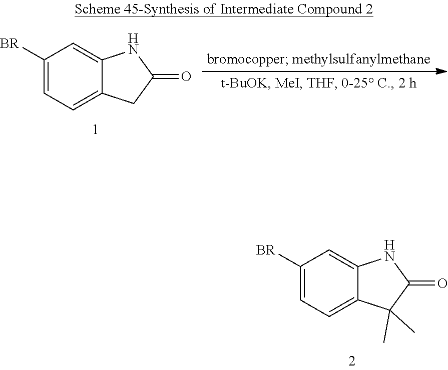

To a solution of t-BuOK (4.06 g, 36.22 mmol, 2.56 eq) in THE (30 mL) was added bromocopper;methylsulfanylmethane (290.86 mg, 1.41 mmol, 0.1 eq) and Compound 1 (3 g, 14.15 mmol, 1 eq) at 0° C., then was added Mel (2.41 g, 16.98 mmol, 1.06 mL, 1.2 eq) at 8° C. for 1 h. The mixture was stirred at 25° C. for 1 h. Acidify the reaction mixture by adding, with shaking, 50 mL of citric acid until pH around 8, and then extracted with EtOAc (50 mL×3). The combined organic layers were dried over anhydrous Na₂SO₄, filtered and concentrated to give a residue. The water-course was quenched by addition aq. NaClO (50 mL) and discarded. The residue was purified by flash silica gel chromatography (ISCO®; 40 g SepaFlash® Silica Flash Column, Eluent of 0˜20% Ethylacetate/Petroleum ether gradient @50 mL/min) to obtain Compound 2 (1.17 g, 4.87 mmol, 34.44% yield) as a pink solid.

LCMS: [M+H]⁺=239.7

¹H NMR (400 MHz, DMSO-d₆) δ=10.45 (s, 1H), 7.25 (d, J=7.6 Hz, 1H), 7.14 (dd, J=8.0, 2.0 Hz, 1H), 6.98 (d, J=1.6 Hz, 1H), 1.23 (s, 6H).

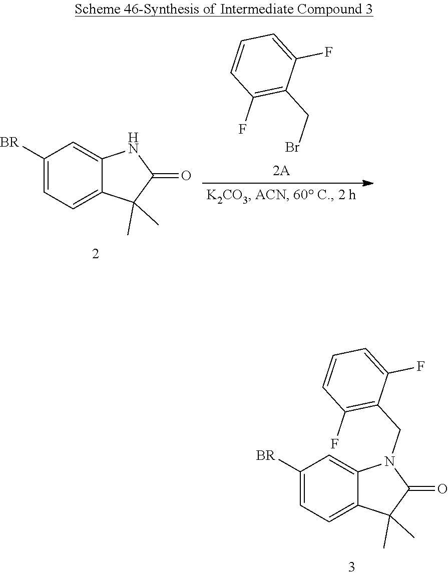

To a solution of Compound 2 (1 g, 4.16 mmol, 1 eq) and Compound 2A (1.12 g, 5.41 mmol, 1.3 eq) in ACN (15 mL) was added K₂CO₃ (2.88 g, 20.82 mmol, 5 eq). The mixture was stirred at 60° C. for 2 h. The reaction mixture was filtered and the filtrate was concentrated under the reduced pressure to give a residue. The residue was purified by flash silica gel chromatography (ISCO®; 20 g SepaFlash® Silica Flash Column, Eluent of 0˜12% Ethylacetate/Petroleum ether gradient @60 mL/min) to yield Compound 3 (1.45 g, 3.96 mmol, 95.07% yield) as a red solid.

LCMS: [M+H]⁺=365.9

¹H NMR (400 MHz, DMSO-d₆) δ=7.42 (m, 1H), 7.32 (d, J=7.6 Hz, 1H), 7.21 (dd, J=7.6, 1.2 Hz, 1H), 7.17-7.00 (m, 3H), 4.98 (s, 2H), 1.25 (s, 6H).

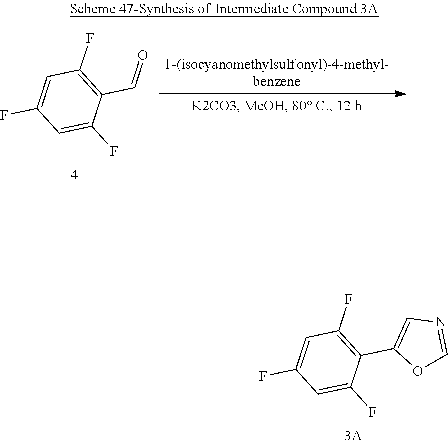 [SMILES: O=Cc1c(F)cc(F)cc1F ; Fc1cc(F)c(-c2cnco2)c(F)c1]

To a solution of Compound 4 (4 g, 24.99 mmol, 1 eq) in MeOH (20 mL) was added 1-(isocyanomethylsulfonyl)-4-methyl-benzene (4.88 g, 24.99 mmol, 1 eq) and K₂CO₃ (5.18 g, 37.48 mmol, 1.5 eq). The mixture was stirred at 80° C. for 12 h. The residue was filtered and the filtrate was concentrated under reduced pressure to give a residue. The residue was purified by column chromatography (SiO₂, Petroleum ether/Ethyl acetate=10/1=5:1) to obtain Compound 3A (2.5 g, 12.55 mmol, 50.25% yield) as a white solid.

¹H NMR (400 MHz, CHLOROFORM-d) δ=8.03 (s, 1H), 7.49 (s, 1H), 7.03-6.62 (m, 2H).

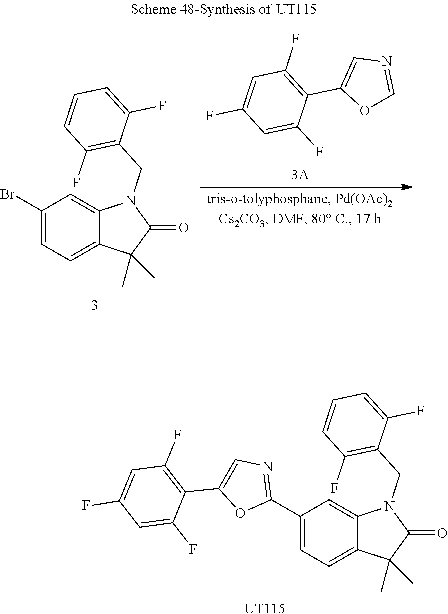

A mixture of Compound 3 (183.90 mg, 502.19 umol, 1 eq) and Compound 3A (100 mg, 502.19 umol, 1 eq) Pd(OAC)₂ (11.27 mg, 50.22 μmol, 0.1 eq), Cs₂CO₃ (327.24 mg, 1.00 mmol, 2 eq) and tris-o-tolylphosphane (30.57 mg, 100.44 umol, 0.2 eq) in DMF (2 mL) was degassed and purged with N₂ for 3 times, and then the mixture was stirred at 80° C. for 17 h under N₂ atmosphere. The reaction mixture was filtered and the filtrate was concentrated under the reduced pressure to give a residue. The residue was purified by prep-HPLC (62%-92% MeCN in water (FA), 9 min), to obtain UT115 (92.65 mg, 190.95 mol, 38.02% yield, 99.84% purity) as an off-white solid.

LCMS: [M+H]⁺=485.1

¹H NMR (400 MHz, DMSO-d₆) δ=7.77-7.67 (m, 2H), 7.56 (d, J=7.6 Hz, 1H), 7.54-7.45 (m, 3H), 7.45-7.37 (m, 1H), 7.13 (t, J=8.0 Hz, 2H), 5.06 (s, 2H), 1.33 (s, 6H).

### Example 21: Synthesis of STING Modulator—UT117

Compound UT117 was synthesized according to FIG. 27, which is reproduced as Scheme 49 below.

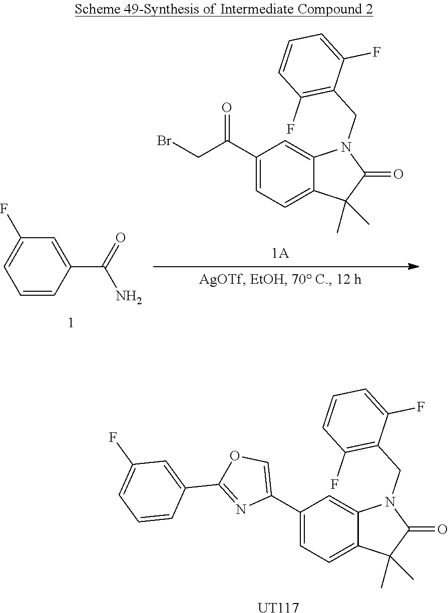 [SMILES: NC(=O)c1cccc(F)c1 ; CC1(C)C(=O)N(Cc2c(F)cccc2F)c2cc(C(=O)CBr)ccc21 ; CC1(C)C(=O)N(Cc2c(F)cccc2F)c2cc(-c3coc(-c4cccc(F)c4)n3)ccc21 ; "UT117"]

To a solution of Compound 1 (49.08 mg, 352.74 umol, 1 eq) and Compound 1A (180 mg, 440.92 umol, 1.25 eq) in EtOAc (3 mL) was added AgOTf (113.29 mg, 440.92 umol, 1.25 eq). The mixture was stirred at 70° C. for 12 h under dark. The mixture was cooled to 20° C. and diluted with ethyl acetate (3 mL) and was added brine (3 mL), the mixture was stirred at 25° C. for 4 h. The mixture was filtered and the filtrate was stratified with a separating funnel. The organic layer was washed with water (4 mL) and 5% NaHCO₃ (4 mL). The organic layer was dried over anhydrous Na₂SO₄, filtered and concentrated under reduced pressure and purified by prep-HPLC (column: Phenomenex luna C18 150*25 mm*10 um; mobile phase: [water (FA)-ACN]; B %: 66%-96%, 9 min) and lyophilized to obtain UT117 (8.76 mg, 19.24 μmol, 5.45% yield, 98.467% purity) as a white solid.

LCMS: [M+H]⁺=449.0

¹H NMR (400 MHz, DMSO-d₆) δ=8.66 (s, 1H), 7.89 (d, J=7.6 Hz, 1H), 7.78 (td, J=9.2, 2.4 Hz, 1H), 7.65 (dt, J=8.0, 5.8 Hz, 1H), 7.57-7.50 (m, 1H), 7.48-7.35 (m, 4H), 7.19-7.04 (m, 2H), 5.04 (s, 2H), 1.30 (s, 6H).

### Example 22: Synthesis of STING Modulator—UT118

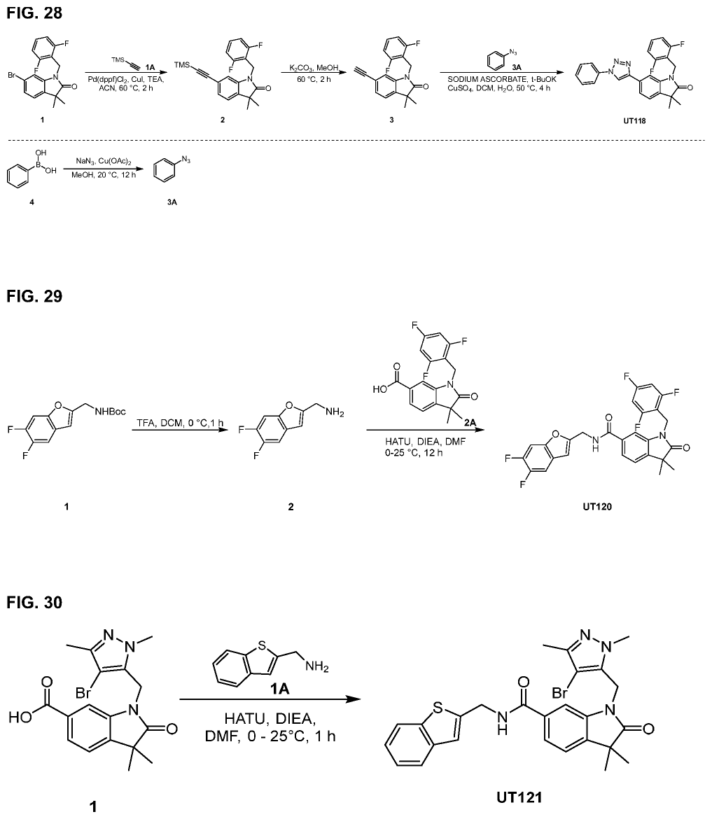

Compound UT118 was synthesized according to FIG. 28, which is reproduced as Scheme 50-53 below.

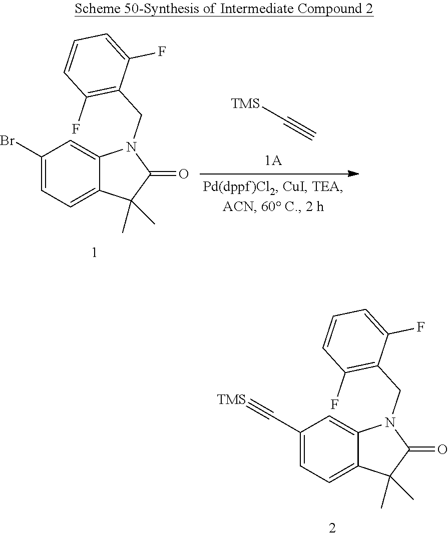

A mixture of Compound 1 (400 mg, 1.09 mmol, 1 eq), Compound 1A (128.74 mg, 1.31 mmol, 181.58 uL, 1.2 eq), Pd(dppf)Cl₂ (79.92 mg, 109.23 umol, 0.1 eq), TEA (221.06 mg, 2.18 mmol, 304.07 uL, 2 eq) and CuI (10.40 mg, 54.62 umol, 0.05 eq) in ACN (2 mL) was degassed and purged with N₂ for 3 times, and then the mixture was stirred at 60° C. for 2 h under N₂ atmosphere. The reaction mixture was filtered and the filtrate was concentrated under the reduced pressure to give a residue. The residue was purified by flash silica gel chromatography (ISCO®; 4 g SepaFlash® Silica Flash Column, Eluent of 0˜20% Ethylacetate/Petroleum ether gradient @50 mL/min) to obtain Compound 2 (310 mg, 654.75 μmol, 14.99% yield, 81% purity) as a black brown oil.

LCMS: [M+H]⁺=384.5

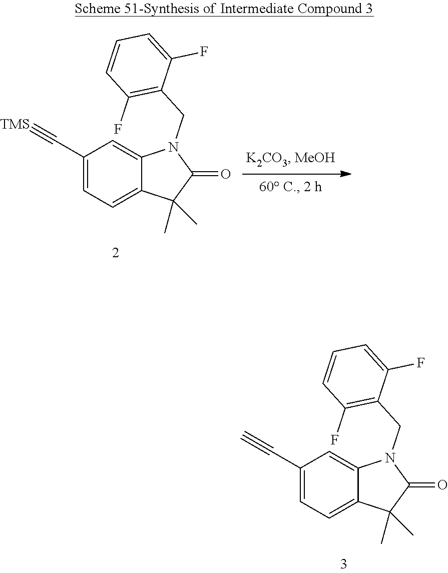

To a solution of Compound 3 (310 mg, 654.75 umol, 81% purity, 1 eq) in MeOH (3 mL) was added K₂CO₃ (271.47 mg, 1.96 mmol, 3 eq). The mixture was stirred at 60° C. for 2 h. The reaction mixture was filtered and the filtrate was concentrated under the reduced pressure to give a residue. The residue was purified by prep-TLC (SiO₂, Petroleum ether:Ethyl acetate=3:1, Rf=0.3) and eluted with ethyl acetate (20 mL) to obtain Compound 3 (190 mg, 610.30 μmol, 93.21% yield) as a white solid.

LCMS: [M+H]⁺=312.3

¹H NMR (400 MHz, CHLOROFORM-d) δ=7.27-7.22 (m, 1H), 7.21-7.16 (m, 1H), 7.16-7.12 (m, 1H), 6.98-6.83 (m, 3H), 5.02 (s, 2H), 3.04 (s, 1H), 1.39 (s, 6H).

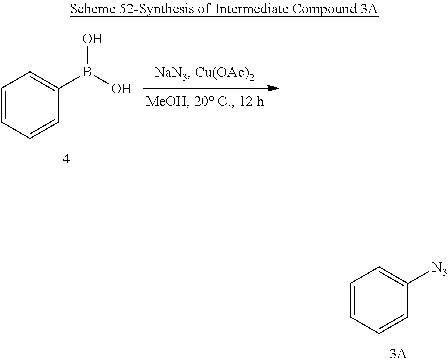 [SMILES: OB(O)c1ccccc1 ; [N-]=[N+]=Nc1ccccc1]

To a solution of Compound 4 (300 mg, 2.46 mmol, 1 eq) in MeOH (5 mL) was added NaN₃ (300 mg, 4.61 mmol, 1.88 eq) and Cu(OAc)₂ (44.69 mg, 246.04 μmol, 0.1 eq). The reaction was stirred at 20° C. for 12 h. The reaction mixture was diluted with ethyl acetate (20 mL) and water (20 mL). The layers were separated and the aqueous layer was extracted with ethyl acetate (10 mL). The combined organic layers were washed with brine (20 mL), dried, filtered and concentrated till half amount of solvent remained. EtOH (20 mL) was added and the organic solution was concentrated half amount of solvent again. The solvent replacement was repeat for 3 times to obtain Compound 3A (167 mg, 1.40 mmol, 56.98% yield) as a yellow liquid.

GCMS: [M+H]⁺=119.1

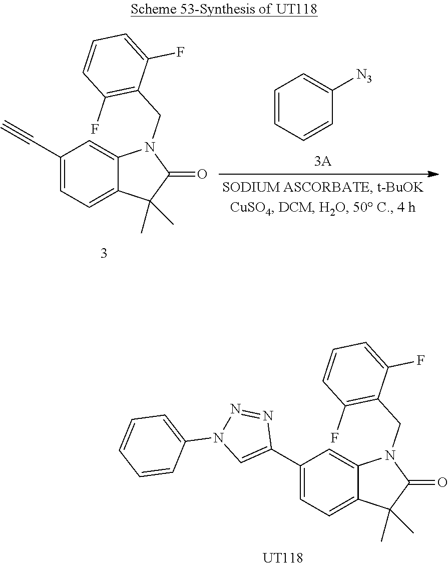 [SMILES: C#Cc1ccc2c(c1)N(Cc1c(F)cccc1F)C(=O)C2(C)C ; [N-]=[N+]=Nc1ccccc1 ; CC1(C)C(=O)N(Cc2c(F)cccc2F)c2cc(-c3cn(-c4ccccc4)nn3)ccc21 ; "UT118"]

To a solution of Compound 3 (57.40 mg, 481.81 umol, 1.5 eq) in DCM (0.5 mL), t-BuOH (0.5 mL) and H₂O (0.5 mL) was added SODIUM ASCORBATE (15.27 mg, 77.09 umol, 0.24 eq), Compound 3A (100 mg, 321.21 umol, 1 eq) and CuSO₄ (4.10 mg, 25.70 umol, 3.94 uL, 0.08 eq). The mixture was stirred at 50° C. for 4 h. The reaction mixture was filtered and the filtrate was concentrated under the reduced pressure to give a residue. The residue was purified by prep-HPLC (52%-82% MeCN in water (FA), 9 min), to obtain UT118 (94.79 mg, 219.53 μmol, 68.34% yield, 99.69% purity) as a white solid.

LCMS: [M+H]⁺=431.1

¹H NMR (400 MHz, DMSO-d₆) δ=9.22 (s, 1H), 8.01-7.89 (m, 2H), 7.64 (t, J=7.6 Hz, 2H), 7.59-7.46 (m, 4H), 7.45-7.35 (m, 1H), 7.12 (t, J=8.0 Hz, 2H), 5.05 (s, 2H), 2.07 (s, 1H), 1.31 (s, 6H).

### Example 23: Synthesis of STING Modulator—UT120

Compound UT120 was synthesized according to FIG. 29, which is split into Scheme 54-55 below.

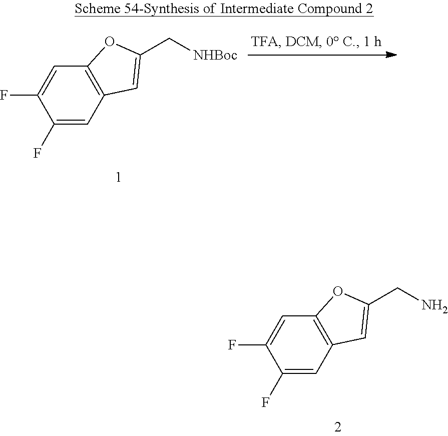 [SMILES: CC(C)(C)OC(=O)NCc1cc2cc(F)c(F)cc2o1 ; NCc1cc2cc(F)c(F)cc2o1]

To a solution of Compound 1 (200 mg, 706.04 umol, 1 eq) in DCM (1 mL) was added TFA (1.54 g, 13.51 mmol, 1 mL, 19.13 eq). The mixture was stirred at 0° C. for 1 h. The reaction mixture was added NaHCO₃ until pH=8, and then extracted with EtOAc (10 ml×3). The combined organic layers were dried over anhydrous Na₂SO₄, filtered and concentrated to obtain Compound 2 (232 mg, crude) as a yellow oil.

LCMS: [M+H]⁺=167.0

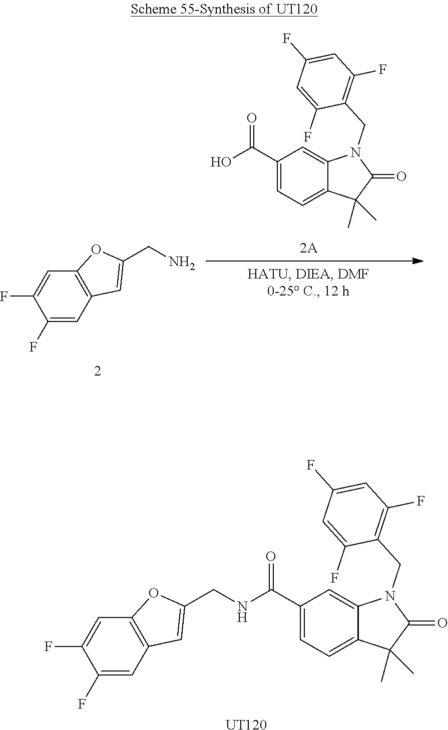 [SMILES: CC1(C)C(=O)N(Cc2c(F)cc(F)cc2F)c2cc(C(=O)O)ccc21 ; NCc1cc2cc(F)c(F)cc2o1 ; CC1(C)C(=O)N(Cc2c(F)cc(F)cc2F)c2cc(C(=O)NCc3cc4cc(F)c(F)cc4o3)ccc21 ; "UT120"]

To a solution of Compound 2A (200 mg, 572.57 umol, 1 eq) in DMF (2 mL) was added HATU (326.56 mg, 858.85 umol, 1.5 eq) at 0° C. The mixture was stirred at this temperature for 30 min. Compound 2 was then added (104.87 mg, 572.57 umol, 1 eq) and DIEA (222.00 mg, 1.72 mmol, 299.19 uL, 3 eq) at 0° C. The mixture was stirred at 25° C. for 11.5 h. The residue was purified by prep-HPLC (column: Phenomenex luna C18 150*25 mm*10 um; mobile phase: [water (HCl)-ACN]; B %: 51%-81%, 10 min) and lyophilized to obtain UT120 (199.55 mg, 386.55 μmol, 67.51% yield, 99.653% purity) as an off-white solid.

LCMS: [M+H]⁺=515.0

¹H NMR (400 MHz, DMSO-d₆) δ=9.07 (t, J=5.2 Hz, 1H), 7.80 (dd, J=6.4, 10.0 Hz, 1H), 7.72-7.55 (m, 2H), 7.54-7.37 (m, 2H), 7.36-7.12 (m, 2H), 6.90-6.65 (m, 1H), 4.96 (s, 2H), 4.61 (d, J=5.2 Hz, 2H), 1.28 (s, 6H).

### Example 24: Synthesis of STING Modulator—UT121

Compound UT121 was synthesized according to FIG. 30, which is reproduced as scheme 56 below.

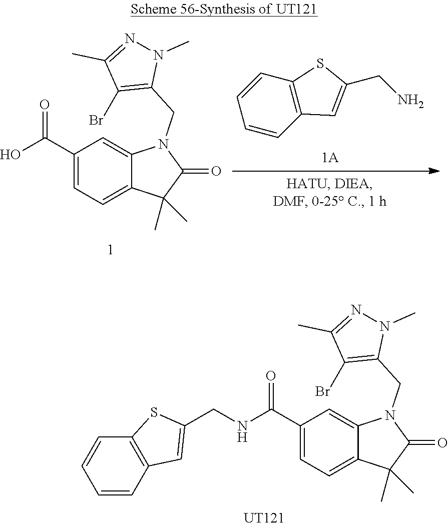 [SMILES: Cc1nn(C)c(CN2C(=O)C(C)(C)c3ccc(C(=O)O)cc32)c1Br ; NCc1cc2ccccc2s1 ; Cc1nn(C)c(CN2C(=O)C(C)(C)c3ccc(C(=O)NCc4cc5ccccc5s4)cc32)c1Br ; "UT121"]

To a solution of Compound 1 (103.71 mg, 264.40 umol, 1 eq) in DMF (1.5 mL) was added HATU (150.80 mg, 396.59 umol, 1.5 eq) at 0° C., and the mixture was stirred at 0° C. for 30 min. After was added Compound 1A (43.16 mg, 264.40 umol, 1 eq) and DIEA (102.51 mg, 793.19 umol, 138.16 uL, 3 eq), and the mixture was stirred at 25° C. for 30 min. The residue was purified by Prep-HPLC (46%-76% MeCN in water (FA), 9 min, and 37%-67% MeCN in water (NH₃H₂O), 8 min), to obtain UT121 (9.33 mg, 17.72 μmol, 6.95% yield, 99.21% purity) was obtained as a white solid.

LCMS: [M+H]⁺=539.0

¹H NMR (400 MHz, DMSO-d₆) δ=9.15 (t, J=6.0 Hz, 1H), 7.89 (d, J=8.0 Hz, 1H), 7.77 (d, J=7.6 Hz, 1H), 7.60 (dd, J=7.6, 1.2 Hz, 1H), 7.49 (d, J=7.6 Hz, 1H), 7.45 (s, 1H), 7.40-7.22 (m, 3H), 4.97 (s, 2H), 4.72 (d, J=5.6 Hz, 2H), 3.75 (s, 3H), 2.06 (s, 3H), 1.33 (s, 6H).

### Example 25: Synthesis of STING Modulator—UT126

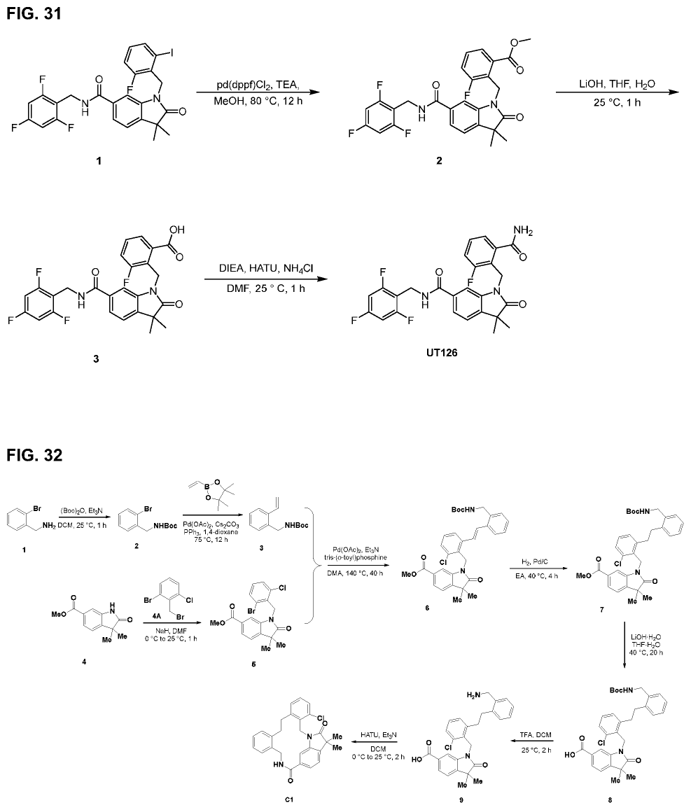

Compound UT126 was synthesized according to FIG. 31, which is split into Schemes 57-59 below.

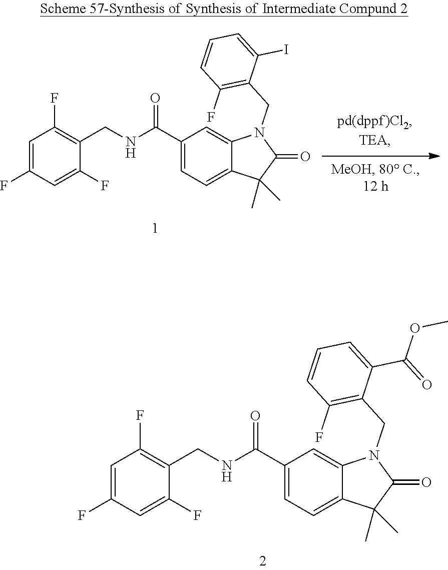 [SMILES: CC1(C)C(=O)N(Cc2c(F)cccc2I)c2cc(C(=O)NCc3c(F)cc(F)cc3F)ccc21 ; COC(=O)c1cccc(F)c1CN1C(=O)C(C)(C)c2ccc(C(=O)NCc3c(F)cc(F)cc3F)cc21]

A mixture of Compound 1 (400 mg, 686.90 umol, 1 eq), Pd(dppf)Cl₂ (50.26 mg, 68.69 umol, 0.1 eq) and TEA (139.01 mg, 1.37 mmol, 191.21 uL, 2 eq) in MeOH (4 mL) was degassed and purged with CO for 3 times, and then the mixture was stirred under 15 psi at 80° C. for 12 h under CO atmosphere. The reaction mixture was concentrated under reduced pressure to remove solvent. The residue was diluted with H₂O (3 mL) and extracted with EtOAc (4 mL×3), dried over Na₂SO₄, filtered and concentrated under reduced pressure and purified by flash silica gel chromatography (ISCO®; 4 g SepaFlash® Silica Flash Column, Eluent of 0˜20% Ethyl acetate/Petroleum ether gradient @50 mL/min) to obtain Compound 2 (220 mg, 427.63 μmol, 62.25% yield) as a white solid.

¹H NMR (400 MHz, CHLOROFORM-d₆) δ=7.58 (d, J=7.6 Hz, 1H), 7.46 (dd, J=7.6, 1.6 Hz, 1H), 7.34 (dt, J=8.0, 5.2 Hz, 1H), 7.28 (s, 1H), 7.24-7.11 (m, 2H), 6.70 (t, J=8.2 Hz, 2H), 6.38 (t, J=5.2 Hz, 1H), 5.32 (s, 2H), 4.66 (d, J=5.6 Hz, 2H), 3.94 (s, 3H), 1.37 (s, 6H).

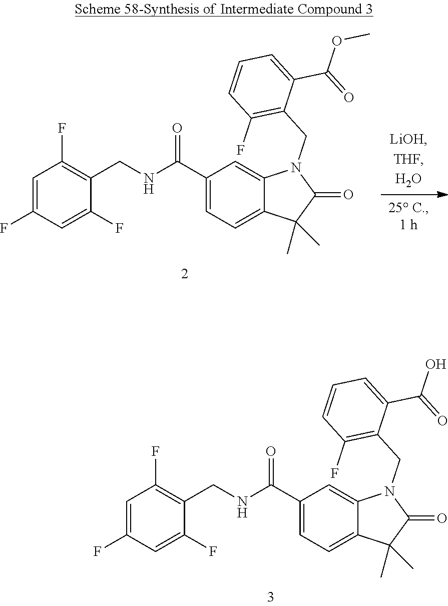 [SMILES: COC(=O)c1cccc(F)c1CN1C(=O)C(C)(C)c2ccc(C(=O)NCc3c(F)cc(F)cc3F)cc21 ; CC1(C)C(=O)N(Cc2c(F)cccc2C(=O)O)c2cc(C(=O)NCc3c(F)cc(F)cc3F)ccc21]

To a solution of Compound 2 (220 mg, 427.63 μmol, 1 eq) in THE (2 mL) and H2O (1 mL) was added LiOH (35.89 mg, 855.25 umol, 2 eq). The mixture was stirred at 25° C. for 1 h. The residue was purified by flash silica gel chromatography (ISCO®; 4 g SepaFlash® Silica Flash Column, Eluent of 0˜45% Ethyl acetate/Petroleum ether gradient @80 mL/min) to obtain Compound 3 (200 mg, 398.86 μmol, 93.27% yield, 99.802% purity) as a white solid.

LCMS: [M+H]⁺=500.9

¹H NMR (400 MHz, DMSO-d₆) δ=12.13 (s, 1H), 8.72 (t, J=4.8 Hz, 1H), 7.63 (d, J=7.6 Hz, 1H), 7.49 (dd, J=8.0, 1.2 Hz, 1H), 7.46-7.30 (m, 4H), 7.17 (t, J=8.8 Hz, 2H), 5.29 (s, 2H), 4.43 (d, J=54.8 Hz, 2H), 1.24 (s, 6H).

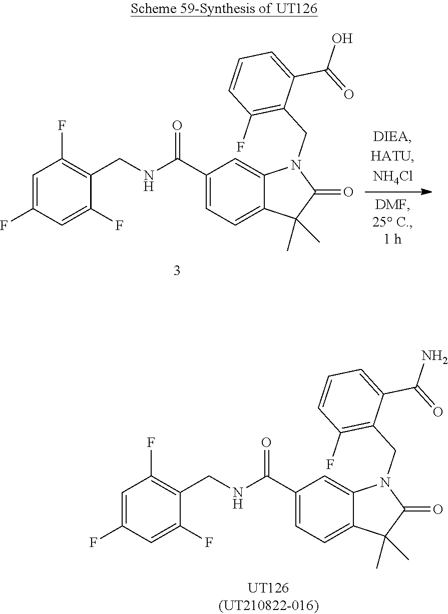 [SMILES: CC1(C)C(=O)N(Cc2c(F)cccc2C(=O)O)c2cc(C(=O)NCc3c(F)cc(F)cc3F)ccc21 ; CC1(C)C(=O)N(Cc2c(F)cccc2C(N)=O)c2cc(C(=O)NCc3c(F)cc(F)cc3F)ccc21]

To a solution of Compound 3 (180 mg, 359.68 umol, 1 eq) in DMF (4 mL) was added NH₄Cl (28.86 mg, 539.52 umol, 1.5 eq), DIEA (139.46 mg, 1.08 mmol, 187.95 uL, 3 eq) and HATU (205.14 mg, 539.52 umol, 1.5 eq). The mixture was stirred at 25° C. for 1 h. The residue was purified by prep-HPLC (column: Phenomenex luna C18 150*25 mm*10 um; mobile phase: [water (FA)-ACN]; B %: 32%-62%, 9 min) and lyophilized to obtain UT126 (40.16 mg, 79.98 μmol, 22.24% yield, 99.469% purity) as a white solid.

LCMS: [M+H]⁺=500.3

¹H NMR (400 MHz, DMSO-d₆) δ=8.64 (t, J=5.2 Hz, 1H), 8.04 (s, 1H), 7.65 (s, 1H), 7.52-7.44 (m, 2H), 7.43-7.33 (m, 2H), 7.26 (dd, J=7.6, 0.8 Hz, 1H), 7.24-7.12 (m, 3H), 5.10 (s, 2H), 4.44 (d, J=5.2 Hz, 2H), 1.26 (s, 6H).

### Example 26: Synthesis of STING Modulator—C1

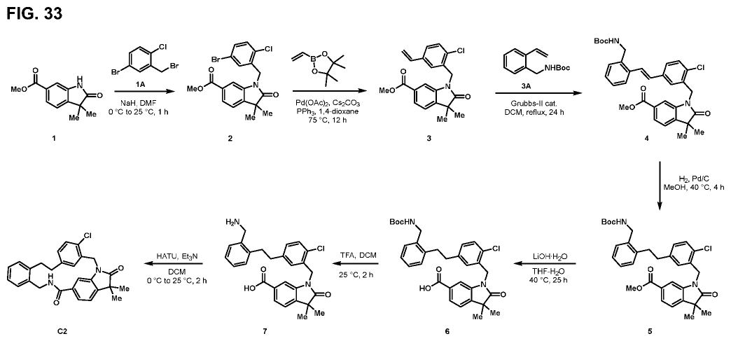

Compound C1 was synthesized according to FIG. 32, which is split into Schemes 60-67 below.

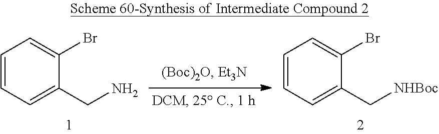 [SMILES: NCc1ccccc1Br ; CC(C)(C)OC(=O)NCc1ccccc1Br]

To a solution of Compound 1 (578.0 mg, 3.11 mmol, 1.0 eq) in DCM (6 mL) was added (Boc)₂O (1.1 mL, 4.66 mmol, 1.5 eq) at 25° C., followed by Et₃N (0.87 mL, 6.21 mmol, 2.0 eq). The reaction mixture was stirred at the same temperature for 1 hour. The reaction mixture was concentrated under the reduced pressure to give the crude product, which was purified by flash column chromatography (SiliaFlash® Irregular Silica Gel, P60 40-63 μm, 60 Å, 10%˜20% Ethyl acetate/Petroleum ether gradient, manually) to obtain Compound 2 (675.0 mg, 2.36 mmol, 75.84% yield) as a light-yellow oil.

LCMS: [M+Na]⁺=308.1 ¹H NMR (400 MHz, CDCl₃) δ=7.54 (dd, J=8.0, 1.3 Hz, 1H), 7.42-7.34 (m, 1H), 7.29 (td, J=7.5, 1.3 Hz, 1H), 7.14 (td, J=7.6, 1.8 Hz, 1H), 5.01 (s, 1H), 4.39 (d, J=6.3 Hz, 2H), 1.45 (s, 9H).

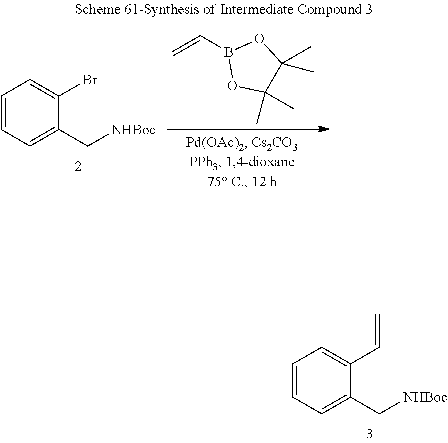 [SMILES: CC(C)(C)OC(=O)NCc1ccccc1Br ; C=CB1OC(C)(C)C(C)(C)O1 ; C=Cc1ccccc1CNC(=O)OC(C)(C)C]

To a stirred solution of Compound 2 (550.0 mg, 1.92 mmol, 1.0 eq) in 1,4-dioxane (20 mL) was added pinacol vinylboronate (342.8 mg, 2.11 mmol, 1.1 eq) at 25° C. Then, Cs₂CO₃ (1.252 g, 3.84 mmol, 2.0 eq) and PPh₃ (100.8 mg, 0.38 mmol, 0.2 eq) were added to the reaction mixture, followed by Pd(OAc)₂ (21.6 mg, 0.10 mmol, 5 mol %). The reaction mixture was degassed with nitrogen five times and heated to 75° C. After 12 hours, the resulting mixture was allowed to cool to 25° C. and filtered through a pad of Celite. The filtrate was washed with H₂O (20 mL) and extracted with EtOAc (2×20 mL). The combined organic layers were washed with brine (20 mL), dried over anhydrous Na₂SO₄, and concentrated under reduced pressure. The residue was purified by flash column chromatography (SiliaFlash® Irregular Silica Gel, P60 40-63 μm, 60 Å, 10%˜20% Ethyl acetate/Petroleum ether gradient, manually) to obtain Compound 3 (443.3 mg, 1.90 mmol, 98.96% yield) as a light-yellow oil.

LCMS: [M+Na]⁺=256.2

¹H NMR (400 MHz, CDCl₃) δ=7.50-7.41 (m, 1H), 7.34-7.27 (m, 0.4H), 7.26-7.15 (m, 2.5H), 7.11-7.03 (m, 0.5H), 6.95-6.84 (m, 0.6H), 5.67 (dd, J=17.3, 1.4 Hz, 0.6H), 5.35 (dd, J=11.0, 1.4 Hz, 0.6H), 5.01-4.86 (m, 0.4H), 4.68-4.51 (m, 0.4H), 4.37-4.21 (m, 2H), 1.41-1.36 (m, 9H).

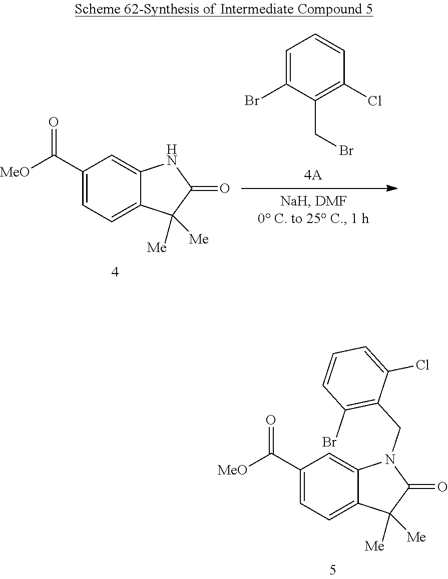 [SMILES: COC(=O)c1ccc2c(c1)NC(=O)C2(C)C ; Clc1cccc(Br)c1CBr ; COC(=O)c1ccc2c(c1)N(Cc1c(Cl)cccc1Br)C(=O)C2(C)C]

To a stirred solution of Compound 4 (109.9 mg, 0.50 mmol, 1.0 eq) in DMF (2 mL) was added NaH (20.0 mg, 0.50 mmol, 1.0 eq, 60% suspension in mineral oil) at 0° C. The resulting mixture was stirred for 15 min at the same temperature before addition of Compound 4A (142.6 mg, 0.50 mmol, 1.0 eq). The reaction mixture was slowly warmed to 25° C. and stirred for 1 hour. The reaction was monitored by TLC. After completion, NH₄Cl (1 mL) was added to the mixture, followed by EtOAc (2 mL). The organic phase was separated, washed with H₂O (2×1 mL) and brine (2 mL), dried over anhydrous Na₂SO₄, and concentrated under reduced pressure. The residue was purified by flash column chromatography (SiliaFlash® Irregular Silica Gel, P60 40-63 μm, 60 Å, 25%˜50% Ethyl acetate/Petroleum ether gradient, manually) to obtain Compound 5 (188.1 mg, 0.44 mmol, 88.77% yield) as a light-yellow solid.

LCMS: [M+H]⁺=422.1

¹H NMR (400 MHz, CDCl₃) δ=7.72 (dd, J=7.7, 1.4 Hz, 1H), 7.57 (dd, J=8.1, 1.2 Hz, 1H), 7.41 (d, J=1.4 Hz, 1H), 7.39 (dd, J=8.3, 1.4 Hz, 1H), 7.24 (d, J=7.7 Hz, 1H), 7.13 (t, J=8.0 Hz, 1H), 5.27 (s, 2H), 3.87 (s, 3H), 1.42 (s, 6H).

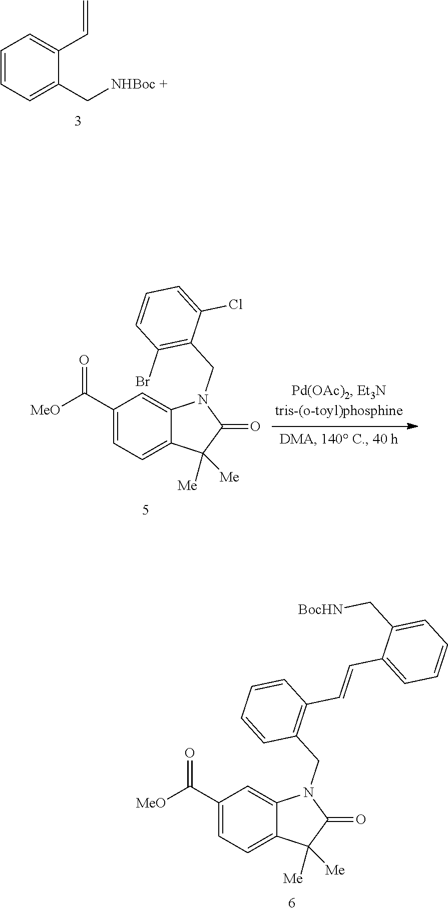

To a stirred solution of Compound 5 (30.2 mg, 0.071 mmol, 1.0 eq) in DMA (1 mL) was added Compound 3 (25.0 mg, 0.107 mmol, 1.5 eq) at 25° C. Then, Et₃N (20.0 μL, 0.142 mmol, 2.0 eq) and Tri-o-tolylphosphine (1.3 mg, 0.004 mmol, 6 mol %) were added to the reaction mixture, followed by Pd(OAc)₂ (0.5 mg, 0.002 mmol, 3 mol %). The reaction mixture was degassed with nitrogen five times and heated to 140° C. After 40 hours, the resulting mixture was allowed to cool to 25° C. and filtered through a pad of Celite. The filtrate was washed with H2O (20 mL) and extracted with EtOAc (2×20 mL). The combined organic layers were washed with brine (20 mL), dried over anhydrous Na₂SO₄, and concentrated under reduced pressure. The residue was purified by flash column chromatography (SiliaFlash® Irregular Silica Gel, P60 40-63 μm, 60 Å, 20%˜50% Ethyl acetate/Petroleum ether gradient, manually) to yield Compound 6 (18.2 mg, 0.032 mmol, 44.29% yield) as a white solid.

LCMS: [M+Na]⁺=597.3

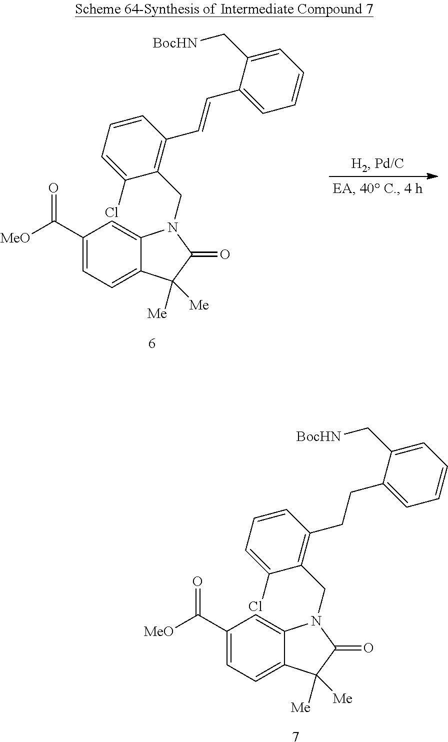

To a stirred solution of Compound 6 (18.2 mg, 0.032 mmol, 1.0 eq) in ethyl acetate (2 mL) was added Pd/C (1.8 mg, 10 wt %) at 25° C. The reaction mixture was degassed three times with a H₂ balloon before heating to 40° C. After 4 hours, the resulting mixture was allowed to cool to 25° C. and filtered through a pad of Celite. The filtrate was concentrated under reduced pressure to obtain Compound 7 (14.4 mg, 0.025 mmol, 78.62% yield) as a white solid.

LCMS: [M+Na]⁺=599.3

¹H NMR (400 MHz, CDCl₃) δ=7.70 (dd, J=7.7, 1.4 Hz, 1H), 7.37 (d, J=1.4 Hz, 1H), 7.32 (dd, J=7.6, 1.7 Hz, 1H), 7.29-7.24 (m, 1H), 7.23-7.14 (m, 5H), 7.08 (dt, J=5.1, 3.5 Hz, 1H), 5.06 (s, 2H), 4.93 (s, 1H), 4.27 (d, J=5.6 Hz, 2H), 3.85 (s, 3H), 3.07-3.00 (m, 2H), 2.92 (dd, J=9.1, 6.0 Hz, 2H), 1.41 (s, 9H), 1.32 (s, 6H).

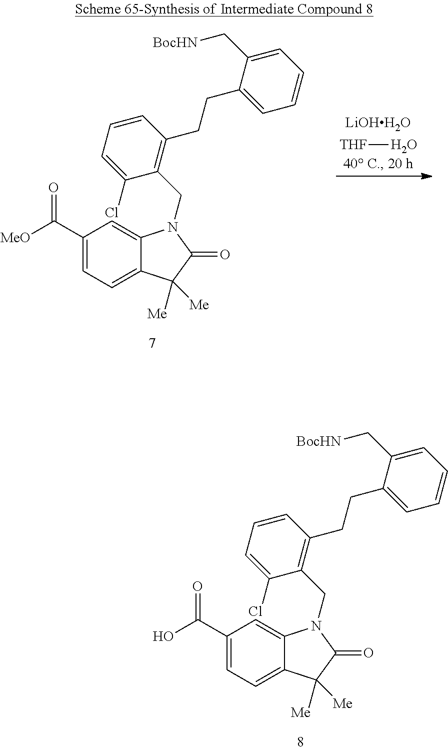

To a stirred solution of Compound 7 (14.4 mg, 0.025 mmol, 1.0 eq) in a mixture of THE (0.2 mL) and H₂O (0.1 mL) was added LiOH·H₂O (4.2 mg, 0.100 mmol, 4.0 eq) at 25° C. The resulting mixture was stirred at 40° C. for 20 hours. The reaction was monitored by TLC and LC-MS. After completion, H₂O (0.5 mL) was added to the mixture, followed by EtOAc (0.5 mL). The aqueous phase was separated and acidified with 1N HCl to pH=5. The aqueous mixture was extracted with EtOAc (2×0.5 mL). The combined organic layers were washed with brine (0.5 mL), dried over anhydrous Na₂SO₄, and concentrated under reduced pressure to yield Compound 8 (12.6 mg, 0.022 mmol, 89.68% yield) as a white solid, which was used for the next step without any further purification.

LCMS: [M+Na]⁺=585.3

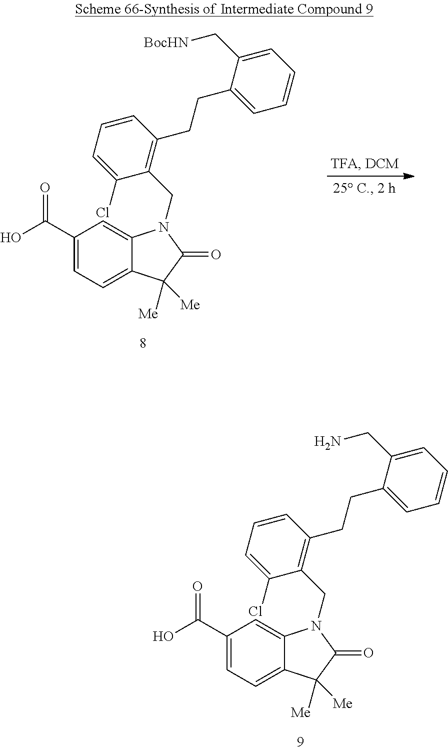

To a stirred solution of Compound 8 (12.6 mg, 0.022 mmol, 1.0 eq) in DCM (1 mL) was added TFA (0.2 mL). The resulting mixture was stirred at 25° C. for 2 hours and concentrated under reduced pressure to obtain Compound 9 (12.3 mg, 0.021 mmol, 95.00% yield) as a white solid, which was used for the next step without any further purification.

LCMS: [M+H]⁺=463.2

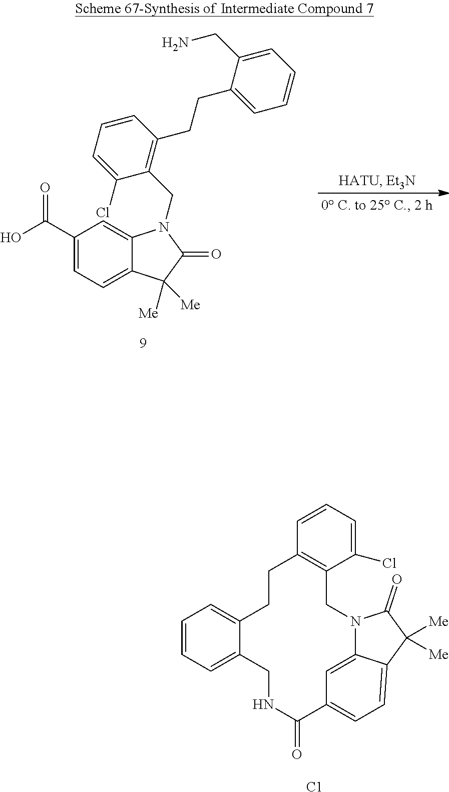

To a stirred solution of Compound 9 (12.3 mg, 0.021 mmol, 1.0 eq) in DCM (0.5 mL) was added Et₃N (8.9 μL, 0.064 mmol, 3.0 eq) and HATU (9.7 mg, 0.026 mmol, 1.2 eq) at 0° C. The reaction mixture was allowed to warm to 25° C. and stirred a further 1 hour. The reaction was monitored by TLC and LC-MS. After completion, H₂O (0.5 mL) was added to the mixture. The organic phase was separated, washed with H₂O (2×0.5 mL) and brine (0.5 mL), dried over anhydrous Na₂SO₄, and concentrated under reduced pressure. The residue was purified by prep-HPLC (C18 column) to obtain C1 (2.1 mg, 0.0047 mmol, 22.14% yield) as a white solid.

LCMS: [M+H]⁺=445.2

¹H NMR (600 MHz, CD₃CN) δ=7.61 (dd, J=21.1, 7.8 Hz, 2H), 7.33 (tt, J=24.8, 7.4 Hz, 5H), 7.21 (q, J=7.5 Hz, 2H), 7.08-6.96 (m, 1H), 5.29 (d, J=15.3 Hz, 1H), 4.70-4.55 (m, 2H), 4.48 (s, 1H), 4.19 (d, J=13.6 Hz, 1H), 3.63 (t, J=11.7 Hz, 2H), 3.54 (q, J=12.6, 11.1 Hz, 1H), 3.01 (t, J=11.8 Hz, 1H), 1.37-1.26 (m, 6H).

### Example 27: Synthesis of STING Modulator—C2

Compound C2 was synthesized according to FIG. 33, which is split into Schemes 68XX below.

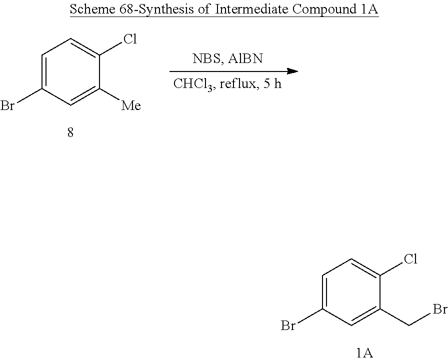 [SMILES: Cc1cc(Br)ccc1Cl ; Clc1ccc(Br)cc1CBr]

To a solution of Compound 8 (457.9 mg, 2.23 mmol, 1.0 eq) in chloroform (2 mL) was added NBS (436.3 mg, 2.45 mmol, 1.1 eq) at 25° C., followed by AlBN (73.2 mg, 0.45 mmol, 0.2 eq). The reaction mixture was refluxed for 5 hours. After cooling to 25° C., the reaction mixture was concentrated under the reduced pressure to give the crude product, which was purified by flash column chromatography (SiliaFlash® Irregular Silica Gel, P60 40-63 μm, 60 Å, 0%˜10% Ethyl acetate/Petroleum ether gradient, manually) to obtain Compound 1A (560.2 mg, 1.97 mmol, 88.40% yield) as a yellow solid.

LCMS: [M+H]⁺=282.3

¹H NMR (400 MHz, CDCl₃) δ=7.59 (d, J=2.4 Hz, 1H), 7.38 (dd, J=8.5, 2.4 Hz, 1H), 7.26 (d, J=8.5 Hz, 1H), 4.52 (s, 2H).

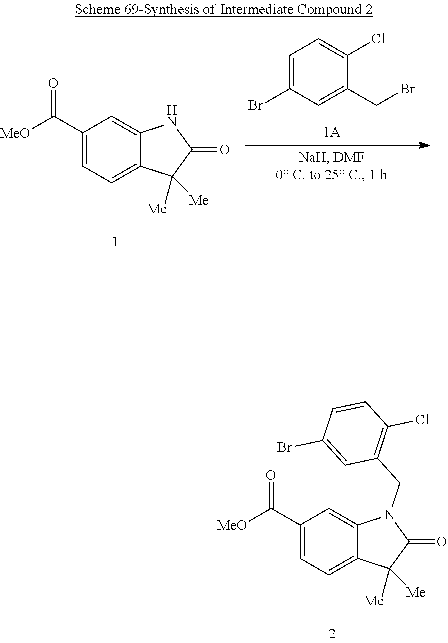 [SMILES: COC(=O)c1ccc2c(c1)NC(=O)C2(C)C ; Clc1ccc(Br)cc1CBr ; COC(=O)c1ccc2c(c1)N(Cc1cc(Br)ccc1Cl)C(=O)C2(C)C]

To a stirred solution of Compound 1 (70.0 mg, 0.32 mmol, 1.0 eq) in DMF (2 mL) was added NaH (12.8 mg, 0.32 mmol, 1.0 eq, 60% suspension in mineral oil) at 0° C. The resulting mixture was stirred for 15 min at the same temperature before addition of Compound 1A (90.8 mg, 0.32 mmol, 1.0 eq). The reaction mixture was slowly warmed to 25° C. and stirred for 1 hour. The reaction was monitored by TLC. After completion, NH₄Cl (1 mL) was added to the mixture, followed by EtOAc (2 mL). The organic phase was separated, washed with H₂O (2×1 mL) and brine (2 mL), dried over anhydrous Na₂SO₄, and concentrated under reduced pressure. The residue was purified by flash column chromatography (SiliaFlash® Irregular Silica Gel, P60 40-63 μm, 60 Å, 25%˜50% Ethyl acetate/Petroleum ether gradient, manually) to yield Compound 2 (104.9 mg, 0.25 mmol, 77.73% yield) as a light-yellow solid.

LCMS: [M+H]⁺=422.1

¹H NMR (400 MHz, CDCl₃) δ=7.82 (dd, J=7.7, 1.4 Hz, 1H), 7.36 (d, J=1.5 Hz, 1H), 7.34 (d, J=2.5 Hz, 1H), 7.31 (d, J=5.5 Hz, 1H), 7.29-7.26 (m, 1H), 7.09 (d, J=2.3 Hz, 1H), 5.01 (s, 2H), 3.89 (s, 3H), 1.48 (s, 6H).

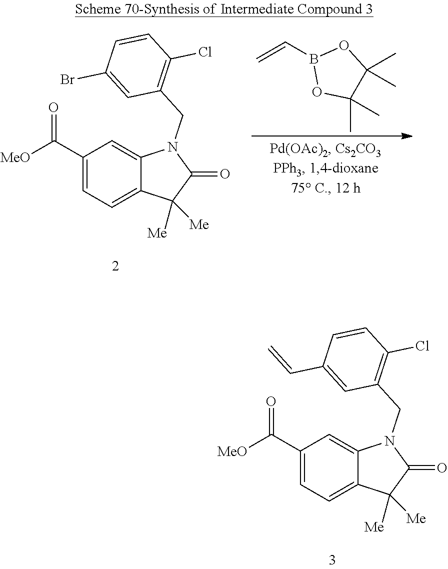 [SMILES: COC(=O)c1ccc2c(c1)N(Cc1cc(Br)ccc1Cl)C(=O)C2(C)C ; C=CB1OC(C)(C)C(C)(C)O1 ; C=Cc1ccc(Cl)c(CN2C(=O)C(C)(C)c3ccc(C(=O)OC)cc32)c1]

To a stirred solution of Compound 2 (35.6 mg, 0.084 mmol, 1.0 eq) in 1,4-dioxane (1 mL) was added pinacol vinylboronate (15.0 mg, 0.093 mmol, 1.1 eq) at 25° C. Then, Cs₂CO₃ (54.7 mg, 0.17 mmol, 2.0 eq) and PPh₃ (4.4 mg, 0.017 mmol, 0.2 eq) were added to the reaction mixture, followed by Pd(OAc)₂ (1.0 mg, 0.0042 mmol, 5 mol %). The reaction mixture was degassed with nitrogen five times and heated to 75° C. After 12 hours, the resulting mixture was allowed to cool to 25° C. and filtered through a pad of Celite. The filtrate was washed with H₂O (1 mL) and extracted with EtOAc (2×1 mL). The combined organic layers were washed with brine (1 mL), dried over anhydrous Na₂SO₄, and concentrated under reduced pressure. The residue was purified by flash column chromatography (SiliaFlash® Irregular Silica Gel, P60 40-63 μm, 60 Å, 10%˜20% Ethyl acetate/Petroleum ether gradient, manually) to yield Compound 3 (27.4 mg, 0.074 mmol, 87.96% yield) as a light-yellow solid.

LCMS: [M+H]⁺=370.2

¹H NMR (400 MHz, CDCl₃) δ=7.79 (dd, J=7.7, 1.4 Hz, 1H), 7.40 (d, J=1.4 Hz, 1H), 7.36 (d, J=8.2 Hz, 1H), 7.31 (d, J=7.7 Hz, 1H), 7.27-7.23 (m, 1H), 7.03 (d, J=2.1 Hz, 1H), 6.53 (dd, J=17.6, 10.9 Hz, 1H), 5.56 (dd, J=17.6, 0.7 Hz, 1H), 5.18 (dd, J=10.8, 0.6 Hz, 1H), 5.05 (s, 2H), 3.87 (s, 3H), 1.48 (s, 6H).

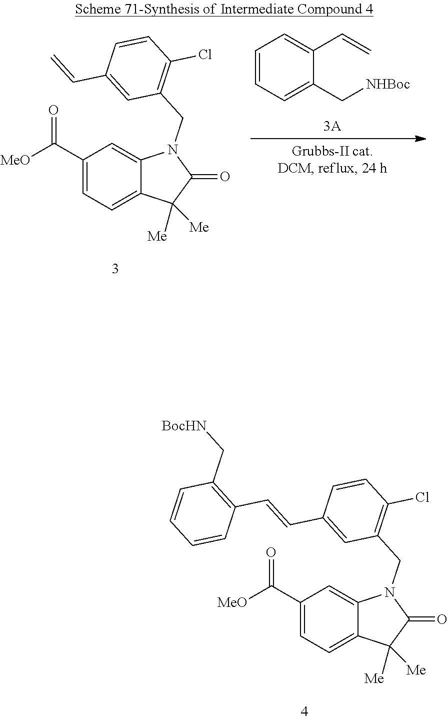 [SMILES: C=Cc1ccc(Cl)c(CN2C(=O)C(C)(C)c3ccc(C(=O)OC)cc32)c1 ; C=Cc1ccccc1CNC(=O)OC(C)(C)C ; COC(=O)c1ccc2c(c1)N(Cc1cc(/C=C/c3ccccc3CNC(=O)OC(C)(C)C)ccc1Cl)C(=O)C2(C)C]

To a stirred solution of Compound 3 (21.2 mg, 0.057 mmol, 1.0 eq) in DCM (6 mL) was added Compound 3A (33.4 mg, 0.14 mmol, 2.5 eq) at 25° C., followed by Grubbs-II catalyst (2.4 mg, 0.0029 mmol, 5 mol %). The reaction mixture was refluxed for 24 hours. After cooling to 25° C., the reaction mixture was filtered through a pad of Celite. The filtrate was concentrated under the reduced pressure to give the crude product, which was purified by flash column chromatography (SiliaFlash® Irregular Silica Gel, P60 40-63 μm, 60 Å, 10%˜50% Ethyl acetate/Petroleum ether gradient, manually) to yield Compound 4 (15.1 mg, 0.026 mmol, 45.81% yield) as a yellow solid.

LCMS: [M+Na]⁺=597.3

¹H NMR (400 MHz, CDCl₃) δ=7.80 (dd, J=7.7, 1.4 Hz, 1H), 7.52 (t, J=8.6 Hz, 2H), 7.44-7.37 (m, 2H), 7.31 (d, J=7.7 Hz, 1H), 7.29-7.21 (m, 4H), 7.08 (d, J=2.2 Hz, 1H), 6.80 (d, J=16.0 Hz, 1H), 5.07 (s, 2H), 4.67 (s, 1H), 4.39 (d, J=5.8 Hz, 2H), 3.87 (s, 3H), 1.50 (s, 6H), 1.38 (s, 9H).

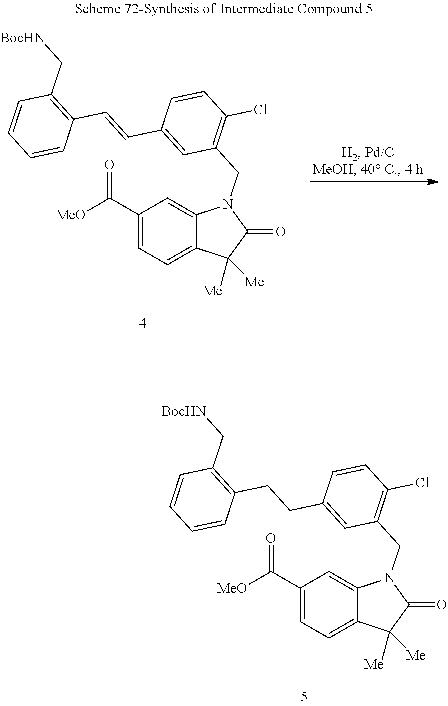 [SMILES: COC(=O)c1ccc2c(c1)N(Cc1cc(/C=C/c3ccccc3CNC(=O)OC(C)(C)C)ccc1Cl)C(=O)C2(C)C ; COC(=O)c1ccc2c(c1)N(Cc1cc(CCc3ccccc3CNC(=O)OC(C)(C)C)ccc1Cl)C(=O)C2(C)C]

To a stirred solution of Compound 4 (15.1 mg, 0.026 mmol, 1.0 eq) in ethyl acetate (2 mL) was added Pd/C (1.5 mg, 10 wt %) at 25° C. The reaction mixture was degassed three times with a H₂ balloon before heating to 40° C. After 4 hours, the resulting mixture was allowed to cool to 25° C. and filtered through a pad of Celite. The filtrate was concentrated under reduced pressure to yield Compound 5 (12.9 mg, 0.022 mmol, 85.13% yield) as a white solid.

LCMS: [M+Na]⁺=599.3

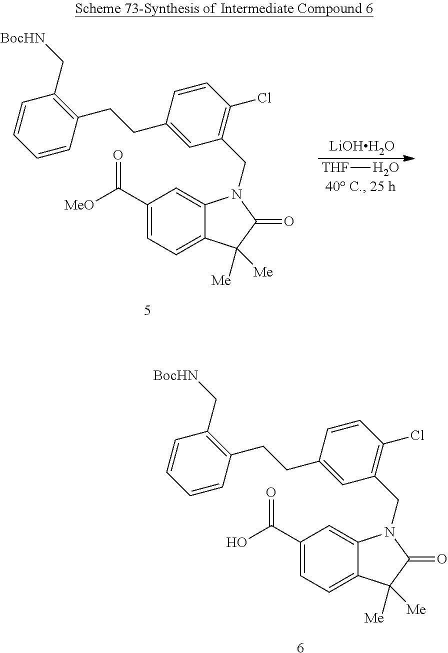

To a stirred solution of Compound 5 (12.9 mg, 0.022 mmol, 1.0 eq) in a mixture of THE (0.2 mL) and H₂O (0.1 mL) was added LiOH·H₂O (3.8 mg, 0.089 mmol, 4.0 eq) at 25° C. The resulting mixture was stirred at 40° C. for 25 hours. The reaction was monitored by TLC and LC-MS. After completion, H₂O (0.5 mL) was added to the mixture, followed by EtOAc (0.5 mL). The aqueous phase was separated and acidified with 1N HCl to pH=5. The aqueous mixture was extracted with EtOAc (2×0.5 mL). The combined organic layers were washed with brine (0.5 mL), dried over anhydrous Na₂SO₄, and concentrated under reduced pressure to yield Compound 6 (11.8 mg, 0.021 mmol, 93.75% yield) as a white solid, which was used for the next step without any further purification.

LCMS: [M+Na]⁺=585.3

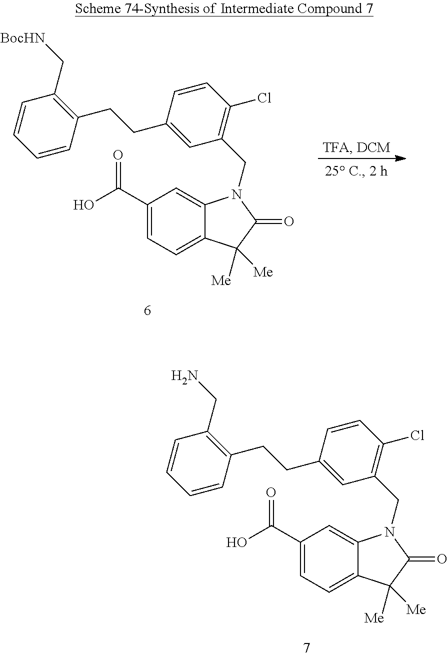

To a stirred solution of Compound 6 (11.8 mg, 0.021 mmol, 1.0 eq) in DCM (1 mL) was added TFA (0.2 mL). The resulting mixture was stirred at 25° C. for 2 hours and concentrated under reduced pressure to obtain Compound 7 (11.0 mg, 0.019 mmol, 90.97% yield) as a white solid, which was used for the next step without any further purification.

LCMS: [M+H]⁺=463.2

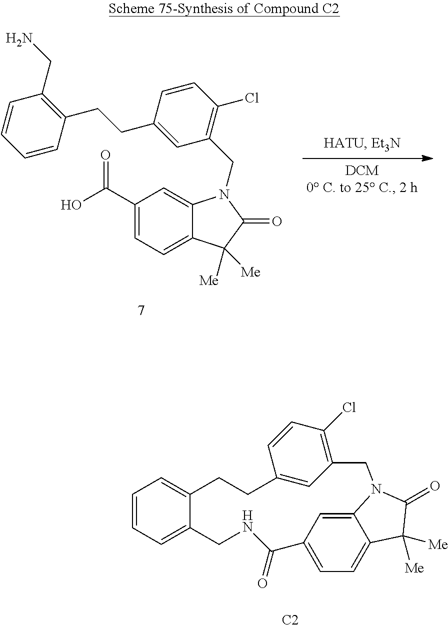

To a stirred solution of Compound 7 (11.0 mg, 0.019 mmol, 1.0 eq) in DCM (0.5 mL) was added Et₃N (8.0 μL, 0.057 mmol, 3.0 eq) and HATU (8.7 mg, 0.023 mmol, 1.2 eq) at 0° C. The reaction mixture was allowed to warm to 25° C. and stirred a further 1 hour. The reaction was monitored by TLC and LC-MS. After completion, H₂O (0.5 mL) was added to the mixture. The organic phase was separated, washed with H₂O (2×0.5 mL) and brine (0.5 mL), dried over anhydrous Na₂SO₄, and concentrated under reduced pressure. The residue was purified by prep-HPLC (C18 column) to obtain C2 (4.6 mg, 0.010 mmol, 54.23% yield) as a white solid.

LCMS: [M+H]⁺=445.2

¹H NMR (400 MHz, CDCl₃) δ=7.67 (d, J=7.8 Hz, 1H), 7.63 (d, J=2.2 Hz, 1H), 7.37-7.46 (m, 2H), 7.24-7.14 (m, 3H), 6.99 (d, J=8.1 Hz, 1H), 6.75 (dd, J=8.2, 2.1 Hz, 1H), 6.69 (d, J=1.5 Hz, 1H), 5.02 (s, 2H), 4.90 (s, 1H), 4.41 (d, J=4.0 Hz, 2H), 3.34-3.28 (m, 2H), 3.26-3.18 (m, 2H), 1.39 (s, 6H).

### Example 28: Comparison of Activities of UT009 with STING Agonist C53

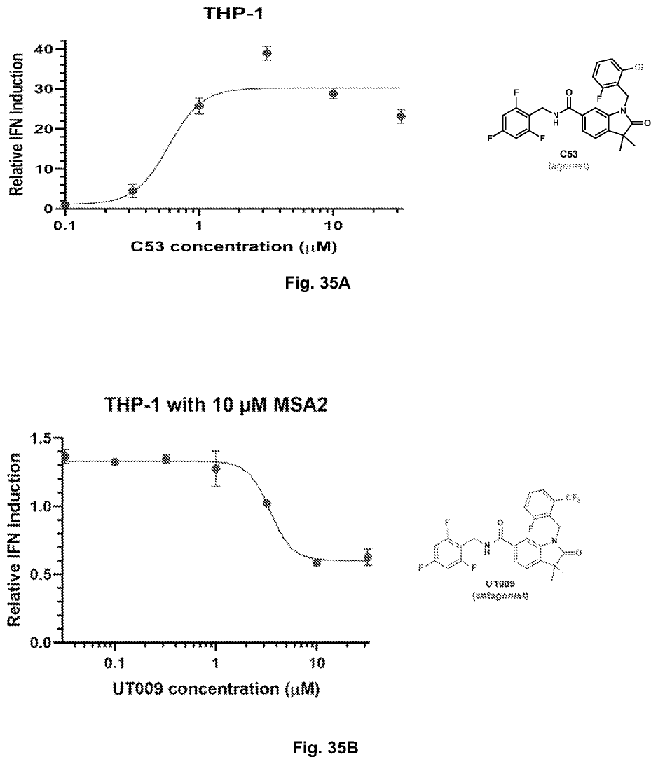

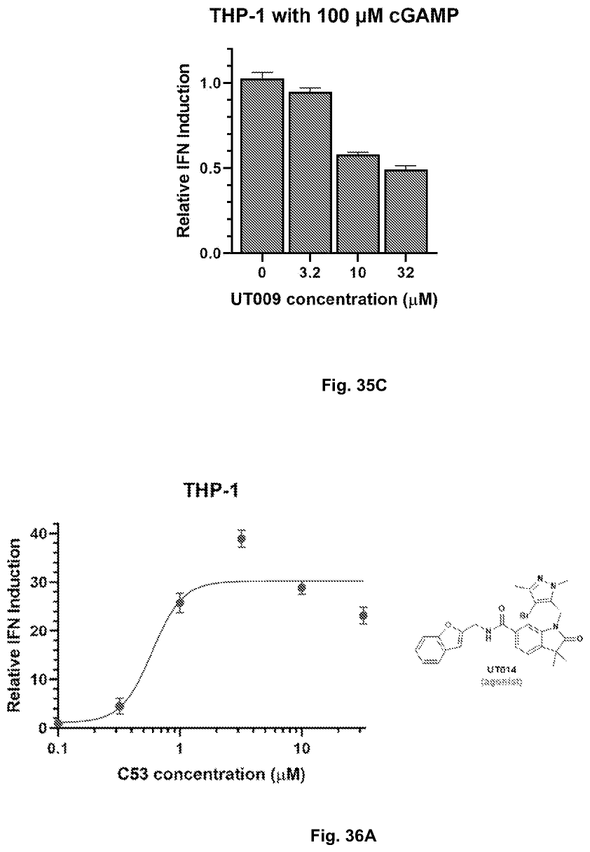

Compounds C53 and UT009 were tested for their ability to interfere STING signaling. As shown in FIG. 35A and FIG. 35B, the structures of UT009 and C53 differ by a single moiety (CI in C53 and —CF₃ in UT009). IFN induction was measured in THP-1 cell lines, treated with increasing concentrations of C53 or increasing concentrations of UT009 in the presence of with 10 μM MSA2, a known STING agonist. While the relative induction of interferon production was found to be enhanced in a dose-dependent manner, for C53 (FIG. 35A), UT009 inhibited the induction of interferon production in a dose dependent manner (FIG. 35B). UT009 was further tested in the presence of 100 μM cGAMP, for its effect on interferon induction in THP-1 cells. Dose response curves exhibited reduction in interferon production with increasing concentration of compound UT009 in the presence of cGAMP (FIG. 35C). This indicated that substitution of the Cl moiety in C53, surprisingly switched the activity of the compound from being an agonist to an antagonist.

### Example 29: Comparison of Activities of STING Agonist UT014 and UT112

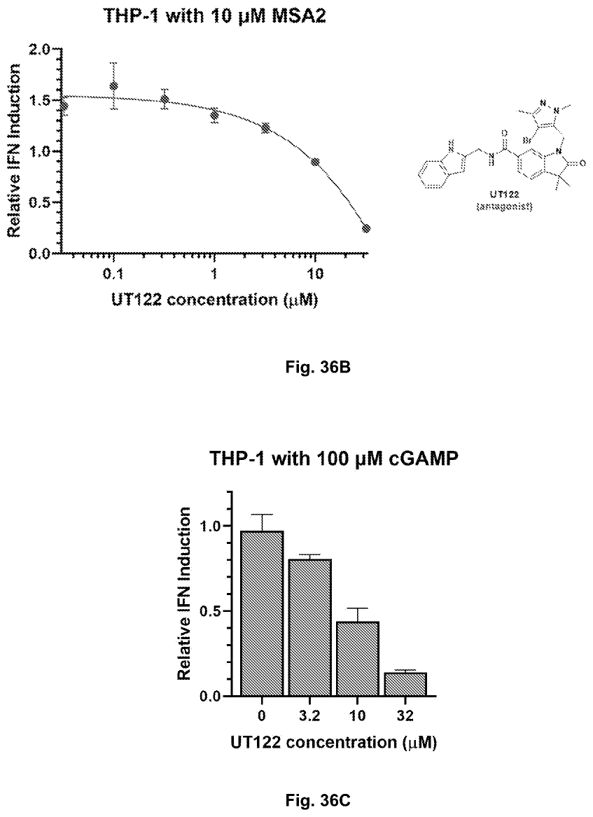

Compounds UT014 and UT122 were tested for its ability to regulate STING signaling. Activities of the compounds were analyzed by measuring interferon levels in the presence of increasing concentration of the compounds in THP-1 cells. UT014 has a O moiety in the R₂ group, while UT122 has a NH moiety at the same position in R₂ group. UT014 was found to exhibit agonistic activity, by enhancing the induction of interferon levels in a dose dependent manner (FIG. 36A), while UT112 was found to exhibit antagonistic activity with an inhibition of interferon production in a dose dependent manner, in the presence of 10 μM of a known agonist, MSA2 (FIG. 36B), or 100 μM cGAMP (FIG. 36C). This suggested that a simple heteroatom substitution can surprisingly affect the activity of the compound, by converting a STING agonist to a STING antagonist.

### Example 30: Cryo-EM Structure of Human STING in Complex with Agonist Compound C53 and Antagonist Compound UT009

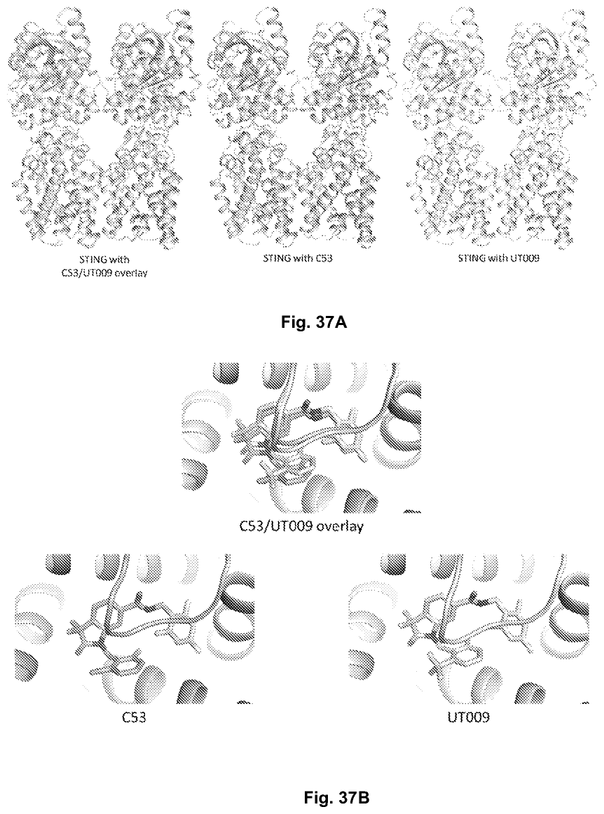

High-resolution cryo-EM structure of human STING oligomer in complex with the agonist C53 or the antagonist compound UT009 were analyzed to understand the conformation and position of the target binding site of C53 and UT009. As shown in FIG. 37A and FIG. 37B, C53 was found to bind to the cryptic site in human STING-TMD through an induced-fit mechanism, as described in Example 4. Further, antagonist compound UT009 appeared to bind at the same binding pocket as C53 (FIG. 37A and FIG. 37B). Structure comparison using images of STING bound to C53 overlaid with STING bound to UT009 further confirmed that the agonist C53 and the antagonist UT009 bound to STING in a similar conformation and manner (FIG. 37B).

### Example 31: Activity of UT017, UT018, UT019 and UT122

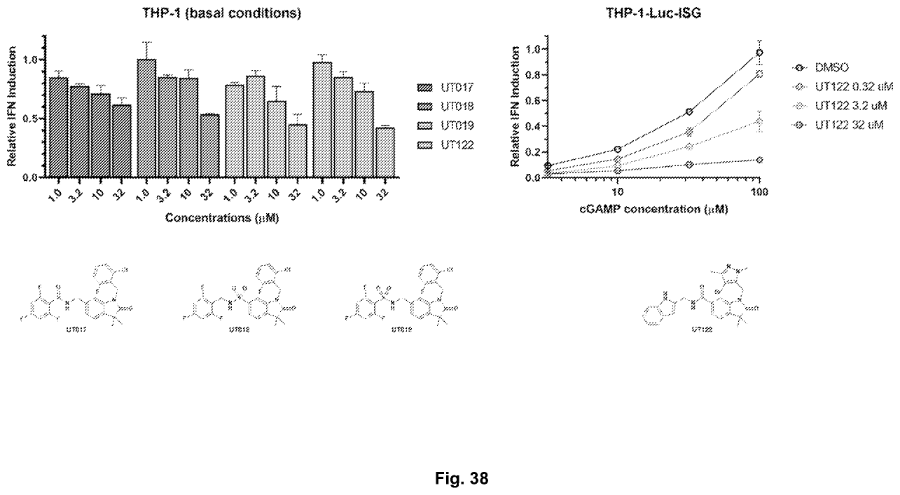

Compounds UT017, UT018, UT019 and UT0122 were tested for their ability to interfere with STING signaling. The structures of each of these compounds is shown in FIG. 38. Interferon production in the presence of increasing concentrations of UT017, UT018, UT019 or UT0122 were measured in THP-1 cell lines under basal conditions. The relative induction of interferon production was found to be suppressed at the highest concentration tested for UT017, UT018, UT019 and UT0122 (FIG. 38) with compounds UT019 and UT0122 exhibiting the highest antagonistic effect. Compound UT0122 was further tested in THP1-Lucia™ ISG cells for interferon production at varying concentration, in the presence of an increasing dose of a STING agonist cGAMP. Induction of interferon by cGAMP was suppressed in a dose-dependent manner by compound UT0122 (FIG. 38).

### Example 32: Activity of UT141, UT142, UT157, UT153, UT156 and UT158

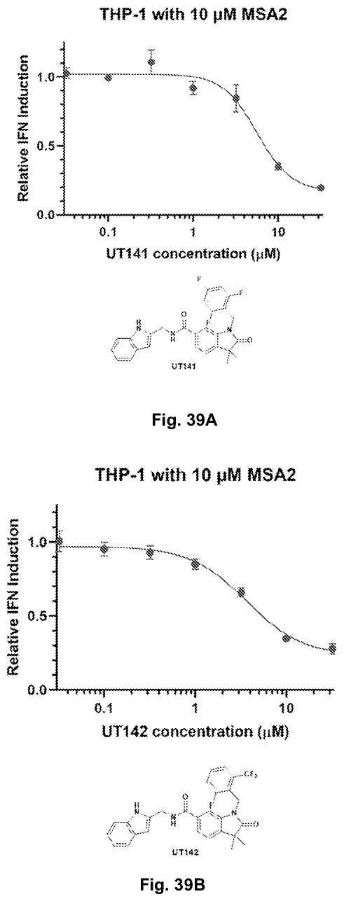

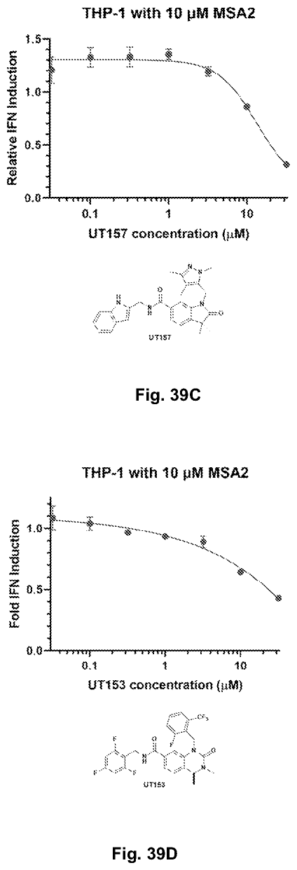

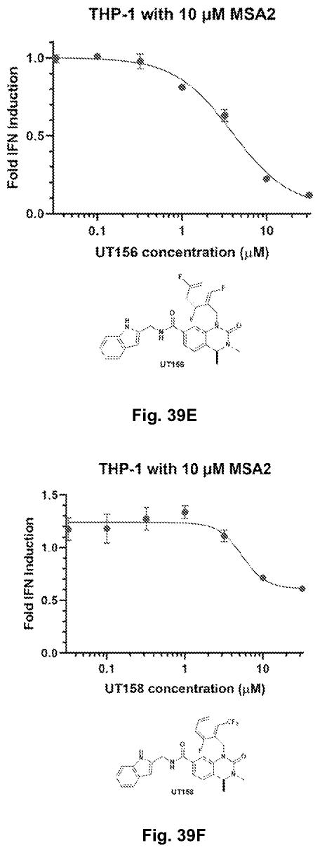

Further, compounds UT141, UT142, UT157, UT153, UT156 and UT158 were tested for their ability to interfere with STING signaling. Structures of each of these compounds are shown in FIG. 39A-FIG. 39F. Interferon production in the presence of increasing concentrations of UT141, UT142, UT157, UT153, UT156 and UT158 were measured in THP-1 cell lines treated with 10 μM MS2. The relative induction of interferon production was found to be inhibited in a dose dependent manner for UT141, UT142, UT157, UT153, UT156 and UT158 (FIGS. 39A-F) with compound UT156 showing the highest suppression of interferon production. Compound UT0156 was further tested in THP1 cells for interferon production at varying concentration, in the presence of a STING agonist MS2. For comparison, a small molecule STING inhibitor H151 was simultaneously tested in the presence of MS2. As shown in FIG. 39G, UT0156 reduced the induction of interferon by MSA2 in a dose-dependent manner at both of the MSA2 doses tested. H151 suppressed the induction of interferon in a dose dependent manner at lower concentration tested, but was less effective in suppressing interferon production at a higher MSA2 dose.

### Summary of Examples 28-32

The results from the above described examples showed that substituting moieties across the groups of the disclosed formulas may result in surprisingly different effects on the activity of the STING protein. Conversion of a STING agonist to a STING antagonist can be made with as little as a single substitution to the moiety in a structural group. Additionally, even though STING agonists and antagonists have opposite modulatory activities, the STING antagonist was found to bind STING in a similar conformation and manner, as an agonist. The STING modulators described herein therefore provide a range of compounds that are useful in a wide range of therapeutic applications, as STING agonists or antagonists.

### Example 33: Synthesis of UT156

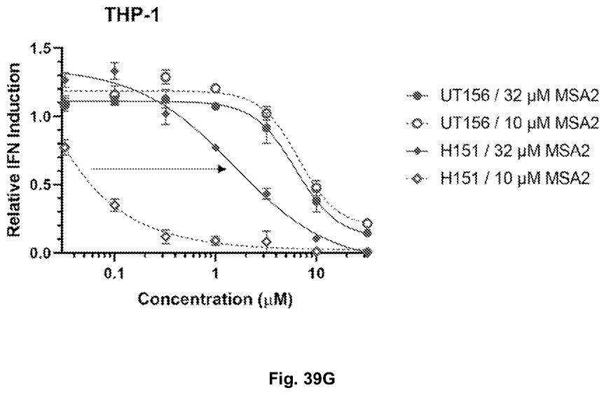

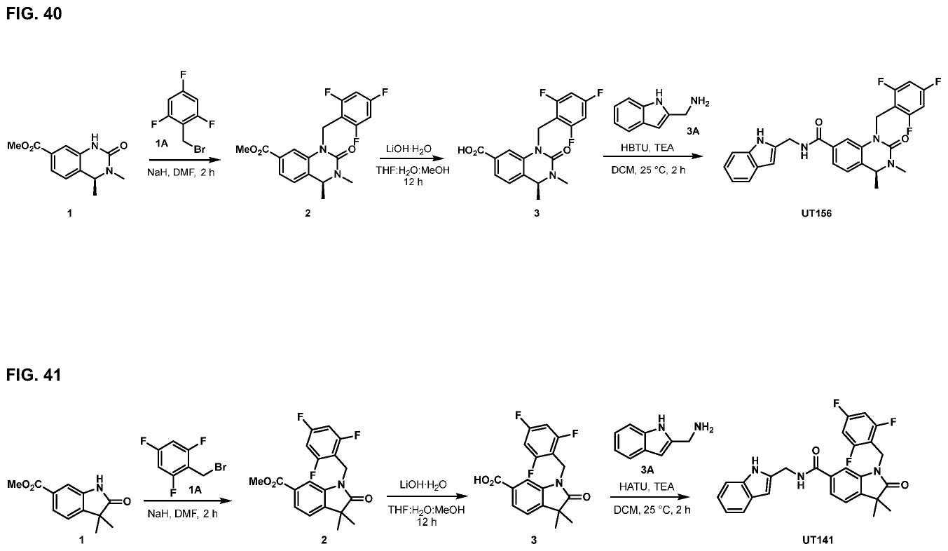

FIG. 40 depicts a representative synthesis scheme to prepare compound UT156 by generating two intermediate compounds (intermediate Compound 2 and intermediate Compound 3).

### Synthesis of Intermediate Compound 2

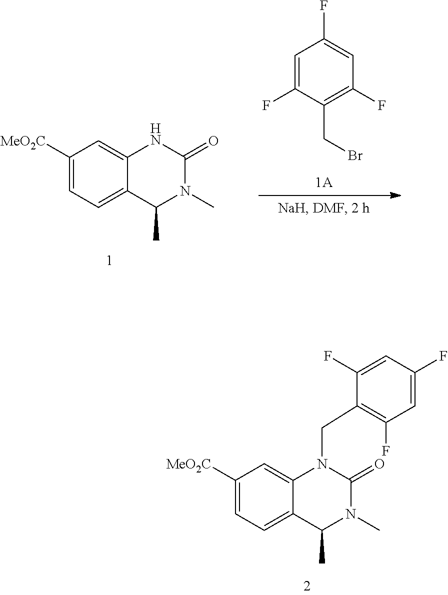 [SMILES: COC(=O)c1ccc2c(c1)NC(=O)N(C)[C@H]2C ; Fc1cc(F)c(CBr)c(F)c1 ; COC(=O)c1ccc2c(c1)N(Cc1c(F)cc(F)cc1F)C(=O)N(C)[C@H]2C]

To a solution of Compound 1 (30 mg, 0.128 mmol, 1 eq) in DMF (0.5 mL) was added NaH (6.1 mg, 0.154 mmol, 60% purity, 1.2 eq) and stirred at 0° C. for 15 min. Then Compound 1A (18.5 uL, 0.14 mmol, 1.1 eq) was added to the mixture. The mixture was stirred at 25° C. for 2 h. The reaction mixture was diluted with water (1 mL) and extracted with EtOAc (1 mL×3). The combined organic layers were washed with brine (1 mL), dried over anhydrous Na₂SO₄, filtered and concentrated under reduced pressure to give a residue which was purified by flash silica gel chromatography to give Compound 2 (39 mg, 0.103 mmol, 80% yield). LCMS: [M+H]⁺=379.1 ¹H NMR (400 MHz, CDCl₃) δ 7.66-7.61 (m, 2H), 7.06 (d, J=8.1 Hz, 1H), 6.63 (t, J=8.4 Hz, 2H), 5.79 (dd, J=15.9, 1.4 Hz, 1H), 4.80 (d, J=15.8 Hz, 1H), 4.38 (q, J=6.6 Hz, 1H), 3.90 (s, 3H), 3.09 (s, 3H), 1.30 (d, J=6.6 Hz, 3H). ¹⁹F NMR (376 MHz, CDCl₃) δ −108.49 (s, 1F), −111.32 (d, J=6.5 Hz, 2F).

### Synthesis of Intermediate Compound 3

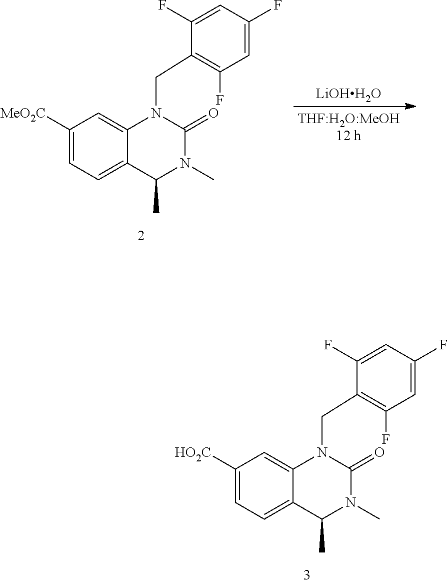 [SMILES: COC(=O)c1ccc2c(c1)N(Cc1c(F)cc(F)cc1F)C(=O)N(C)[C@H]2C ; "LiOH·H2O" ; C[C@H]1c2ccc(C(=O)O)cc2N(Cc2c(F)cc(F)cc2F)C(=O)N1C]

To a solution of Compound 2 (10 mg, 0.026 mmol, 1 eq) in THE (1 mL) and MeOH (0.5 mL) was added LiOH·H₂O (3.3 mg, 0.079 mmol, 3 eq) in H₂O (0.5 mL). The mixture was stirred at 25° C. for 12 h. The reaction mixture was concentrated under the reduced pressure to give a residue which was purified by flash silica gel chromatography to give Compound 3 (9.5 mg, 0.026 mmol, 99% yield). LCMS: [M+H]⁺=365.1 ¹H NMR (600 MHz, CDCl₃) δ 7.75-7.67 (m, 2H), 7.11 (d, J=7.8 Hz, 1H), 6.65 (t, J=8.3 Hz, 2H), 5.82 (dd, J=15.8, 1.5 Hz, 1H), 4.82 (d, J=15.8 Hz, 1H), 4.41 (q, J=6.6 Hz, 1H), 3.11 (s, 3H), 1.32 (d, J=6.6 Hz, 3H). ¹⁹F NMR (565 MHz, CDCl₃) δ −108.39 (p, J=7.5 Hz, 1F), −111.24 (t, J=7.4 Hz, 2F).

### Synthesis of UT156

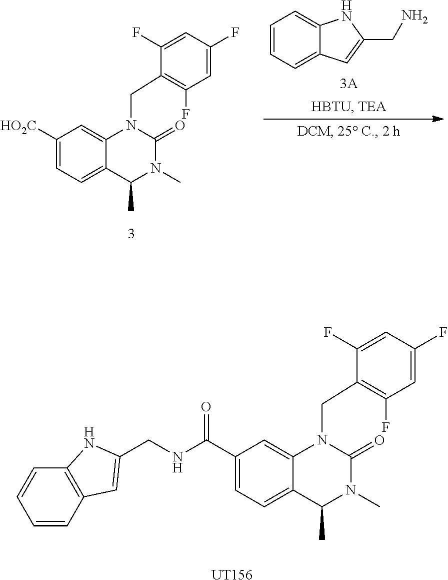

To a solution of Compound 3 (13.9 mg, 0.038 mmol, 1 eq) in DCM (1 mL) was added Et₃N (16 uL, 0.11 mmol, 3 eq) and HBTU (18.8 mg, 0.05 mmol, 1.3 eq). The mixture was stirred at 25° C. for 5 min, then Compound 3A (6.7 mg, 0.046 mmol, 1.2 eq) was added. The mixture was stirred at 25° C. for 2 h. The reaction mixture was concentrated under the reduced pressure to give a residue which was purified by flash silica gel chromatography to give UT156 (18.2 mg, 0.037 mmol, 97% yield). LCMS: [M+H]⁺=493.2

¹H NMR (400 MHz, CD₃CN) δ 9.36 (s, 1H), 7.53-7.43 (m, 2H), 7.38 (td, J=7.9, 1.3 Hz, 2H), 7.17 (d, J=7.8 Hz, 1H), 7.10 (ddd, J=8.2, 7.1, 1.2 Hz, 1H), 7.02 (ddd, J=8.0, 7.1, 1.1 Hz, 1H), 6.76 (t, J=8.8 Hz, 2H), 6.36 (dd, J=2.2, 0.9 Hz, 1H), 5.74-5.51 (m, 1H), 4.85 (d, J=16.0 Hz, 1H), 4.71-4.58 (m, 2H), 4.48 (q, J=6.6 Hz, 1H), 2.99 (s, 3H), 1.24 (d, J=6.6 Hz, 3H). ¹⁹F NMR (376 MHz, CD₃CN) δ −110.65 (t, J=6.5 Hz, 1F), −112.53 (d, J=6.3 Hz, 2F).

### Example 34 Synthesis of UT141

FIG. 41 depicts a representative synthesis scheme to generate a Compound UT141 as discussed above. The reaction scheme describes generating UT141 via two intermediate compounds (Compound 2 and Compound 3).

### Synthesis of Intermediate Compound 2

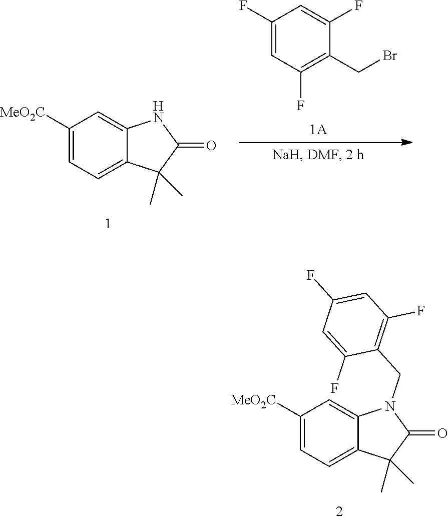 [SMILES: Fc1cc(F)c(CBr)c(F)c1 ; COC(=O)c1ccc2c(c1)NC(=O)C2(C)C ; COC(=O)c1ccc2c(c1)N(Cc1c(F)cc(F)cc1F)C(=O)C2(C)C]

To a solution of Compound 1 (31 mg, 0.14 mmol, 1 eq) in DMF (0.5 mL) was added NaH (6.7 mg, 0.17 mmol, 60% purity, 1.2 eq) and stirred at 0° C. for 15 min. Then Compound 1A (20.3 uL, 0.15 mmol, 1.1 eq) was added to the mixture. The mixture was stirred at 25° C. for 2 h. The reaction mixture was diluted with water (1 mL) and extracted with EtOAc (1 mL×3). The combined organic layers were washed with brine (1 mL), dried over anhydrous Na₂SO₄, filtered and concentrated under reduced pressure to give a residue which was purified by flash silica gel chromatography to give Compound 2 (51 mg, 0.14 mmol, 99% yield). LCMS: [M+H]⁺=364.1. ¹H NMR (400 MHz, CDCl₃) δ 7.78 (dd, J=7.7, 1.4 Hz, 1H), 7.49 (d, J=1.4 Hz, 1H), 7.28 (s, 1H), 6.70 (dd, J=8.7, 7.8 Hz, 2H), 5.02 (s, 2H), 3.92 (s, 3H), 1.41 (s, 6H). ¹⁹F NMR (376 MHz, CDCl₃) δ −107.17 (t, J=6.7 Hz, 1F), −110.52 (d, J=6.9 Hz, 2F).

### Synthesis of Intermediate Compound 3

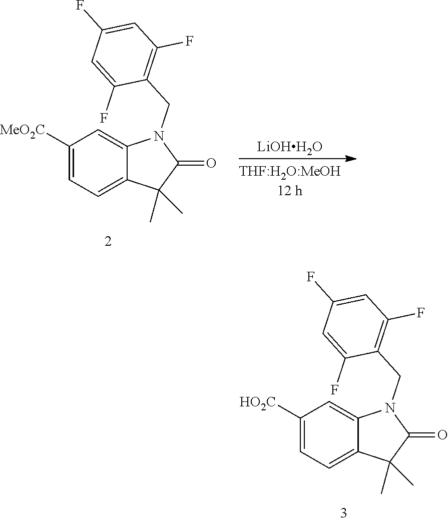

To a solution of Compound 2 (51 mg, 0.14 mmol, 1 eq) in THE (1 mL) and MeOH (0.5 mL) was added LiOH·H₂O (17.7 mg, 0.42 mmol, 3 eq) in H₂O (0.5 mL). The mixture was stirred at 25° C. for 12 h. The reaction mixture was concentrated under the reduced pressure to give a residue which was purified by flash silica gel chromatography to give Compound 3 (48 mg, 0.14 mmol, 98% yield). LCMS: [M+H]⁺=350.1. ¹H NMR (400 MHz, CDCl₃) δ 7.86 (dt, J=7.7, 1.2 Hz, 1H), 7.54 (d, J=1.4 Hz, 1H), 7.31 (d, J=7.7 Hz, 1H), 6.70 (t, J=8.2 Hz, 2H), 5.03 (s, 2H), 1.42 (s, 6H). ¹⁹F NMR (376 MHz, cdcl₃) δ −107.03 (p, J=7.6 Hz, 1F), −110.50 (t, J=7.4 Hz, 2F).

### Synthesis of UT141

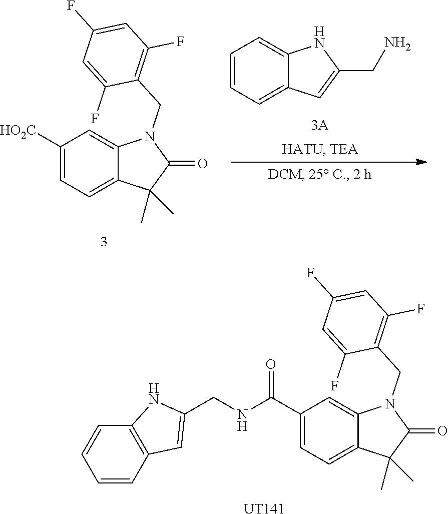 [SMILES: CC1(C)C(=O)N(Cc2c(F)cc(F)cc2F)c2cc(C(=O)O)ccc21 ; NCc1cc2ccccc2[nH]1 ; CC1(C)C(=O)N(Cc2c(F)cc(F)cc2F)c2cc(C(=O)NCc3cc4ccccc4[nH]3)ccc21 ; "UT141"]

To a solution of Compound 3 (6.8 mg, 0.020 mmol, 1 eq) in DCM (1 mL) was added Et₃N (8.1 uL, 0.058 mmol, 3 eq) and HATU (9.6 mg, 0.025 mmol, 1.3 eq). The mixture was stirred at 25° C. for 5 min, then Compound 3A (3.4 mg, 0.023 mmol, 1.2 eq) was added. The mixture was stirred at 25° C. for 2 h. The reaction mixture was concentrated under the reduced pressure to give a residue which was purified by flash silica gel chromatography to give UT141 (9.0 mg, 0.019 mmol, 97% yield). LCMS: [M+H]⁺=478.1. ¹H NMR (400 MHz, DMSO) δ 10.92 (s, 1H), 8.93 (t, J=5.7 Hz, 1H), 7.63 (dd, J=7.7, 1.5 Hz, 1H), 7.51-7.41 (m, 3H), 7.33 (dq, J=8.1, 0.9 Hz, 1H), 7.26-7.16 (m, 2H), 7.02 (ddd, J=8.2, 7.0, 1.3 Hz, 1H), 6.94 (ddd, J=8.0, 7.0, 1.1 Hz, 1H), 6.26 (dd, J=2.2, 1.0 Hz, 1H), 4.96 (s, 2H), 4.60 (d, J=5.6 Hz, 2H), 1.29 (s, 6H). ¹⁹F NMR (376 MHz, DMSO) δ −109.62 (t, J=6.8 Hz, 1F), −112.24 (d, J=6.8 Hz, 2F).

### Example 35 Synthesis of Compound UT142

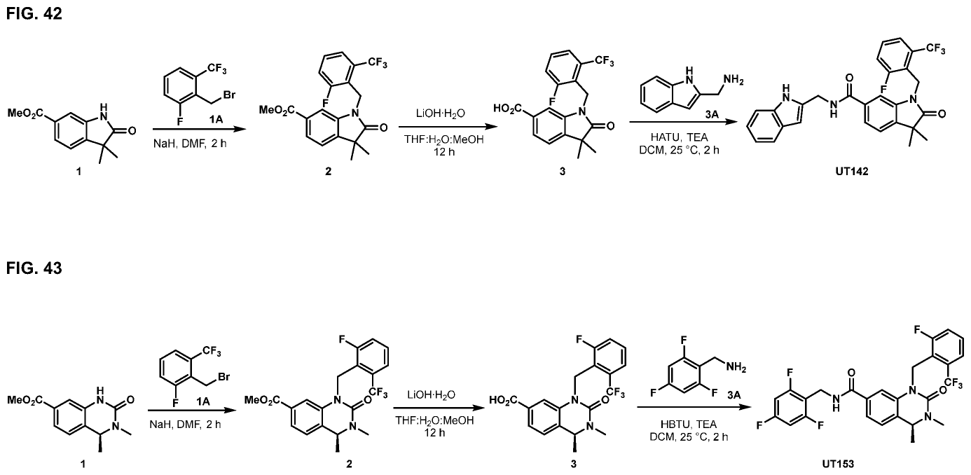

FIG. 42 depicts a representative reaction scheme to generate Compound UT142 via two intermediates (intermediate Compound 2 and intermediate Compound 3).

### Synthesis of Intermediate Compound 2

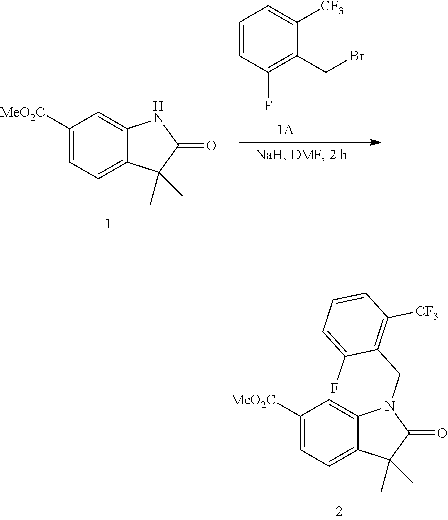 [SMILES: Fc1cccc(C(F)(F)F)c1CBr ; COC(=O)c1ccc2c(c1)NC(=O)C2(C)C ; COC(=O)c1ccc2c(c1)N(Cc1c(F)cccc1C(F)(F)F)C(=O)C2(C)C]

To a solution of Compound 1 (22.1 mg, 0.10 mmol, 1 eq) in DMF (0.5 mL) was added NaH (4.8 mg, 0.12 mmol, 60% purity, 1.2 eq) and stirred at 0° C. for 15 min. Then Compound 1A (28.5 mg, 0.11 mmol, 1.1 eq) was added to the mixture. The mixture was stirred at 25° C. for 2 h. The reaction mixture was diluted with water (1 mL) and extracted with EtOAc (1 mL×3). The combined organic layers were washed with brine (1 mL), dried over anhydrous Na₂SO₄, filtered and concentrated under reduced pressure to give a residue which was purified by flash silica gel chromatography to give Compound 2 (37.9 mg, 0.095 mmol, 95% yield). LCMS: [M+H]⁺=396.1 ¹H NMR (400 MHz, CDCl₃) δ 7.76 (dd, J=7.7, 1.4 Hz, 1H), 7.59 (d, J=7.9 Hz, 1H), 7.47-7.39 (m, 1H), 7.33 (d, J=1.4 Hz, 1H), 7.30-7.27 (m, 1H), 7.22 (ddd, J=10.0, 8.4, 1.2 Hz, 1H), 5.24 (s, 2H), 3.87 (s, 3H), 1.44 (s, 6H). ¹⁹F NMR (376 MHz, CDCl₃) δ −58.62 (s, 3F), −112.65 (s, 1F).

### Synthesis of Intermediate Compound 3

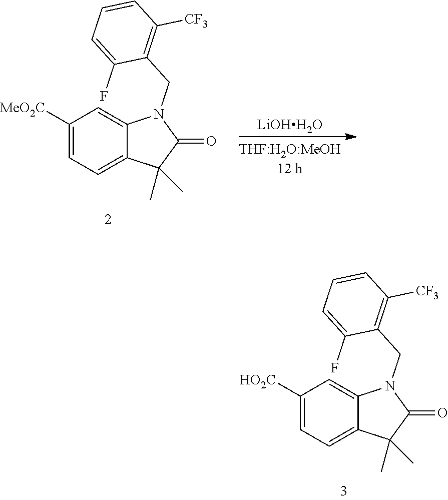

To a solution of Compound 2 (31.3 mg, 0.079 mmol, 1 eq) in THE (1 mL) and MeOH (0.5 mL) was added LiOH·H₂O (10.0 mg, 0.24 mmol, 3 eq) in H₂O (0.5 mL). The mixture was stirred at 25° C. for 12 h. The reaction mixture was concentrated under the reduced pressure to give a residue which was purified by flash silica gel chromatography to give Compound 3 (22.8 mg, 0.060 mmol, 76% yield). LCMS: [M+H]⁺=382.1. ¹H NMR (400 MHz, CDCl₃) δ 7.82 (dd, J=7.7, 1.4 Hz, 1H), 7.58 (d, J=7.9 Hz, 1H), 7.42 (td, J=8.1, 5.1 Hz, 1H), 7.34 (d, J=1.4 Hz, 1H), 7.30 (d, J=7.7 Hz, 1H), 7.24-7.17 (m, 1H), 5.23 (s, 2H), 1.44 (s, 6H). ¹⁹F NMR (376 MHz, CDCl₃) δ −58.64 (s, 3F), −112.71 (s, 1F).

### Synthesis of UT142

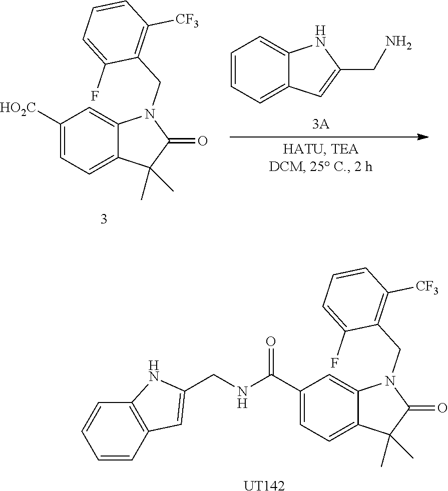 [SMILES: CC1(C)C(=O)N(Cc2c(F)cccc2C(F)(F)F)c2cc(C(=O)O)ccc21 ; NCc1cc2ccccc2[nH]1 ; CC1(C)C(=O)N(Cc2c(F)cccc2C(F)(F)F)c2cc(C(=O)NCc3cc4ccccc4[nH]3)ccc21 ; "UT142"]

To a solution of Compound 3 (20.6 mg, 0.054 mmol, 1 eq) in DCM (1 mL) was added Et₃N (22.6 uL, 0.16 mmol, 3 eq) and HATU (26.7 mg, 0.070 mmol, 1.3 eq). The mixture was stirred at 25° C. for 5 min, then Compound 3A (9.5 mg, 0.065 mmol, 1.2 eq) was added. The mixture was stirred at 25° C. for 2 h. The reaction mixture was concentrated under the reduced pressure to give a residue which was purified by flash silica gel chromatography to give UT142 (20.1 mg, 0.039 mmol, 73% yield). LCMS: [M+H]⁺=510.2. ¹H NMR (400 MHz, CD₃CN) δ 9.34 (s, 1H), 7.61 (d, J=7.9 Hz, 1H), 7.54-7.41 (m, 3H), 7.37-7.27 (m, 3H), 7.22 (d, J=1.4 Hz, 1H), 7.09 (ddd, J=8.2, 7.0, 1.3 Hz, 1H), 7.01 (ddd, J=8.0, 7.1, 1.1 Hz, 1H), 6.33 (dd, J=2.1, 1.0 Hz, 1H), 5.15 (s, 2H), 4.61 (dd, J=5.8, 0.8 Hz, 2H), 1.33 (s, 6H). ¹⁹F NMR (376 MHz, CD₃CN) δ −59.10 (s, 3F), −114.00 (s, 1F).

### Example 36. Synthesis of Compound UT153

FIG. 43 depicts a representative synthesis scheme to generate Compound UT153 via two intermediates (intermediate Compound 2 and intermediate Compound 3). Synthesis of intermediate Compound 2

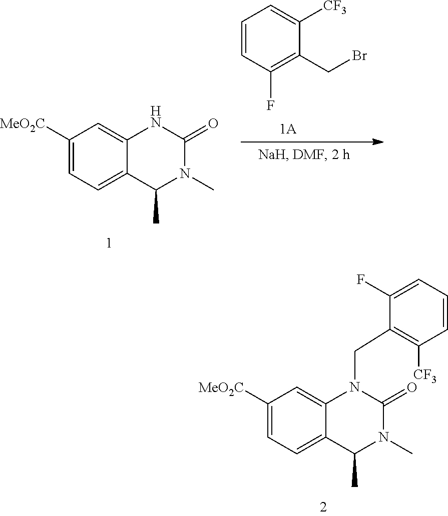 [SMILES: Fc1cccc(C(F)(F)F)c1CBr ; COC(=O)c1ccc2c(c1)NC(=O)N(C)[C@H]2C ; COC(=O)c1ccc2c(c1)N(Cc1c(F)cccc1C(F)(F)F)C(=O)N(C)[C@H]2C]

To a solution of Compound 1 (18.3 mg, 0.078 mmol, 1 eq) in DMF (0.5 mL) was added NaH (3.7 mg, 0.094 mmol, 60% purity, 1.2 eq) and stirred at 0° C. for 15 min. Then Compound 1A (22.1 mg, 0.086 mmol, 1.1 eq) was added to the mixture. The mixture was stirred at 25° C. for 2 h. The reaction mixture was diluted with water (1 mL) and extracted with EtOAc (1 mL×3). The combined organic layers were washed with brine (1 mL), dried over anhydrous Na₂SO₄, filtered and concentrated under reduced pressure to give a residue which was purified by flash silica gel chromatography to give Compound 2 (30.9 mg, 0.075 mmol, 96% yield). LCMS: [M+H]⁺=411.1 ¹H NMR (400 MHz, CDCl₃) δ 7.63 (dd, J=7.8, 1.5 Hz, 1H), 7.53 (d, J=7.9 Hz, 1H), 7.50 (d, J=1.5 Hz, 1H), 7.33 (td, J=8.1, 5.1 Hz, 1H), 7.18-7.10 (m, 1H), 7.09 (d, J=7.8 Hz, 1H), 5.79 (d, J=17.0 Hz, 1H), 5.18 (d, J=17.0 Hz, 1H), 4.48 (q, J=6.6 Hz, 1H), 3.83 (s, 3H), 3.10 (s, 3H), 1.41 (d, J=6.6 Hz, 3H). ¹⁹F NMR (376 MHz, CDCl₃) δ −59.15 (s, 3F), −114.29 (s, 1F).

### Synthesis of Intermediate Compound 3

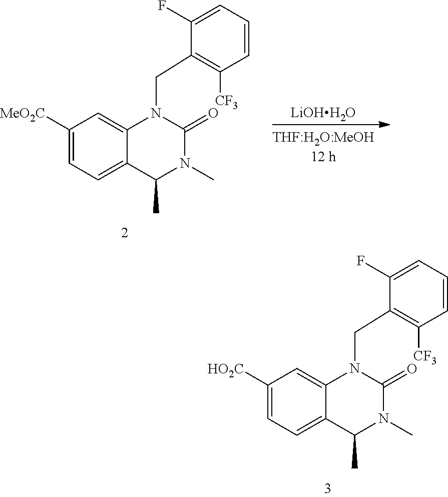 [SMILES: COC(=O)c1ccc2c(c1)N(Cc1c(F)cccc1C(F)(F)F)C(=O)N(C)[C@H]2C ; "LiOH·H2O" ; C[C@H]1c2ccc(C(=O)O)cc2N(Cc2c(F)cccc2C(F)(F)F)C(=O)N1C]

To a solution of Compound 2 (30.9 mg, 0.075 mmol, 1 eq) in THE (1 mL) and MeOH (0.5 mL) was added LiOH·H₂O (9.5 mg, 0.226 mmol, 3 eq) in H₂O (0.5 mL). The mixture was stirred at 25° C. for 12 h. The reaction mixture was concentrated under the reduced pressure to give a residue which was purified by flash silica gel chromatography to give Compound 3 (27.7 mg, 0.070 mmol, 93% yield). LCMS: [M+H]⁺=397.1 ¹H NMR (400 MHz, CDCl₃) δ 7.69 (dd, J=7.8, 1.4 Hz, 1H), 7.57-7.51 (m, 2H), 7.33 (td, J=8.1, 4.9 Hz, 1H), 7.19-7.09 (m, 2H), 5.77 (d, J=16.9 Hz, 1H), 5.21 (d, J=17.0 Hz, 1H), 4.49 (q, J=6.6 Hz, 1H), 3.11 (s, 3H), 1.42 (d, J=6.7 Hz, 3H). ¹⁹F NMR (376 MHz, CDCl₃) δ −59.22 (s, 3F), −114.43 (s, 1F).

### Synthesis of UT153

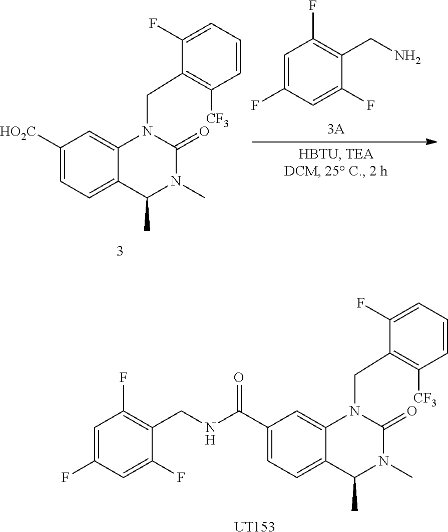

To a solution of Compound 3 (13.8 mg, 0.035 mmol, 1 eq) in DCM (1 mL) was added Et₃N (14.6 uL, 0.10 mmol, 3 eq) and HBTU (17.2 mg, 0.045 mmol, 1.3 eq). The mixture was stirred at 25° C. for 5 min, then Compound 3A (5.1 uL, 0.042 mmol, 1.2 eq) was added. The mixture was stirred at 25° C. for 2 h. The reaction mixture was concentrated under the reduced pressure to give a residue which was purified by flash silica gel chromatography to give UT153 (16.8 mg, 0.031 mmol, 89% yield). LCMS: [M+H]⁺=540.2 ¹H NMR (400 MHz, CDCl₃) δ 7.47 (d, J=7.9 Hz, 1H), 7.39 (dd, J=7.7, 1.5 Hz, 1H), 7.32 (td, J=8.1, 5.0 Hz, 1H), 7.17-7.03 (m, 3H), 6.77-6.62 (m, 2H), 6.15 (t, J=5.7 Hz, 1H), 5.78 (d, J=17.1 Hz, 1H), 5.12 (d, J=17.0 Hz, 1H), 4.60 (d, J=5.6 Hz, 2H), 4.45 (q, J=6.6 Hz, 1H), 3.09 (s, 3H), 1.38 (d, J=6.6 Hz, 3H). ¹⁹F NMR (376 MHz, CDCl₃) δ −59.13 (s, 3F), −107.72 (t, J=6.3 Hz, 1F), −111.75 (d, J=6.4 Hz, 2F), −113.99 (s, 1F).

### Example 37 Synthesis of UT151

FIG. 44 depicts a representative synthesis procedure to generate Compound UT151 via the creation of three intermediates (intermediate Compound 1A, intermediate Compound 2 and intermediate Compound 3).

### Synthesis of Intermediate Compound 1A

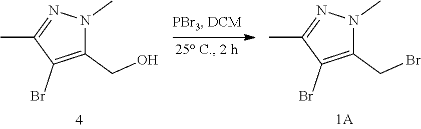

To a solution of Compound 4 (213.5 mg, 1.04 mmol, 1 eq) in DCM (4 mL) was added PBr₃ (98 uL, 1.04 mmol, 1 eq) dropwise at 25° C. The reaction mixture was stirred at 25° C. for 2 h. The reaction mixture was diluted with water (1 mL) and extracted with DCM (1 mL×3). The combined organic layers were washed with brine (1 mL), dried over anhydrous Na₂SO₄, filtered and concentrated under reduced pressure to give a residue which was purified by flash silica gel chromatography to give Compound 1A (234.4 mg, 0.87 mmol, 84% yield). LCMS: [M+H]⁺=267.0. ¹H NMR (400 MHz, CDCl₃) δ 4.44 (s, 2H), 3.86 (s, 3H), 2.21 (s, 3H).

### Synthesis of Intermediate Compound 2

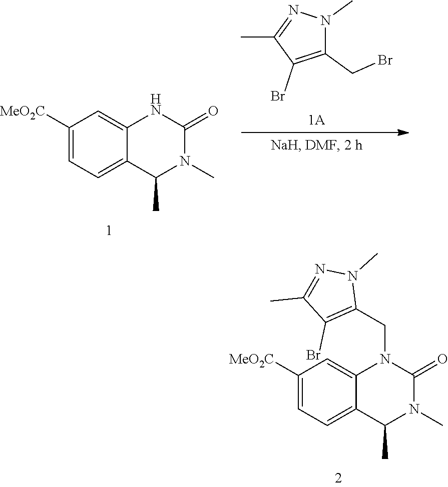

To a solution of Compound 1 (26.7 mg, 0.114 mmol, 1 eq) in DMF (1 mL) was added NaH (5.5 mg, 0.137 mmol, 60% purity, 1.2 eq) and stirred at 0° C. for 15 min. Then Compound 1A (33.6 mg, 0.125 mmol, 1.1 eq) was added to the mixture. The mixture was stirred at 25° C. for 2 h. The reaction mixture was diluted with water (1 mL) and extracted with EtOAc (1 mL×3). The combined organic layers were washed with brine (1 mL), dried over anhydrous Na₂SO₄, filtered and concentrated under reduced pressure to give a residue which was purified by flash silica gel chromatography to give Compound 2 (44.9 mg, 0.107 mmol, 94% yield). LCMS: [M+H]⁺=421.1. ¹H NMR (400 MHz, CDCl₃) δ 7.69 (td, J=4.0, 1.4 Hz, 2H), 7.13-7.06 (m, 1H), 5.72 (d, J=16.3 Hz, 1H), 4.78 (d, J=16.3 Hz, 1H), 4.46 (q, J=6.6 Hz, 1H), 3.90 (s, 3H), 3.83 (s, 3H), 3.11 (s, 3H), 2.20 (s, 3H), 1.37 (d, J=6.6 Hz, 3H).

### Synthesis of Intermediate Compound 3

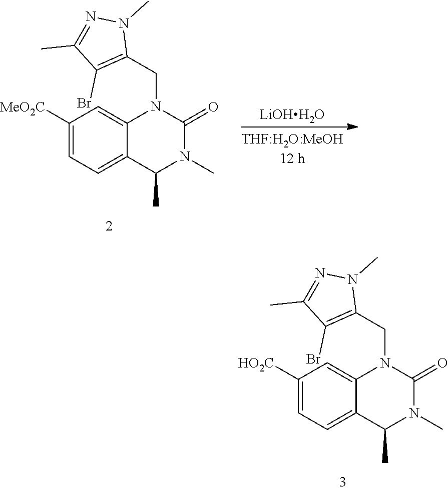

To a solution of Compound 2 (40.0 mg, 0.095 mmol, 1 eq) in THE (1 mL) and MeOH (0.5 mL) was added LiOH·H₂O (11.2 mg, 0.285 mmol, 3 eq) in H₂O (0.5 mL). The mixture was stirred at 25° C. for 12 h. The reaction mixture was concentrated under the reduced pressure to give a residue which was purified by flash silica gel chromatography to give Compound 3 (38.3 mg, 0.094 mmol, 99% yield). LCMS: [M+H]⁺=407.1. ¹H NMR (400 MHz, CDCl₃) δ 7.73 (td, J=4.0, 1.4 Hz, 2H), 7.13-7.08 (m, 1H), 5.69 (d, J=16.4 Hz, 1H), 4.79 (d, J=16.3 Hz, 1H), 4.46 (q, J=6.7 Hz, 1H), 3.86 (s, 3H), 3.10 (s, 3H), 2.18 (s, 3H), 1.38 (d, J=6.6 Hz, 3H).

### Synthesis of UT151

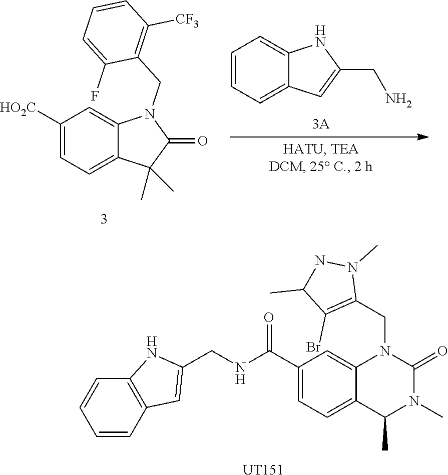 [SMILES: CC1(C)C(=O)N(Cc2c(F)cccc2C(F)(F)F)c2cc(C(=O)O)ccc21 ; NCc1cc2ccccc2[nH]1 ; CC1NN(C)C(CN2C(=O)N(C)[C@@H](C)c3ccc(C(=O)NCc4cc5ccccc5[nH]4)cc32)=C1Br ; "UT151"]

To a solution of Compound 3 (8.4 mg, 0.021 mmol, 1 eq) in DCM (1 mL) was added Et3N (8.6 uL, 0.062 mmol, 3 eq) and HBTU (10.2 mg, 0.027 mmol, 1.3 eq). The mixture was stirred at 25° C. for 5 min, then Compound 3A (3.6 mg, 0.025 mmol, 1.2 eq) was added. The mixture was stirred at 25° C. for 2 h. The reaction mixture was concentrated under the reduced pressure to give a residue which was purified by flash silica gel chromatography to give UT151 (11.6 mg, 0.022 mmol, 99% yield). LCMS: [M+H]⁺=535.2. ¹H NMR (400 MHz, CD₃CN) δ 9.38 (s, 1H), 7.50 (dq, J=7.9, 0.9 Hz, 1H), 7.47-7.40 (m, 2H), 7.37 (dq, J=8.1, 0.9 Hz, 1H), 7.19 (dt, J=7.6, 0.6 Hz, 1H), 7.10 (ddd, J=8.2, 7.1, 1.2 Hz, 1H), 7.01 (ddd, J=8.0, 7.1, 1.1 Hz, 1H), 6.36 (dq, J=1.6, 0.8 Hz, 1H), 5.52 (d, J=16.4 Hz, 1H), 4.78 (d, J=16.4 Hz, 1H), 4.64 (dt, J=5.9, 1.0 Hz, 2H), 4.54 (q, J=6.6 Hz, 1H), 3.78 (s, 3H), 3.00 (s, 3H), 2.03 (s, 3H), 1.32 (d, J=6.6 Hz, 3H).

### Example 38 Synthesis of Compound UT157

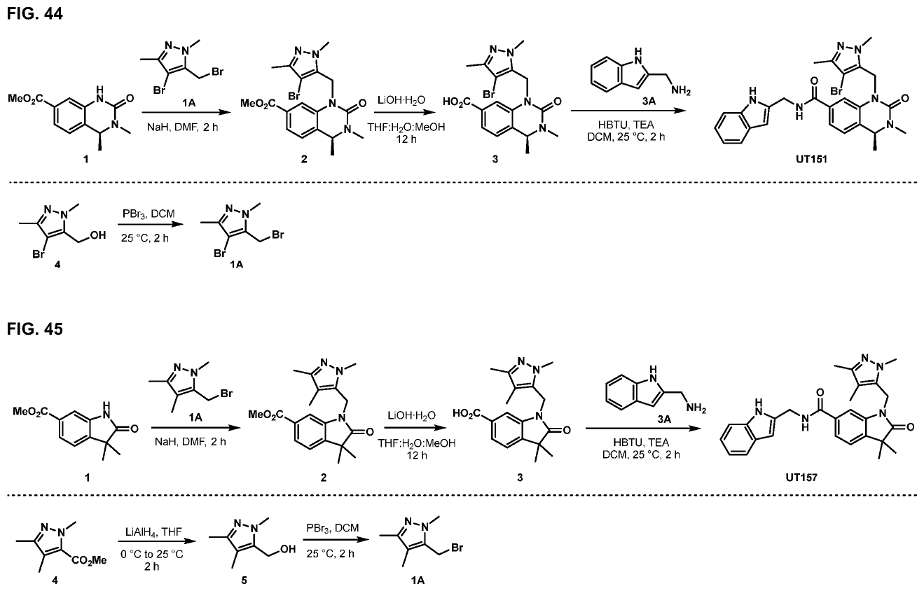

FIG. 45 depicts an illustrative synthesis scheme to generate Compound UT157 via two intermediates (intermediate Compound 2 and intermediate Compound 3) and one newly synthesized reactant (intermediate Compound 1A).

### Synthesis of Intermediate Compound 1A

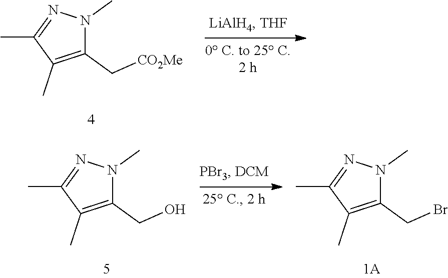

To a solution of Compound 4 (228.0 mg, 1.36 mmol, 1 eq) in THE (1.5 mL) was added LiAlH₄ (51.5 mg, 1.36 mmol, 1 eq) at 0° C. The reaction mixture was warmed to 25° C. and stirred for 2 h. H₂O (0.05 mL) was added, followed by 15% NaOH (0.05 mL). Additional H₂O (0.15 mL) was added. The mixture was extracted with EtOAc (1 mL×3). The combined organic layers were washed with brine (1 mL), dried over anhydrous Na₂SO₄, filtered and concentrated under reduced pressure to give crude Compound 5 which was used directly for the next step. LCMS: [M+H]⁺=141.2

To a solution of crude Compound 5 (1.36 mmol, 1 eq) in DCM (5 mL) was added PBr₃ (128 uL, 1.36 mmol, 1 eq) dropwise at 25° C. The reaction mixture was stirred at 25° C. for 2 h. The reaction mixture was diluted with water (1 mL) and extracted with DCM (1 mL×3). The combined organic layers were washed with brine (1 mL), dried over anhydrous Na₂SO₄, filtered and concentrated under reduced pressure to give a residue which was purified by flash silica gel chromatography to give Compound 1A (165.5 mg, 0.81 mmol, 60% yield for 2 steps). LCMS: [M+H]⁺=203.1. ¹H NMR (400 MHz, CDCl₃) δ 4.41 (s, 2H), 3.79 (s, 3H), 2.15 (s, 3H), 1.96 (s, 3H).

### Synthesis of Intermediate Compound 2

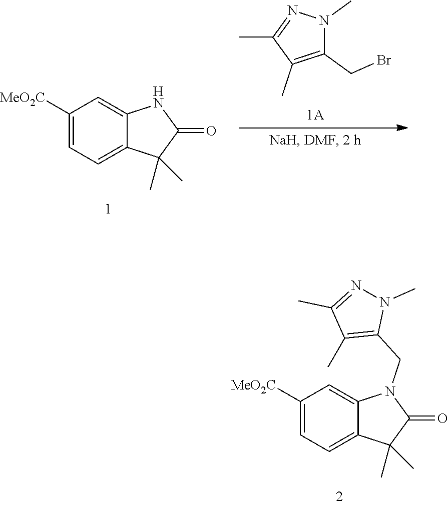

To a solution of Compound 1 (23.7 mg, 0.11 mmol, 1 eq) in DMF (1 mL) was added NaH (5.2 mg, 0.13 mmol, 60% purity, 1.2 eq) and stirred at 0° C. for 15 min. Then Compound 1A (24.1 mg, 0.12 mmol, 1.1 eq) was added to the mixture. The mixture was stirred at 25° C. for 2 h. The reaction mixture was diluted with water (1 mL) and extracted with EtOAc (1 mL×3). The combined organic layers were washed with brine (1 mL), dried over anhydrous Na₂SO₄, filtered and concentrated under reduced pressure to give a residue which was purified by flash silica gel chromatography to give Compound 2 (29.5 mg, 0.086 mmol, 80% yield). LCMS: [M+H]⁺=342.2. ¹H NMR (400 MHz, CDCl₃) δ 7.79 (dd, J=7.7, 1.4 Hz, 1H), 7.43 (d, J=1.4 Hz, 1H), 7.28 (d, J=1.1 Hz, 1H), 4.90 (s, 2H), 3.91 (s, 3H), 3.74 (s, 3H), 2.20 (d, J=2.2 Hz, 6H), 1.43 (s, 6H).

### Synthesis of Intermediate Compound 3

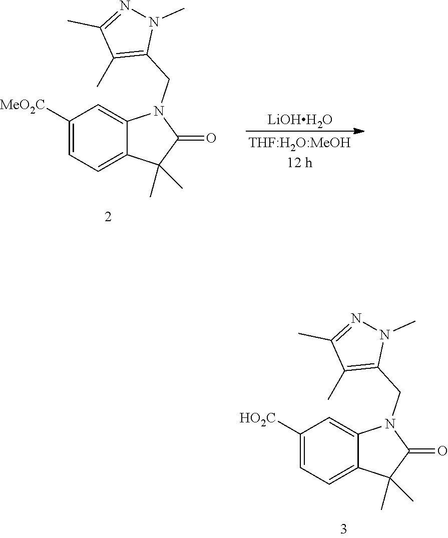 [SMILES: COC(=O)c1ccc2c(c1)N(Cc1c(C)c(C)nn1C)C(=O)C2(C)C ; "LiOH·H2O" ; Cc1nn(C)c(CN2C(=O)C(C)(C)c3ccc(C(=O)O)cc32)c1C]

To a solution of Compound 2 (29.4 mg, 0.086 mmol, 1 eq) in THE (1 mL) and MeOH (0.5 mL) was added LiOH·H₂O (10.8 mg, 0.258 mmol, 3 eq) in H₂O (0.5 mL). The mixture was stirred at 25° C. for 12 h. The reaction mixture was concentrated under the reduced pressure to give a residue which was purified by flash silica gel chromatography to give Compound 3 (27.9 mg, 0.085 mmol, 99% yield). LCMS: [M+H]⁺=328.2. ¹H NMR (400 MHz, CDCl₃) δ 7.86 (dd, J=7.7, 1.3 Hz, 1H), 7.44 (d, J=1.4 Hz, 1H), 7.30 (d, J=7.7 Hz, 1H), 4.91 (s, 2H), 3.80 (s, 3H), 2.21 (s, 3H), 2.17 (s, 3H), 1.43 (s, 6H).

### Synthesis of UT157

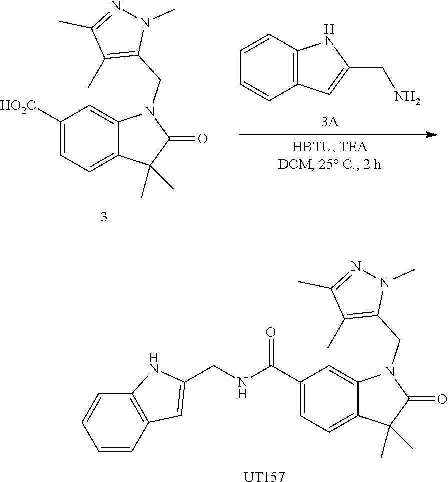 [SMILES: Cc1nn(C)c(CN2C(=O)C(C)(C)c3ccc(C(=O)O)cc32)c1C ; NCc1cc2ccccc2[nH]1 ; Cc1nn(C)c(CN2C(=O)C(C)(C)c3ccc(C(=O)NCc4cc5ccccc5[nH]4)cc32)c1C ; "UT157"]

To a solution of Compound 3 (6.4 mg, 0.0195 mmol, 1 eq) in DCM (1 mL) was added Et₃N (8.2 uL, 0.0586 mmol, 3 eq) and HATU (9.7 mg, 0.0254 mmol, 1.3 eq). The mixture was stirred at 25° C. for 5 min, then Compound 3A (3.4 mg, 0.0235 mmol, 1.2 eq) was added. The mixture was stirred at 25° C. for 2 h. The reaction mixture was concentrated under the reduced pressure to give a residue which was purified by flash silica gel chromatography to give UT157 (8.4 mg, 0.0184 mmol, 94% yield). LCMS: [M+H]⁺=456.2. ¹H NMR (400 MHz, CD₃CN) δ 9.39 (s, 1H), 7.48 (ddd, J=13.9, 7.8, 1.3 Hz, 2H), 7.39-7.32 (m, 2H), 7.26 (d, J=1.5 Hz, 1H), 7.09 (ddd, J=8.2, 7.1, 1.2 Hz, 1H), 7.01 (ddd, J=8.1, 7.0, 1.1 Hz, 1H), 6.36 (dt, J=2.2, 0.9 Hz, 1H), 4.86 (s, 2H), 4.64 (dd, J=5.8, 0.8 Hz, 2H), 3.64 (s, 3H), 2.03 (s, 3H), 2.01 (s, 3H), 1.35 (s, 6H).

### Example 39. Synthesis of Compound UT158

Compound UT158 was synthesized according to the following reaction scheme.

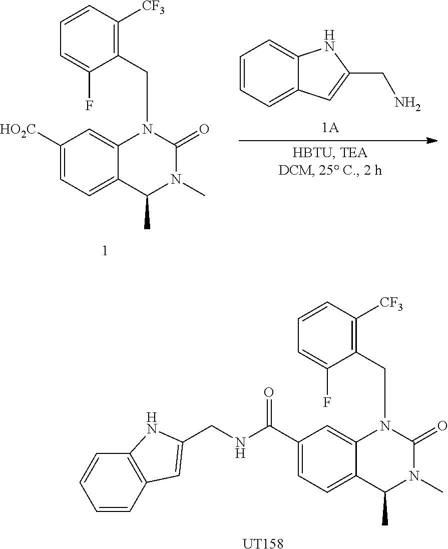 [SMILES: C[C@H]1c2ccc(C(=O)O)cc2N(Cc2c(F)cccc2C(F)(F)F)C(=O)N1C ; NCc1cc2ccccc2[nH]1 ; C[C@H]1c2ccc(C(=O)NCc3cc4ccccc4[nH]3)cc2N(Cc2c(F)cccc2C(F)(F)F)C(=O)N1C ; "UT158"]

To a solution of Compound 1 (13.9 mg, 0.035 mmol, 1 eq) in DCM (1 mL) was added Et₃N (14.7 uL, 0.11 mmol, 3 eq) and HBTU (17.3 mg, 0.046 mmol, 1.3 eq). The mixture was stirred at 25° C. for 5 min, then Compound 1A (6.2 mg, 0.042 mmol, 1.2 eq) was added. The mixture was stirred at 25° C. for 2 h. The reaction mixture was concentrated under the reduced pressure to give a residue which was purified by flash silica gel chromatography to give UT158 (13.3 mg, 0.025 mmol, 72% yield). LCMS: [M+H]⁺=525.2 ¹H NMR (400 MHz, CD₃CN) δ 9.36 (s, 1H), 7.55 (d, J=7.9 Hz, 1H), 7.51-7.30 (m, 5H), 7.27-7.15 (m, 2H), 7.08 (ddd, J=8.2, 7.1, 1.3 Hz, 1H), 7.00 (ddd, J=8.0, 7.0, 1.1 Hz, 1H), 6.32 (dd, J=2.1, 1.0 Hz, 1H), 5.53 (d, J=16.9 Hz, 1H), 5.19 (d, J=16.9 Hz, 1H), 4.59 (ddd, J=5.8, 1.6, 0.8 Hz, 2H), 4.53 (q, J=6.6 Hz, 1H), 2.97 (s, 3H), 1.33 (d, J=6.6 Hz, 3H). ¹⁹F NMR (376 MHz, CD₃CN) δ −59.53 (s, 3F), −115.97 (s, 1F).

### Example 40. Synthesis of Compound UT160

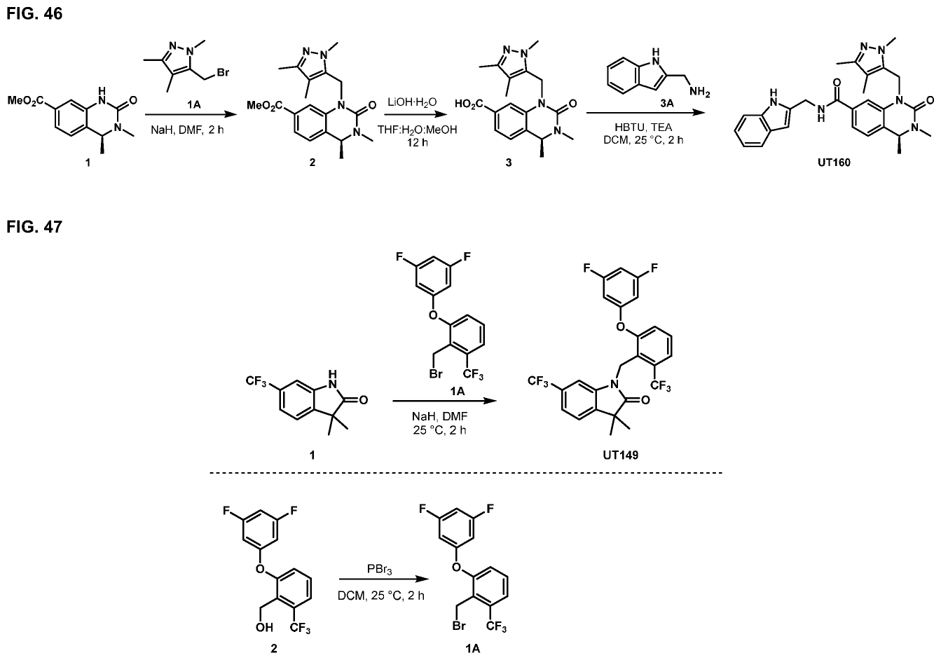

FIG. 46 depicts a representative synthesis procedure to generate Compound UT160 via two intermediates (intermediate Compound 2 and intermediate Compound 3).

### Synthesis of Intermediate Compound 2

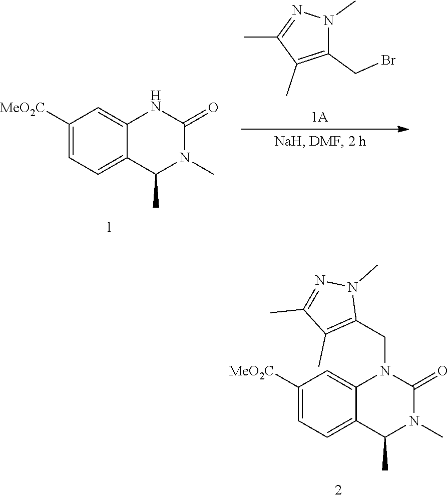

To a solution of Compound 1 (30.0 mg, 0.128 mmol, 1 eq) in DMF (1 mL) was added NaH (6.1 mg, 0.154 mmol, 60% purity, 1.2 eq) and stirred at 0° C. for 15 min. Then Compound 1A (28.6 mg, 0.141 mmol, 1.1 eq) was added to the mixture. The mixture was stirred at 25° C. for 2 h. The reaction mixture was diluted with water (1 mL) and extracted with EtOAc (1 mL×3). The combined organic layers were washed with brine (1 mL), dried over anhydrous Na₂SO₄, filtered and concentrated under reduced pressure to give a residue which was purified by flash silica gel chromatography to give Compound 2 (43.3 mg, 0.122 mmol, 95% yield). LCMS: [M+H]⁺=357.2. ¹H NMR (400 MHz, CDCl₃) δ 7.66 (dd, J=7.8, 1.5 Hz, 1H), 7.53 (d, J=1.5 Hz, 1H), 7.08 (d, J=7.8 Hz, 1H), 5.69 (d, J=16.3 Hz, 1H), 4.66 (d, J=16.3 Hz, 1H), 4.45 (q, J=6.6 Hz, 1H), 3.89 (s, 3H), 3.80 (s, 3H), 3.10 (s, 3H), 2.15 (s, 3H), 2.11 (s, 3H), 1.35 (d, J=6.6 Hz, 3H).

### Synthesis of Intermediate Compound 3

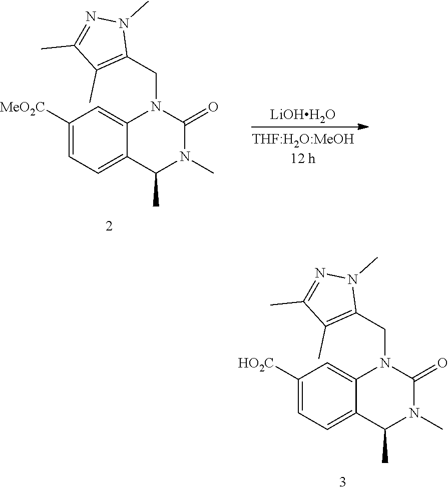

To a solution of Compound 2 (30.7 mg, 0.086 mmol, 1 eq) in THE (1 mL) and MeOH (0.5 mL) was added LiOH·H₂O (10.8 mg, 0.259 mmol, 3 eq) in H₂O (0.5 mL). The mixture was stirred at 25° C. for 12 h. The reaction mixture was concentrated under the reduced pressure to give a residue which was purified by flash silica gel chromatography to give Compound 3 (29.2 mg, 0.085 mmol, 99% yield). LCMS: [M+H]⁺=343.2

### Synthesis of UT160

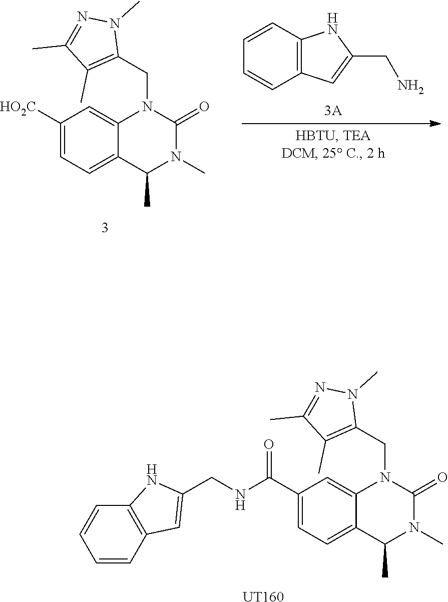

To a solution of Compound 3 (8.1 mg, 0.024 mmol, 1 eq) in DCM (1 mL) was added Et₃N (9.9 uL, 0.071 mmol, 3 eq) and HBTU (11.7 mg, 0.031 mmol, 1.3 eq). The mixture was stirred at 25° C. for 5 min, then Compound 3A (4.2 mg, 0.028 mmol, 1.2 eq) was added. The mixture was stirred at 25° C. for 2 h. The reaction mixture was concentrated under the reduced pressure to give a residue which was purified by flash silica gel chromatography to give UT160 (9.3 mg, 0.020 mmol, 83% yield). LCMS: [M+H]⁺=471.2. ¹H NMR (400 MHz, CD₃CN) δ 9.43 (s, 1H), 7.54 (s, 1H), 7.50 (dq, J=7.7, 0.9 Hz, 1H), 7.41-7.32 (m, 3H), 7.16 (dt, J=7.4, 0.7 Hz, 1H), 7.09 (ddd, J=8.2, 7.1, 1.2 Hz, 1H), 7.01 (ddd, J=8.1, 7.1, 1.1 Hz, 1H), 6.35 (dq, J=1.7, 0.8 Hz, 1H), 5.46 (d, J=16.4 Hz, 1H), 4.74 (d, J=16.4 Hz, 1H), 4.64 (ddd, J=5.8, 2.6, 0.8 Hz, 2H), 4.53 (q, J=6.6 Hz, 1H), 3.68 (s, 3H), 3.01 (s, 3H), 1.97 (s, 3H), 1.90 (s, 3H), 1.28 (d, J=6.6 Hz, 3H).

### Example 41. Synthesis of Compound UT149

FIG. 47 depicts a representative reaction scheme to generate a compound UT149 via an intermediate reactant (intermediate Compound 1A).

### Synthesis of Intermediate Compound 1A

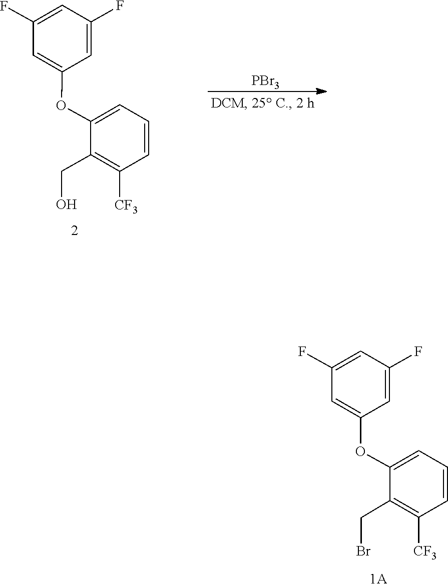 [SMILES: OCc1c(Oc2cc(F)cc(F)c2)cccc1C(F)(F)F ; "PBr3" ; Fc1cc(F)cc(Oc2cccc(C(F)(F)F)c2CBr)c1]

To a solution of Compound 2 (53.5 mg, 0.176 mmol, 1 eq) in DCM (1 mL) was PBr₃ (16.5 uL, 0.176 mmol, 1 eq). The mixture was stirred at 25° C. for 2 h. The reaction mixture was diluted with water (1 mL) and extracted with DCM (1 mL×3). The combined organic layers were washed with brine (1 mL), dried over anhydrous Na₂SO₄, filtered and concentrated under reduced pressure to give a residue which was purified by flash silica gel chromatography to give Compound 1A (25.2 mg, 0.069 mmol, 39% yield). ¹H NMR (400 MHz, CDCl₃) δ 7.50 (dd, J=8.0, 1.3 Hz, 1H), 7.46-7.39 (m, 1H), 7.13 (dd, J=8.2, 1.3 Hz, 1H), 6.68-6.55 (m, 3H), 4.68 (s, 2H). ¹⁹F NMR (376 MHz, CDCl₃) δ −59.48 (s, 3F), −107.58 (s, 2F).

### Synthesis of UT149

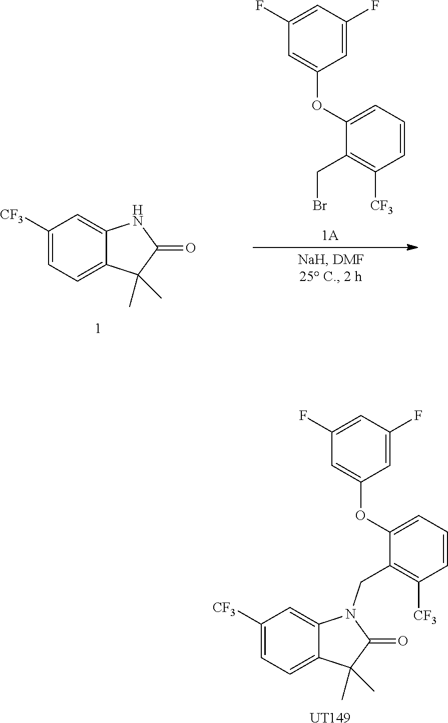

To a solution of Compound 1 (2.6 mg, 0.011 mmol, 1 eq) in DMF (0.5 mL) was added NaH (0.5 mg, 0.014 mmol, 60% purity, 1.2 eq) and stirred at 0° C. for 15 min. Then Compound 1A (4.5 mg, 0.012 mmol, 1.1 eq) was added to the mixture. The mixture was stirred at 25° C. for 2 h. The reaction mixture was diluted with water (1 mL) and extracted with EtOAc (1 mL×3). The combined organic layers were washed with brine (1 mL), dried over anhydrous Na₂SO₄, filtered and concentrated under reduced pressure to give a residue which was purified by flash silica gel chromatography to give UT149 (5.7 mg, 0.011 mmol, 97% yield). LCMS: [M+H]⁺=516.1. ¹H NMR (400 MHz, CDCl₃) δ 7.66-7.58 (m, 1H), 7.50-7.39 (m, 1H), 7.29-7.25 (m, 1H), 7.20 (d, J=7.7 Hz, 1H), 7.09 (dd, J=8.3, 1.2 Hz, 1H), 6.91-6.84 (m, 1H), 6.49 (tt, J=8.8, 2.3 Hz, 1H), 6.23-6.14 (m, 2H), 5.19 (s, 2H), 1.23 (s, 6H). ¹⁹F NMR (376 MHz, CDCl₃) δ −58.03 (s, 3F), −62.60 (s, 3F), −107.49 (s, 2F).

### Example 42. Synthesis of Compound UT202

Compound UT202 was synthesized according to the reaction scheme depicted below.

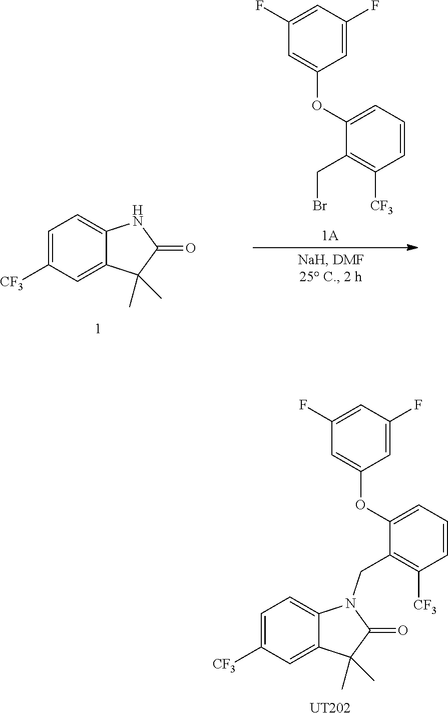

To a solution of Compound 1 (2.6 mg, 0.011 mmol, 1 eq) in DMF (0.5 mL) was added NaH (0.5 mg, 0.014 mmol, 60% purity, 1.2 eq) and stirred at 0° C. for 15 min. Then Compound 1A (4.5 mg, 0.012 mmol, 1.1 eq) was added to the mixture. The mixture was stirred at 25° C. for 2 h. The reaction mixture was diluted with water (1 mL) and extracted with EtOAc (1 mL×3). The combined organic layers were washed with brine (1 mL), dried over anhydrous Na₂SO₄, filtered and concentrated under reduced pressure to give a residue which was purified by flash silica gel chromatography to give UT202 (4.2 mg, 0.008 mmol, 72% yield). LCMS: [M+H]⁺=516.1. ¹H NMR (400 MHz, CD₃CN) δ 7.71-7.66 (m, 1H), 7.56-7.49 (m, 2H), 7.45 (ddd, J=8.4, 2.1, 1.0 Hz, 1H), 7.20 (d, J=8.3 Hz, 1H), 6.82 (d, J=8.3 Hz, 1H), 6.62 (tt, J=9.3, 2.3 Hz, 1H), 6.30-6.16 (m, 2H), 5.15 (s, 2H), 1.16 (s, 6H). ¹⁹F NMR (376 MHz, CD₃CN) δ −58.42 (s, 3F), −61.93 (s, 3F), −109.95 (s, 2F).

### Example 43. Synthesis of Compound UT200

Compound UT200 was synthesized according to the reaction scheme depicted below.

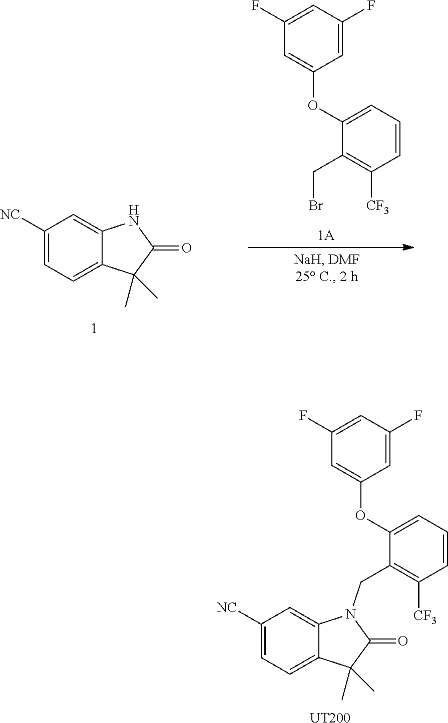

To a solution of Compound 1 (2.2 mg, 0.012 mmol, 1 eq) in DMF (0.5 mL) was added NaH (0.6 mg, 0.014 mmol, 60% purity, 1.2 eq) and stirred at 0° C. for 15 min. Then Compound 1A (4.8 mg, 0.013 mmol, 1.1 eq) was added to the mixture. The mixture was stirred at 25° C. for 2 h. The reaction mixture was diluted with water (1 mL) and extracted with EtOAc (1 mL×3). The combined organic layers were washed with brine (1 mL), dried over anhydrous Na₂SO₄, filtered and concentrated under reduced pressure to give a residue which was purified by flash silica gel chromatography to give UT200 (5.2 mg, 0.011 mmol, 93% yield). LCMS: [M+H]⁺=473.1. ¹H NMR (400 MHz, CDCl₃) δ 7.64 (dd, J=7.9, 1.2 Hz, 1H), 7.48 (t, J=8.1 Hz, 1H), 7.32 (dd, J=7.6, 1.4 Hz, 1H), 7.20 (d, J=7.6 Hz, 1H), 7.10 (d, J=8.2 Hz, 1H), 6.81 (d, J=1.3 Hz, 1H), 6.51 (tt, J=8.9, 2.3 Hz, 1H), 6.24-6.16 (m, 2H), 5.18 (s, 2H), 1.22 (s, 6H). ¹⁹F NMR (376 MHz, CDCl₃) δ −57.94 (s, 3F), −107.18 (s, 2F).

### Example 44. Synthesis of Compound UT201

Compound UT201 was synthesized according to the reaction scheme depicted below.

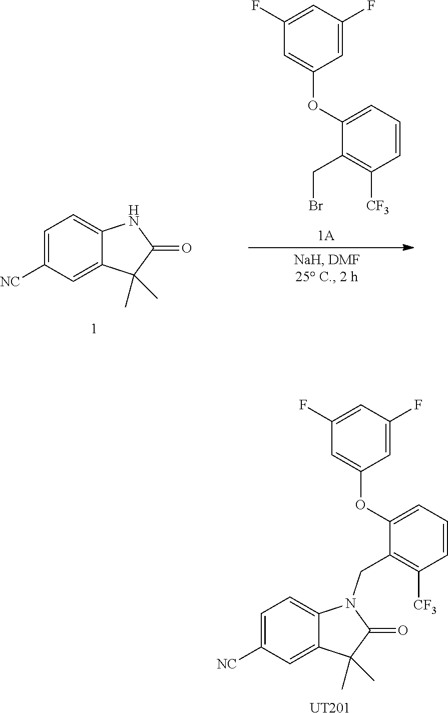

To a solution of Compound 1 (2.4 mg, 0.013 mmol, 1 eq) in DMF (0.5 mL) was added NaH (0.6 mg, 0.015 mmol, 60% purity, 1.2 eq) and stirred at 0° C. for 15 min. Then Compound 1A (5.1 mg, 0.014 mmol, 1.1 eq) was added to the mixture. The mixture was stirred at 25° C. for 2 h. The reaction mixture was diluted with water (1 mL) and extracted with EtOAc (1 mL×3). The combined organic layers were washed with brine (1 mL), dried over anhydrous Na₂SO₄, filtered and concentrated under reduced pressure to give a residue which was purified by flash silica gel chromatography to give UT201 (5.6 mg, 0.012 mmol, 92% yield). LCMS: [M+H]⁺=473.1. ¹H NMR (400 MHz, CDCl₃) δ 7.62 (dd, J=8.0, 1.2 Hz, 1H), 7.51-7.39 (m, 2H), 7.37 (d, J=1.6 Hz, 1H), 7.08 (dd, J=8.3, 1.2 Hz, 1H), 6.66 (d, J=8.2 Hz, 1H), 6.52 (tt, J=8.8, 2.2 Hz, 1H), 6.23-6.15 (m, 2H), 5.20 (s, 2H), 1.23 (s, 6H). ¹⁹F NMR (376 MHz, CDCl₃) δ −57.89 (s, 3F), −107.09 (s, 2F).

### Example 45. Synthesis of Compound UT203

Compound UT203 was synthesized according to the reaction scheme depicted below.

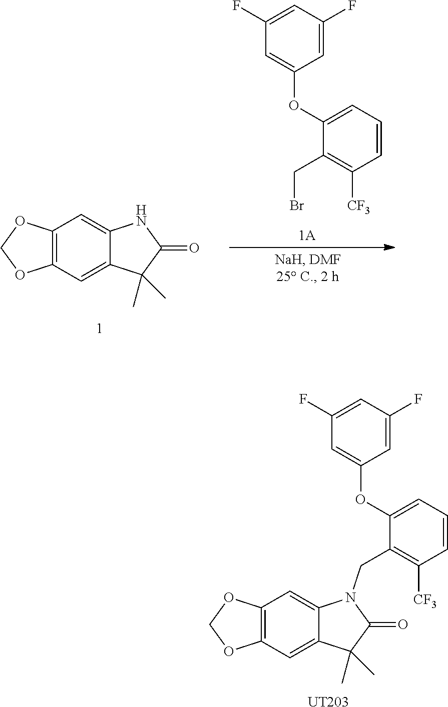

To a solution of Compound 1 (2.2 mg, 0.011 mmol, 1 eq) in DMF (0.5 mL) was added NaH (0.5 mg, 0.013 mmol, 60% purity, 1.2 eq) and stirred at 0° C. for 15 min. Then Compound 1A (4.5 mg, 0.012 mmol, 1.1 eq) was added to the mixture. The mixture was stirred at 25° C. for 2 h. The reaction mixture was diluted with water (1 mL) and extracted with EtOAc (1 mL×3). The combined organic layers were washed with brine (1 mL), dried over anhydrous Na₂SO₄, filtered and concentrated under reduced pressure to give a residue which was purified by flash silica gel chromatography to give UT203 (4.5 mg, 0.009 mmol, 85% yield). LCMS: [M+H]⁺=492.2. ¹H NMR (400 MHz, CD₃CN) δ 7.67 (d, J=7.9 Hz, 1H), 7.52 (t, J=8.1 Hz, 1H), 7.20 (d, J=8.3 Hz, 1H), 6.77 (s, 1H), 6.64 (tt, J=9.3, 2.3 Hz, 1H), 6.33-6.25 (m, 3H), 5.85 (s, 2H), 5.06 (s, 2H), 1.09 (s, 6H). ¹⁹F NMR (376 MHz, CD₃CN) δ −58.39 (s, 3F), −110.01 (s, 2F).

### Example 46—Activity of Exemplary STING Inhibitors

The STING pathway activity was evaluated in THP1-Dual™ cells (Invivogen). 1.5×10⁵ THP1-Dual™ cells were seeded in 96-well plates and treated with the test compound followed by cGAMP (100 uM) or MSA-2 (10 uM, Science 2020, 369, eaba6098) 2 hours later. After incubating at 37° C. for 16 hours, the luciferase activity was measured by QUANTI-Luc™ (Invivogen).

Table 1 depicts compounds analyzed and their relative activity (see below for Activity codes).

TABLE 1

| Compound | Structure | INPADOC Name | Example | Activity |
|---|---|---|---|---|
| UT009 | 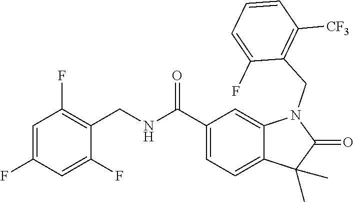 [SMILES: CC1(C)C(=O)N(Cc2c(F)cccc2C(F)(F)F)c2cc(C(=O)NCc3c(F)cc(F)cc3F)ccc21] | 1-(2-fluoro-6- (trifluoromethyl)benzyl)-3,3- dimethyl-2-oxo-N-(2,4,6- trifluorobenzyl)indoline-6- carboxamide | 6 | A |
| UT122 | 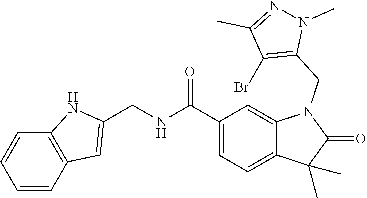 [SMILES: Cc1nn(C)c(CN2C(=O)C(C)(C)c3ccc(C(=O)NCc4cc5ccccc5[nH]4)cc32)c1Br] | N-((1H-indol-2-yl)methyl)- 1-((4-bromo-1,3-dimethyl- 1H-pyrazol-5-yl)methyl)-3,3- dimethyl-2-oxoindoline-6- carboxamide | 9 | A |
| UT141 | 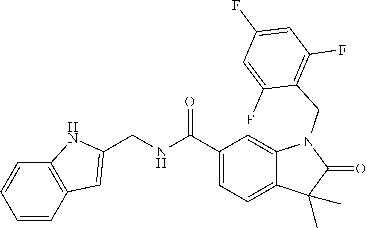 [SMILES: CC1(C)C(=O)N(Cc2c(F)cc(F)cc2F)c2cc(C(=O)NCc3cc4ccccc4[nH]3)ccc21] | N-((1H-indol-2-yl)methyl)- 3,3-dimethyl-2-oxo-1-(2,4,6- trifluorobenzyl)indoline-6- carboxamide | 34 | A |
| UT142 | 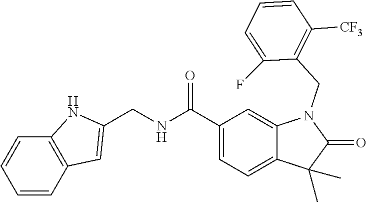 [SMILES: CC1(C)C(=O)N(Cc2c(F)cccc2C(F)(F)F)c2cc(C(=O)NCc3cc4ccccc4[nH]3)ccc21] | N-((1H-indol-2-yl)methyl)- 1-(2-fluoro-6- (trifluoromethyl)benzyl)-3,3- dimethyl-2-oxoindoline-6- carboxamide | 35 | A |
| UT200 | 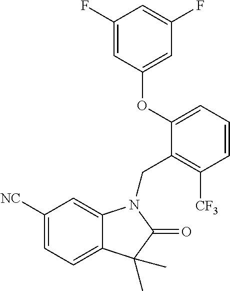 [SMILES: CC1(C)C(=O)N(Cc2c(Oc3cc(F)cc(F)c3)cccc2C(F)(F)F)c2cc(C#N)ccc21] | 1-(2-(3,5-difluorophenoxy)- 6-(trifluoromethyl)benzyl)- 3,3-dimethyl-2-oxoindoline- 6-carbonitrile | 43 | A |
| UT203 | 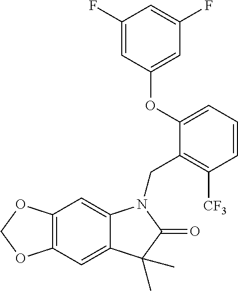 [SMILES: CC1(C)C(=O)N(Cc2c(Oc3cc(F)cc(F)c3)cccc2C(F)(F)F)c2cc3c(cc21)OCO3] | 5-(2-(3,5-difluorophenoxy)- 6-(trifluoromethyl)benzyl)- 7,7-dimethyl-5,7-dihydro- 6H-[1,3]dioxolo[4,5-f]indol- 6-one | 45 | A |
| UT156 | 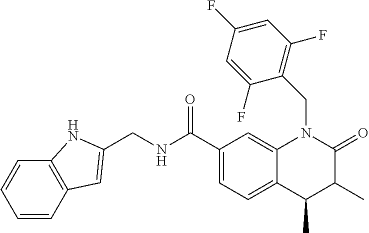 [SMILES: CC1C(=O)N(Cc2c(F)cc(F)cc2F)c2cc(C(=O)NCc3cc4ccccc4[nH]3)ccc2[C@@H]1C] | (4R)-N-((1H-indol-2-yl) methyl)-3,4-dimethyl-2-oxo- 1-(2,4,6-trifluorobenzyl)- 1,2,3,4-tetrahydroquinoline- 7-carboxamide | 33 | A |
| UT126 | 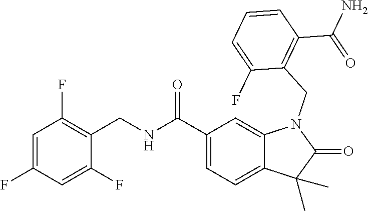 [SMILES: CC1(C)C(=O)N(Cc2c(F)cccc2C(N)=O)c2cc(C(=O)NCc3c(F)cc(F)cc3F)ccc21] | 1-(2-carbamoyl-6- fluorobenzyl)-3,3-dimethyl-2- oxo-N-(2,4,6-trifluorobenzyl) indoline-6-carboxamide | 25 | B |
| UT157 | 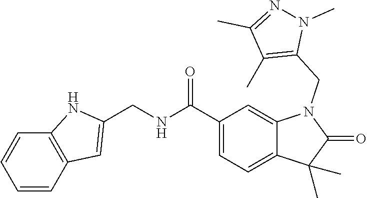 [SMILES: Cc1nn(C)c(CN2C(=O)C(C)(C)c3ccc(C(=O)NCc4cc5ccccc5[nH]4)cc32)c1C] | N-((1H-indol-2-yl)methyl)- 3,3-dimethyl-2-oxo-1-((1,3,4- trimethyl-1H-pyrazol-5-yl) methyl)indoline-6- carboxamide | 38 | B |
| UT073 | 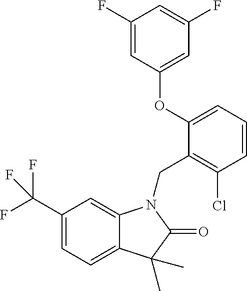 [SMILES: CC1(C)C(=O)N(Cc2c(Cl)cccc2Oc2cc(F)cc(F)c2)c2cc(C(F)(F)F)ccc21] | 1-(2-chloro-6-(3,5- difluorophenoxy)benzyl)-3,3- dimethyl-6-(trifluoromethyl) indolin-2-one | 8 | B |
| UT149 | 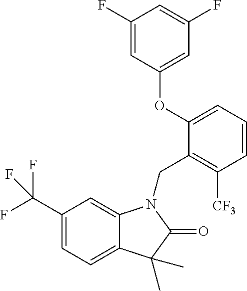 [SMILES: CC1(C)C(=O)N(Cc2c(Oc3cc(F)cc(F)c3)cccc2C(F)(F)F)c2cc(C(F)(F)F)ccc21] | 1-(2-(3,5-difluorophenoxy)- 6-(trifluoromethyl)benzyl)- 3,3-dimethyl-6- (trifluoromethyl)indolin-2- one | 41 | B |
| UT202 | 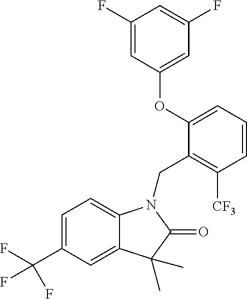 [SMILES: CC1(C)C(=O)N(Cc2c(Oc3cc(F)cc(F)c3)cccc2C(F)(F)F)c2ccc(C(F)(F)F)cc21] | 1-(2-(3,5-difluorophenoxy)- 6-(trifluoromethyl)benzyl)- 3,3-dimethyl-5- (trifluoromethyl)indolin-2- one | 42 | B |
| UT201 | 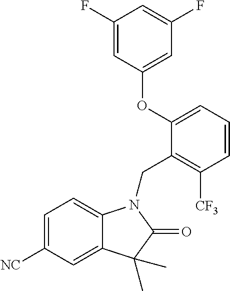 [SMILES: CC1(C)C(=O)N(Cc2c(Oc3cc(F)cc(F)c3)cccc2C(F)(F)F)c2ccc(C#N)cc21] | 1-(2-(3,5-difluorophenoxy)- 6-(trifluoromethyl)benzyl)- 3,3-dimethyl-2-oxoindoline- 5-carbonitrile | 44 | B |
| UT153 | 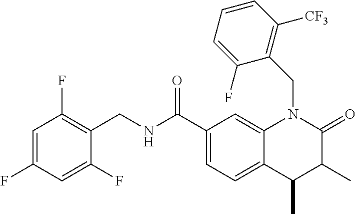 [SMILES: CC1C(=O)N(Cc2c(F)cccc2C(F)(F)F)c2cc(C(=O)NCc3c(F)cc(F)cc3F)ccc2[C@@H]1C] | (4R)-1-(2-fluoro-6- (trifluoromethyl)benzyl)-3,4- dimethyl-2-oxo-N-(2,4,6- trifluorobenzyl)-1,2,3,4- tetrahydroquinoline-7- carboxamide | 36 | B |
| UT160 | 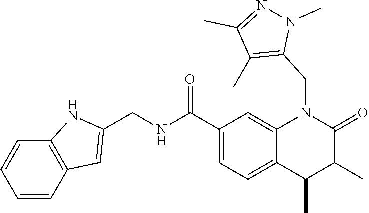 [SMILES: Cc1nn(C)c(CN2C(=O)C(C)[C@@H](C)c3ccc(C(=O)NCc4cc5ccccc5[nH]4)cc32)c1C] | (4R)-N-((1H-indol-2-yl) methyl)-3,4-dimethyl-2-oxo- 1-((1,3,4-trimethyl-1H- pyrazol-5-yl)methyl)-1,2,3,4- tetrahydroquinoline-7- carboxamide | 40 | B |
| UT151 | 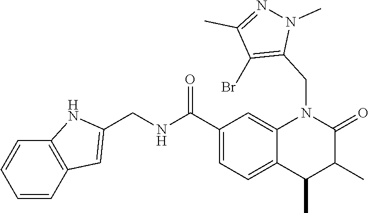 [SMILES: Cc1nn(C)c(CN2C(=O)C(C)[C@@H](C)c3ccc(C(=O)NCc4cc5ccccc5[nH]4)cc32)c1Br] | (4R)-N-((1H-indol-2-yl) methyl)-1-((4-bromo-1,3- dimethyl-1H-pyrazol-5-yl) methyl)-3,4-dimethyl-2-oxo- 1,2,3,4-tetrahydroquinoline- 7-carboxamide | 37 | B |
| UT158 | 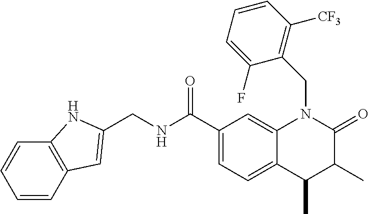 [SMILES: CC1C(=O)N(Cc2c(F)cccc2C(F)(F)F)c2cc(C(=O)NCc3cc4ccccc4[nH]3)ccc2[C@@H]1C] | (4R)-N-((1H-indol-2-yl) methyl)-1-(2-fluoro-6- (trifluoromethyl)benzyl)-3,4- dimethyl-2-oxo-1,2,3,4- tetrahydroquinoline-7- carboxamide | 39 | B |
| UT019 | 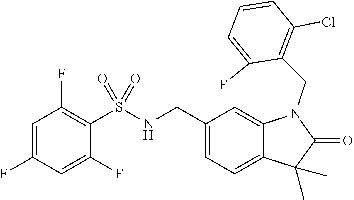 [SMILES: CC1(C)C(=O)N(Cc2c(F)cccc2Cl)c2cc(CNS(=O)(=O)c3c(F)cc(F)cc3F)ccc21] | N-((1-(2-chloro-6- fluorobenzyl)-3,3-dimethyl- 2-oxoindolin-6-yl)methyl)- 2,4,6- trifluorobenzenesulfonamide | 7 | C |
| UT071a | 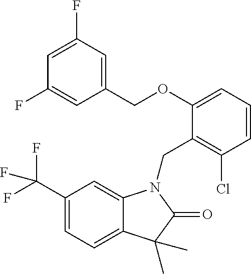 [SMILES: CC1(C)C(=O)N(Cc2c(Cl)cccc2OCc2cc(F)cc(F)c2)c2cc(C(F)(F)F)ccc21] | 1-(2-chloro-6-((3,5- difluorobenzyl)oxy)benzyl)- 3,3-dimethyl-6- (trifluoromethyl)indolin-2- one | 16 | D |
| UT072 | 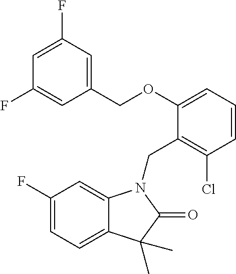 [SMILES: CC1(C)C(=O)N(Cc2c(Cl)cccc2OCc2cc(F)cc(F)c2)c2cc(F)ccc21] | 1-(2-chloro-6-((3,5- difluorobenzyl)oxy)benzyl)- 6-fluoro-3,3-dimethylindolin- 2-one | 17 | D |
| UT074 | 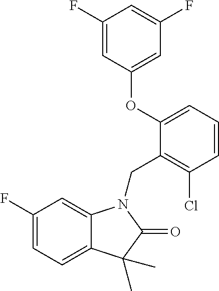 [SMILES: CC1(C)C(=O)N(Cc2c(Cl)cccc2Oc2cc(F)cc(F)c2)c2cc(F)ccc21] | 1-(2-chloro-6-(3,5- difluorophenoxy)benzyl)-6- fluoro-3,3-dimethylindolin- 2-one | 18 | D |
| UT065 | 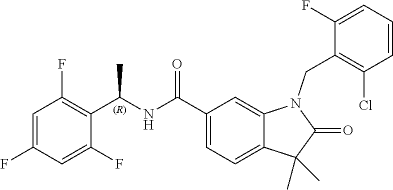 | (R)-1-(2-chloro-6- fluorobenzyl)-3,3-dimethyl- 2-oxo-N-(1-(2,4,6- trifluorophenyl)ethyl) indoline-6-carboxamide | 14 | D* |
| UT066 | 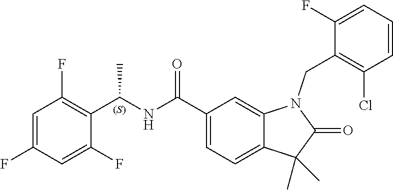 | (S)-1-(2-chloro-6- fluorobenzyl)-3,3-dimethyl- 2-oxo-N-(1-(2,4,6- trifluorophenyl)ethyl) indoline-6-carboxamide | 15 | D* |
| UT114 | 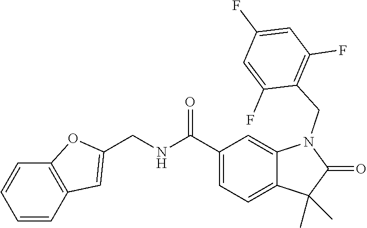 [SMILES: CC1(C)C(=O)N(Cc2c(F)cc(F)cc2F)c2cc(C(=O)NCc3cc4ccccc4o3)ccc21] | N-(benzofuran-2-ylmethyl)- 3,3-dimethyl-2-oxo-1-(2,4,6- trifluorobenzyl)indoline-6- carboxamide | 19 | D* |
| UT120 | 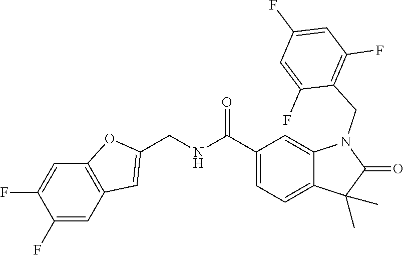 [SMILES: CC1(C)C(=O)N(Cc2c(F)cc(F)cc2F)c2cc(C(=O)NCc3cc4cc(F)c(F)cc4o3)ccc21] | N-((5,6-difluorobenzofuran- 2-yl)methyl)-3,3-dimethyl-2- oxo-1-(2,4,6-trifluorobenzyl) indoline-6-carboxamide | 23 | D* |
| UT121 | 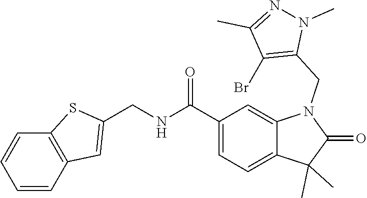 [SMILES: Cc1nn(C)c(CN2C(=O)C(C)(C)c3ccc(C(=O)NCc4cc5ccccc5s4)cc32)c1Br] | N-(benzo[b]thiophen-2- ylmethyl)-1-((4-bromo-1,3- dimethyl-1H-pyrazol-5-yl) methyl)-3,3-dimethyl-2- oxoindoline-6-carboxamide | 24 | D* |
| UT115 | 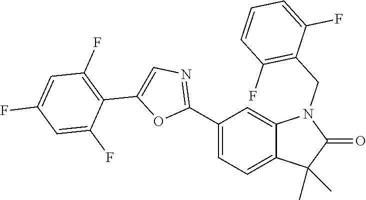 [SMILES: CC1(C)C(=O)N(Cc2c(F)cccc2F)c2cc(-c3ncc(-c4c(F)cc(F)cc4F)o3)ccc21] | 1-(2,6-difluorobenzyl)-3,3- dimethyl-6-(5-(2,4,6- trifluorophenyl)oxazol-2- yl)indolin-2-one | 20 | D* |
| UT117 | 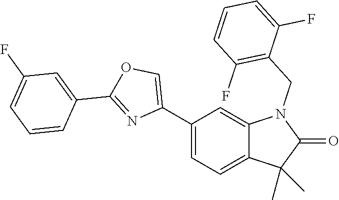 [SMILES: CC1(C)C(=O)N(Cc2c(F)cccc2F)c2cc(-c3coc(-c4cccc(F)c4)n3)ccc21] | 1-(2,6-difluorobenzyl)-6-(2- (3-fluorophenyl)oxazol-4- yl)-3,3-dimethylindolin-2- one | 21 | D* |
| UT118 | 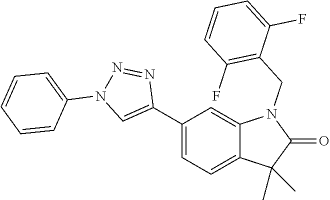 [SMILES: CC1(C)C(=O)N(Cc2c(F)cccc2F)c2cc(-c3cn(-c4ccccc4)nn3)ccc21] | 1-(2,6-difluorobenzyl)-3,3- dimethyl-6-(1-phenyl-1H- 1,2,3-triazol-4-yl)indolin-2- one | 22 | D* |

A >50% inhibition at 10 μM upon cGAMP (100 μM) or MSA-2 (10 μM) stimulation in THP-1 cells
B <50% inhibition at 10 μM upon cGAMP (100 μM) or MSA-2 (10 μM) stimulation in THP-1 cells.
C >50% inhibition at 32 μM under basal conditions (no external stimulation) in THP-1 cells.
D <50% inhibition at 32 μM under basal conditions (no external stimulation) in THP-1 cells.
*These compounds had activity levels too weak to be practical STING antagonists

### REFERENCES

1 Zhang, X., Bai, X. C. & Chen, Z. J. Structures and Mechanisms in the cGAS-STING Innate Immunity Pathway. Immunity 53, 43-53, doi:10.1016/j.immuni.2020.05.013 (2020).
2 Ishikawa, H. & Barber, G. N. STING is an endoplasmic reticulum adaptor that facilitates innate immune signalling. Nature 455, 674-678, doi:10.1038/nature07317 (2008).
3 Zhong, B. et al. The adaptor protein MITA links virus-sensing receptors to IRF3 transcription factor activation. Immunity 29, 538-550, doi:10.1016/j.immuni.2008.09.003 (2008).
4 Jin, L. et al. MPYS, a novel membrane tetraspanner, is associated with major histocompatibility complex class II and mediates transduction of apoptotic signals. Molecular and cellular biology 28, 5014-5026, doi:10.1128/MCB.00640-08 (2008).
5 Sun, W. et al. ERIS, an endoplasmic reticulum IFN stimulator, activates innate immune signaling through dimerization. Proc Natl Acad Sci USA 106, 8653-8658, doi:10.1073/pnas.0900850106 (2009).
6 Wu, J. et al. Cyclic GMP-AMP is an endogenous second messenger in innate immune signaling by cytosolic DNA. Science 339, 826-830, doi:10.1126/science.1229963 (2013).
7 Sun, L., Wu, J., Du, F., Chen, X. & Chen, Z. J. Cyclic GMP-AMP synthase is a cytosolic DNA sensor that activates the type I interferon pathway. Science 339, 786-791, doi:10.1126/science.1232458(2013).
8 Shang, G., Zhang, C., Chen, Z. J., Bai, X. C. & Zhang, X. Cryo-EM structures of STING reveal its mechanism of activation by cyclic GMP-AMP. Nature 567, 389-393, doi:10.1038/s41586-019-0998-5 (2019).
9 Zhang, C. et al. Structural basis of STING binding with and phosphorylation by TBK1. Nature 567, 394-398, doi:10.1038/s41586-019-1000-2 (2019).
10 Pryde, D. C. et al. The discovery of potent small molecule activators of human STING. Eur J Med Chem 209, 112869, doi:10.1016/j.ejmech.2020.112869 (2020).
11 Burdette, D. L. et al. STING is a direct innate immune sensor of cyclic di-GMP. Nature 478, 515-518, doi:10.1038/nature10429 (2011).
12 Dou, Z. et al. Cytoplasmic chromatin triggers inflammation in senescence and cancer. Nature 550, 402-406, doi:10.1038/nature24050 (2017).
13 Gluck, S. et al. Innate immune sensing of cytosolic chromatin fragments through cGAS promotes senescence. Nature cell biology 19, 1061-1070, doi:10.1038/ncb3586 (2017).
14 Mackenzie, K. J. et al. cGAS surveillance of micronuclei links genome instability to innate immunity. Nature 548, 461-465, doi:10.1038/nature23449 (2017).
15 Yang, H., Wang, H., Ren, J., Chen, Q. & Chen, Z. J. cGAS is essential for cellular senescence. Proc Natl Acad Sci USA 114, E4612-E4620, doi:10.1073/pnas.1705499114 (2017).
16 Motedayen Aval, L., Pease, J. E., Sharma, R. & Pinato, D. J. Challenges and Opportunities in the Clinical Development of STING Agonists for Cancer Immunotherapy. J Clin Med 9, doi:10.3390/jcm9103323 (2020).
17 Zhao, B. et al. A conserved PLPLRT/SD motif of STING mediates the recruitment and activation of TBK1. Nature 569, 718-722, doi:10.1038/s41586-019-1228-x (2019).
18 Tanaka, Y. & Chen, Z. J. STING specifies IRF3 phosphorylation by TBK1 in the cytosolic DNA signaling pathway. Science signaling 5, ra20, doi:10.1126/scisignal.2002521 (2012).
19 Liu, S. et al. Phosphorylation of innate immune adaptor proteins MAVS, STING, and TRIF induces IRF3 activation. Science 347, aaa2630, doi:10.1126/science.aaa2630 (2015).
20 Ergun, S. L., Fernandez, D., Weiss, T. M. & Li, L. STING Polymer Structure Reveals Mechanisms for Activation, Hyperactivation, and Inhibition. Cell 178, 290-301 e210, doi:10.1016/j.cell.2019.05.036 (2019).
21 Dobbs, N. et al. STING Activation by Translocation from the ER Is Associated with Infection and Autoinflammatory Disease. Cell Host Microbe 18, 157-168, doi:10.1016/j.chom.2015.07.001 (2015).
22 Saitoh, T. et al. Atg9a controls dsDNA-driven dynamic translocation of STING and the innate immune response. Proc Natl Acad Sci USA 106, 20842-20846, doi:10.1073/pnas.0911267106 (2009).
23 Gui, X. et al. Autophagy induction via STING trafficking is a primordial function of the cGAS pathway. Nature 567, 262-266, doi:10.1038/s41586-019-1006-9 (2019).
24 Zhang, X. et al. Cyclic GMP-AMP Containing Mixed Phosphodiester Linkages Is An Endogenous High-Affinity Ligand for STING. Molecular cell 51, 226-235, doi:10.1016/j.molcel.2013.05.022 (2013).
Kranzusch, P. J. et al. Ancient Origin of cGAS-STING Reveals Mechanism of Universal 2′,3′ cGAMP Signaling. Molecular cell 59, 891-903, doi:10.1016/j.molcel.2015.07.022 (2015).
26 Shi, H., Wu, J., Chen, Z. J. & Chen, C. Molecular basis for the specific recognition of the metazoan cyclic GMP-AMP by the innate immune adaptor protein STING. Proc Natl Acad Sci USA 112, 8947-8952, doi:10.1073/pnas.1507317112 (2015).
27 Gao, P. et al. Structure-function analysis of STING activation by c[G(2′,5′)pA(3′,5′)p] and targeting by antiviral DMXAA. Cell 154, 748-762, doi:10.1016/j.cell.2013.07.023 (2013).
28 Ernst, A. M. et al. S-Palmitoylation Sorts Membrane Cargo for Anterograde Transport in the Golgi. Developmental cell 47, 479-493 e477, doi:10.1016/j.devcel.2018.10.024 (2018).
29 Cong, X. et al. Crystal structures of porcine STING(CBD)-CDN complexes reveal the mechanism of ligand recognition and discrimination of STING proteins. The Journal of biological chemistry 294, 11420-11432, doi:10.1074/jbc.RA119.007367 (2019).
30 Conlon, J. et al. Mouse, but not human STING, binds and signals in response to the vascular disrupting agent 5,6-dimethylxanthenone-4-acetic acid. J Immunol 190, 5216-5225, doi:10.4049/jimmunol.1300097 (2013).
31 Morales-Perez, C. L., Noviello, C. M. & Hibbs, R. E. Manipulation of Subunit Stoichiometry in Heteromeric Membrane Proteins. Structure 24, 797-805, doi:10.1016/j.str.2016.03.004 (2016).
32 Zheng, S. Q. et al. MotionCor2: anisotropic correction of beam-induced motion for improved cryo-electron microscopy. Nat Methods 14, 331-332, doi:10.1038/nmeth.4193 (2017).
33 Zhang, K. Gctf: Real-time CTF determination and correction. J Struct Biol 193, 1-12, doi:10.1016/j.jsb.2015.11.003 (2016).
34 Bepler, T. et al. Positive-unlabeled convolutional neural networks for particle picking in cryo-electron micrographs. Nat Methods 16, 1153-1160, doi:10.1038/s41592-019-0575-8 (2019).
35 Zivanov, J. et al. New tools for automated high-resolution cryo-EM structure determination in RELION-3. Elife 7, doi:10.7554/eLife.42166 (2018).
36 Emsley, P., Lohkamp, B., Scott, W. G. & Cowtan, K. Features and development of Coot. Acta crystallographica 66, 486-501, doi:10.1107/S0907444910007493 (2010).
37 Gao, P. et al. Cyclic [G(2′,5′)pA(3′,5′)p] is the metazoan second messenger produced by DNA-activated cyclic GMP-AMP synthase. Cell 153, 1094-1107, doi:10.1016/j.cell.2013.04.046 (2013).
38 Ablasser, A. et al. cGAS produces a 2′-5′-linked cyclic dinucleotide second messenger that activates STING. Nature 498, 380-384, doi:10.1038/nature12306 (2013).
39 Adams, P. D. et al. PHENIX: a comprehensive Python-based system for macromolecular structure solution. Acta crystallographica 66, 213-221, doi:10.1107/S0907444909052925 (2010).
40 Chen, V. B. et al. MolProbity: all-atom structure validation for macromolecular crystallography. Acta crystallographica 66, 12-21, doi:10.1107/S0907444909042073 (2010).
41 Pettersen, E. F. et al. UCSF Chimera—a visualization system for exploratory research and analysis. J Comput Chem 25, 1605-1612, doi:10.1002/jcc.20084 (2004).
42 Robert, X. & Gouet, P. Deciphering key features in protein structures with the new ENDscript server. Nucleic Acids Res 42, W320-324, doi:10.1093/nar/gku316 (2014).
43 Laskowski, R. A. & Swindells, M. B. LigPlot+: multiple ligand-protein interaction diagrams for drug discovery. J Chem Inf Model 51, 2778-2786, doi:10.1021/ci200227u (2011).
44 Woodward, J. J., Iavarone, A. T. & Portnoy, D. A. c-di-AMP secreted by intracellular Listeria monocytogenes activates a host type I interferon response. Science 328, 1703-1705, doi:10.1126/science.1189801 (2010).

1. A method of allosterically inhibiting activity of a stimulator of interferon genes protein (STING), the method comprising contacting a compound of Formula (I), Formula (II) or Formula III to STING: 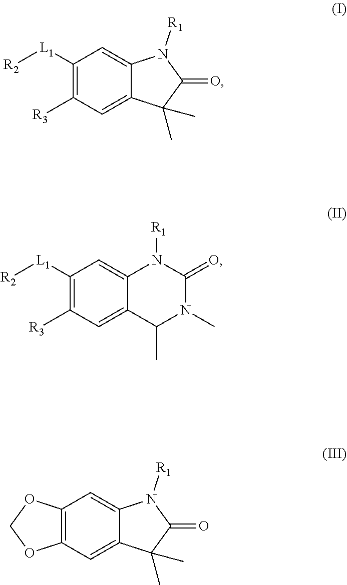
or a salt thereof,
wherein L₁ is absent or is selected from the group consisting of 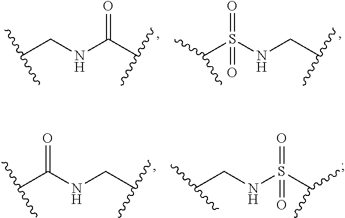 [SMILES: *CNC(*)=O ; *CNS(*)(=O)=O ; *CNC(*)=O ; *CNS(*)(=O)=O]
R₁ is selected from the group consisting of 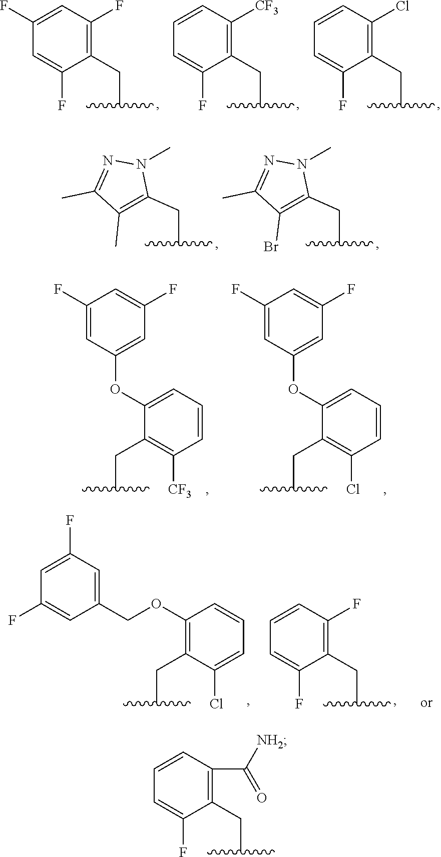
R₂ is selected from the group consisting of H, CF₃, F, CN, 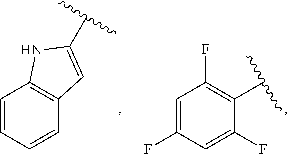 [SMILES: *c1cc2ccccc2[nH]1 ; *c1c(F)cc(F)cc1F]
and
R₃ is selected from the group consisting of H, CN or CF₃;
with the provisos that
(i) at least one of R₂ and R₃ is not hydrogen and
(ii) R₁ is not 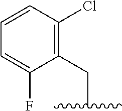 [SMILES: *Cc1c(F)cccc1Cl]
when L1 is 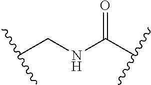 [SMILES: *CNC(*)=O]
and R₂ is

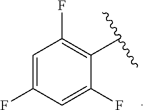 [SMILES: *c1c(F)cc(F)cc1F]

2. The method of claim 1, wherein L1 is absent or

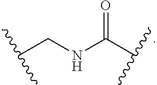 [SMILES: *CNC(*)=O]

3. The method of claim 1 or 2, wherein R₁ is selected from the group consisting of

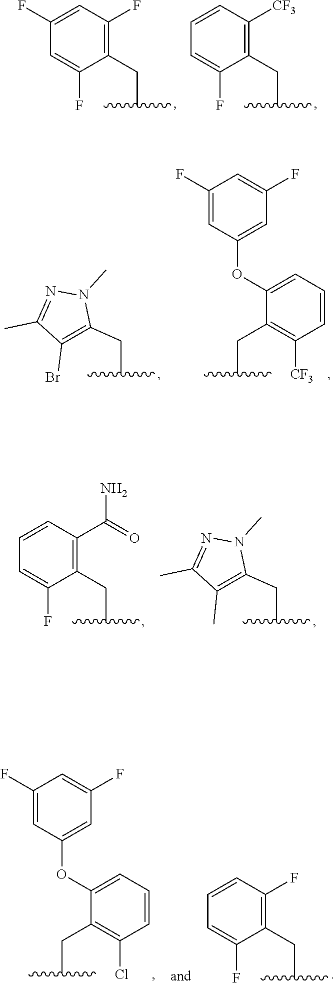

4. The method of any one of claims 1 to 3, wherein R₁ is selected from the group consisting of

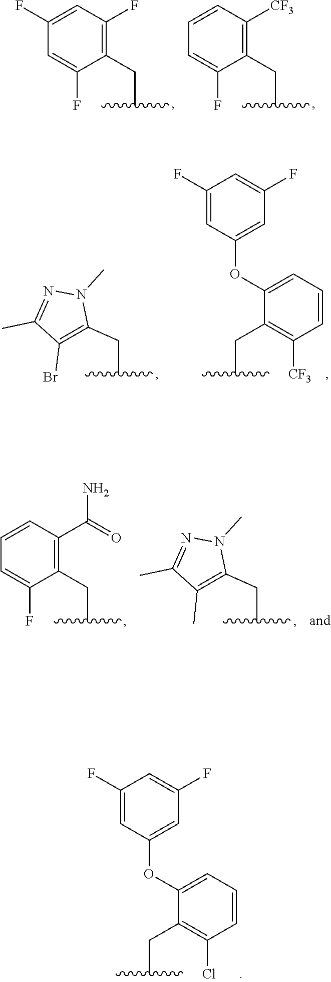

5. The method of claim 3 or 4, wherein R₁ is selected from the group consisting of

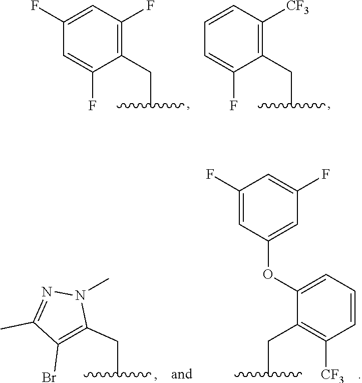

6. The method of any one of claims 1 to 5, wherein R₂ is selected from the group consisting of 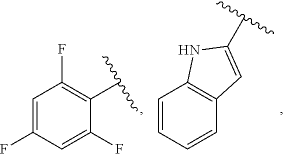
—CN, H, and —CF₃.

7. The method of any one of claims 1 to 6, wherein R₂ is selected from the group consisting of 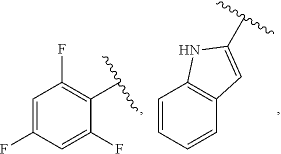
and —CN.

8. The method of any one of claims 1 to 7, wherein R₃ is hydrogen.

9. The method of any one of claims 1 to 8, wherein the compound is selected from the group consisting of: 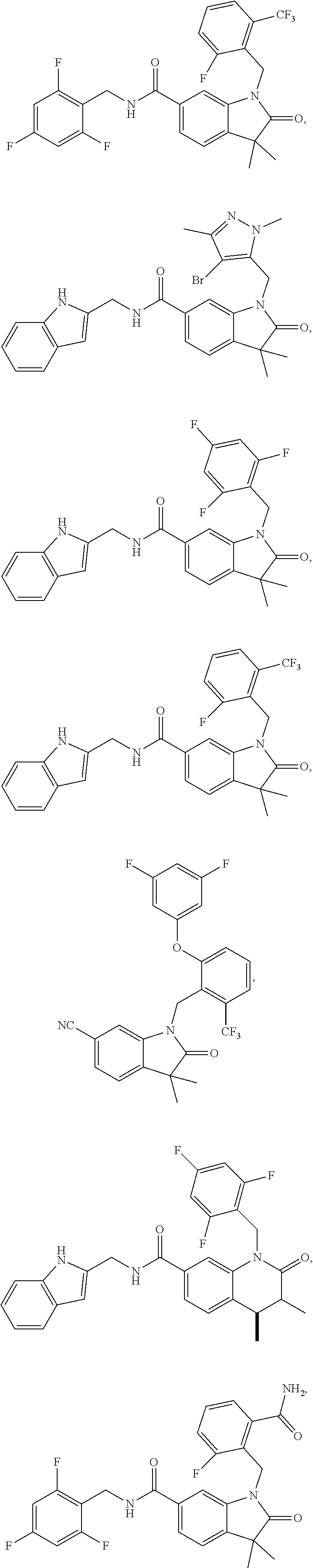 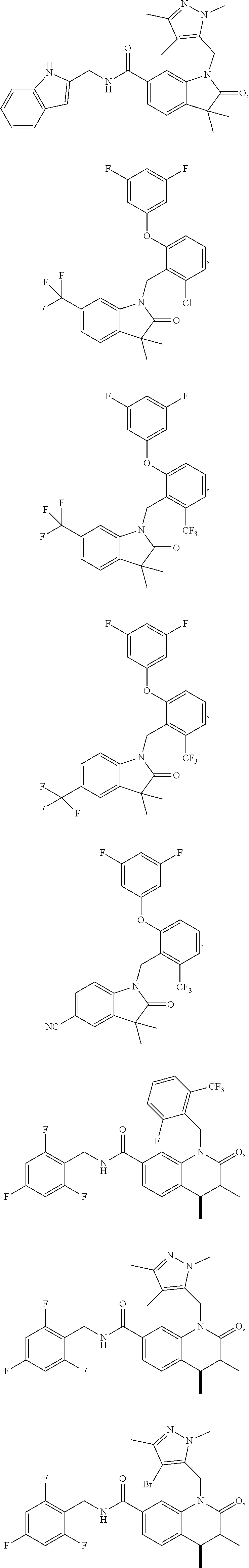 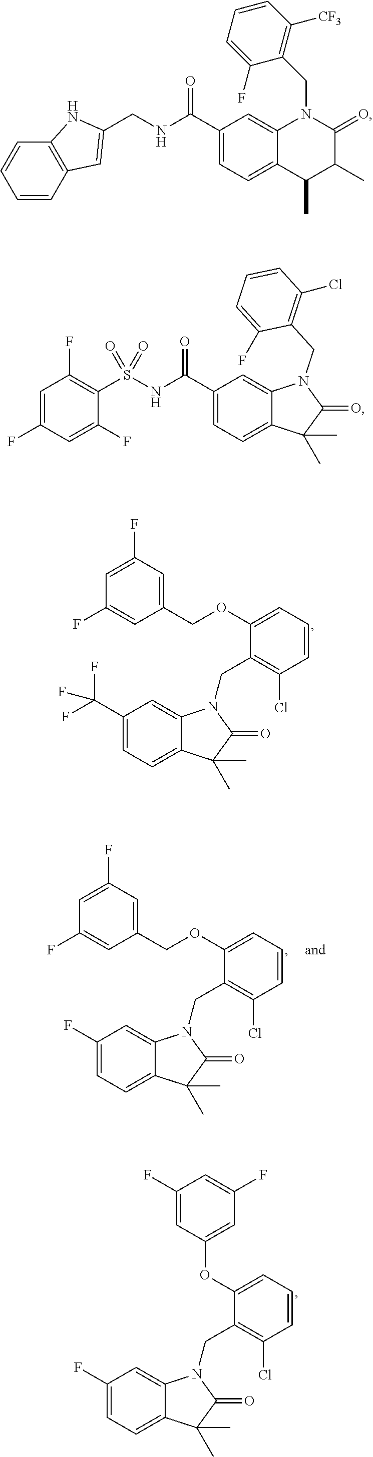 [SMILES: CC1C(=O)N(Cc2c(F)cccc2C(F)(F)F)c2cc(C(=O)NCc3cc4ccccc4[nH]3)ccc2[C@@H]1C ; CC1(C)C(=O)N(Cc2c(F)cccc2Cl)c2cc(C(=O)NS(=O)(=O)c3c(F)cc(F)cc3F)ccc21 ; CC1(C)C(=O)N(Cc2c(Cl)cccc2OCc2cc(F)cc(F)c2)c2cc(C(F)(F)F)ccc21 ; CC1(C)C(=O)N(Cc2c(Cl)cccc2OCc2cc(F)cc(F)c2)c2cc(F)ccc21 ; CC1(C)C(=O)N(Cc2c(Cl)cccc2Oc2cc(F)cc(F)c2)c2cc(F)ccc21]
and any salt thereof.

10. The method of any one of claims 1 to 9, wherein the compound is selected from the group consisting of: 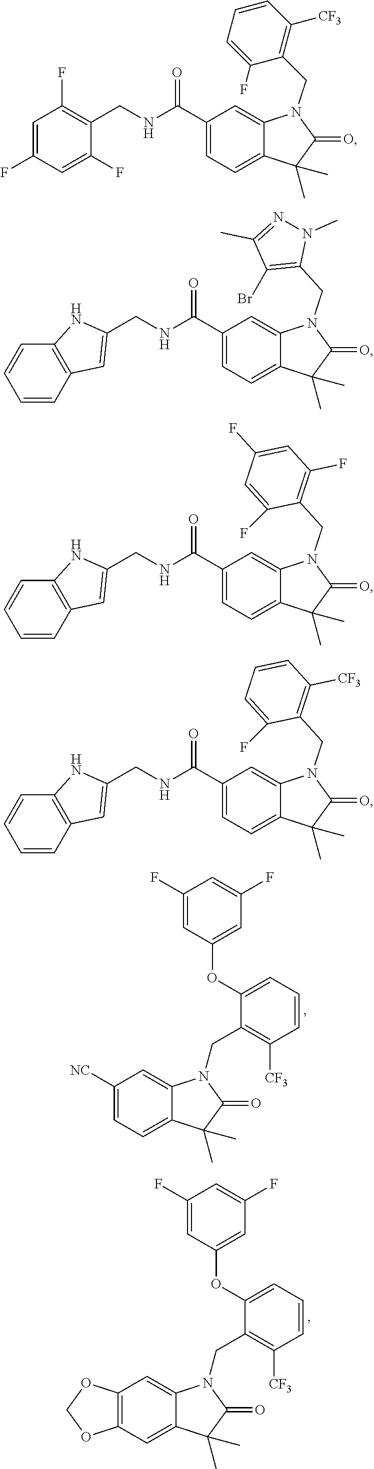 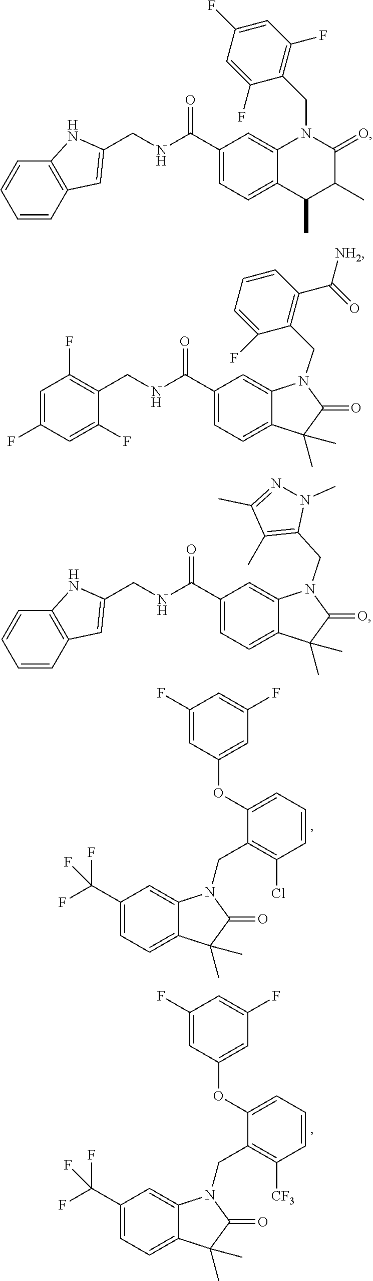 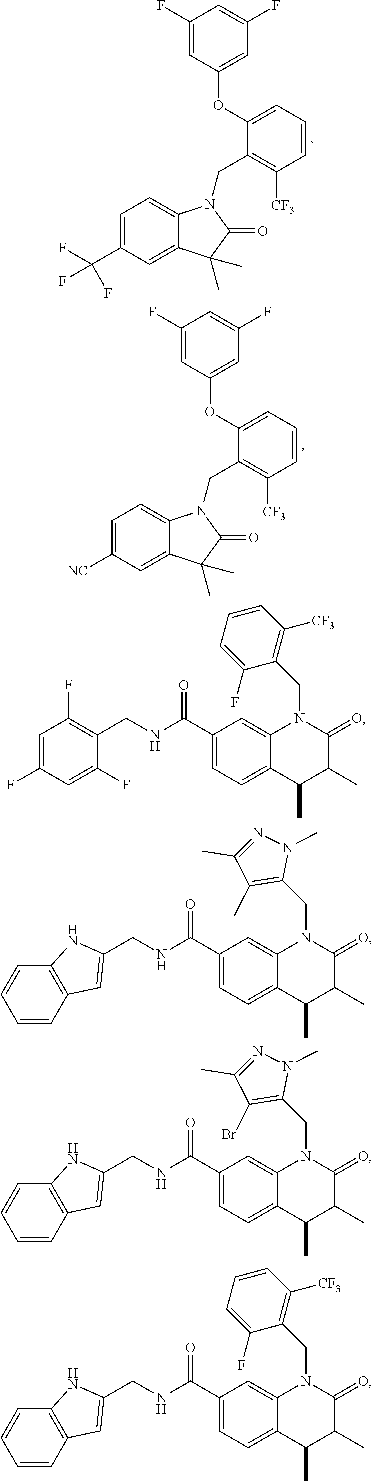 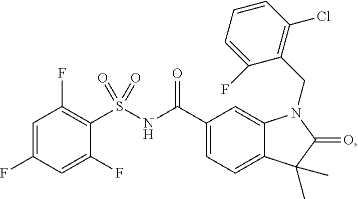 [SMILES: CC1(C)C(=O)N(Cc2c(F)cccc2Cl)c2cc(C(=O)NS(=O)(=O)c3c(F)cc(F)cc3F)ccc21]
and any salt thereof.

11. The method of any one of claims 1 to 10, wherein the compound is selected from the group consisting of: 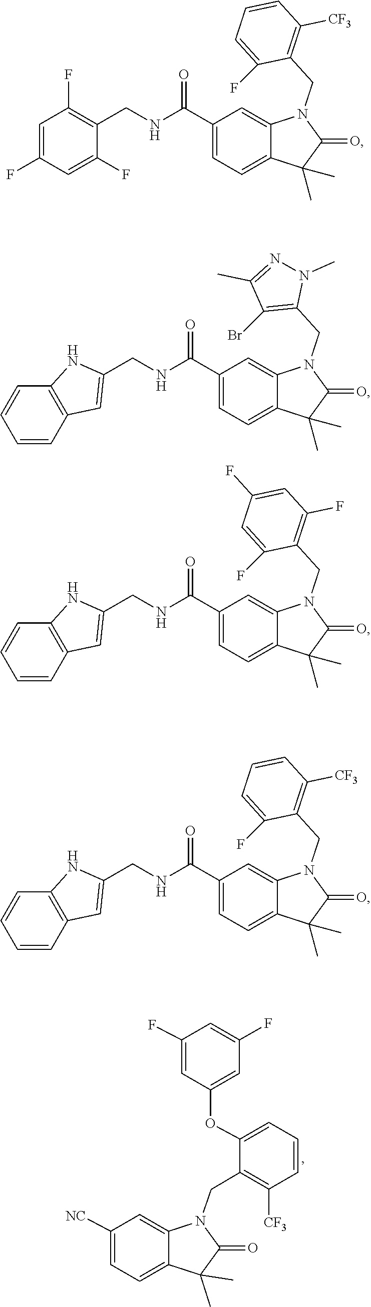 [SMILES: CC1(C)C(=O)N(Cc2c(F)cccc2C(F)(F)F)c2cc(C(=O)NCc3c(F)cc(F)cc3F)ccc21 ; Cc1nn(C)c(CN2C(=O)C(C)(C)c3ccc(C(=O)NCc4cc5ccccc5[nH]4)cc32)c1Br ; CC1(C)C(=O)N(Cc2c(F)cc(F)cc2F)c2cc(C(=O)NCc3cc4ccccc4[nH]3)ccc21 ; CC1(C)C(=O)N(Cc2c(F)cccc2C(F)(F)F)c2cc(C(=O)NCc3cc4ccccc4[nH]3)ccc21 ; CC1(C)C(=O)N(Cc2c(Oc3cc(F)cc(F)c3)cccc2C(F)(F)F)c2cc(C#N)ccc21] 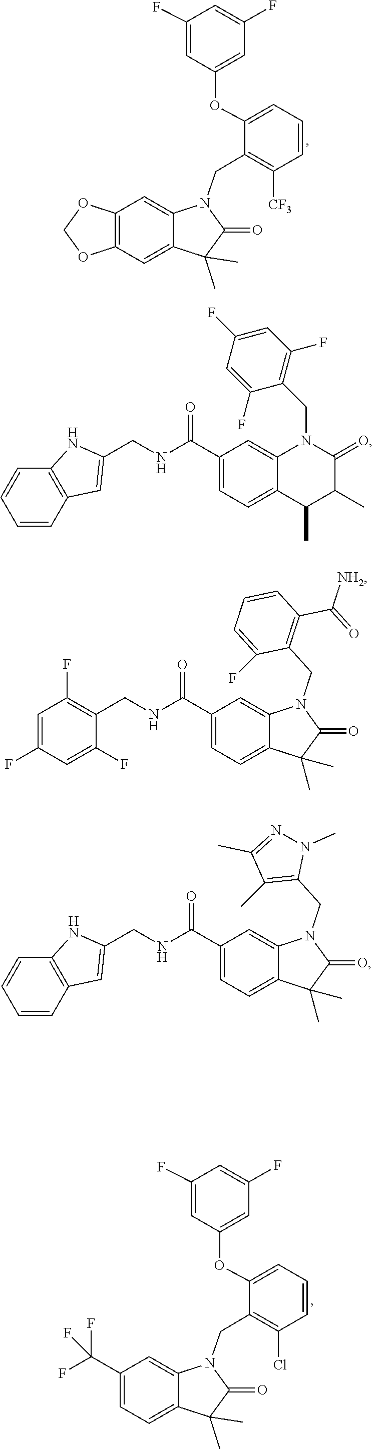 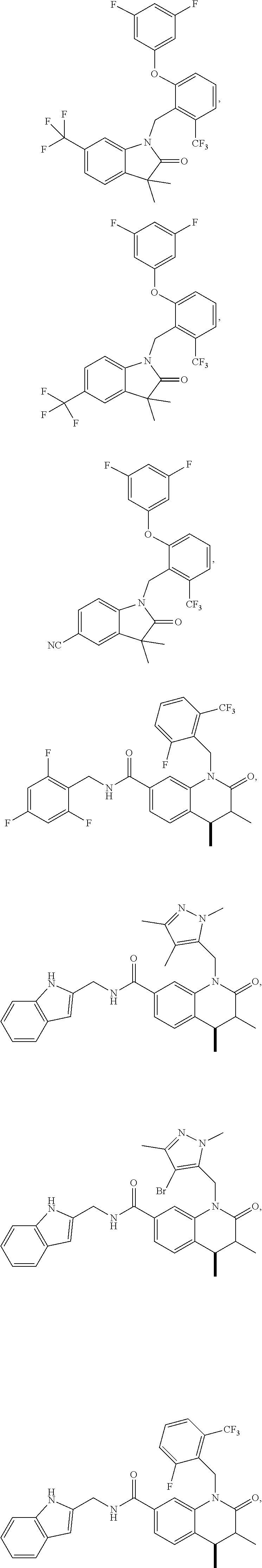
and any salt thereof.

12. The method of any one of claims 1 to 11, wherein the compound is selected from the group consisting of: 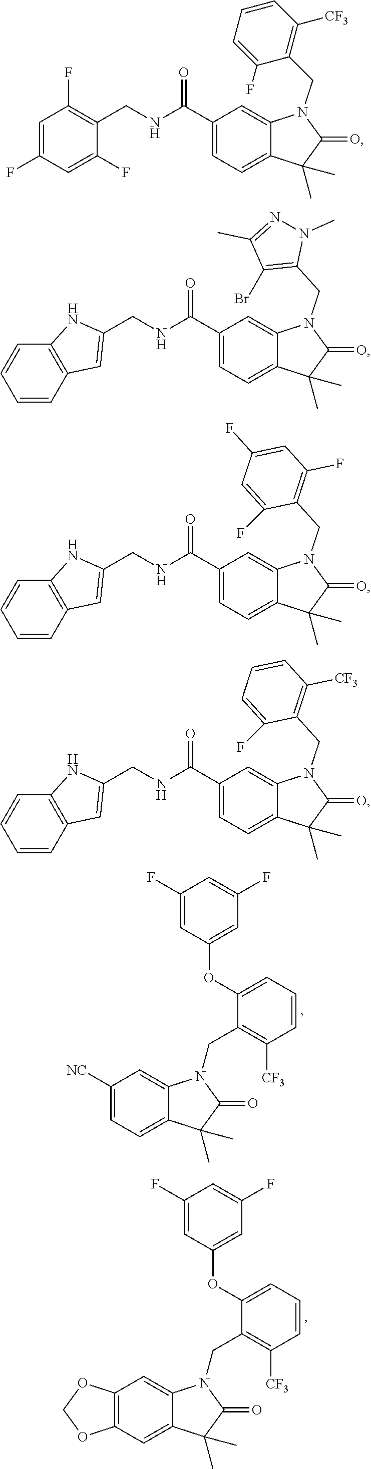 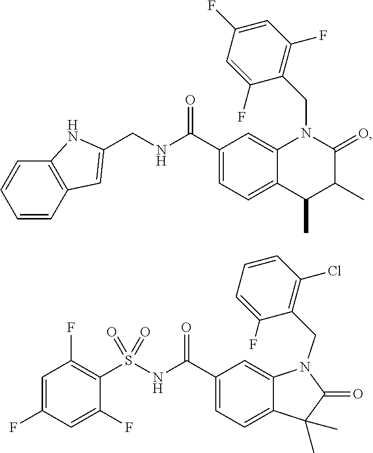
and any salt thereof.

13. The method of any one of claims 1 to 12, wherein the compound is selected from the group consisting of: 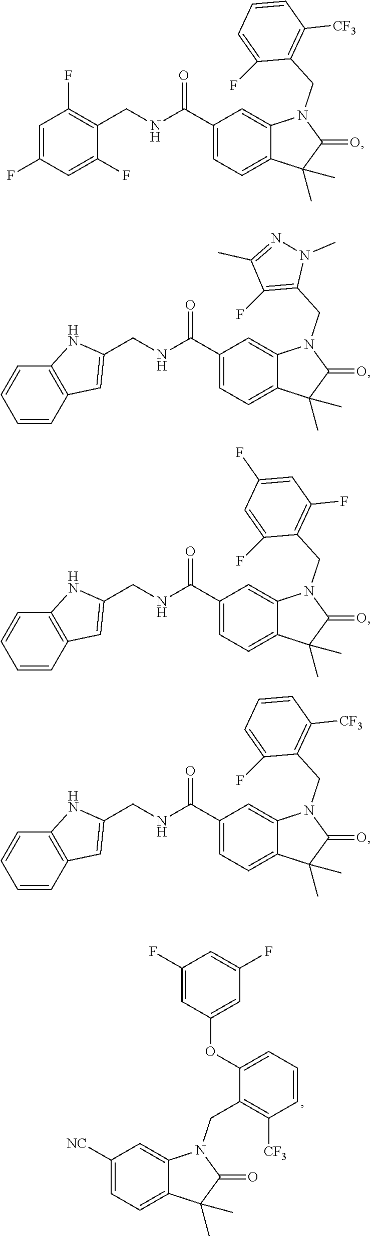 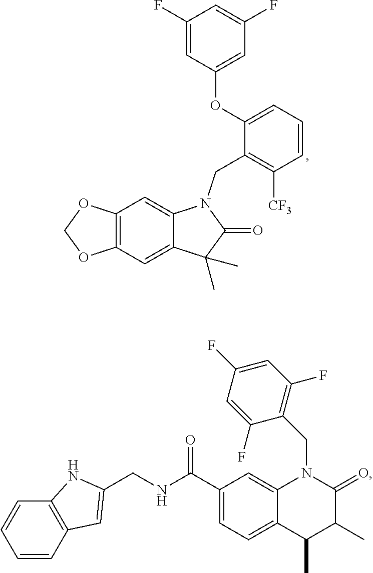 [SMILES: CC1(C)C(=O)N(Cc2c(Oc3cc(F)cc(F)c3)cccc2C(F)(F)F)c2cc3c(cc21)OCO3 ; CC1C(=O)N(Cc2c(F)cc(F)cc2F)c2cc(C(=O)NCc3cc4ccccc4[nH]3)ccc2[C@@H]1C]
and any salt thereof.

14. The method of any one of claims 1 to 13, wherein the method is in vitro.

15. The method of any one of claims 1 to 14, wherein the method is in vivo.

16. A method of treating a condition caused or related to disrupted STING signaling in a subject in need thereof, the method comprising administering a STING antagonist to the subject, wherein the STING antagonist is a compound of Formula (I), Formula (II) or Formula III: 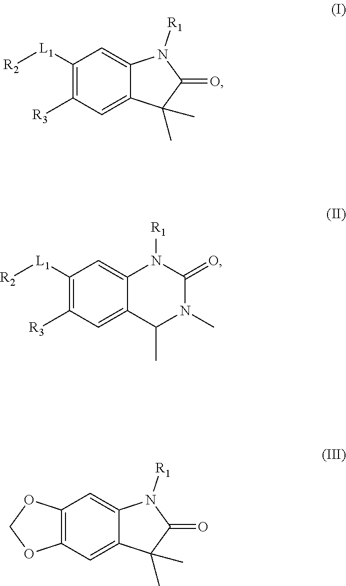
or a salt thereof,
wherein L₁ is absent or is selected from the group consisting of 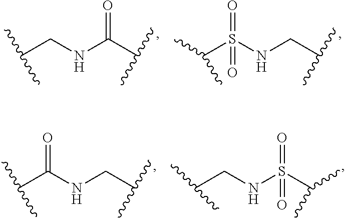 [SMILES: *CNC(*)=O ; *CNS(*)(=O)=O ; *CNC(*)=O ; *CNS(*)(=O)=O]
and;
R₁ is selected from the group consisting of 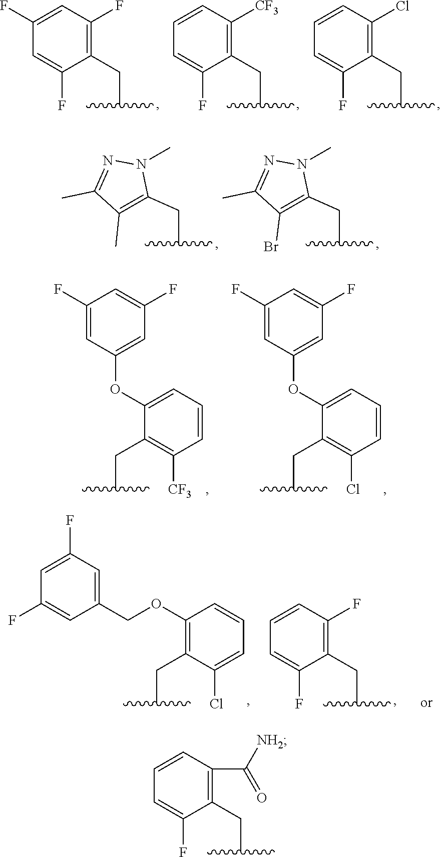
R₂ is selected from the group consisting of H, CF₃, F, CN, 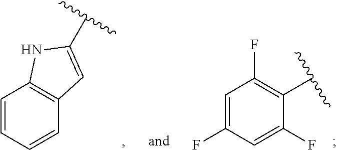 [SMILES: *c1cc2ccccc2[nH]1 ; *c1c(F)cc(F)cc1F]
and
R₃ is selected from the group consisting of H, CN or CF₃;
with the provisos that
(i) at least one of R₂ and R₃ is not hydrogen and
(ii) R₁ is not 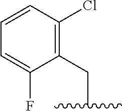 [SMILES: *Cc1c(F)cccc1Cl]
when L1 is 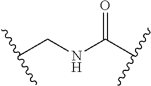 [SMILES: *CNC(*)=O]
and R₂ is

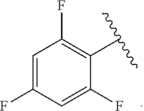 [SMILES: *c1c(F)cc(F)cc1F]

16. The method of claim 15, wherein L1 is absent or

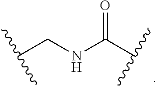 [SMILES: *CNC(*)=O]

17. The method of claim 15 or 16, wherein R₁ is selected from the group consisting of

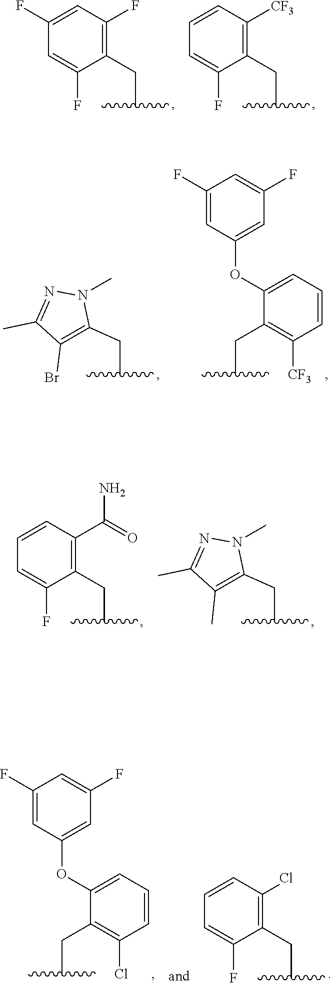

18. The method of any one of claims 15 to 17, wherein R₁ is selected from the group consisting of

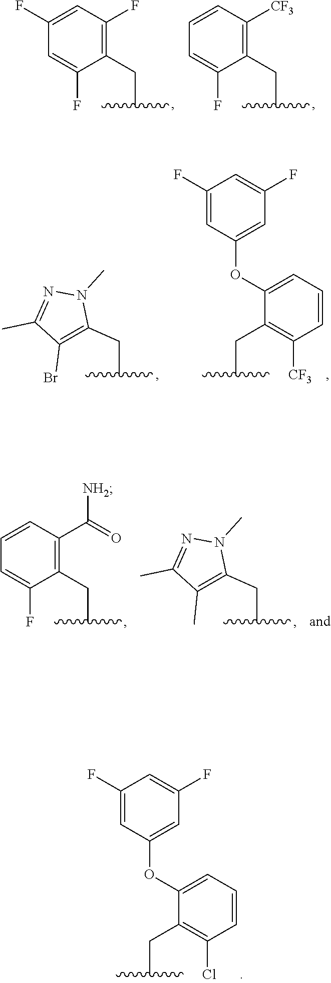

19. The method of claim 15 to 18, wherein R₁ is selected from the group consisting of

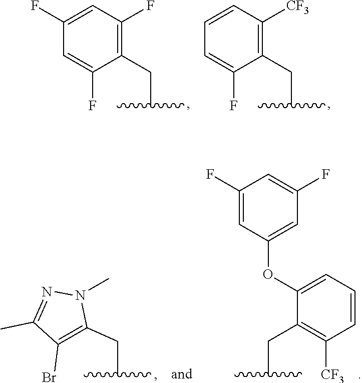

20. The method of any one of claims 15 to 19, wherein R₂ is selected from the group consisting of 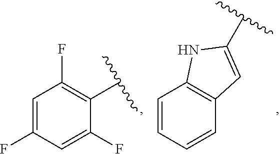
—CN, H, and —CF₃.

21. The method of any one of claims 15 to 20, wherein R₂ is selected from the group consisting of 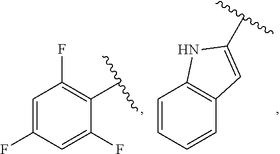
and —CN.

22. The method of any one of claims 15 to 21, wherein R₃ is hydrogen.

23. The method of any one of claims 15 to 22, wherein the STING antagonist is selected from the group consisting of: 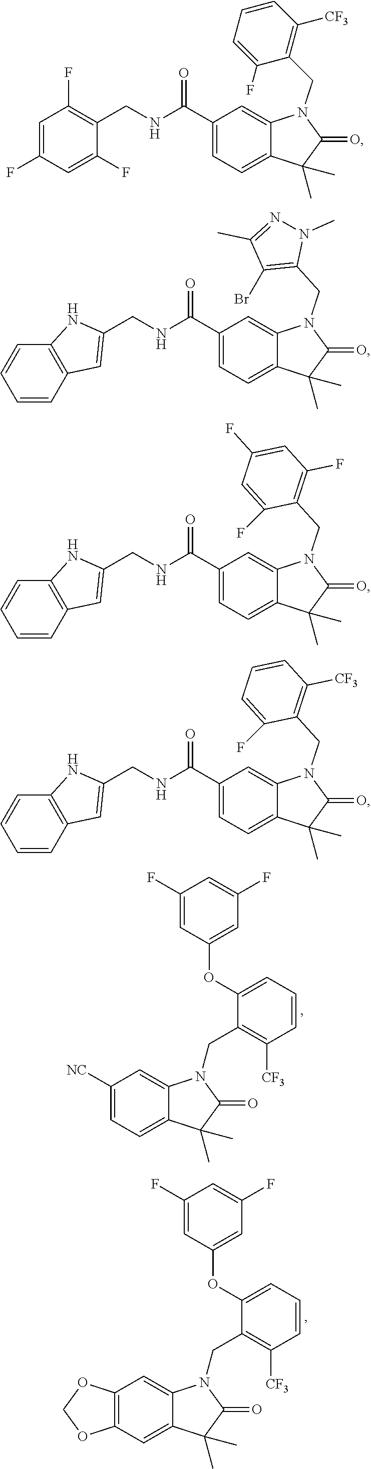 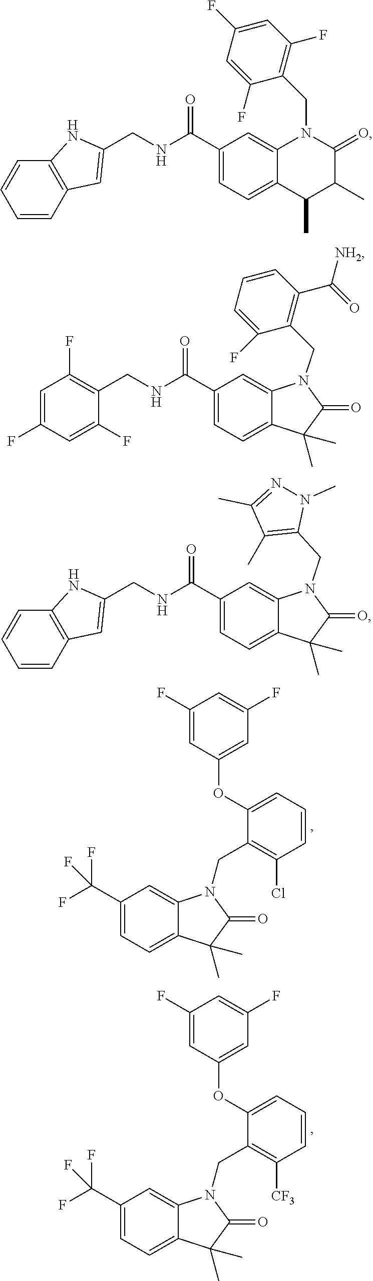 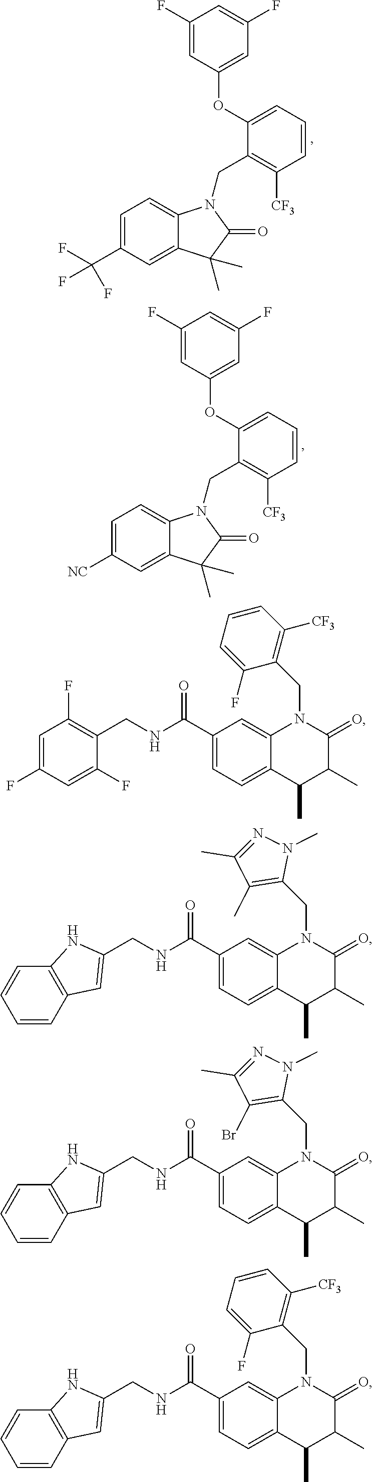 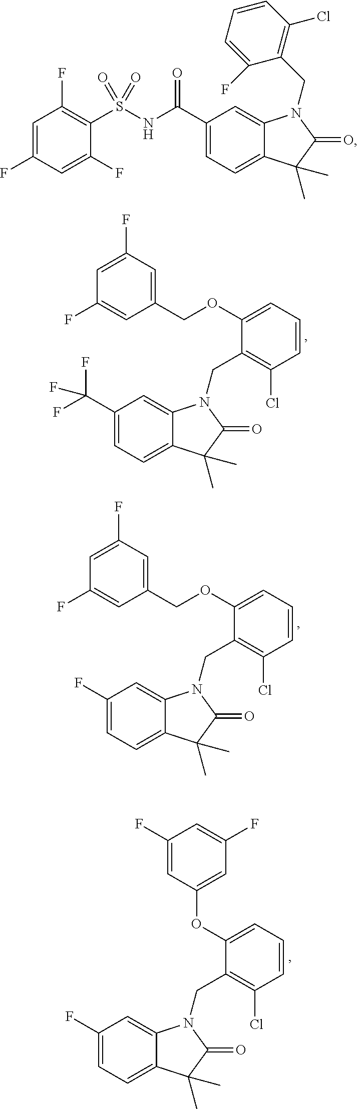 [SMILES: CC1(C)C(=O)N(Cc2c(F)cccc2Cl)c2cc(C(=O)NS(=O)(=O)c3c(F)cc(F)cc3F)ccc21 ; CC1(C)C(=O)N(Cc2c(Cl)cccc2OCc2cc(F)cc(F)c2)c2cc(C(F)(F)F)ccc21 ; CC1(C)C(=O)N(Cc2c(Cl)cccc2OCc2cc(F)cc(F)c2)c2cc(F)ccc21 ; CC1(C)C(=O)N(Cc2c(Cl)cccc2Oc2cc(F)cc(F)c2)c2cc(F)ccc21]
and any salt thereof.

24. The method of any one of claims 15 to 23, wherein the STING antagonist is selected from the group consisting of: 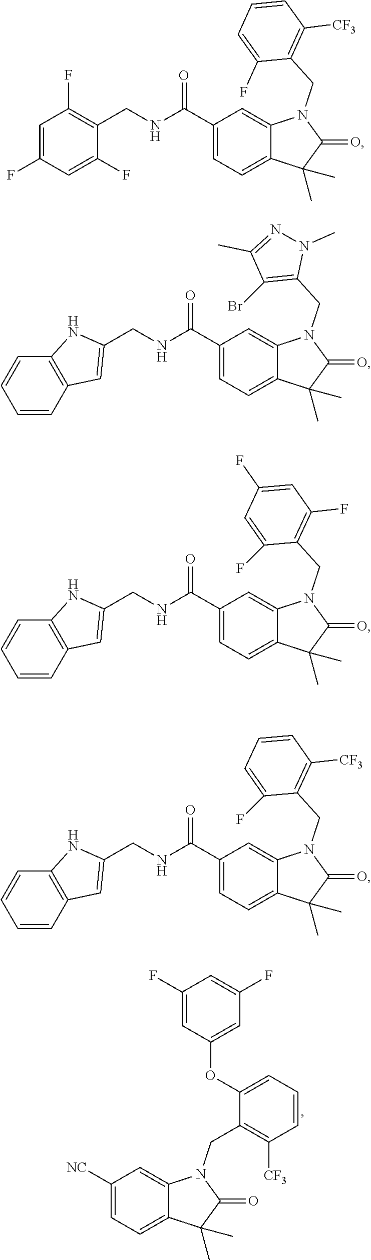 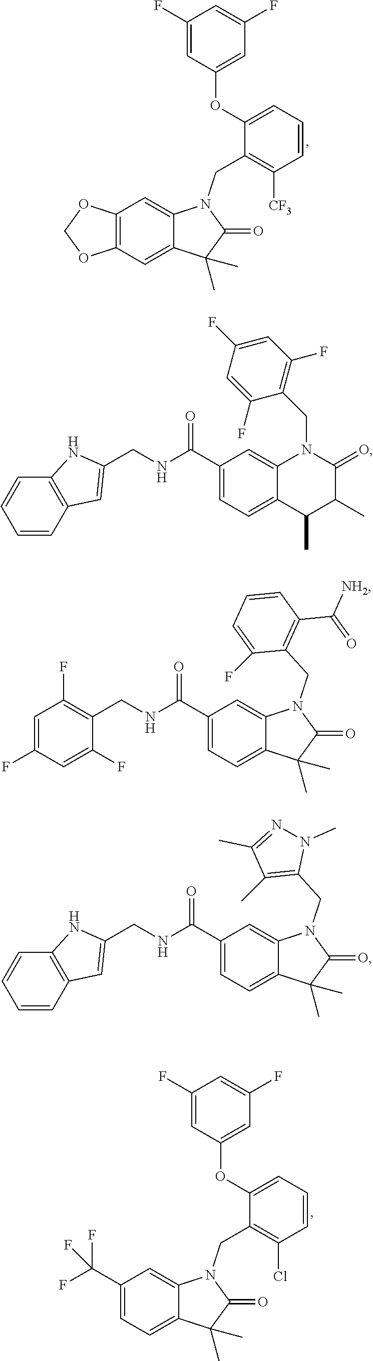 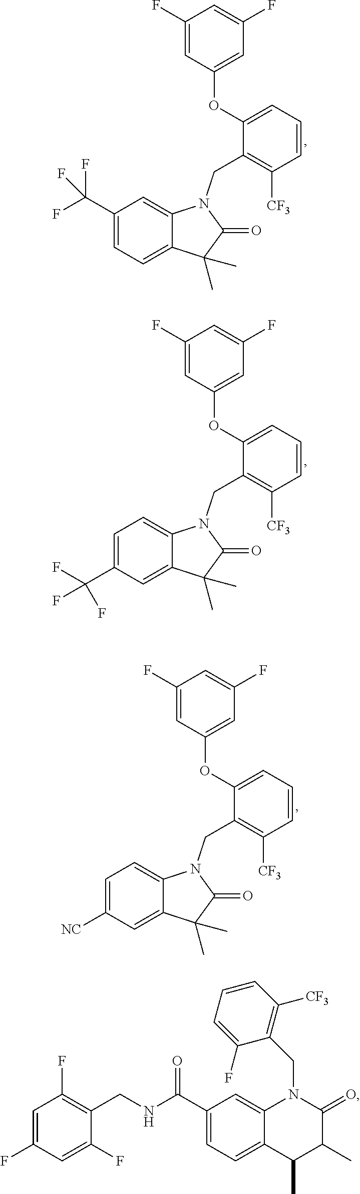 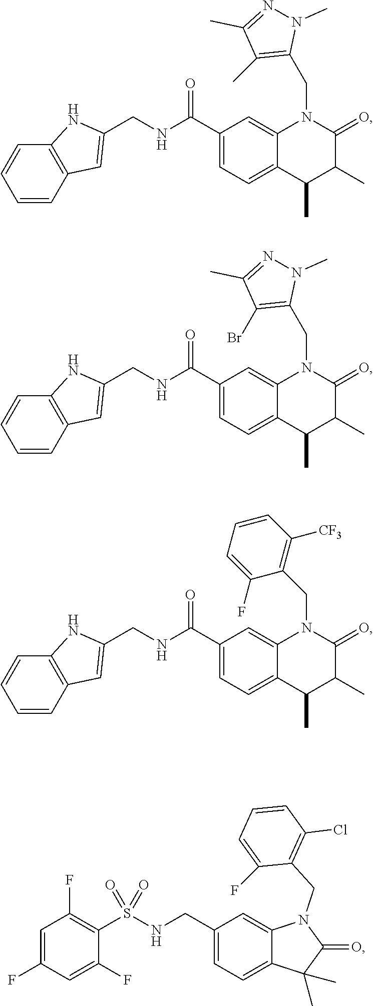 [SMILES: Cc1nn(C)c(CN2C(=O)C(C)[C@@H](C)c3ccc(C(=O)NCc4cc5ccccc5[nH]4)cc32)c1C ; Cc1nn(C)c(CN2C(=O)C(C)[C@@H](C)c3ccc(C(=O)NCc4cc5ccccc5[nH]4)cc32)c1Br ; CC1C(=O)N(Cc2c(F)cccc2C(F)(F)F)c2cc(C(=O)NCc3cc4ccccc4[nH]3)ccc2[C@@H]1C ; CC1(C)C(=O)N(Cc2c(F)cccc2Cl)c2cc(CNS(=O)(=O)c3c(F)cc(F)cc3F)ccc21]
and any salt thereof.

25. The method of any one of claims 15 to 24, wherein the STING antagonist is selected from the group consisting of: 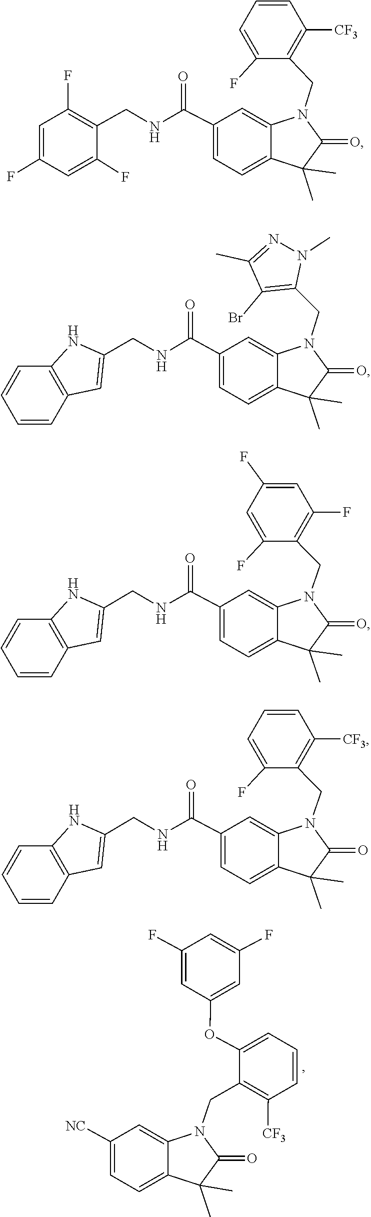 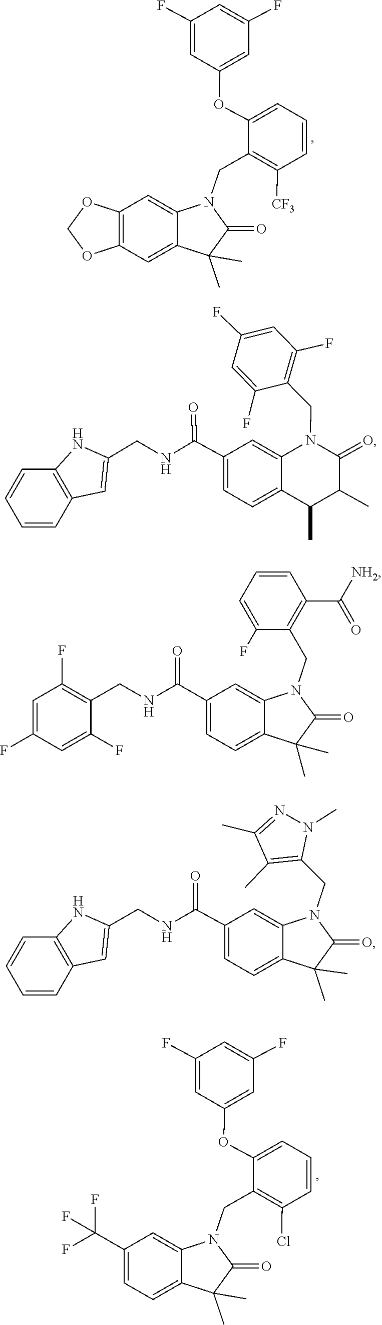 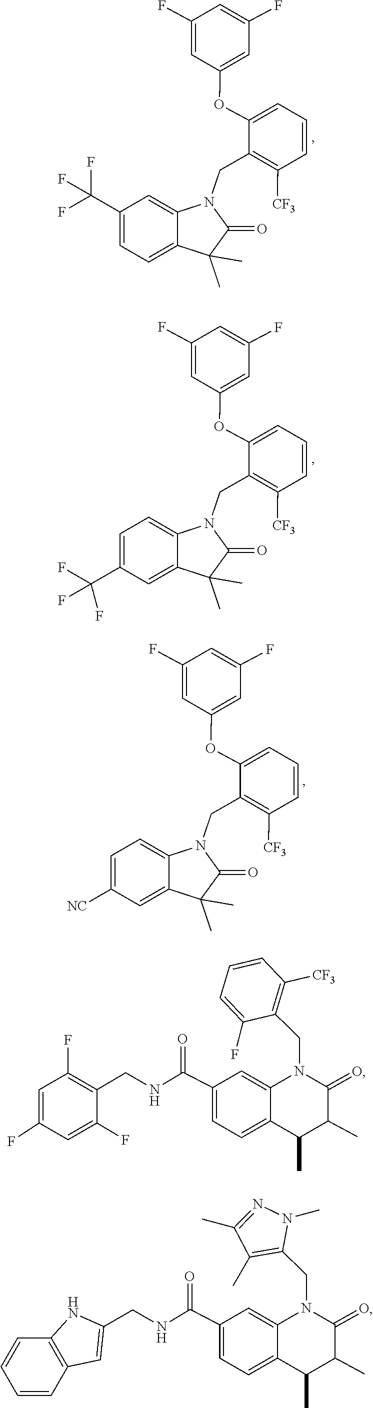 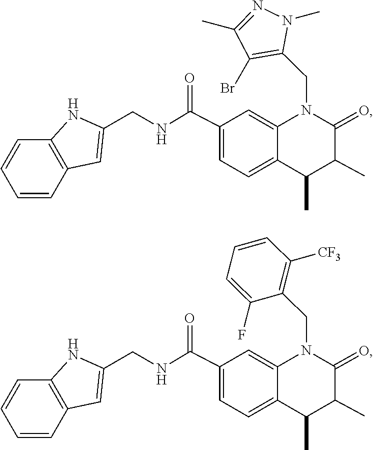
and any salt thereof.

26. The method of any one of claims 15 to 25, wherein the STING antagonist is selected from the group consisting of: 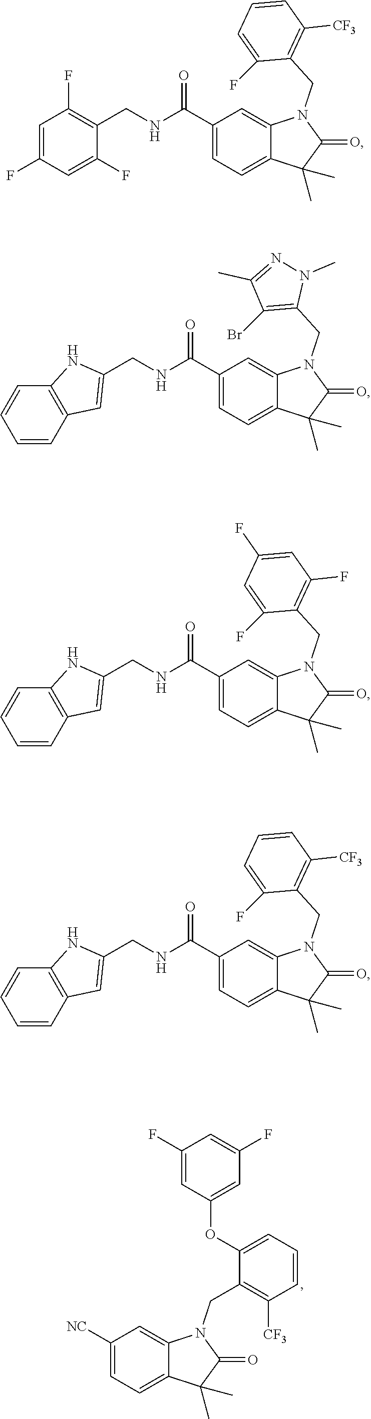 [SMILES: CC1(C)C(=O)N(Cc2c(F)cccc2C(F)(F)F)c2cc(C(=O)NCc3c(F)cc(F)cc3F)ccc21 ; Cc1nn(C)c(CN2C(=O)C(C)(C)c3ccc(C(=O)NCc4cc5ccccc5[nH]4)cc32)c1Br ; CC1(C)C(=O)N(Cc2c(F)cc(F)cc2F)c2cc(C(=O)NCc3cc4ccccc4[nH]3)ccc21 ; CC1(C)C(=O)N(Cc2c(F)cccc2C(F)(F)F)c2cc(C(=O)NCc3cc4ccccc4[nH]3)ccc21 ; CC1(C)C(=O)N(Cc2c(Oc3cc(F)cc(F)c3)cccc2C(F)(F)F)c2cc(C#N)ccc21] 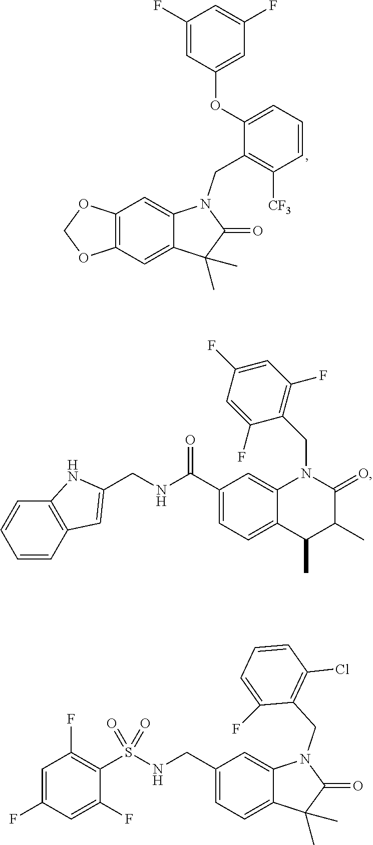 [SMILES: CC1(C)C(=O)N(Cc2c(Oc3cc(F)cc(F)c3)cccc2C(F)(F)F)c2cc3c(cc21)OCO3 ; CC1C(=O)N(Cc2c(F)cc(F)cc2F)c2cc(C(=O)NCc3cc4ccccc4[nH]3)ccc2[C@@H]1C ; CC1(C)C(=O)N(Cc2c(F)cccc2Cl)c2cc(CNS(=O)(=O)c3c(F)cc(F)cc3F)ccc21]
and any salt thereof.

27. The method of any one of claims 15 to 26, wherein the STING antagonist is selected from the group consisting of 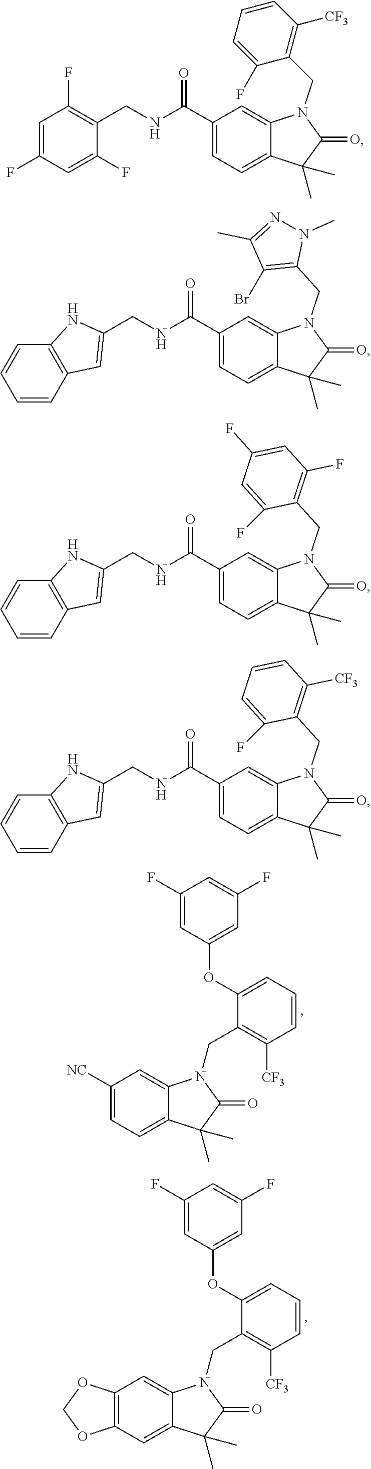 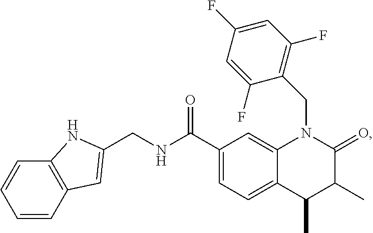 [SMILES: CC1C(=O)N(Cc2c(F)cc(F)cc2F)c2cc(C(=O)NCc3cc4ccccc4[nH]3)ccc2[C@@H]1C]
and any salt thereof.

28. The method of any one of claims 15 to 27, wherein the condition caused or related to disrupted STING signaling is inflammation, allergies, an autoimmune condition, an infectious disease, a neurodegenerative disease, a liver disease, a cancer and/or a renal disease.

29. The method of any one of claims 15 to 28, wherein the STING antagonist is administered in a pharmaceutical composition comprising at least one carrier or excipient.

30. The method of any one of claims 15 to 29, wherein the subject in need thereof is a mammal.

31. The method of claim 30, wherein the subject in need thereof is human.

32. A compound of Formula I, Formula II or Formula III: 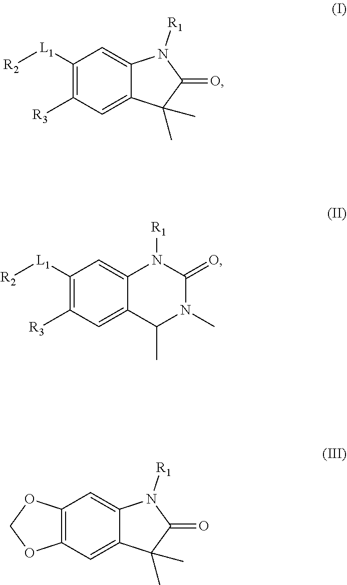
or a salt thereof,
wherein L₁ is absent or is selected from the group consisting of 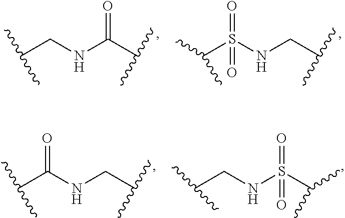 [SMILES: *CNC(*)=O ; *CNS(*)(=O)=O ; *CNC(*)=O ; *CNS(*)(=O)=O]
and;
R₁ is selected from the group consisting of 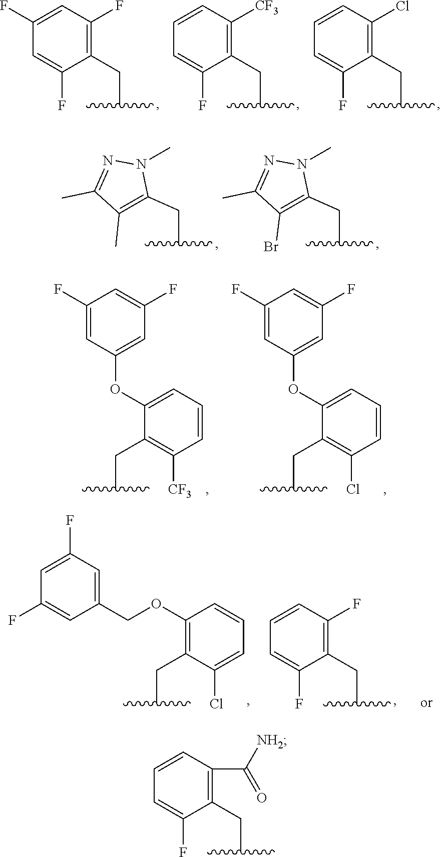
R₂ is selected from the group consisting of H, CF₃, F, CN, 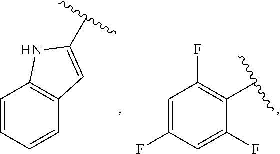 [SMILES: *c1cc2ccccc2[nH]1 ; *c1c(F)cc(F)cc1F]
and
R₃ is selected from the group consisting of H, CN or CF₃;
with the provisos that
(i) at least one of R₂ and R₃ is not hydrogen and
(ii) R₁ is not 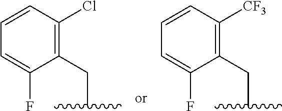 [SMILES: *Cc1c(F)cccc1Cl ; *Cc1c(F)cccc1C(F)(F)F]
when L1 is 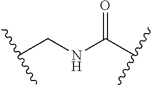 [SMILES: *CNC(*)=O]
and R₂ is 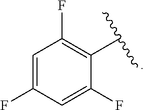 [SMILES: *c1c(F)cc(F)cc1F]
The method of claim 15, wherein L1 is absent,

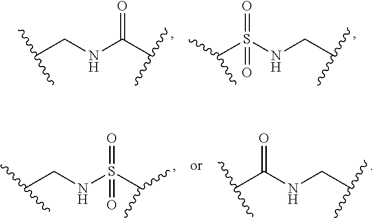 [SMILES: *CNC(*)=O ; *CNS(*)(=O)=O ; *CNS(*)(=O)=O ; *CNC(*)=O]

33. The compound of claim 32, wherein L1 is absent or

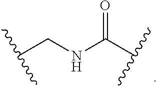 [SMILES: *CNC(*)=O]

34. The compound of claim 32 or 33, wherein R₁ is selected from the group consisting of

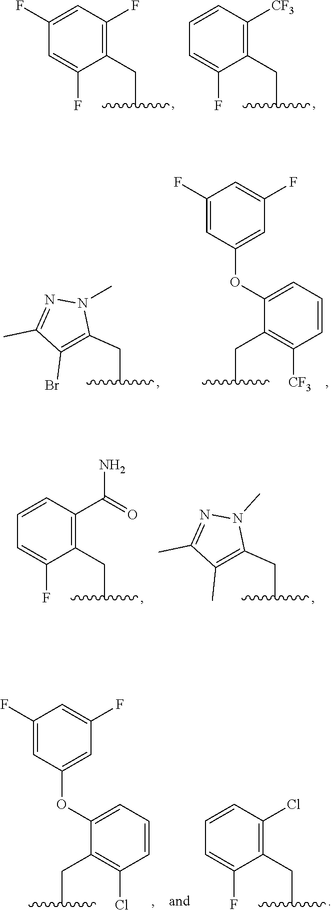

35. The compound of any one of claims 32 to 34, wherein R₁ is selected from the group consisting of

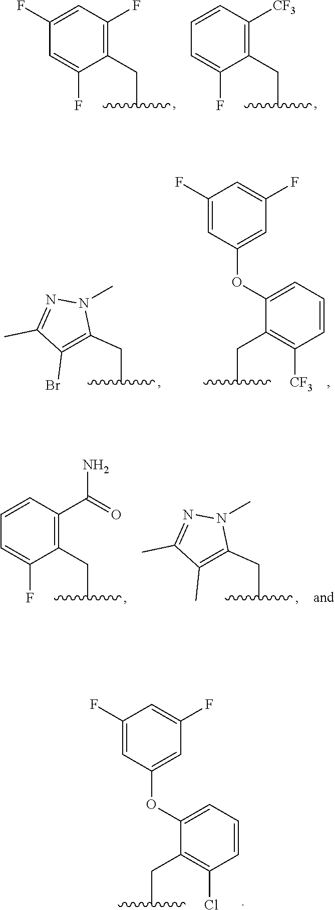

36. The compound of claim 32 to 35, wherein R₁ is selected from the group consisting of

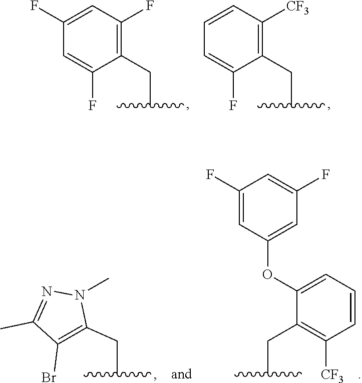

37. The compound of any one of claims 32 to 36, wherein R₂ is selected from the group consisting of 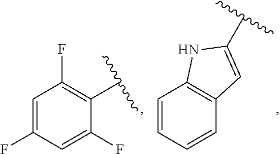
—CN, H, and —CF₃.

38. The compound of any one of claims 32 to 37, wherein R₂ is selected from the group consisting of 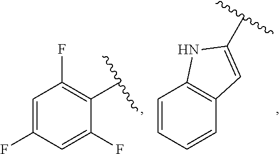
and —CN.

39. The compound of any one of claims 32 to 38, wherein R₃ is hydrogen.

40. The compound of any one of claims 32 to 39, wherein the compound is selected from the group consisting of: 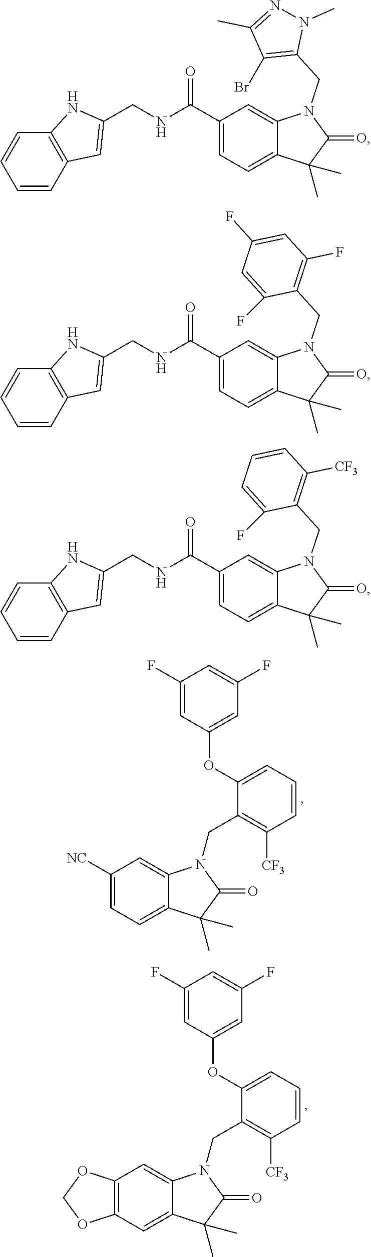 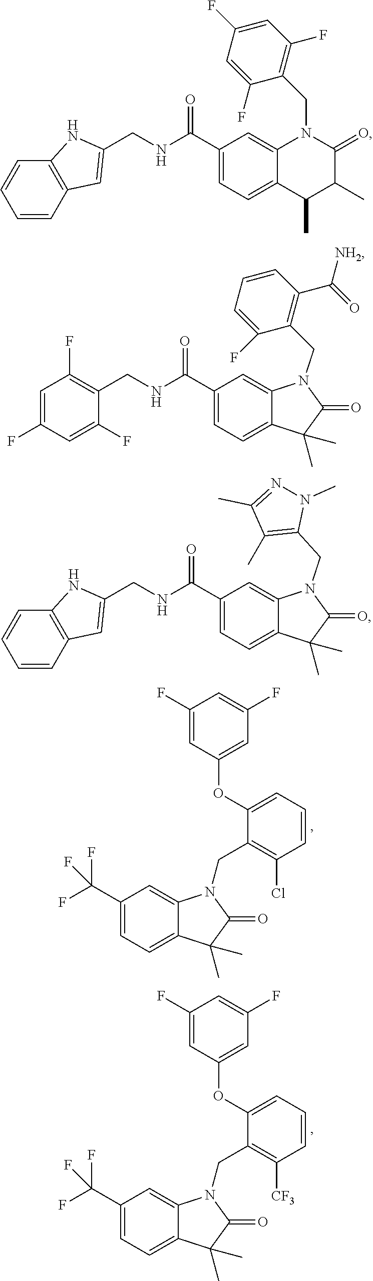 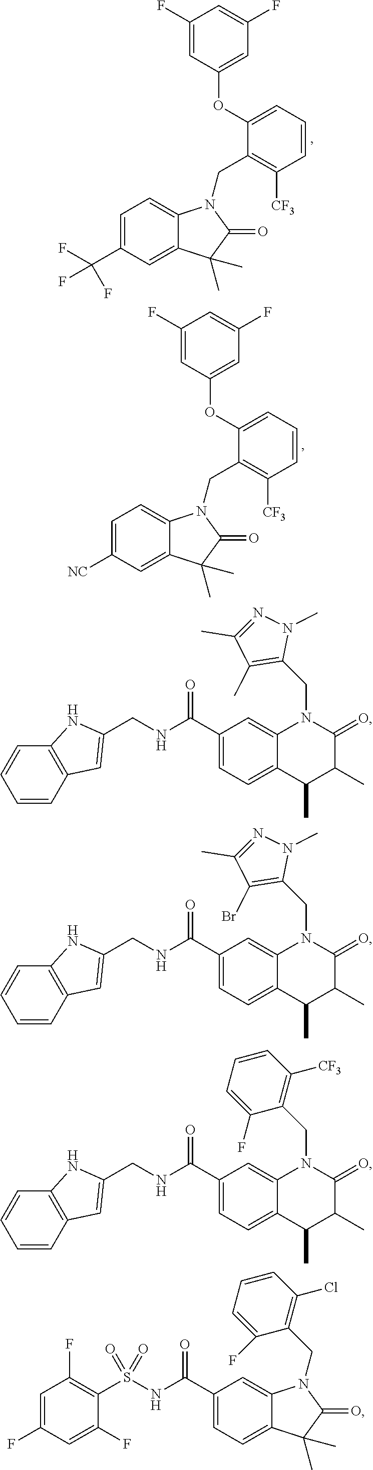 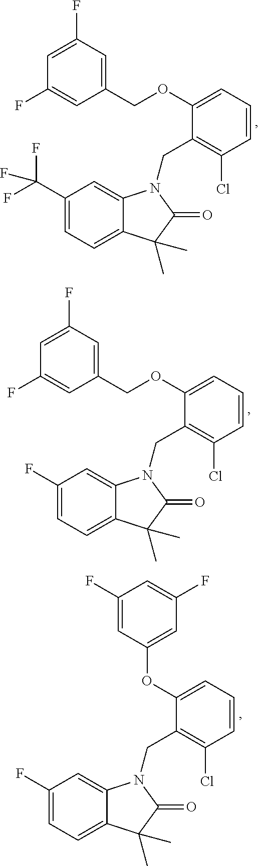
and any salt thereof.

41. A pharmaceutical composition comprising a compound of any one of claims 32 to 40 and at least one carrier or excipient.
